(19) United States
(12) Patent Application Publication
(10) Pub. No.: US 2024/0150484 A1
(43) Pub. Date: May 9, 2024

(54) NON-ACTIVATING ANTIBODY VARIANTS

(71) Applicant: GENMAB A/S, Valby (DK)

(72) Inventors: Bart-Jan DE KREUK, Utrecht (NL); Richard HIBBERT, GN Wassenaar (NL); Janine SCHUURMAN, Diemen (NL); Aran F. LABRIJN, Nigtevecht (NL)

(21) Appl. No.: 18/281,372

(22) PCT Filed: Mar. 11, 2022
(86) PCT No.: PCT/EP2022/056416

§ 371 Date: Sep. 11, 2023

(30) Foreign Application Priority Data
Mar. 12, 2021 (EP) 21162341.8

(51) Int. Cl. C07K 16/28; C07K 1/107; C07K 16/18; C07K 16/32

(52) U.S. Cl. CPC C07K 16/2887; C07K 1/1075; C07K 16/18; C07K 16/2809; C07K 16/2833; C07K 16/32; C07K 2317/31; C07K 2317/54; C07K 2317/92; C07K 2317/94

(57) ABSTRACT

Described herein are proteins comprising an Fc region or the like, such as monoclonal, bispecific and multispecific antibodies, wherein the Fc region has been modified to eliminate or strongly reduce Fc-mediated effector functions, while at the same time allow for good developability, for therapeutic purposes and where such effector functions are undesired.

### FIELD OF THE INVENTION

The present invention relates to polypeptides comprising an Fc region or the like, such as monoclonal, bispecific and multispecific antibodies, wherein the Fc region has been modified to eliminate or strongly reduce Fc-mediated effector functions, while at the same time allow for i.e. good developability, for therapeutic purposes and where such effector functions are undesired.

### INTRODUCTION

Antibodies have proven to be successful as therapeutic molecules, in particular for the treatment of cancer and immune modulation. Tumor target-specific antibodies can effectuate tumor cells cytotoxicity, typically via Fc-mediated effector functions, such as complement-dependent cytotoxicity (CDC), antibody-dependent cell-mediated cytotoxicity (ADCC), or antibody-dependent cell-mediated phagocytosis (ADCP). Immune cell-targeting antibodies can boost cells of the immune system, such as T cells and macrophages, which can in turn promote tumor cells cytotoxicity. Antibodies targeting components of the immune system can be used to modulate immune system function.

In certain scenarios, activation of the immune system, or components thereof, through antibody therapy may be undesirable, such as when applied to (i) systemically neutralize cytokines, (ii) blocking specific immune cell receptors or (iii) when using bispecific antibodies for redirecting effector cells to target diseased tissue (Kang et al. Exp. Mol. Med. 2019; 51(11):1-9). For example, engagement of immune cell-targeted antibodies via their Fc regions with complement component C1q may initiate activation of the classical complement system resulting in CDC of, for example, target immune cells, which is undesirable. Also, antibody Fc regions may also bind Fc receptors expressed on a range of immune cells resulting in unwanted depletion of effector cells or induce immune-related toxicity through high-level cytokine secretion.

To avoid undesirable Fc-mediated effector functions, antibodies have been engineered to harbor mutations in the Fc portion (also referred to as non-activating mutations) which suppress or eliminate some or all Fc-mediated effector mechanisms.

A wide range of different non-activating antibody formats have been developed in which amino acid substitutions, and combinations thereof, have been introduced in the constant heavy chain region of an IgG1 isotype antibody to eliminate Fc-mediated effector functions (e.g. Chiu et al., Antibodies 2019 December; 8(4): 55; Liu et al., Antibodies, 2020 Nov. 17; 9(4):64; 29(10):457-66; Shields et al., J Biol Chem., 2001 Mar. 2; 276(9):6591-604). Examples of such substitutions include the introduction of the N297G non-activating mutation (Tao and Morrison, J Immunol 1989; 143(8):2595-601), introduction of E233P-L234V-L235A-deIG236-S267K non-activating mutations (Moore at al., Methods 2019; 154:38-50), introduction of L234A-L235A-P329G non-activating mutations (Schlothauer et al., Protein Eng. Design and Selection 2016; 29(10):457-66), or L234F-L235E-D265A non-activating mutations (Also referred to as FEA or FEA format herein, Engelberts et al., EBioMedicine 2020; 52:102625; U.S. Ser. No. 10/590,206B2). Other non-activating formats were developed using IgG4, one of the IgG subclasses with reduced effector functions, in combination with amino acid substitutions in the constant heavy chain region of the antibody to further eliminate Fc-mediated effector functions (e.g. introduction of E233P-F234V-L235A-G236del non-activating mutations described in WO2015/143079, or introduction of F234A-L235A non-activating mutations described by (Vafa et al. Methods 2014; 65: 114-126).

However, as a result of such antibody engineering, developability and manufacturability issues may arise which may limit the potential development of antibodies as a therapeutic. For example, viral inactivation constitutes a crucial step in the development of a potential antibody therapeutic. A standard viral inactivation protocol requires a low pH hold. However, engineered proteins, such as antibodies, may respond differently to low pH conditions, potentially resulting in protein instability. Other conditions that for example need to be assessed in antibody development and manufacturability are repeated freeze-thaw cycles and variations in storage temperature. Therefore, it is desired to provide engineering of antibody therapeutics which allow for retaining protein stability when exposed to such conditions and the like, as it enables ease of manufacturing and distribution, and clinical use of such products.

Hence, there is a need to provide for further non-activating formats suitable for antibodies that lack Fc-mediated effector functions, and which can readily be developed and manufactured for therapeutic use.

### SUMMARY OF THE INVENTION

The L234F-L235E-D265A non-activating mutations (also referred to herein as FEA or FEA format), have been shown to have an excellent safety profile and ability to strongly suppress Fc-mediated effector function. Nevertheless, it was observed that for IgG1 antibodies that are potent inducers of complement-dependent cytotoxicity (CDC), harboring the FEA mutations can show some residual CDC (see i.a. examples 3 and 5). Furthermore, it was observed that recombinantly expressed antibodies with the FEA format may exhibit increased glycosylation heterogeneity as a result of additional processing of their N-glycans as compared with a wild-type IgG1 Fc region (data not shown, and see also Example 14) and were also shown to be more susceptible to aggregation induced by low pH conditions (see e.g. example 20).

Hence, the current inventors sought to provide for an improved non-activating format that can avoid residual CDC activity, can provide a wild-type like glycosylation profile and/or can be more tolerant to low pH conditions. Surprisingly, when the inventors combined the mutations L234F, L235E and G236R (also referred to herein as FER, or FER format) in IgG1 antibodies this resulted in an improved inert format capable of avoiding potential residual CDC activity, providing wild-type like glycosylation and having improved tolerance to low pH conditions. Thereby, the current inventors now provide for a highly advantageous further non-activating antibody format that is well suitable for clinical development and clinical use. As shown in the example section, this further non-activating format, which may be useful in contexts other than antibodies as well, such as e.g. fusion proteins, can be regarded to be a best-in-class non-activating format.

Accordingly, the current invention provides for a new inert format for polypeptides having a human IgG1 Fc region, said region having substitutions at positions 234, 235 and 236, preferably having substitutions F, E, and R, respectively, in accordance with Eu-numbering. This inert format is in particular useful for monospecific and bispecific antibodies. Such a polypeptide, e.g. a monospecific or bispecific antibody, having such substitutions in accordance with the invention may be referred to as having a non-activating IgG1 Fc region.

For bispecific antibodies, this inert format may also be combined in a heterodimeric format with respect to inert format substitutions, for example, a bispecific antibody may be composed of one chain carrying the inert format substitutions in accordance with the invention, whereas the other chain may comprise different inert format substitutions, e.g. FEA. Hence, the inert format in accordance with the invention is well suitable to be combined e.g. with existing candidate antibodies which have already undergone development for clinical use without the need to redesign and redo all the assays required, thereby allowing to quickly generate bispecific antibodies therewith utilizing technologies such as controlled Fab-arm exchange.

Hence, in one embodiment, a protein is provided, comprising a first polypeptide and a second polypeptide, wherein said first and second polypeptide each comprise at least a hinge region, a CH2 region and a CH3 region, respectively, of a human IgG1 immunoglobulin heavy chain, wherein at least one of said first and second polypeptides is modified and comprises a substitution of amino acids corresponding with amino acids at the positions L234, L235 and G236, wherein amino acid positions are as defined by Eu numbering. As said, preferably, the amino acids at positions L234, L235 and G236 in at least one of said first and second polypeptide are substituted with F, E and R, respectively.

In another embodiment, said protein in accordance with the invention is provided, wherein one of the first and second polypeptides comprises said substitution of amino acids corresponding with amino acids at positions L234, L235 and G236, and the other is modified and comprises a substitution of amino acids corresponding with amino acids at positions L234, L235, and D265, wherein preferably, said substitutions are F, E and A, respectively.

In another further embodiment, said protein in accordance with the invention is provided, wherein both first and second polypeptides comprise said substitution of amino acids corresponding with amino acids L234, L235 and G236.

It is understood that the protein in accordance with the invention may be any protein that may benefit from having an Fc-region which is non-activating. This may include fusion proteins, wherein e.g. a functional domain is fused with an Fc region, thereby providing the functional domain with e.g. an improved plasma half-life.

As said, proteins in accordance with the invention preferably comprise a first and a second binding region. Exemplary and preferred proteins in accordance with the invention having a first and second binding region are antibodies.

Hence, in another further embodiment, the protein in accordance with the invention is an antibody. In another further embodiment, the first and second polypeptide of the protein are identical in the protein or antibody in accordance with the invention. In another embodiment, such antibodies, as these typically may have the same binding domains, are monospecific antibodies. Such monospecific antibodies may subsequently be used for generating a bispecific antibody in accordance with the invention.

Hence, in still another further embodiment, the protein in accordance with the invention is a bispecific antibody. Preferably, a bispecific antibody in accordance with the invention is provided, wherein said first and second polypeptide comprise further substitutions in said respective CH2 and CH3 regions such that the sequences of the respective CH2 and CH3 regions from said first and second polypeptides are different, said substitutions allowing to obtain said polypeptide comprising said first and second polypeptide. Preferred examples of such substitutions include having one of the first and second polypeptides comprising a substitution of the amino acid at position F405 with an L, and the other at position K409 with an R. Alternatively, other substitutions or methods that allow to provide for heterodimers (i.a. combining different first and second polypeptides for providing a bispecific antibody) may be contemplated in accordance with the invention.

Hence, in one embodiment, a method is provided for preparing a bispecific antibody in accordance with the invention, said method comprising:
a) providing a first antibody, comprising
   a. an immunoglobulin heavy chain comprising at least a hinge region, a CH2 region and a CH3 region, respectively of a human IgG1 immunoglobulin heavy chain, comprising substitutions of amino acids at positions L234, L235 and G236, with F, E and R, respectively,
   b. an immunoglobulin light chain;
b) providing a second antibody, comprising
   a. an immunoglobulin heavy chain comprising at least a hinge region, a CH2 region and a CH3 region, respectively, of a human IgG1 immunoglobulin heavy chain, comprising substitutions of amino acids at
      positions L234, L235, and D265, wherein preferably, said substitutions are F, E and A, respectively, or,
      comprising substitutions of amino acids at positions L234, L235 and G236, with F, E and R, respectively,
   b. an immunoglobulin light chain;
c) wherein the sequences of said first and second CH3 regions of said respective first and second antibodies are different and are such that the heterodimeric interaction between said first and second CH3 regions is stronger than each of the homodimeric interactions of said first and second CH3 regions;
d) incubating said first antibody together with said second antibody under reducing conditions sufficient to allow the cysteines in the hinge regions to undergo disulfide-bond isomerization; and
e) obtaining said bispecific antibody comprising said first immunoglobulin heavy chain and said first immunoglobulin light chain of said first antibody and said second immunoglobulin heavy chain and said second immunoglobulin light chain of said second antibody.

### BRIEF DESCRIPTION OF THE DRAWINGS

FIG. 1 shows target binding by anti-human CD20 antibody variants. Binding to CD20, present on Raji cells, by IgG1 and IgG4 antibody variants harboring non-activating mutations in the heavy chain is shown. Binding is presented as antibody concentration vs Median MFI-PE (dataset split up into A and B). Data are mean values±SEM obtained from four independent replicates. Variants tested are IgG1-FEA-K409R, IgG1-FER-K409R, IgG1-AAG-K409R, IgG1-RR-K409R, IgG1lh2-S267K-K409R, IgG1-N297G-K409R, IgG1-AEASS-K409R, IgG1, IgG1-K409R, IgG1-b12, IgG4lh2-S228P, IgG4-PAA, IgG4, IgG4-S228P wherein FEA: L234F-L235E-D265A, FER: L234F-L235E-G236R, AAG: L234A-L235A-P329G, RR: G236R-L328R, IgG1lh2: E233P-L234V-L235A-G236del, AEASS: L234A-L235E-G237A-A330S-P331S, IgG4lh2: E233P-F234V-L235A-G236del, and PAA: 5228P-F234A-L235A.

FIG. 2 shows CDC of Raji cells by anti-human CD20 antibody variants harboring non-activating mutations in the constant heavy chain region. CDC of CD20-positive Raji cells induced by IgG1- and IgG4_antibody variants harboring non-activating mutations in the constant heavy chain region was assessed using NHS as a source for complement. Cell lysis is determined by analysis of the percentage of PI-positive cells by flow cytometry. CDC is presented as Area Under Curve (AUC) normalized to non-binding control antibody IgG1-b12 (0%) and wild-type IgG1 (100%). Data are mean values ±SEM obtained from three independent replicates. Variants tested are IgG1-FEA-K409R, IgG1-FER-K409R, IgG1-AAG-K409R, IgG1-RR-K409R, IgG1lh2-5267K-K409R, IgG1-N297G-K409R, IgG1-AEASS-K409R, IgG1, IgG1-K409R, IgG1-b12, IgG4lh2-S228P, IgG4-PAA, IgG4, IgG4-S228P wherein FEA: L234F-L235E-D265A, FER: L234F-L235E-G236R, AAG: L234A-L235A-P329G, RR: G236R-L328R, IgG1lh2: E233P-L234V-L235A-G236del, AEASS: L234A-L235E-G237A-A330S-P331S, IgG4lh2: E233P-F234V-L235A-G236del, and PAA: 5228P-F234A-L235A.

FIG. 3 shows C1q binding by anti-human CD20 IgG1-antibody variants harboring non-activating mutations in the constant heavy chain region. Binding is presented as antibody concentration vs Median MFI-FITC. Data are mean values (±SD) obtained from triplicate measurements of a single experiment. Variants tested are IgG1-FEA-K409R, IgG1-FER-K409R, IgG1, IgG1-b12 wherein FEA: L234F-L235E-D265A and FER: L234F-L235E-G236R.

FIG. 4 shows CDC of Raji cells induced by anti-human HLA-DR antibody variants harboring non-activating mutations in the constant heavy chain region. CDC of Raji cells induced by IgG1-HLA-DR-4 (A) and IgG1-HLA-DR-1D09C3 (B) antibody variants harboring non-activating mutations in the constant heavy chain region, as well as an HLA-DR-targeting F(ab′)2 fragment was assessed in an in vitro CDC assay using NHS as a source for complement. Cell lysis is determined by analysis of the percentage of PI-positive cells by flow cytometry. CDC is presented as Area Under Curve (AUC) normalized to IgG1 antibody harboring K409R mutation (IgG1-K409R, 100%). Data are mean values ±SEM from five (wild-type and L234F-L235E-D265A-K409R variants) or two (L234F-L235E-G236R-K409R variants, or the F(ab′)2 fragment) independent replicates. Variants tested are IgG1-FEA-K409R, IgG1-FER-K409R, IgG1-K409R, F(ab′)2 wherein FEA: L234F-L235E-D265A and FER: L234F-L235E-G236R.

FIG. 5 shows capture of anti-human CD20 IgG1 and IgG4 antibody variants to ELISA plates. Immobilization of IgG1 and IgG4 variants with non-activating mutations in the heavy chain region by anti-human-IgG F(ab′)2 fragments to ELISA plates. Binding is presented as Area Under Curve (AUC) normalized to wild-type IgG1 (100%). Data are mean values (±SEM) obtained from three independent replicates. Detection was performed using anti-human-IgG-Fcγ-HRP and ABTS. Variants tested are IgG1-FEA-K409R, IgG1-FER-K409R, IgG1-AAG-K409R, IgG1-RR-K409R, IgG1lh2-S267K-K409R, IgG1-N297G-K409R, IgG1-AEASS-K409R, IgG1, IgG1-K409R, IgG4lh2-S228P, IgG4-PAA, IgG4, IgG4-S228P wherein FEA: L234F-L235E-D265A, FER: L234F-L235E-G236R, AAG: L234A-L235A-P329G, RR: G236R-L328R, IgG1lh2: E233P-L234V-L235A-G236del, AEASS: L234A-L235E-G237A-A330S-P331S, IgG4lh2: E233P-F234V-L235A-G236del, and PAA: 5228P-F234A-L235A.

FIG. 6 shows binding of anti-human CD20 IgG1 and IgG4 antibody variants harboring non-activating mutations in the constant heavy chain region to FcγRIa, FcγRIIa (allotypes 131H and 131R), FcγRIIb, and FcγRIIIa (allotype 158F and 158V). Binding of immobilized IgG1 and IgG4 variants with non-activating mutations to monomeric and dimeric His-tagged biotinylated extracellular domains (ECDs) of FcγRIa (A), FcγRIIa allotype 131H (B), FcγRIIa allotype 131R (C), FcγRIIb (D), FcγRIIIa allotype 158F (E), and FcγRIIIa allotype 158V (F) as tested in ELISA assays. Binding is presented as Area Under Curve (AUC) normalized to wild-type IgG1 (100%). Data are mean values (±SEM) obtained from three independent replicates. Detection was performed using Streptavidin-polyHRP and ABTS. Variants tested are IgG1-FEA-K409R, IgG1-FER-K409R, IgG1-AAG-K409R, IgG1-RR-K409R, IgG1lh2-S267K-K409R, IgG1-N297G-K409R, IgG1-AEASS-K409R, IgG1, IgG1-K409R, IgG4lh2-S228P, IgG4-PAA, IgG4, IgG4-S228P wherein FEA: L234F-L235E-D265A, FER: L234F-L235E-G236R, AAG: L234A-L235A-P329G, RR: G236R-L328R, IgG1lh2: E233P-L234V-L235A-G236del, AEASS: L234A-L235E-G237A-A330S-P331S, IgG4lh2: E233P-F234V-L235A-G236del, and PAA: 5228P-F234A-L235A.

FIG. 7 shows FcγR activation by anti-human CD20 IgG1 and IgG4 antibody variants harboring non-activating mutations in the heavy chain region as measured using target-expressing Raji cells and FcγR-expressing reporter cells. (A-F) Activation of Jurkat reporter cell lines stably expressing either (A) FcγRIa, (B) FcγRIIa allotype 131H, (C) FcγRIIa allotype 131R, (D) FcγRIIb, (E) FcγRIIIa allotype 158F, or (F) FcγRIIIa allotype 158V, as measured by the level of luminescence (RLU), upon co-culturing with Raji cells, expressing CD20, and different concentrations of IgG1 and IgG4 antibody variants. Activation is presented as Area Under the dose-response Curve (AUC) normalized to non-binding control IgG1-b12 (0%) and wild-type IgG1 (100%) per experimental replicate. Data mean values (±SEM) from three independent experimental replicates. Variants tested are IgG1-FEA-K409R, IgG1-FER-K409R, IgG1-AAG-K409R, IgG1-RR-K409R, IgG1lh2-S267K-K409R, IgG1-N297G-K409R, IgG1-AEASS-K409R, IgG1, IgG1-K409R, IgG1-b12, IgG4lh2-S228P, IgG4-PAA, IgG4, IgG4-S228P wherein FEA: L234F-L235E-D265A, FER: L234F-L235E-G236R, AAG: L234A-L235A-P329G, RR: G236R-L328R, IgG1lh2: E233P-L234V-L235A-G236del, AEASS: L234A-L235E-G237A-A330S-P331S, IgG4lh2: E233P-F234V-L235A-G236del, and PAA: 5228P-F234A-L235A.

FIG. 8 shows ADCC induced by anti-human CD20 IgG1 and IgG4 antibody variants harboring non-activating mutations in the heavy chain region as measured using the DELFIA® EuTDA TRF cytotoxicity assay. (A-B) NK-cell-mediated ADCC was measured by the level of release of EuTDA reagent from BATDA labeled CD20-expressing Raji cells, upon co-incubation with peripheral blood mononuclear cells (PBMC) and anti-human CD20 IgG1 and IgG4 antibody variants. For some variants ADCC is presented (A) as Area Under Curve (AUC) normalized to non-binding control IgG1-b12 (0%) and wild-type IgG1 (100%) per experimental replicate. Data are mean values (±SEM) obtained from four (wild-type and K409R variants) or two (L234F-L235E-D265A-K409R and L234F-L235E-G236R-K409R variants) independent replicates. For some variants ADCC is presented as (B) percentage lysis at 10 μg/ml antibody concentration relative to non-binding control IgG1-b12 (0%) and wild-type IgG1 (100%) per experimental replicate. Data are mean values (±SEM) obtained from six donors from 2 independent experiments. Variants tested are IgG1-FEA-K409R, IgG1-FER-K409R, IgG1-AAG-K409R, IgG1-RR-K409R, IgG1lh2-S267K-K409R, IgG1-N297G-K409R, IgG1-AEASS-K409R, IgG1, IgG1-K409R, IgG1-b12, IgG4lh2-S228P, IgG4-PAA, IgG4, IgG4-S228P wherein FEA: L234F-L235E-D265A, FER: L234F-L235E-G236R, AAG: L234A-L235A-P329G, RR: G236R-L328R, IgG1lh2: E233P-L234V-L235A-G236del, AEASS: L234A-L235E-G237A-A330S-P331S, IgG4lh2: E233P-F234V-L235A-G236del, and PAA: 5228P-F234A-L235A.

FIG. 9 shows T-cell activation by variants of the anti-human CD3 antibodies IgG1- or IgG4-huCLB-T3/4 harboring non-activating mutations in the constant heavy chain region. (A-B) Upregulation of CD69 expression (measured by flow cytometry analysis), as a measure for early T-cell activation, on T cells in a PBMC co-culture induced by anti-human CD3 IgG1 and IgG4 antibody variants harboring the indicated mutations. CD69 upregulation is presented as (A) dose-response vs. percentage of CD69⁺ T cells (CD28±) or as (B) Area under the dose-response Curve (AUC) normalized to wild-type antibody variant IgG1-F405L (100%) per donor and experimental replicate. Data are mean values (±SEM) from 3 independent experimental replicates (2 independent donors per experimental replicate). Variants tested are IgG1-FEA-F405L, IgG1-FER-F405L, IgG1-AAG-F405L, IgG1-RR-F405L, IgG1lh2-S267K-F405L, IgG1-N297G-F405L, IgG1-AEASS-F405L, IgG1-F405L, IgG4lh2-S228P-F405L-R409K, IgG4-PAA-F405L-R409K, IgG4-S228P wherein FEA: L234F-L235E-D265A, FER: L234F-L235E-G236R, AAG: L234A-L235A-P329G, RR: G236R-L328R, IgG1lh2: E233P-L234V-L235A-G236del, AEASS: L234A-L235E-G237A-A330S-P331S, IgG4lh2: E233P-F234V-L235A-G236del, and PAA: 5228P-F234A-L235A.

FIG. 10 shows in vitro T-cell-mediated cytotoxicity by non-activating bispecific antibody variants. (A-C) T-cell mediated cytotoxicity of HER2-positive SK-OV-3 cells in a PBMC co-culture was assessed using bispecific antibody variants, CD3×HER2 (A), CD3×b12 (B; no binding to target cell), or b12×HER2 (C; no binding to T cells), harboring non-activating mutations in the constant heavy chain region using Alamar blue. Absorbance at 590 nm was measured using an Envision plate reader and the percentage viable cells was calculated per donor and experimental replicate with Staurosporin-treated cells representing 100% cytotoxicity and medium control (no antibody, no PBMC) representing 0% cytotoxicity. Data is presented as dose-response curve vs. % viable SK-OV-3 cells. Data are mean values (±SEM) from 3 independent experimental replicates (2 independent donors per experimental replicate). Bispecific antibody variants tested are BisG1 F405L×K409R, BisG1 FEA-F405L×FEA-K409R, BisG1 FER-F405L×FER-K409R, BisG1 AAG-F405L×AAG-K409R, BisG1 RR-F405L×RR-K409R, BisG1lh2 S267K-F405L×5267K-K409R, BisG4lh2 S228P-F405L-R409K×S228P, BisG1 N297G-F405L×N297G-K409R, BisG1 AEASS-F405L×AEASS-K409R, BisG4 PAA-F405L-R409K×PAA wherein FEA: L234F-L235E-D265A, FER: L234F-L235E-G236R, AAG: L234A-L235A-P329G, RR: G236R-L328R, G1lh2: E233P-L234V-L235A-G236del, AEASS: L234A-L235E-G237A-A330S-P331S, G4lh2: E233P-F234V-L235A-G236del, and PAA: 5228P-F234A-L235A.

FIG. 11 shows total human IgG (hIgG) concentrations as measured in blood samples collected from mice injected with anti-human CD20 IgG1 or anti-human CD3 IgG1 (huCLB-T3/4) antibody variants. (A) Total hIgG concentration in blood samples collected from mice injected with wild-type anti-human CD20 IgG1, IgG1-CD20-K409R, IgG1-CD20-L234F-L235E-D265A-K409R, and IgG1-CD20-L234F-L235E-G236R-K409R at different time points after injection. Data are mean values (±SEM) obtained from 3 mice per group, except IgG1-FER-K409R (2 mice). (B) Total hIgG concentration in blood samples collected from mice injected with wild-type anti-human CD3 IgG1, IgG1-CD3-F405L, IgG1-CD3-L234F-L235E-D265A-F405L, and IgG1-CD3-L234F-L235E-G236R-F405L at different time points after injection. Data are mean values (±SEM) obtained from 3 mice per group. (C) Clearance until day 21 after administration of the antibody was determined following the formula D*1000/AUC with D, injected dose and AUC, area under the curve of the concentration-time curve. In all figures, the dotted line represents the predicted IgG1 concentration in time for wild-type IgG1 antibodies in SCID mice. Data represented is obtained from three mice per group, except for IgG1-FER-K409R (2 mice). Variants tested are IgG1-FEA-K409R, IgG1-FER-K409R, IgG1-K409R, IgG1-FEA-F405L, IgG1-FER-F405L, IgG1-F405L, IgG1 wherein FEA: L234F-L235E-D265A and FER: L234F-L235E-G236R.

FIG. 12 shows schematic representations of different glycan species detected on IgG1 antibody variants tested in Example 14.

FIG. 13 shows efficiency of controlled Fab-arm-exchange (cFAE) for generation of bsAb variants. Bispecific antibodies (indicated as BisG1) are generated by cFAE where one monospecific antibody (indicated as IgG1-A) bears a F405L mutation, and another monospecific antibody (indicated as IgG1-B) bears a K409R mutation. Efficiency of cFAE for generation of bsAb variants was assessed for variants where (A; 20 data points) both monospecific antibodies harbor L234F-L235E-G236R (FER) non-activating mutations in addition to F405L and K409R mutations, (B; 16 data points) the first monospecific antibody harbors L234F-L235E-G236R (FER) mutations in addition to a F405L mutation and the second monospecific antibody harbors L234F-L235E-D265A (FEA) mutations in addition to a K409R mutations, (C; 12 data points) the first monospecific antibody harbors L234F-L235E-D265A (FEA) mutations in addition to a F405L mutation and the second monospecific antibody harbors L234F-L235E-G236R (FER) mutations in addition to a K409R mutations. Percentage (%) of bsAb or residual monospecific antibody variants (IgG1-A or IgG1-B) is shown and was determined by using an Orbitrap Q-Exactive Plus mass spectrometer. FEA: L234F-L235E-D265A and FER: L234F-L235E-G236R.

FIG. 14 shows production levels of antibody variants harboring either L234F-L235E-G236R (FER) or L234F-L235E-D265A (FEA) non-activating mutations in the constant heavy chain region in addition to either F405L or K409R. Antibody variants were produced in Expi293F cells. Production titer is represented as mg/L in scatter dot plot with mean values (±SEM) indicated. Each dot represents production yield data of a particular antibody clone (average values if more production data was available for that particular clone) harboring the indicated mutations. To allow comparison, production titers of matched clones for L234F-L235E-D265A-F405L antibody variants (FEA-F405L; open circles) and L234F-L235E-G236R-F405L antibody variants (FER-F405L; closed circles) is shown. Similarly, production titers of matched clones for L235E-D265A-K409R antibody variants (FEA-K409R, open squares) and L234F-L235E-G236R-K409R antibody variants (FER-K409R, closed squares) is shown.

FIG. 15 shows an exemplary schematic of a monospecific antibody (A) and a bispecific antibody (B). (A) The Heavy chains as depicted in black; light chains as depicted in white. Individual antibody heavy and light chain domains are indicated as C_{H}1, C_{H}2, C_{H}3 and V_{H} (constant heavy (H1, H2, H3) and variable heavy (VH) chain domains), C_{L} and V_{L} (CL, VL, constant and variable light chain domains). (B) Bispecific antibody consisting of 2 half-molecules (1 half-molecule presented as black and white heavy and light chains, respectively; 1 half-molecule presented in striped pattern of heavy and light chains), such as generated through controlled Fab-arm exchange, with two different specificities of the Fab arms. Hinge region, Fab arms and Fc domain are as indicated.

FIG. 16 shows C1q binding by anti-human CD20 IgG1 antibody variants harboring non-activating mutations in the heavy chain constant region. Binding is presented as Area Under Curve (AUC) normalized to non-binding control antibody IgG1-b12 (0%) and wild-type IgG1-CD20 (100%). Data are mean values ±SEM obtained from 3 independent experiments. Antibody variants tested are IgG1-CD20 wild-type (IgG1) and the variants thereof IgG1-FEA-F405L, IgG1-FEA-K409R, IgG1-FER-F405L, IgG1-FER-K409R, BisG1 FEA-F405L×FEA-K409R, BisG1 FER-F405L×FER-K409R, BisG1 FER-F405L×FEA-K409R, BisG1 FEA-F405L×FER-K409R, wherein FER: L234F-L235E-G236R and FEA: L234F-L235E-D265A.

FIG. 17 shows CDC of Raji cells by anti-human CD20 IgG1 antibody variants harboring non-activating mutations in the heavy chain constant region. CDC of CD20-positive Raji cells induced by IgG1-CD20 antibody variants harboring non-activating mutations in the heavy chain constant region was assessed using NHS as a source for complement. Cell lysis is determined by analysis of the percentage of PI-positive cells by flow cytometry. CDC is presented as Area Under Curve (AUC) normalized to non-binding control antibody IgG1-b12 (0%) and wild-type IgG1-CD20 (100%). Data are mean values ±SEM obtained from three independent replicates. Antibody variants tested are IgG1-CD20 wild-type (IgG1) and the variants thereof IgG1-FEA-F405L, IgG1-FEA-K409R, IgG1-FER-F405L, IgG1-FER-K409R, BisG1 FEA-F405L×FEA-K409R, BisG1 FER-F405L×FER-K409R, BisG1 FER-F405L×FEA-K409R, BisG1 FEA-F405L×FER-K409R, wherein FER: L234F-L235E-G236R and FEA: L234F-L235E-D265A.

FIG. 18 shows in vitro T-cell-mediated cytotoxicity by non-activating bispecific antibody variants. (A-B) Using Alamar blue, T-cell mediated cytotoxicity of HER2-positive SK-OV-3 cells in a PBMC co-culture was assessed using the bispecific antibody variants CD3×HER2 (A) or CD3×b12 (B; no binding to target cell) harboring asymmetric non-activating mutations in the Fc region. Using an Envision plate reader, absorbance at 590 nm was measured and the percentage viable cells was calculated per donor and experimental replicate with Staurosporin-treated SK-OV-3 cells representing 100% cytotoxicity and medium control (SK-OV-3 cell, no antibody, no PBMC) representing 0% cytotoxicity. Data is presented as dose-response curve vs. percentage viable SK-OV-3 cells. Data are mean values ±SEM obtained from four donors from two independent experiments (2 donors per independent experiment). CD3×HER2 and CD3×b12 bispecific antibody variants tested are BisG1 F405L×K409R, BisG1 FER-F405L×K409R, BisG1 FER-F405L×FEA-K409R, BisG1 FER-F405L×AAG-K409R, and BisG1 FER-F405L×N297G-K409R wherein FER: L234F-L235E-G236R, FEA: L234F-L235E-D265A, and AAG: L234A-L235A-P329G.

FIG. 19 shows T-cell activation by non-activating bispecific CD3×HER2 antibody variants. Upregulation of CD69 expression (measured by flow cytometry analysis), as a measure for early T-cell activation, on T cells in a human PBMC co-culture was assessed using the wild-type like CD3×Her2 bispecific antibody variant and variants thereof harboring the indicated symmetric or asymmetric non-activating mutations in the Fc region. CD69 upregulation is presented as Area under the dose-response Curve (AUC) normalized to the AUC value measured for the non-binding negative control IgG1-b12 (0%) and the wild-type like IgG1 bispecific antibody variant (BisG1 F405L×K409R, 100%) per donor and experimental replicate. Data are mean values (±SEM) obtained from four donors in two independent experiments (2 donors per independent experiment). CD3×HER2 bispecific antibody variants tested are BisG1 F405L×K409R, BisG1 FER-F405L×K409R, BisG1 FER-F405L×FEA-K409R, BisG1 FER-F405L×AAG-K409R, and BisG1 FER-F405L×N297G-K409R wherein FER: L234F-L235E-G236R, FEA: L234F-L235E-D265A, and AAG: L234A-L235A-P329G.

FIG. 20 shows CDC of Raji cells induced by anti-human CD20 antibody variants harboring non-activating mutations in the heavy chain constant region as assessed in an in vitro CDC assay using NHS as a source for complement. The capacity to induce CDC was compared between variants produced to either contain or lack a C-terminal lysine in the heavy chain constant region. Cell lysis is determined by analysis of the percentage of PI-positive cells by flow cytometry. CDC is presented as Area Under Curve (AUC) normalized to wild-type IgG1-CD20 antibody (IgG1; 100%) and no antibody control samples (0%). Data are mean values ±SEM from three independent experiments. Variants tested are IgG1, IgG1-delK, IgG1-FEA, IgG1-FEA-delK, IgG1-FER, IgG1-FER-delK wherein FEA: L234F-L235E-D265A, FER: L234F-L235E-G236R, delK: recombinant deletion of the HC C-terminal lysine.

FIG. 21 shows human FcγR activation by anti-human CD20 antibody variants harboring non-activating mutations in the heavy chain constant region, as measured using target-expressing Raji cells and FcγR-expressing reporter cells. The capacity to induce FcγR activation was compared between variants produced to either contain or lack a C-terminal lysine in the heavy chain constant region. (A-D) Activation of Jurkat reporter cell lines stably expressing either (A) FcγRIa, (B) FcγRIIa allotype 131H, (C) FcγRIIb, or (D) FcγRIIIa allotype 158V, as measured by the level of luminescence upon co-culturing with Raji cells, expressing CD20, and different concentrations of IgG1-CD20 or IgG1-CD20-delK antibody variants. Activation is presented as Area Under the dose-response Curve (AUC) normalized to non-binding control IgG1-b12 (0%) and wild-type IgG1 (100%) per experimental replicate. Data shown are mean values ±SEM of 2 independent replicates. Variants tested are IgG1, IgG1-delK, IgG1-FEA, IgG1-FEA-delK, IgG1-FER, IgG1-FER-delK wherein FEA: L234F-L235E-D265A, FER: L234F-L235E-G236R, delK: recombinant deletion of the HC C-terminal lysine.

FIG. 22 shows CDC of Raji cells induced by allotypic variants of wild-type anti-human CD20 IgG1 antibody and variants thereof harboring non-activating mutations in the heavy chain constant region, as assessed in an in vitro CDC assay using NHS as a source for complement. Cell lysis is determined by analysis of the percentage of PI-positive cells by flow cytometry. CDC is presented as Area Under Curve (AUC) normalized to wild-type anti-human CD20 antibody (allotype IgG1(f); 100%) and no antibody control samples (0%). Data are mean values ±SEM from three independent experiments. Variants tested are IgG1(fa), IgG1(zax), IgG1(zav), IgG1(za), and IgG1(f) wherein FEA: L234F-L235E-D265A, FER: L234F-L235E-G236R.

FIG. 23 shows human FcγR activation by anti-human CD20 IgG1 antibody variants harboring non-activating mutations in the heavy chain constant region of different IgG1 allotypic variants as measured using target-expressing Raji cells and FcγR-expressing reporter cells. (A-D) Activation of Jurkat reporter cell lines stably expressing either (A) FcγRIa, (B) FcγRIIa allotype 131H, (C) FcγRIIb, or (D) FcγRIIIa allotype 158V, as measured by the level of luminescence, upon co-culturing with Raji cells, expressing CD20, and different concentrations of IgG1-CD20 antibody variants. Activation is presented as Area Under the dose-response Curve (AUC) normalized to non-binding control IgG1-b12 (0%) and wild-type IgG1(f) (100%) per experimental replicate. Data shown are mean values ±SEM of 2 independent replicates. Variants tested are IgG1(f), IgG1(za), IgG1(zav), IgG1(zax), IgG1(fa), and variants thereof harboring FER or FEA mutations wherein FER: L234F-L235E-G236R and FEA: L234F-L235E-D265A.

FIG. 24 shows CDC of Raji cells induced by subclass variants of wild-type anti-human CD20 antibodies and variants thereof harboring non-activating mutations in the heavy chain constant region, as assessed in an in vitro CDC assay using NHS as a source for complement. (A) CDC induced by wild-type anti-human CD20 IgG1 and IgG3 antibodies (allotypes IGHG3*01 [IgG3] and IGHG3*04 [IgG3rch2]) and non-activating variants thereof. (B) CDC induced by wild-type anti-human CD20 IgG1 and IgG4 antibodies and non-activating variants thereof. Cell lysis is determined by analysis of the percentage of PI-positive cells by flow cytometry. CDC is presented as Area Under Curve (AUC) normalized to wild-type anti-human CD20 IgG1 antibody (IgG1; 100%) and no antibody control samples (0%). Data are mean values ±SEM from three independent experiments. FEA: L234F-L235E-D265A, FER: L234F-L235E-G236R, EA: L235E-D265A, and ER: L235E-G236R.

FIG. 25 shows human FcγR activation by anti-human CD20 IgG1, IgG3, and IgG4 antibody variants harboring non-activating mutations in the heavy chain constant region as measured using target-expressing Raji cells and FcγR-expressing reporter cells. (A-D) Activation of Jurkat reporter cell lines stably expressing either (A) FcγRIa, (B) FcγRIIa allotype 131H, (C) FcγRIIb, or (D) FcγRIIIa allotype 158V, as measured by the level of luminescence upon co-culturing with Raji cells that express CD20, and different concentrations of IgG-CD20 antibody variants. Activation is presented as Area Under the dose-response Curve (AUC) normalized to non-binding control IgG1-b12 (0%) and the wild-type IgG1 (100%) per experimental replicate. Data shown are mean values ±SEM of 2 independent replicates. Variants tested are IgG1, IgG3 (IGHG3*01), IgG3rch2 (IGHG3*04), IgG4, and variants thereof harboring ER, EA, FER, or FEA mutations wherein ER: L235E-G236R, EA: L235E-D265A, FER: L234F-L235E-G236R, and FEA: L234F-L235E-D265A.

FIG. 26 shows human FcγR activation by anti-human CD20 murine IgG2a antibody variants harboring non-activating mutations in the heavy chain constant region as measured using target-expressing Raji cells and FcγR-expressing reporter cells. (A-D) Activation of Jurkat reporter cell lines stably expressing either (A) FcγRIa, (B) FcγRIIa allotype 131H, (C) FcγRIIb, or (D) FcγRIIIa allotype 158V, as measured by the level of luminescence upon co-culturing with Raji cells, expressing CD20, and different concentrations of murine IgG2a-CD20 antibody variants. Activation is presented as Area Under the dose-response Curve (AUC) normalized to non-binding control IgG2a-b12 (0%) and wild-type IgG2a-CD20 (100%) per experimental replicate. Data shown are mean values ±SEM of 2 independent replicates. Variants tested are IgG2a, IgG2a-FER, IgG2a-LALA, and IgG2a-LALAPG wherein FER: L234F-L235E-G236R, LALA: L234A-L235A, and LALAPG: L234A-L235A-P329G.

FIG. 27 shows C1q binding by anti-human CD20 murine IgG2a antibody variants harboring non-activating mutations in the heavy chain constant region upon opsonization of CD20-positive Raji cells with normal human serum (NHS) as a source for C1q. Binding is presented as Area Under Curve (AUC) normalized to non-binding control antibody IgG2a-b12 (0%) and wild-type murine IgG2a-CD20 (100%). Data are mean values ±SEM obtained from 3 independent experiments. Antibody variants tested are wild-type IgG2a, IgG2a-FER, IgG2a-LALA, and IgG2a-LALAPG wherein FER: L234F-L235E-G236R, LALA: L234A-L235A, and LALAPG: L234A-L235A-P329G.

FIG. 28 shows CDC of Raji cells by anti-human CD20 murine IgG2a antibody variants harboring non-activating mutations in the heavy chain constant region. CDC of CD20-positive Raji cells induced by IgG2a-CD20 antibody variants harboring non-activating mutations in the heavy chain constant region was assessed using normal human serum (NHS) as a source for complement. Cell lysis is determined by analysis of the percentage of PI-positive cells by flow cytometry. CDC is presented as Area Under Curve (AUC) normalized to non-binding control antibody IgG2a-b12 (0%) and wild-type murine IgG2a-CD20 (100%). Data are mean values ±SEM obtained from three independent replicates. Antibody variants tested are wild-type IgG2a, IgG2a-FER, IgG2a-LALA, and IgG2a-LALAPG wherein FER: L234F-L235E-G236R, LALA: L234A-L235A, and LALAPG: L234A-L235A-P329G.

### DETAILED DESCRIPTION

As described herein, specific modifications in amino acid positions in the Fc region of an antibody have proven to be non-activating modifications making the protein basically inert with regard to Fc function, while at the same time having advantageous properties from a manufacturing perspective.

The term “non-activating” as used herein, is intended to refer to the inhibition or abolishment of the interaction of the protein in accordance with the invention with Fc Receptors (FcRs) present on a wide range of effector cells, such as monocytes, or with C1q to activate the complement pathway. “Non-activating” as used herein includes reduced CDC activity, reduced C1q-binding, reduced ADCC, reduced or absence of binding to human FcγRIa, FcγRIIa(H), FcγRIIa(R), FcγRIIb, FcγRIIIa(F), and FcγRIIIa(V), reduced or absence of activation and signaling via human FcγRIa, FcγRIIa(H), FcγRIIa(R), FcγRIIb, FcγRIIIa(F), and FcγRIIIa(V). “Non-activating” also includes to not induce T-cell activation when used in the context of targeting CD3 (e.g. when used in monospecific antibodies targeting CD3, or in the context of bispecific or multispecific antibodies in a tumor-associated antigen-independent fashion). It is understood that such “non-activating” features are preferably to be assessed relative to a protein which is not “non-activating”, e.g. comparing an antibody, having an unmodified Fc region having a wild-type like functionality with a modified Fc region in accordance with the invention, such as described herein. The term “Fc region” as used herein, is intended to refer to a region comprising, in the direction from the N- to C-terminal, at least a hinge region, a CH2 region and a CH3 region.

The term “protein” as used herein is intended to refer to large biological molecules comprising one or more chains of amino acids covalently linked to one another. Such linkage may be via a peptide bond and/or a disulfide bridge. A single chain of amino acids may also be termed “polypeptide”. Thus, a protein in the context of the present invention may consist of one or more polypeptides. The protein according to the invention may be any type of protein, such as an antibody or a variant of a parent antibody, or a fusion protein.

The term “antibody” as used herein is intended to refer to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The binding region (or binding domain which may be used herein, both having the same meaning) which interacts with an antigen, comprises variable regions of both the heavy and light chains of the immunoglobulin molecule. The constant regions of the antibodies (Abs) may mediate the binding of the immunoglobulin to host tissues or factors, including various cells of the immune system (such as effector cells) and components of the complement system such as C1q, the first component in the classical pathway of complement activation.

As indicated above, the term antibody herein, unless otherwise stated or clearly contradicted by context, includes fragments of an antibody that retain the ability to specifically interact, such as bind, to the antigen. It has been shown that the antigen-binding function of an antibody may be performed by fragments of a full-length antibody. Examples of binding fragments encompassed within the term “antibody” include (i) a Fab′ or Fab fragment, a monovalent fragment consisting of the V_{L}, V_{H}, C_{L} and C_{H}1 domains, or a monovalent antibody as described in WO2007059782 (Genmab A/S); (ii) F(ab′)2 fragments, bivalent fragments comprising two Fab fragments linked by a disulfide bridge at the hinge region; (iii) a Fd fragment consisting essentially of the V_{H} and C_{H}1 domains; (iv) a Fv fragment consisting essentially of the V_{L} and V_{H} domains of a single arm of an antibody, (v) a dAb fragment (Ward et al., Nature 341, 544-546 (1989)), which consists essentially of a V_{H} domain and also called domain antibodies (Holt et al; Trends Biotechnol. 2003 November; 21(11):484-90); (vi) camelid or nanobodies (Revets et al; Expert Opin Biol Ther. 2005 January; 5(1):111-24) and (vii) an isolated complementarity determining region (CDR). Furthermore, although the two domains of the Fv fragment, V_{L} and V_{H}, are coded for by separate genes, they may be joined, using recombinant methods, by a synthetic linker that enables them to be made as a single protein chain in which the V_{L} and V_{H} regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques. An antibody as generated can possess any isotype.

When the antibody is a fragment, such as a binding fragment, it is to be understood within the context of the present invention that said fragment is fused to an Fc region as herein described. Thereby, the antibody may be a fusion protein which falls within the scope of the invention. Thus, in one embodiment, the protein is a fusion protein.

The term “humanized” as used herein in the context of antibodies, refers to a genetically engineered non-human antibody, which contains human antibody constant domains and non-human variable domains modified to contain a high level of sequence homology to human variable domains. This can be achieved by grafting of non-human antibody complementarity-determining regions (CDRs), which together form the antigen binding site, onto a homologous human acceptor framework region (FR) (see i.a. WO92/22653 and EP0629240). In order to fully reconstitute the binding affinity and specificity of the parental antibody binding region, substitution of framework residues from the parental antibody (i.e. the non-human antibody) into the human framework regions (back-mutations) may be required. Structural homology modeling may help to identify the amino acid residues in the framework regions that are important for the binding properties of the antibody binding region. Thus, a humanized variable region or antibody may comprise non-human CDR sequences, primarily human framework regions optionally comprising one or more amino acid back-mutations to the non-human amino acid sequence. Optionally, additional amino acid modifications, which are not necessarily back-mutations, may be applied to obtain a humanized antibody or humanized variable region with preferred characteristics, such as particular useful affinity and biochemical properties, e.g. to include modifications that avoid deamidation, provide an “inert Fc region”, enhance heterodimeration and/or improve manufacturing.

The term “human”, as used herein in the context of variable regions of antibodies, and antibodies, is intended to include antibodies, which may be genetically engineered, having variable and framework regions derived from human germline immunoglobulin sequences and a constant domain derived from a human immunoglobulin constant domain Human variable regions or human antibodies of the invention may include amino acid residues not encoded by human germline immunoglobulin sequences (e.g., mutations, insertions or deletions introduced by random or site-specific mutagenesis in vitro or by somatic mutation in vivo). A “human antibody” can incorporate VH and VL sequences that have been generated from human germline immunoglobulin sequences in a human, in a transgenic animal such as described e.g. in Lee et al. Nature Biotech, 32(4) 2014 pp 355-63 and Macdonald et al., PNAS Apr. 8, 2014 111 (14) 5147-52, or the like. Such VH and VL sequences are considered human VH and VL sequences, which can be fused to constant domains derived from a human immunoglobulin constant domain. Optionally, additional amino acid modifications, which are not necessarily back-mutations, may be applied to obtain a human antibody or human variable region with preferred characteristics, such as particular useful affinity and biochemical properties, e.g. to include modifications that avoid deamidation, provide an “inert Fc region”, enhance heterodimeration and/or improve manufacturing. Hence, “human antibodies” may comprise engineered antibodies.

The term “complement-dependent cytotoxicity” (“CDC”), as used herein, is intended to refer to the process of antibody-mediated complement activation leading to lysis of the cell or virion as a result of pores in the membrane that are created by MAC assembly when the antibody is bound to its target on a cell or virion.

The term “antibody-dependent cell-mediated cytotoxicity” (“ADCC”) as used herein, is intended to refer to a mechanism of killing of antibody-coated target cells or virions by cells expressing Fc receptors that recognize the constant region of the bound antibody.

The term “immunoglobulin heavy chain” or “heavy chain of an immunoglobulin” as used herein is intended to refer to one of the heavy chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The CH1 and CH2 are typically linked via a hinge region.

The term “immunoglobulin” as used herein is intended to refer to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized (see for instance Fundamental Immunology Ch. 7 (Paul, W., ed., 2nd ed. Raven Press, N.Y. (1989)). Within the structure of the immunoglobulin, the two heavy chains are inter-connected via disulfide bonds in the so-called “hinge region”. Equally to the heavy chains each light chain is typically comprised of several regions; a light chain variable region (abbreviated herein as V_{L}) and a light chain constant region. The light chain constant region typically is comprised of one domain, C_{L}. Furthermore, the V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each V_{H} and V_{L} is typically composed of three CDRs and four FRs, arranged from amino-terminus to carboxy-terminus in the following order: FR1, CDR1, FR2, CDR2, FR3, CDR3, FR4 (see also Lefranc M P et al, Dev Comp Immunol January: 27(1):55-77 (2003)).

The term “first polypeptide” and “second polypeptide” as used herein refers to a set of polypeptides which may be identical or different in amino acid sequence. The first and second polypeptide may thus form a homodimer or a heterodimer. The first and second polypeptide may associate with further polypeptides.

The term “isotype” as used herein refers to the immunoglobulin isotype (for instance IgG, IgD, IgA, IgE, or IgM) or subclasses thereof (IgG1, IgG2, IgG3, IgG4) or any allotypes thereof, encoded by heavy chain constant region genes. Examples of an allotype of IgG1 include IgG1m(za) and IgG1m(f). Thus, in one embodiment, the protein comprises a heavy chain of an immunoglobulin of the IgG1 class or any allotype thereof. Further, each heavy chain isotype can be combined with a kappa (κ) and/or lambda (λ) light chain, or any allotypes thereof.

The term “hinge region” as used herein refers to the hinge region of an immunoglobulin heavy chain. Thus, for example the hinge region of a human IgG1 antibody corresponds to amino acids 216-230 according to the Eu numbering as set forth in Kabat (described in Kabat, E. A. et al., Sequences of proteins of immunological interest. 5^{th} Edition—US Department of Health and Human Services, NIH publication No. 91-3242, pp 662,680,689 (1991)).

VH and VL regions may be “human VH and VL regions” or “humanized VH and VL regions”. It is understood that with regard to a human VH and/or human VL region, such a region is typically composed of three CDRs and four FRs, arranged from amino-terminus to carboxy-terminus in the following order: FR1, CDR1, FR2, CDR2, FR3, CDR3, FR4 as derived from or found in human germline sequences. Such VH and VL regions may be derived from humanized animal models or humans. For example, human monoclonal antibodies can be produced by a hybridoma which includes a B cell obtained from a transgenic or transchromosomal non-human animal, such as a transgenic mouse, having a genome comprising a human heavy chain transgene and a light chain transgene, fused to an immortalized cell. Human monoclonal antibodies may be derived from human B cells or plasma cells. The term “humanized VH and VL region” as used herein, refers to a genetically engineered VH and VL regions derived from a non-human antibody, modified to contain a high level of sequence homology to human variable domains. This can be achieved by grafting of non-human antibody complementarity-determining regions (CDRs), which together form the antigen binding site, onto a homologous human acceptor framework region (FR) (see i.a. WO92/22653 and EP0629240). In order to fully reconstitute the binding affinity and specificity of the parental antibody, substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, humanized VH and VL regions may comprise non-human CDR sequences, primarily human framework regions optionally comprising one or more amino acid back-mutations to the non-human amino acid sequence. Optionally, additional amino acid modifications, which are not necessarily back-mutations, may be applied to obtain humanized or human VH and VL regions with preferred characteristics, such as particular useful affinity and biochemical properties, e.g. to include modifications to avoid deamidation, and/or improve manufacturing.

The term “CH2 region” or “CH2 domain” as used herein refers to the CH2 region of an immunoglobulin heavy chain. Thus, for example the CH2 region of a human IgG1 antibody corresponds to amino acids 231-340 according to the Eu numbering system. However, the CH2 region may also be any of the other subtypes as described herein.

The term “CH3 region” or “CH3 domain” as used herein refers to the CH3 region of an immunoglobulin heavy chain. Thus, for example the CH3 region of a human IgG1 antibody corresponds to amino acids 341-447 according to the Eu numbering system. However, the CH3 region may also be any of the other subtypes as described herein.

The term “full-length antibody” as used herein, refers to an antibody (e.g., a parent or variant antibody) which contains all heavy and light chain constant and variable domains corresponding to those that are normally found in a wild-type antibody, i.e. having respectively VH, CH1, linker, CH2, CH3 regions in a heavy chain, and having respectively VL and CL regions in a light chain, such as e.g. a human (or humanized) IgG1 heavy chain or the like, or a human (or humanized) kappa or lambda light chain. It is understood that a bispecific antibody may also be a full-length antibody, i.e. comprising different heavy and/or light chains such as normally found in a wild-type antibody or the like. Full-length antibodies may be engineered, comprising e.g. substitutions or modifications as defined herein in accordance with the invention.

The term “amino acid corresponding to positions” as used herein refers to an amino acid position number in a human IgG1 heavy chain. Unless otherwise stated or contradicted by context, the amino acids of the constant region sequences are herein numbered according to the Eu-index of numbering (described in Kabat, E. A. et al., Sequences of proteins of immunological interest. 5^{th} Edition—US Department of Health and Human Services, NIH publication No. 91-3242, pp 662,680,689 (1991)). Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present invention.

In the context of the present invention, the amino acid may be defined by a conservative or non-conservative class. Thus, classes of amino acids may be reflected in one or more of the following tables:

### Amino Acid Residue of Conservative Class

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

### Alternative Physical and Functional Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P, and T |
| Flexible residues | Q, T, K, S, G, P, D, E, and R |

In the context of the present invention, a substitution in a protein is indicated as:
Original amino acid—position—substituted amino acid;

Referring to the well-recognized nomenclature for amino acids, the three-letter code, or one letter code, is used, including the codes Xaa and X to indicate any amino acid residue. Accordingly, the notation “L234F” or “Leu234Phe” means, that the protein comprises a substitution of Leucine with Phenylalanine in the protein amino acid position corresponding to the amino acid in position 234 in the wild-type protein.

Substitution of an amino acid at a given position to any other amino acid is referred to as:
Original amino acid—position; or e.g. “L234”.

For a modification where the original amino acid(s) and/or substituted amino acid(s) may comprise more than one, but not all amino acid(s), the more than one amino acid may be separated by “,” or “/”. E.g. the substitution of Leucine for Phenylalanine, Arginine, Lysine or Tryptophan in position 234 is:
“Leu234Phe,Arg,Lys,Trp” or “L234F,R,K,W” or “L234F/R/K/W” or “L234 to F, R, K or W”

Such designation may be used interchangeably in the context of the invention but have the same meaning and purpose.

Furthermore, the term “a substitution” or “mutation”, which can be used interchangeably, embraces a substitution into any one of the other nineteen natural amino acids, or into other amino acids, such as non-natural amino acids. For example, a substitution of amino acid L in position 234 includes each of the following substitutions: 234A, 234C, 234D, 234E, 234F, 234G, 234H, 234I, 234K, 234M, 234N, 234Q, 234R, 234S, 234T, 234V, 234W, 234P, and 234Y. This is, by the way, equivalent to the designation 234X, wherein the X designates any amino acid other than the original amino acid. These substitutions can also be designated L234A, L234C, etc., or L234A,C,etc., or L234A/C/etc. The same applies by analogy to each and every position mentioned herein, to specifically include herein any one of such substitutions. It is well-known in the art when an amino acid sequence comprises an “X” or “Xaa”, said X or Xaa represents any amino acid. Thus, X or Xaa may typically represent any of the 20 naturally occurring amino acids. The term “naturally occurring” as used herein, refers to any one of the following amino acid residues; glycine, alanine, valine, leucine, isoleucine, serine, threonine, lysine, arginine, histidine, aspartic acid, asparagine, glutamic acid, glutamine, proline, tryptophan, phenylalanine, tyrosine, methionine, and cysteine. The terms “amino acid” and “amino acid residue” may be used interchangeably. For purposes of the present invention, the sequence identity between two amino acid sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment).

The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1).

In one embodiment, in at least one of said first and second polypeptides the amino acid in the positions corresponding to positions L234, L235 and G236 in a human IgG1 heavy chain, are not L, L, and G, respectively. A protein is provided comprising a first polypeptide and a second polypeptide, wherein said first and second polypeptide each comprise at least a hinge region, a CH2 region and a CH3 region, respectively, of a human IgG1 immunoglobulin heavy chain, wherein at least one of said first and second polypeptides is modified and comprises a substitution of amino acids corresponding with amino acids at the positions L234, L235 and G236, wherein amino acid positions are as defined by EU numbering. Preferably, the said amino acids at positions L234, L235 and G236 in at least one of said first and second polypeptide are substituted with F, E and R, respectively.

With regard to amino acid positions as used herein, these are numbered in accordance with Eu-numbering, this is in accordance with the Eu-index of numbering as described in Kabat, E. A. et al., Sequences of proteins of immunological interest. 5th Edition—US Department of Health and Human Services, NIH publication No. 91-3242, pp 662,680,689 (1991). Sequences of useful amino acid sequences in accordance with the invention are also provided herein with the indicated amino acid modifications in bold (see table 1).

As shown, e.g. in the example section, an example of a protein in accordance with the invention may be an antibody, consisting of two identical heavy chains (which correspond with the first and second polypeptide) and two identical light chains. However, it is not a requirement that both first and second polypeptides have the same substitutions, e.g. one of the first and second polypeptides may have said substitutions at L234, L235 and G236 positions, and the other may e.g. have different substitutions. Hence, the other chain may have e.g. substitutions of another inert format, e.g. of the FEA format. In the context of the present invention, it is considered a further advantage of the non-activating substitutions at positions L234, L235 and G236 that they effectively suppress Fc-mediated effector function, even when present in an “asymmetric” manner, where the other polypeptide has a different inert format, such as the FEA format. This makes it possible to produce multispecific antibodies by combining a newly developed antibody with the FER inert format with previously produced antibodies having other non-activating substitutions, such as the FEA format.

Hence, in a further embodiment, a protein in accordance with the invention is provided, wherein one of the first and second polypeptides comprises said substitution of amino acids corresponding with amino acids at positions L234, L235 and G236, and the other is modified and comprises a substitution of amino acids corresponding with amino acids at positions L234, L235, and D265, wherein preferably, said substitutions are F, E and A, respectively.

In another embodiment, in the protein in accordance with the invention, both of said first and second polypeptides comprise said substitutions of amino acids corresponding with amino acids L234, L235 and G236, which is preferably the substitution with F, E and R, respectively.

In a further embodiment, each of said first and second polypeptides comprises an immunoglobulin CH1 region. Said CH1 region is preferably linked to the hinge region, i.e. providing said polypeptides with a CH1 region, hinge region, a CH2 region and a CH3 region, respectively, of a human IgG1 immunoglobulin heavy chain. Preferably the CH1 region is of a human IgG1 immunoglobulin heavy chain. For example, a CH1 region may be a sequence having the sequence as listed in SEQ ID NO: 4. A CH1 region, hinge region, CH2 region and CH3 region as defined herein may be a sequence as listed in SEQ ID NO: 5. Such a sequence may have substitutions as described herein, e.g. be provided with the FER substitutions and/or further substitutions as defined herein.

In another embodiment, the protein in accordance with the invention comprises a first and a second binding region. Any binding region may suffice, it may however be preferred that the binding regions are derived from immunoglobulin binding regions, such as from human or humanized antibodies.

The term “binding region” or “binding domain” as used herein, refers to a region of a protein which is capable of binding to an antigen, such as a polypeptide, e.g. present on a cell, e.g. on a cancer cell, bacterium, or virion. The binding region may be a polypeptide sequence, such as a protein, protein ligand, receptor, an antigen-binding region, or a ligand-binding region capable of binding to a cell, bacterium, or virion. Specifically, the binding region is an antigen-binding region. If the binding region is e.g. a receptor the protein may have been prepared as a fusion protein of an Fc-domain of an immunoglobulin and said receptor. If the binding region is an antigen-binding region the protein in accordance with the invention may be an antibody, a chimeric antibody, or an antibody having a humanized, or human binding region antibody or a heavy chain only antibody or a ScFv-Fc-fusion.

The term “binding” as used herein refers to the binding of an antibody to a predetermined antigen or target, typically with a binding affinity corresponding to a K_{D} of 1E⁻⁶ M or less, e.g. 5E⁻⁷ M or less, 1E⁻⁷ M or less, such as 5E⁻⁸ M or less, such as 1E⁻⁸ M or less, such as 5E⁻⁹ M or less, or such as 1E⁻⁹ M or less, when determined by biolayer interferometry using the antibody as the ligand and the antigen as the analyte and binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen.

Hence, in a further embodiment, the protein in accordance with the invention comprises said first and second binding region, which comprise respectively a first immunoglobulin heavy chain variable region (VH) and a first immunoglobulin light chain variable region (VL), and wherein said second binding region comprises a second immunoglobulin heavy chain variable region and a second immunoglobulin light chain variable region. It is understood that these regions preferably are human or humanized VH and VL regions. Hence, VH and VL regions have framework regions that are human, or human derived, and may have CDR1-3 sequences that are human or human derived or of other species origin, such as mouse or rat. Hence, in another further embodiment, said immunoglobulin heavy and light chain variable regions are human or humanized immunoglobulin heavy and light chain variable regions.

As said, proteins in accordance with the invention include antibodies. Hence, in another embodiment, said first and second polypeptides are immunoglobulin heavy chains, wherein said first and second polypeptides comprise said respective first and second immunoglobulin heavy chain variable regions. It is understood that such heavy chains may be preferred to be human heavy chains or humanized heavy chains, when comprising human variable regions, which is understood to comprise human or humanized variable regions and human constant regions. Furthermore, said protein may comprise a first immunoglobulin light chain constant region and a second immunoglobulin light chain constant region, more preferably wherein said protein comprises first and second immunoglobulin light chains, said immunoglobulin light chains comprising said respective first and second immunoglobulin light chain variable regions and said respective first and second immunoglobulin constant light chain regions. It is understood that such light chains are highly preferred to be human light chains or humanized light chains when comprising non-human derived CDR regions. Light chains may have a light chain variable region and a human kappa light chain constant region or a human lambda light chain constant region. In a further embodiment, the human kappa light chain constant region is as listed in SEQ ID NO: 6. In another further embodiment, the human lambda light chain constant region is as listed in SEQ ID NO: 7. Such light chains may comprise kappa or lambda light chains, or both, e.g. wherein the protein comprises one kappa light chain and one lambda light chain, as the protein in accordance with the invention may comprise two different light chains. Hence, such light chains may be either human or humanized kappa or lambda light chains, or both, e.g. wherein the protein comprises one human kappa light chain and one human lambda light chain, as the protein in accordance with the invention may comprise two different light chains.

It is understood that the human or humanized heavy and light chains may comprise in addition to the mutations as described herein, further modifications to provide for preferred characteristics, e.g. to provide for particular useful affinity and biochemical properties, including modifications to avoid deamidation and/or enhance heterodimerization, improve manufacturing and separation, or the like.

Hence, in a preferred embodiment, the protein in accordance with the invention which comprises said first and second polypeptides, consists or comprises a first and second immunoglobulin light chain, and a first and second heavy chain, the latter corresponding with the first and second polypeptides. Such a protein in accordance with the invention may have the first immunoglobulin light chain connected with said first immunoglobulin heavy chain via disulfide bridges and said second immunoglobulin light chain connected with said second immunoglobulin heavy chain via disulfide bridges, thereby forming said first binding region and said second binding region, respectively, and wherein said first and second immunoglobulin heavy chains are connected via disulfide bridges as well. The term “disulfide bridges” as used herein refers to the covalent bond between two Cysteine residues, i.e. said interaction may also be designated a Cys-Cys interaction.

In a further embodiment, which may be a preferred embodiment, the protein in accordance with the invention is an antibody, which is preferably a full-length antibody. In yet another further embodiment, the full-length antibody is of, or is derived of, a human IgG1 isotype.

It is understood that in accordance with the invention, substitutions of said positions are to result in reduced Fc-mediated effector functions, e.g. when included in an antibody. Such reduced Fc-mediated effector functions include reduced CDC activity (see i.a. examples 3 and 5), reduced C1q-binding (i.a. example 4), reduced ADCC (i.a. example 9), no detectable binding to human FcγRIa, FcγRIIa(H), FcγRIIa(R), FcγRIIb, FcγRIIIa(F), and FcγRIIIa(V) (i.a. example 6) and no detectable activation and signaling via human FcγRIa, FcγRIIa(H), FcγRIIa(R), FcγRIIb, FcγRIIIa(F), and FcγRIIIa(V) (example 7), and not to induce tumor-associated-antigen-independent T-cell activation in the context of targeting CD3 (i.a. example 10), as well as having pharmacokinetics, i.a. due to having similar human FcRn binding properties as wild-type human IgG1, and glycosylation highly similar to wild-type human IgG1 antibodies, or the like, when such substations are included in the context of an antibody (see e.g. example 12 and 14). Moreover, these substitutions can provide for improved pH stability at low pH (see i.a. example 20-23). Not only are the proteins in accordance with the invention useful in the context of antibodies, which may be highly preferred embodiments, proteins of other formats, such as fusion proteins, having the said first and second polypeptide in accordance with the invention comprising at least a hinge region, a CH2 region and a CH3 region wherein at least one of said first and second polypeptides is modified and comprises a substitution of amino acids corresponding with amino acids at the positions L234, L235 and G236, wherein amino acid positions are as defined by EU numbering, are contemplated as well.

The terms “reduced” or “non-detectable” as used herein when referring to Fc-mediated effector functions relates to the ability of a protein in accordance with the invention, such as an antibody, to induce CDC activity, ADCC, C1q binding, FcγRIa, FcγRIIa(H), FcγRIIa(R), FcγRIIb, FcγRIIIa(F), and FcγRIIIa(V) binding, activate and signal via FcγRIa, FcγRIIa(H), FcγRIIa(R), FcγRIIb, FcγRIIIa(F), and FcγRIIIa(V), and activate T-cells in the context of targeting CD3, as compared with the same protein having a wild-type IgG1 Fc-region, or the like, which has full capacity to induce said effector functions, e.g. in the context of antibodies or the like such as described in the example section. Exemplary assays for determining “reduced” or “non-detectable binding” for these features are known in the art and are well described e.g. in the example section throughout. The example section describes the properties of these features in the context of antibodies, such as full-length antibodies. It is understood that highly preferable properties are determined in a context such as described in the example section, which does not mean that the proteins in accordance with the invention are understood to be limited to antibodies, as the advantageous properties of the modified hinge region, CH2 region and CH3 region, respectively, of a human IgG1 immunoglobulin heavy chain in accordance with the invention may also be useful in other formats, such as fusion proteins or the like. Nevertheless, it finds, and it is shown herein, in particular use when applied in the context of antibodies.

The ability of a protein to induce complement-dependent cytotoxicity (CDC) can be determined with methods known in the art. Briefly, a reference protein having an unmodified fully functional Fc region, e.g. an IgG1 antibody which is a potent CDC inducer, is incubated in an in vitro assay with cells presenting a target antigen on its cell-surface, in the presence of human serum, and subsequently the percentage of cell lysis is determined, which is set at 100%. The percentage of cell-lysis of a protein in accordance with the invention, having a modified Fc region, e.g. an antibody with a FER Fc region, is compared with the cell-lysis occurring with a control protein that does not target the cell, or does not have an Fc region (such as e.g. a F(ab′)2), under the same conditions. The percentage lysis is determined as compared with the unmodified reference which is set at 100%. An example of a suitable method that may be used is as described in example 3 or example 5. A protein in accordance with the invention, has reduced CDC activity when compared with the same protein which has an Fc region with a FEA format instead, and/or has a similar CDC activity when compared with e.g. a F(ab′)2).

The ability of a protein to have reduced C1q binding can be determined with methods known in the art. Briefly, a protein having an (unmodified) fully functional Fc region, e.g. an antibody, is incubated in an in vitro assay with cells presenting a target antigen on its cell-surface, in the presence of human serum, and subsequently the percentage of C1q binding is determined by binding with e.g. a polyclonal rabbit anti-human C1q complement FITC antibody (Dako, Cat #F0254, Agilent Technologies) and FACS analysis in accordance with manufacturer's instructions. The signal detected of a protein having a (unmodified) fully functional FC region is compared with a protein having a modified Fc region, e.g. an IgG1 antibody which is a potent CDC inducer. A detailed example of a suitable method in accordance with the invention that may be used is described in example 4. A protein in accordance with the invention, has reduced CDC activity when compared with the same protein which has an Fc region with a FEA format instead, and/or has a similar CDC activity when compared with e.g. non-binding control antibody. A protein in accordance with the invention preferably has a C1q binding activity of 15% or less, when comparing in the context of a full length IgG1 antibody with FER with an antibody having the same sequence but without FER, such as described in example 4.

The ability to reduce antigen-dependent cellular cytotoxicity can be determined with methods known in the art. For example, for determining the ADCC capacity of wild-type antibodies and non-activating variants thereof, the DELFIA® EuTDA TRF (time-resolved fluorescence) cytotoxicity kit (Cat #AD0116, Perkin Elmer) can be used in accordance with manufacturer's instructions. Briefly, cells presenting a target antigen are intracellularly labeled e.g. using bis(acetoxymethyl)2,2′:6′,2″-terpyridine-6,6″-dicarboxylate reagent solution (DELFIA BATDA reagent, Cat #C136-100, Perkin Elmer), in accordance with the manufacturer's instructions. These cells are subsequently incubated with a protein having an (unmodified) fully functional Fc region, e.g. a wild-type antibody against the target antigen, and non-activating variants thereof, with PBMCs cells presenting a target antigen on its cell-surface, in the presence of PBMCs. Assessment of NK-mediated ADCC is determined with reference to a fully functional control IgG1 antibody (set at 100%) and a non-binding negative IgG1 control antibody (set at 0%). A detailed example of a suitable method that may be used is described in example 9. A protein in accordance with the invention preferably has a residual ADCC activity of 35% or less, when comparing e.g. a full length IgG1 antibody with FER with an antibody having the same sequence but without FER, such as described in example 9. A protein in accordance with the invention, has a similar reduced ADCC activity when compared with the same protein which has an Fc region with a FEA format instead.

With regard to binding to human FcγRIa, FcγRIIa(H), FcγRIIa(R), FcγRIIb, FcγRIIIa(F), and FcγRIIIa(V), this can be determined by using ELISA. Binding of a protein in accordance with the invention can be assessed with determining binding to His-tagged, C-terminally biotinylated FcγR, monomeric ECD of FcγRIa (SEQ ID NO: 15) (monomeric), or dimeric ECD of FcγRIIa allotype 131H (SEQ ID NO: 16), FcγRIIa allotype 131R (SEQ ID NO: 17), FcγRIIb (SEQ ID NO: 18), FcγRIIIa allotype 158F (SEQ ID NO: 19), and FcγRIIIa allotype 158V (SEQ ID NO: 20) in ELISA assays. Briefly, and for example, an IgG1 antibody with an Fc region is bound to a plate coated with an anti-human F(ab′)2 antibody, and subsequently incubated with each of the respective extracellular domains, of which binding is subsequently quantified using Streptavidin-polyHRP (CLB, Cat #M2032, 1:10.000). A detailed example of a suitable method in accordance with the invention that may be used is described in example 6. The protein in accordance with the invention, e.g. an antibody, does not detectably bind with said Fcγ receptors in an ELISA assay utilizing said His-tagged, C-terminally biotinylated FcγR, monomeric ECDs. The protein in accordance with the invention has a similar non-detectable binding to said Fcγ receptors as observed when comparing an IgG1 antibody with an Fc region comprising FER with e.g. FEA, such as shown e.g. in example 6.

With regard to activation and signaling via FcγRIa, FcγRIIa(H), FcγRIIa(R), FcγRIIb, FcγRIIIa(F), and FcγRIIIa(V), this can be determined with commercially available reporter assays. For example, reporter assays can be used to determine activation and binding of a protein in accordance with the invention, using target-expressing cells and a Jurkat reporter cell line that expresses the indicated FcγR can used (Promega, FcγRIa: Cat #CS1781C08; FcγRIIa allotype 131H: Cat #G9991; FcγRIIa allotype 131R: Cat #CS1781608; FcγRIIb: Cat #CS1781E04; FcγRIIIa allotype 158F: Cat #G9790; FcγRIIIa allotype 158V: Cat #G7010). For example, for CD20-targeting antibodies, CD20-expressing Raji cells may be used as target cells. The protein in accordance with the invention, such as an antibody, has a similar non-detachable activation and signaling via said Fcγ receptors as observed when comparing e.g. an antibody with an Fc region in accordance with the invention such as FER, with FEA, as shown e.g. in example 7.

With regard to the reduction of activation of T-cells in the context of targeting CD3, it is understood that such applies to a protein in accordance with the invention which comprises a binding region that binds human CD3 on human T-cells, e.g. a typical bivalent monospecific antibody binding human CD3 such as described in the examples herein. A reduction in activation of T-cells can be determined by incubating dose-response series of e.g. an anti-CD3 antibody comprising in a hinge region, a CH2 region and a CH3 region, respectively, of a human IgG1 immunoglobulin heavy chain a substitution of amino acids corresponding with amino acids at the positions L234, L235 and G236, in accordance with EU-numbering, in both of the two polypeptides, in accordance with the invention, with freshly isolated PBMCs, and subsequently staining said cells with mouse-anti-human CD28-PE (Cat #130-092-921; Miltenyi Biotec; T-cell marker) and mouse-anti-human CD69-APC antibody (Cat #340560; BD Biosciences). Therewith, CD69 upregulation is determined of T-cells which is a measure of T-cell activation. Details of such an assay are described in example 10. A protein, e.g. an antibody targeting human CD3, in accordance with the invention can prevent or highly reduce CD69 upregulation as compared with an IgG1 antibody targeting human CD3 having a wild-type like Fc region.

With regard to proteins in accordance with the invention, i.e. comprising a hinge, CH2 and CH3 region of a human IgG1 immunoglobulin heavy chain, having at least a substitution of amino acids corresponding with amino acids at the positions L234, L235 and G236, preferably with F, E, and R, respectively, these are preferably to have a glycosylation very similar to a wild-type IgG1 sequence. More specifically, galactosylation and/or the presence of charged glycans of a protein in accordance with the invention are preferably in the same range as found for a wild-type IgG1 amino acid sequence produced using the same cell line and the same production conditions. Suitable mammalian host cells, which includes CHO cell lines, for manufacturing are well known in the art (see i.a. Butler and Spearman, 2014, Curr Opin Biotechno, December; 30:107-12). The total percentage galactosylation preferably is in the range of plus or minus 20% as compared with the total percentage of galactosylation observed in the same protein comprising a wildtype IgG1 sequence, such as SEQ ID NO.1, or the like. For example, if a wild-type IgG1 sequence or the like has a percentage of galactosylation of 25%, the total percentage can be in the range of 5%-45%. The total percentage galactosylation preferably is in the range of plus or minus 20% as compared with the total percentage galactosylation observed in the protein in accordance with the invention not comprising the FER format. The total percentage of charged glycans preferably is in the range of plus or minus 3% as compared with the total percentage charged glycans observed in protein comprising a wildtype IgG1 sequence, such as SEQ ID NO.1, or the like. For example, if a wild-type IgG1 sequence or the like has a percentage of charged glycans of 1%, the total percentage of charged glycans can be in the range of 0%-4%. The total percentage of charged glycans preferably is in the range of plus or minus 3% as compared with the total percentage charged glycans observed in the protein in accordance with the invention not comprising the FER format. The total percentage of charged glycans and/or the percentage of galactosylation of a protein in accordance with the invention, such as an antibody, preferably is in the range of plus or minus 3% of the total percentage of charged glycans and plus or minus 20% as compared with the total percentage galactosylation as observed in the same protein comprising a wildtype IgG1 sequence, such as SEQ ID NO.1, or the like. The total percentage of charged glycans and/or galactosylation of a protein in accordance with the invention, such as an antibody, preferably is in the range of plus or minus 3% of the total percentage of charged glycans and plus or minus 20% as compared with the total percentage galactosylation as observed in the protein in accordance with the invention not comprising the FER format.

The percentage galactosylation and/or charged glycans, of a protein in accordance with the invention, such as an antibody, can be determined using methods known in the art. Such methods are described e.g. in example 14. Suitable methods include 2-aminobenzamidelabelling and subsequent HPLC analysis, such as described in example 14, or, LC-MC using an Orbitrap Q-Extractive Pluss mass spectrometer. Here, the percentage of galactosylation and charged glycans, respectively, is calculated as the percentage occupancy of galactose or charged glycans in the oligosaccharides relative to all glycans having an A2F glycan structure. The amounts referred to herein as percentages represent molar amounts, i.e. representative of number of molecules and not of mass. Percentages of charged glycans and/or galactosylation can be determined of proteins in accordance with the invention, such as antibodies, when produced in Expi293F cells. For example, as shown in the example section, of a protein produced in Expi293F cells having a wildtype IgG1 sequence the percentage of charged glycans and percentage of galactosylation is about 0.5% and about 15% to 25% respectively. Hence, a protein in accordance with the invention when produced in Expi293F cells preferably has a percentage of charged glycans and percentage of galactosylation which preferably is respectively between 0-4% and 5-45%.

With regard to the protein in accordance with the invention, comprising a hinge, CH2 and CH3 region of a human IgG1 immunoglobulin heavy chain having at least a substitution of amino acids corresponding with amino acids at the positions L234, L235 and G236, preferably with F, E, and R, respectively, these are to preferably have human FcRn binding which is similar to a wild-type human IgG1 Fc region. It is understood that substitutions as selected herein may be substitutions that do not affect the FcRn binding function. Hence, the pharmacokinetics of such a protein is similar to the pharmacokinetics of a corresponding protein having a wild-type IgG1 Fc region, such as described i.a. in the example section. However, it is understood that should it be for example be useful to modify FcRn function, e.g. extending half-life or shortening half-life, in scenarios wherein such would be useful, it may be contemplated to include further substitutions therefor. For antibodies, such as e.g. full-length monospecific and bispecific antibodies, in one embodiment, human FcRn binding properties is not different from the same antibody having a wild-type human IgG1 Fc region. Such binding properties are known in the art and can be determined as described herein in the example section. Hence, FcRn binding at pH 6.0 occurs similar as observed with a corresponding wild-type human IgG1 Fc region, and, at pH 7.4 no detectable binding occurs.

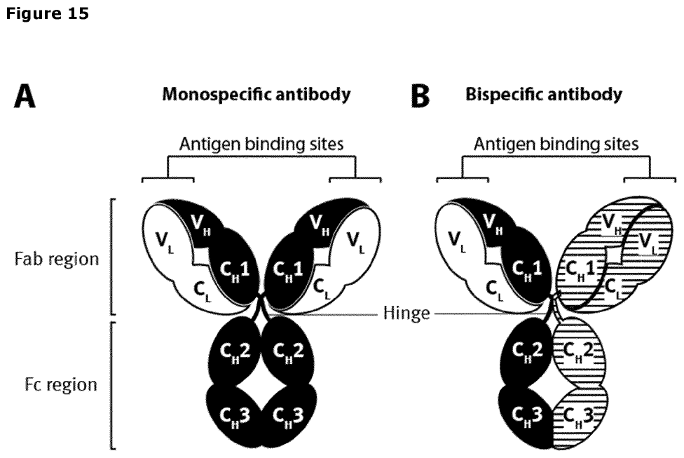

In a further embodiment, the protein in accordance with the invention comprising said first and second polypeptides have an identical amino acid sequence. It is understood that this includes proteins produced from a single expression cassette encoding one polypeptide in a host cell, i.e. the first and second polypeptide can be a homodimer of that one polypeptide. An example of such a protein includes an antibody, e.g. an antibody having two heavy and two light chains (see FIG. 15A), wherein both of the two heavy chains are identical, and both of the light chains as well. Such an antibody is bivalent and has two binding regions that each can bind the same target antigen, i.e. the same epitope. Hence, in a further embodiment, the protein in according with the invention comprises a first and second polypeptide, wherein said first and second polypeptides are immunoglobulin heavy chains, which are identical in amino acid sequence, and further comprises first and second immunoglobulin light chains, which are identical in amino acid sequence.

In a further embodiment, the protein in accordance with the invention comprises further substitutions. Preferred further substitutions in accordance with the invention include modifications that allow for the formation of a heterodimer, i.e. allow to provide for a protein comprising a first and second polypeptide, wherein the first and second polypeptide are different. An example of such a protein includes a bispecific antibody, e.g. an antibody having two heavy and two light chains (see FIG. 15B), wherein at least the two heavy chains are not identical, such that each of the heavy chain and light chain pair in the antibody can target a different antigen. It may not necessarily be required to introduce differentiating modifications in the hinge, CH2 and CH3 region of the IgG1 sequence of the first and second polypeptide, these regions of the first and second polypeptide sequences may be identical. That way, mixtures of homodimers and heterodimers may be formed, which use may be advantageous in itself, or the heterodimers and homodimers may be easily separated from that mixture (e.g. via differences in size and/or charge, affinity, or the like). However, it is preferred to introduce further modifications that enable or drive the generation of a bispecific antibody or the like. Hence, proteins in accordance with the invention may comprise further substitutions in the hinge, CH2 and CH3 region of the IgG1 sequence of the first and second polypeptide. This way, first and second polypeptides comprising sequences that are different with regard to the hinge, CH2 and CH3 region of the IgG1 sequence can advantageously be combined in a protein in accordance with the invention. Examples of such proteins, i.e. immunoglobulin or immunoglobulin like proteins having such substitutions include but are not limited to proteins having complementary CH3 domains such as Triomab/Quadroma (Trion Pharma/Fresenius Biotech; Roche, WO2011069104), the Knobs-into-Holes (Genentech, WO9850431), CrossMAbs (Roche, WO2011117329) and the electrostatically-matched (Amgen, EP1870459 and WO2009089004; Chugai, US201000155133; Oncomed, WO2010129304), the LUZ-Y (Genentech), DIG-body and PIG-body (Pharmabcine), the Strand Exchange Engineered Domain body (SEEDbody)(EMD Serono, WO2007110205), the Biclonics (Merus), FcAAdp (Reqeneron, WO201015792), bispecific IgG1 and IgG2 (Pfizer/Rinat, WO11143545), Azymetric scaffold (Zymeworks/Merck, WO2012058768), mAb-Fv (Xencor, WO2011028952), bivalent bispecific antibodies (Roche) and the DuoBody (Genmab A/S, WO2011131746).

In a particular embodiment, a protein in accordance with the invention, said first and second polypeptide comprise a further amino acid substitution, preferably a substitution of an amino acid selected from the group consisting of T366, L368, K370, D399, F405, Y407, and K409, such as F405L or K409R. It is understood that when the protein is a homodimer, e.g. having identical first and second polypeptides, both may have the same substitutions. Such a protein in accordance with the invention, e.g. a monospecific antibody, can be highly advantageously used for the preparation of a bispecific antibody, such as described in the example section and e.g. in WO2011131746.

Hence, in a further embodiment, the protein in accordance with the invention is a monospecific antibody. In the context of the present invention “monospecific” refers to a protein which binds, i.e. is capable of binding, to the same epitope with its binding regions. In particular, such a monospecific protein or antibody, preferably binds to an antigen selected from target molecules, cellular targets, and pathogens.

Target molecules which may be contemplated include molecules such as cytokines, growth factors, ligands, and the like. Cellular targets that may be contemplated to include molecules at the cell surface such as receptors or adhesion molecules, e.g. as present on cancer cells, tumor cells, effector cells (e.g. macrophages, monocytes, NK cells and T cells). Pathogens that may be targeted include viruses, bacteria, protozoans, parasites, and the like. Hence, proteins in accordance with the invention that may be contemplated include proteins, such as monospecific antibodies, having a binding region for an antigen or a target selected from the group of cytokines, growth factors, ligands, cancer cells, tumor cells, effector cells, viruses and bacteria.

However, the invention is not limited to monospecific proteins, such as monospecific antibodies, but also relates to multispecific proteins, such as bispecific antibodies. In a further embodiment, the protein in accordance with the invention is a bispecific or multispecific antibody. This means that the binding regions of a protein in accordance with the invention, bind different epitopes, instead of the same epitope. Hence, the first and second binding region of the protein in accordance with the invention are different, e.g. with regard to amino acid sequence. The different epitopes may be from the same target entity, e.g. different epitopes presented by the same target molecule and/or as presented by the same target cell, but may also be from different target entities.

A highly advantageous bispecific protein in accordance with the invention, such as a bispecific antibody, involves a protein wherein one of the first and second binding regions targets an effector cell, and the other of the first and second binding regions targets a cancer antigen. By using such a multispecific antibody, a specific class of effector cell may be engaged with a cancer cell thereby e.g. inducing killing of the cancer cell by the effector cell. In one embodiment, a bispecific antibody in accordance with the invention is provided, wherein one of said binding regions binds a cancer antigen. In another embodiment, one of said binding regions binds an effector cell, such as a T-cell, NK cell, macrophage, dendritic call, monocyte or a neutrophil. In yet another embodiment, one of said binding regions binds an effector cell, such as a T-cell or NK cell, and the other binding region binds a cancer antigen.

There is a range of applications, such as receptor inhibition or T-cell recruitment by bispecific antibodies, in which the Fc binding of the Fc region of therapeutic antibodies to effector cells or complement is not required or even is undesired as it may contribute to unwanted cytotoxicity. Thus, a bispecific antibody which binds with one binding region to a human T-cell receptor can advantageously recruit human cytotoxic T-cells. Hence, a bispecific antibody herein is provided which binds with one binding region to human CD3 and which can recruit cytotoxic T-cells. CD3 antibodies, including bispecific antibodies, with an activating IgG Fc region can induce unwanted agonism in the absence of tumor cells through crosslinking by FcγR-expressing cells, inappropriate activation of FcγR-expressing cells and subsequent cytokine storm and associated toxic effects, or platelet aggregation. Thus, CD3 bispecific antibodies with a non-activating Fc region are advantageous to prevent potential unwanted cell activation.

Hence, in one embodiment, at least one of the first and second binding regions bind CD3, i.e. capable of binding CD3. In a particular embodiment, said first binding region binds CD3 and said second binding region binds, i.e. is capable of binding, any other target of interest. Such other target may be a cancer antigen. Such other target may be a tumor-specific target or a cancer-specific target. Preferably, said protein in accordance with the invention is a bispecific antibody. Said protein bispecific antibody preferably having a first binding region capable of binding CD3 and having a second binding region capable of binding a cancer-specific target.

It is understood that antibodies, which include monospecific, bispecific, or multispecific antibodies, may comprise an Fc region, or the like, comprising a hinge region, CH2 and CH3 region of a human IgG1 antibody in accordance with the invention. If an antibody format such as described i.a. below would not comprise such an IgG1 Fc region, such an antibody may be provided therewith, e.g. by replacing an Fc region of such an antibody with a hinge region, CH2 and CH3 region comprising FER in accordance with the invention, or, in case such an antibody does not comprise an Fc region, providing such an antibody therewith, e.g. via fusion and/or conjugation. Hence, any antibody format may be contemplated in accordance with the invention, as long as the antibody comprises a hinge region, CH2 and CH3 region of a human IgG1 antibody in accordance with the invention.

Examples of bispecific antibody molecules which may be used in the present invention comprise (i) a single antibody that has two arms comprising different antigen-binding regions, (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′) 2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In one embodiment, the bispecific antibody of the present invention is a diabody, a cross-body, or a bispecific antibody obtained via a controlled Fab arm exchange (such as described in WO 11/131746) as those described in the present invention.

Examples of different classes of bispecific antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule fused to heavy-chain constant-domains, Fc-regions or parts thereof.

Examples of bispecific IgG-like molecules, or the like, with complementary CH3 domains molecules include but are not limited to the Triomab/Quadroma (Trion Pharma/Fresenius Biotech; Roche, WO2011069104), the Knobs-into-Holes (Genentech, WO9850431), CrossMAbs (Roche, WO2011117329) and the electrostatically-matched (Amgen, EP1870459 and WO2009089004; Chugai, US201000155133; Oncomed, WO2010129304), the LUZ-Y (Genentech), DIG-body and PIG-body (Pharmabcine), the Strand Exchange Engineered Domain body (SEEDbody)(EMD Serono, WO2007110205), the Biclonics (Merus), FcAAdp (Reqeneron, WO201015792), bispecific IgG1 and IgG2 (Pfizer/Rinat, WO11143545), Azymetric scaffold (Zymeworks/Merck, WO2012058768), mAb-Fv (Xencor, WO2011028952), bivalent bispecific antibodies (Roche) and the DuoBody (Genmab A/S, WO2011131746).

Examples of recombinant IgG-like dual targeting molecules include but are not limited to Dual Targeting (DT)-Ig (GSK/Domantis), Two-in-one Antibody (Genentech), Cross-linked Mabs (Karma nos Cancer Center), mAb 2 (F-Star, WO2008003116), Zybodies (Zyngenia), approaches with common light chain (Crucell/Merus, U.S. Pat. No. 7,262,028), KABodies (NovImmune) and CovX-body (CovX/Pfizer).

Examples of IgG fusion molecules include but are not limited to Dual Variable Domain (DVD)-Ig (Abbott, U.S. Pat. No. 7,612,181), Dual domain double head antibodies (Unilever; Sanofi Aventis, WO20100226923), IgG-like Bispecific (ImClone/Eli Lilly), Ts2Ab (MedImmune/AZ) and BsAb (Zymogenetics), HERCULES (Biogen Idec, U.S. Ser. No. 00/795,1918), scFv fusion (Novartis), scFv fusion (Changzhou Adam Biotech Inc, CN 102250246) and TvAb (Roche, WO2012025525, WO2012025530).

Examples of Fc fusion molecules include but are not limited to ScFv/Fc Fusions (Academic Institution), SCORPION (Emergent BioSolutions/Trubion, Zymogenetics/BMS), Dual Affinity Retargeting Technology (Fc-DART) (MacroGenics, WO2008157379, WO2010/080538) and Dual(ScFv)₂-Fab (National Research Center for Antibody Medicine—China).

Examples of Fab fusion bispecific antibodies include but are not limited to F(ab)₂ (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech).

Examples of ScFv-, diabody-based and domain antibodies include but are not limited to Bispecific T Cell Engager (BITE) (Micromet, Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

In a further embodiment, a bispecific antibody is provided in accordance with the invention, wherein said first and second polypeptide comprise further substitutions in said respective CH2 and CH3 regions such that the sequences of the respective CH2 and CH3 regions from said first and second polypeptides are different, said substitutions allowing to obtain said polypeptide comprising said first and second polypeptide. In a particular embodiment, in said first polypeptide at least one of the amino acids in the positions corresponding to a position selected from the group consisting of T366, L368, K370, D399, F405, Y407, and K409 in a human IgG1 heavy chain has been substituted, and in said second polypeptide at least one of the amino acids in the positions corresponding to a position selected from the group consisting of; T366, L368, K370, D399, F405, Y407, and K409 in a human IgG1 heavy chain has been substituted, wherein said substitutions of said first and said second polypeptides are not in the same positions. In this context the term “substituted”, refers to the amino acid in a specific amino acid position which has been substituted with another type of amino acid. Thus, a “substituted” amino acid in a position corresponding to the position in a human IgG1 heavy chain means the amino acid at the particular position is different from the naturally occurring amino acid at that position in an IgG1 heavy chain.

Furthermore, a bispecific antibody in accordance with the invention is provided, wherein in said first polypeptide at least one of the amino acids in the positions corresponding to a position selected from the group consisting of T366, L368, K370, D399, F405, Y407, and K409 in a human IgG1 heavy chain has been substituted, and in said second polypeptide at least one of the amino acids in the positions corresponding to a position selected from the group consisting of; T366, L368, K370, D399, F405, Y407, and K409 in a human IgG1 heavy chain has been substituted, and wherein said substitutions of said first and said second polypeptides are not in the same positions.

In a further embodiment, the amino acid in the position corresponding to F405 in a human IgG1 heavy chain is L in said first polypeptide, and the amino acid in the position corresponding to K409 in a human IgG1 heavy chain is R in said second polypeptide, or vice versa.

In another embodiment, a bispecific antibody in accordance with the invention is provided, wherein the amino acid in the position corresponding to F405 is L in said first polypeptide, and the amino acid in the position corresponding to K409 is R in said second polypeptide, or vice versa. In another embodiment, a bispecific antibody in accordance with the invention is provided, wherein the amino acid in the position corresponding to F405 and K409 is L and K, respectively, in said first polypeptide, and the amino acid in the position corresponding to F405 and K409 is F and R, respectively, in said second polypeptide, or vice versa.

In yet another embodiment, a bispecific antibody in accordance with the invention is provided, wherein said bispecific antibody has modifications in both of said first and second polypeptides comprising substitutions of the amino acids at positions L234, L235 and G236 with F, E and R, and substitutions of the amino acid at position F405 with is L in said first polypeptide, and at K409 with R in said second polypeptide, or vice versa.

In one embodiment, a bispecific antibody in accordance with the invention is provided, wherein said bispecific antibody has modifications in one of said first and second polypeptide comprising substitutions of the amino acids at positions L234, L235 and G236 with F, E and R, and substitutions of the amino acid at position F405 with is L in said first polypeptide, and at K409 with R in said second polypeptide, or vice versa.

In still another embodiment, a bispecific antibody in accordance with the invention is provided, wherein said bispecific antibody has modifications in said first and second polypeptides comprising substitutions of the amino acids at positions L234, L235 and G236 with F, E and R in the first second polypeptide, and substitutions in said second polypeptide of the amino acids at positions L234, L235 and D265 with F, E and A, and substitutions of the amino acid at position F405 with L in said first polypeptide, and K409 with R in said second polypeptide.

In yet another embodiment, a bispecific antibody in accordance with the invention is provided, wherein said bispecific antibody has modifications in said first and second polypeptides comprising substitutions of the amino acids at positions L234, L235 and G236 with F, E and R in the first second polypeptide, and substitutions in said second polypeptide of the amino acids at positions L234, L235 and D265 with F, E and A, and substitutions of the amino acid at position F405 with L in said second polypeptide, and K409 with R in said first polypeptide.

In yet another embodiment, a bispecific antibody in accordance with the invention is provided, wherein said bispecific antibody has modifications in both of said first and second polypeptides consisting of substitutions of the amino acids at positions L234, L235 and G236 with F, E and R, and substitutions of the amino acid at position F405 with is L in said first polypeptide, and at K409 with R in said second polypeptide, or vice versa.

In one embodiment, a bispecific antibody in accordance with the invention is provided, wherein said bispecific antibody has modifications in one of said first and second polypeptides consisting of substitutions of the amino acids at positions L234, L235 and G236 with F, E and R, and substitutions of the amino acid at position F405 with is L in said first polypeptide, and at K409 with R in said second polypeptide, or vice versa.

In still another embodiment, a bispecific antibody in accordance with the invention is provided, wherein said bispecific antibody has modifications in said first and second polypeptides consisting of substitutions of the amino acids at positions L234, L235 and G236 with F, E and R in the first second polypeptide, and substitutions in said second polypeptide of the amino acids at positions L234, L235 and D265 with F, E and A, and substitutions of the amino acid at position F405 with L in said first polypeptide, and K409 with R in said second polypeptide.

In yet another embodiment, a bispecific antibody in accordance with the invention is provided, wherein said bispecific antibody has modifications in said first and second polypeptides consisting of substitutions of the amino acids at positions L234, L235 and G236 with F, E and R in the first second polypeptide, and substitutions in said second polypeptide of the amino acids at positions L234, L235 and D265 with F, E and A, and substitutions of the amino acid at position F405 with L in said second polypeptide, and K409 with R in said first polypeptide.

Said protein in accordance with the invention can be based on a hinge, CH2 and CH3 region of human IgG1 as defined in SEQ ID NO: 1. Said hinge, CH2 and CH3 region of human IgG1 corresponds with the allotype IgG1m(f). Of course, any other human IgG1 allotype within the IgG1 immunoglobulin class (e.g. IgG1m(za), IgG1m(zax), IgG1m(zav), or IgG1m(fa); see i.a. Vidarsson et al., 2014, Front. Immunol., 20 October and as provided in the IMGT database (www.imgt.org)) may be contemplated and are equally suitable.

Fc regions may have at their C-terminus a lysine. The origin of this lysine is a naturally occurring sequence found in humans from which these Fc regions are derived. During cell culture production of recombinant antibodies, this terminal lysine can be cleaved off by proteolysis by endogenous carboxypeptidase(s), resulting in a constant region having the same sequence but lacking the C-terminal lysine. For manufacturing purposes of antibodies, the DNA encoding this terminal lysine can be omitted from the sequence such that antibodies are produced without the lysine. Antibodies produced from nucleic acid sequences that either do, or do not encode a terminal lysine are substantially identical in sequence and in function since the degree of processing of the terminal lysine is typically high when e.g. using antibodies produced in CHO-based production systems (Dick, L. W. et al. Biotechnol. Bioeng. 2008; 100: 1132-1143). The constant region sequences as listed herein list a terminal lysine (K) (see i.a. SEQ ID NO. 1) and sequences encoding a terminal lysine (K) were used in the example section herein. Hence, it is understood that proteins in accordance with the invention, such as antibodies, can be generated without encoding or having a terminal lysine such as listed herein in SEQ ID NO. 1-3, 5, and 9-14.

Accordingly, the protein in accordance with the invention, or bispecific antibody in accordance with the invention, may comprise a first and second polypeptide comprising an amino acid sequence as defined herein in accordance with SEQ ID NO: 1 wherein said first and second proteins have amino acid substitutions as defined herein. The protein in accordance with the invention, or bispecific antibody in accordance with the invention, wherein said first and second polypeptides preferably comprises an amino acid sequence in accordance with SEQ ID NO: 1, wherein said amino acid sequence which is comprised in said first and second polypeptides having amino acid substitutions as defined herein. Furthermore, said amino acid sequences as defined by SEQ ID NO: 1, having substitutions as defined herein, may not comprise a terminal lysine.

Hence, a protein, or monospecific or bispecific antibody, which may be a full-length antibody, in accordance with the invention may comprise an amino acid sequence as defined in SEQ ID NO: 2. The protein in accordance with the invention, in addition to comprising said substitutions of L234, L235 and G236 with F, E and R, within the hinge, CH2 and CH3 region sequence may comprise further substitutions therein. Preferably, the number of further substitutions is in the range of up to 5 additional substitutions within the hinge, CH2 and CH3 region of a human IgG1 immunoglobulin heavy chain. Hence, in one embodiment, a protein comprising a sequence as defined in SEQ ID NO: 2, comprises further substitutions in said sequence as defined by SEQ ID NO: 2, wherein the number of further substitutions consists of up to 5 substitutions. In another embodiment, a protein in accordance with the invention is provided comprising a sequence as defined in SEQ ID NO: 2, comprise further substitutions in said sequence as defined by SEQ ID NO: 2, wherein the number of further substitutions consists of up to 10 substitutions. In another embodiment, a protein in accordance with the invention may comprise a sequence as defined in SEQ ID NO: 1, or a corresponding sequence of another allotype of human IgG1, and be provided with said substitutions of L234, L235 and G236 with F, E and R, within the hinge, CH2 and CH3 region sequence thereof, and may further comprise substitutions as well, e.g. include up to 5 further substitutions. Examples of such a protein as comprising a polypeptide as defined in SEQ ID NO: 2 with further substitutions that are highly suitable having further substitutions, such as having the R/L substitutions as defined herein, are as defined in amino acid sequences as defined in SEQ ID NO: 11 and 12. Furthermore, said amino acid sequences as defined by SEQ ID NO: 2, or 11 and 12, having optional further substitutions as defined herein, may have the terminal lysine deleted.

Furthermore, provided is a protein in accordance with the invention, which may be an antibody or full-length antibody as defined herein, wherein both first and second polypeptides comprise an amino acid sequence as defined in SEQ ID NO: 2, 11, or 12.

In another embodiment, a protein, which may be a bispecific antibody, as defined herein is provided, wherein the first and second polypeptides comprise an amino acid sequence as defined in SEQ ID NO: 2 and 3, respectively. In another embodiment, a protein, which may be a bispecific antibody, as defined herein is provided, wherein the first and second polypeptides comprise an amino acid sequence as defined in SEQ ID NO: 11 and 12, respectively, or 11 and 14, or 12 and 13. Such polypeptides may comprise a CH1 region, adjacent to the hinge region, e.g. a human CH1 region as defined by SEQ ID NO: 4.

In a further embodiment, the protein, or monospecific or bispecific antibody, provided in accordance with the invention comprises an amino acid sequence which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 2, wherein the amino acid residues at the positions corresponding to 234, 235 and 236 as defined by Eu numbering are F, E and R, respectively.

In a further embodiment, the protein, or monospecific or bispecific antibody, provided in accordance with the invention comprises an amino acid sequence which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 11, wherein the amino acid residues at the positions corresponding to 234, 235 and 236 are F, E and R, respectively and the amino acid residue at the position corresponding to 405 is L; amino acid numbers being as defined by Eu numbering.

In a further embodiment, the protein, or monospecific or bispecific antibody, provided in accordance with the invention comprises an amino acid sequence which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 12, wherein the amino acid residues at the positions corresponding to 234, 235 and 236 are F, E and R, respectively and the amino acid residue at the position corresponding to 409 is R; amino acid numbers being as defined by Eu numbering.

In some embodiments, the protein, or bispecific antibody, provided in accordance with the invention comprises a first and second polypeptide, wherein
said first polypeptide comprises an amino acid sequence, which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 2, wherein the amino acid residues at the positions corresponding to 234, 235 and 236 are F, E and R, respectively,
and
said second polypeptide comprising an amino acid sequence, which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 3, wherein the amino acid residues at the positions corresponding to 234, 235 and 265 are F, E and A, respectively;
   amino acid numbers being as defined by Eu numbering.

In still further embodiments, the protein, or bispecific antibody, provided in accordance with the invention comprises a first and second polypeptide, wherein
said first polypeptide comprises an amino acid sequence, which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 11, wherein the amino acid residues at the positions corresponding to 234, 235 and 236 are F, E and R, respectively and the amino acid residue at the position corresponding to 405 is L,
and
said second polypeptide comprising an amino acid sequence, which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 12, wherein the amino acid residues at the positions corresponding to 234, 235 and 236 are F, E and R, respectively and the amino acid residue at the position corresponding to 409 is R;
   amino acid numbers being as defined by Eu numbering.

In still further embodiments, the protein, or bispecific antibody, provided in accordance with the invention comprises a first and second polypeptide, wherein
said first polypeptide comprises an amino acid sequence, which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 12, wherein the amino acid residues at the positions corresponding to 234, 235 and 236 are F, E and R, respectively and the amino acid residue at the position corresponding to 409 is R,
and
said second polypeptide comprising an amino acid sequence, which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 13, wherein the amino acid residues at the positions corresponding to 234, 235 and 236 are F, E and A, respectively and the amino acid residue at the position corresponding to 405 is L;
   amino acid numbers being as defined by Eu numbering.

In still further embodiments, the protein, or bispecific antibody, provided in accordance with the invention comprises a first and second polypeptide, wherein
said first polypeptide comprises an amino acid sequence, which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 11, wherein the amino acid residues at the positions corresponding to 234, 235 and 236 are F, E and R, respectively and the amino acid residue at the position corresponding to 405 is L,
and
said second polypeptide comprising an amino acid sequence, which has at least 85% sequence identity, such as at least 90% sequence identity, at least 95% sequence identity, at least 97% sequence identity, at least 98% sequence identity at least 99% sequence identity or has 100% sequence identity, to the sequence defined in SEQ ID NO: 14, wherein the amino acid residues at the positions corresponding to 234, 235 and 265 are F, E and A, respectively and the amino acid residue at the position corresponding to 409 is R;
   amino acid numbers being as defined by Eu numbering.

The sequence identified as SEQ ID NO: 2 herein comprises the hinge, CH2 and CH3 region of human IgG1 allotype G1m(f) with substitutions of L234, L235 and G236 with F, E and R. Corresponding sequences of constant regions (each including a CH1 region) of other allotypes of human IgG1 provided with said substitutions of L234, L235 and G236 with F, E and R, within the hinge, CH2 and CH3 region sequence thereof are provided as SEQ ID NO: 27 (CH1, hinge, CH2 and CH3 region of human IgG1 allotype G1m(fa) with FER substitutions), SEQ ID NO: 29 (CH1, hinge, CH2 and CH3 region of human IgG1 allotype G1m(za) with FER substitutions), SEQ ID NO: 31 (CH1, hinge, CH2 and CH3 region of human IgG1 allotype G1m(zav) with FER substitutions), SEQ ID NO: 33 (CH1, hinge, CH2 and CH3 region of human IgG1 allotype G1m(zax) with FER substitutions). In another embodiment, a nucleic acid is provided encoding said first or second polypeptide as defined herein, wherein both of said first and second polypeptides comprise said substitution of amino acids corresponding with amino acids L234, L235 and G236, most preferably wherein said substitutions of positions L234, L235 and G236 are with F, E and R, respectively. In another further embodiment, a nucleic acid is provided encoding said first or second polypeptide as defined herein, wherein said first or second polypeptide comprises said substitution of amino acids corresponding with amino acids L234, L235 and G236, preferably wherein said substitutions of positions L234, L235 and G236 are with F, E and R, respectively.

In one embodiment, a nucleic acid is provided encoding a first or second polypeptide, wherein said first or second polypeptide comprises an amino acid sequence as defined by SEQ ID NO: 2, 11, or 12. Such nucleic acids may further encode a polypeptide comprising a CH1 region, adjacent to the hinge region, e.g. a CH1 region as defined by SEQ ID NO: 4. Preferably, said nucleic acid encodes an immunoglobulin heavy chain. Such an immunoglobulin heavy chain comprising most preferably comprises a human or humanized immunoglobulin variable region. Such a nucleic acid encoding a first or second polypeptide, may have the terminal lysine deleted from the encoding sequence. These nucleic acids may be combined with a nucleic acid encoding an immunoglobulin light chain, or the like.

In one embodiment, a method for providing a construct for producing a protein in accordance with the invention with a non-activating Fc region is provided, wherein said protein comprises a first and second polypeptide with an Fc region comprising a hinge region, a CH2 and CH3 region, such as defined in SEQ ID NO: 1, or the like, said method comprising the steps of:
a) providing a construct, or constructs, for expression of a first and/or second polypeptide, comprising a nucleic acid sequence encoding a first and/or second polypeptide sequence comprising a hinge region, a CH2 region and CH3 region;
b) modifying said nucleic acid sequence encoding the amino acids corresponding with amino acids L234, L235 and G236 in the hinge region, CH2 region and CH3 region such that these encode F, E and R, respectively;
c) thereby providing a construct, constructs, for producing a protein with a non-activating Fc region.

In another embodiment, a method for providing a construct for producing a protein with a non-activating Fc region is provided, wherein said protein comprises a first and second polypeptide with an Fc region comprising a hinge region, a CH2 and CH3 region, such as defined in SEQ ID NO: 1, or the like, said method comprising the steps of:
a) providing a nucleic acid sequence encoding a hinge region, a CH2 region and CH3 region, such as defined in SEQ ID NO: 1;
b) modifying said nucleic acid sequence encoding the amino acids corresponding with amino acids L234, L235 and G236 in the hinge region, CH2 region and CH3 region such that these encode F, E and R, respectively;
c) generating a construct using the modified sequence obtained in step b) for expression of a first and/or second polypeptide of protein in accordance with the invention comprising a nucleic acid sequence encoding said first and/or second polypeptide comprising a hinge region, a CH2 region and CH3 region with amino acids L234, L235 and G236 in the hinge region, CH2 region and CH3 region modified to encode F, E and R, respectively, thereby providing a construct for producing a protein in accordance with the invention with a non-activating Fc region.

In a further embodiment, a method for providing a construct for producing an antibody with a non-activating Fc region is provided, wherein said antibody comprises a heavy chain with an Fc region comprising a hinge region, a CH2 and CH3 region, such as defined in SEQ ID NO: 1, or the like, said method comprising the steps of:
a) providing a construct for expression of a heavy chain of an antibody comprising a nucleic acid sequence encoding a heavy chain sequence of an antibody comprising a hinge region, a CH2 region and CH3 region;
b) modifying said nucleic acid sequence encoding the amino acids corresponding with amino acids L234, L235 and G236 in the hinge region, CH2 region and CH3 region such that these encode F, E and R, respectively;
c) thereby providing a construct for producing an antibody with a non-activating Fc region.

In yet another embodiment, a method for providing a construct for producing an antibody with a non-activating Fc region is provided, wherein said antibody comprises a heavy chain with an Fc region comprising a hinge region, a CH2 and CH3 region, such as defined in SEQ ID NO: 1, or the like, said method comprising the steps of:
a) providing a nucleic acid sequence encoding a hinge region, a CH2 region and CH3 region, such as defined in SEQ ID NO: 1;
b) modifying said nucleic acid sequence encoding the amino acids corresponding with amino acids L234, L235 and G236 in the hinge region, CH2 region and CH3 region such that these encode F, E and R, respectively;
c) generating a construct using the modified sequence obtained in step b) for expression of a heavy chain of an antibody comprising a nucleic acid sequence encoding a heavy chain sequence of an antibody comprising a hinge region, a CH2 region and CH3 region with amino acids L234, L235 and G236 in the hinge region, CH2 region and CH3 region modified to encode F, E and R, respectively, thereby providing a construct for producing an antibody with a non-activating Fc region.

It is understood that in the methods for providing the constructs above, the polypeptide or heavy chain with an Fc region comprising a hinge region, a CH2 and CH3 region, may be any such Fc region comprising a hinge region, a CH2 and CH3 region, such as defined herein in accordance with the invention. Such methods are in particularly useful for improving the safety profile or suppressing Fc-mediated effector function of an antibody by introducing the FER substitutions. Such methods are useful for improving the safety profile of a protein, antibody, or the like, by introducing the said FER substitutions. Such methods are also useful suppressing Fc-mediated effector function of a protein, antibody, or the like, by introducing the FER substitutions. Such methods are in particularly useful for improving the safety profile and suppressing Fc-mediated effector function of a protein, antibody, or the like, by introducing the FER substitutions. With said methods, one can provide nucleic acids with which to obtain proteins with an Fc region comprising a hinge region, a CH2 and CH3 region, in accordance with the invention.

Such nucleic acids are in particular useful for producing the proteins in accordance with the invention, such as for example, an antibody comprising heavy and light chains comprising a first and second polypeptide in accordance with the invention as defined herein. Such antibodies may be monospecific antibodies or bispecific antibodies. Hence, in another aspect, nucleic acids encoding said first or second polypeptides in accordance with the invention are provided, for use in expression vectors encoding the sequences of e.g. an antibody. Hence, host cells are provided comprising such expression vectors, including hybridomas which may produce antibodies, and to methods of producing such an antibody by culturing such host cells or hybridomas under appropriate conditions whereby a protein in accordance with the invention antibody, such as an antibody, is produced and, optionally, retrieved.

Hence, a host cell may be provided with a nucleic acid in accordance with the invention, wherein said nucleic acid is incorporated in an expression vector, such as described e.g. in the example section, or the like. An expression vector in the context of the present invention may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral or nonviral nucleic acid (RNA or DNA) vectors. In one embodiment, an antibody-encoding nucleic acid is comprised in a naked DNA or RNA vector, including, for example, a linear expression element (as described in for instance Sykes and Johnston, Nat Biotech 17, 355-S9 (1997)), a compacted nucleic acid vector (as described in for instance U.S. Pat. No. 6,077,835 and/or WO 00/70087), a plasmid vector such as pcDNA3.3 (as described herein), pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaPO₄⁻-precipitated construct (as described in for instance WO 00/46147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression in a bacterial cell. Examples of such vectors include expression vectors such as BlueScript (Stratagene), pIN vectors (Van Heeke & Schuster, J Biol Chem 264, 5503-5509 (1989)), pET vectors (Novagen, Madison WI) and the like. An expression vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

A nucleic acid and/or vector may also comprise a nucleic acid sequence encoding a secretion/localization sequence, which can target a polypeptide, such as a nascent polypeptide chain, to the periplasmic space or into cell culture media. Such sequences are known in the art, and include secretion leader or signal peptides, organelle-targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

In an expression vector of the invention, nucleic acids in accordance with the invention may comprise or be associated with any suitable promoter, enhancer, and other expression-facilitating elements. Examples of such elements include strong expression promoters (e.g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE (the skilled artisan will recognize that such terms are actually descriptors of a degree of gene expression under certain conditions).

A host cell comprising nucleic acid sequences encoding an antibody in accordance with the invention is hence provided, wherein said antibody comprises an immunoglobulin heavy chain comprising at least a hinge region, a CH2 region and a CH3 region, respectively of a human IgG1 immunoglobulin heavy chain, comprising substitutions of amino acids at positions L234, L235 and G236, with F, E and R, respectively, and an immunoglobulin light chain. Such a host cell may in particular useful for the manufacturing of a monospecific antibody, having two identical heavy chains, comprising said first and second polypeptides, and two identical light chains, such as described herein. In particular, when two of such antibodies are provided, wherein the sequences of the heavy chains are different and allow for exchange of the arms, such as described in the example section, such two antibodies can be highly advantageously used for the preparation of a bispecific antibody.

As said, it may not be required that both the first and second polypeptide of the bispecific antibody would have the same substitutions at positions L234, L235 and G236, with F, E and R. For example, one may have different substitutions. This allows e.g. for convenient manufacturing of bispecific antibodies, as one may not necessarily create each and every time e.g. a new construct and/or cell line for screening different combinations of antibodies from which a bispecific antibody may be generated and/or developed as a new product, thereby saving considerable time and effort.

Hence, in one embodiment, a method of preparing a bispecific antibody in accordance with the invention is provided, which method comprises:
a) providing a first antibody, comprising
   a. an immunoglobulin heavy chain comprising at least a hinge region, a CH2 region and a CH3 region, respectively of a human IgG1 immunoglobulin heavy chain, comprising substitutions of amino acids at positions L234, L235 and G236, with F, E and R, respectively,
   b. an immunoglobulin light chain;
b) providing a second antibody, comprising
   a. an immunoglobulin heavy chain comprising at least a hinge region, a CH2 region and a CH3 region, respectively, of a human IgG1 immunoglobulin heavy chain, comprising substitutions of amino acids at
      positions L234, L235, and D265, wherein preferably, said substitutions are F, E and A, respectively,
   or,
      comprising substitutions of amino acids at positions L234, L235 and G236, with F, E and R, respectively,
   b. an immunoglobulin light chain;
c) wherein the sequences of said first and second CH3 regions of said respective first and second antibodies are different and are such that the heterodimeric interaction between said first and second CH3 regions is stronger than each of the homodimeric interactions of said first and second CH3 regions;
d) incubating said first antibody together with said second antibody under reducing conditions sufficient to allow the cysteines in the hinge regions to undergo disulfide-bond isomerization; and
e) obtaining said bispecific antibody comprising said first immunoglobulin heavy chain and said first immunoglobulin light chain of said first antibody and said second immunoglobulin heavy chain and said second immunoglobulin light chain of said second antibody.
   In a further embodiment, instead of a bispecific antibody, multispecific antibodies can be produced.

It is understood that the first and second antibodies as provided in steps a) and b) are preferably monospecific antibodies. More preferably, said monospecific antibodies are full-length antibodies. Most preferably, said antibodies comprise human or humanized variable regions and have human constant regions, comprising substitutions as defined herein. Exemplary first and second antibodies that are highly suitable for the method above are a first and second antibody comprising an amino acid sequence as respectively defined in SEQ ID NO: 11 and 12; 11 and 14, or 12 and 13. Such a method of preparing a bispecific antibody is described i.a. in the examples herein and is also well described in (Labrijn et al., 2014, Nature Protocols October; 9(10):2450-63). Of course, other suitable differences may be contemplated in step c), such as described herein.

### Pharmaceutical Compositions

In one aspect, the invention provides a pharmaceutical composition comprising a protein, such as an antibody, as defined in any of the aspects and embodiments herein described, and a pharmaceutically acceptable carrier.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, P A, 1995.

The pharmaceutically acceptable carriers or diluents as well as any other known adjuvants and excipients should be suitable for the protein, variant or antibody of the present invention and the chosen mode of administration. Suitability for carriers and other components of pharmaceutical compositions is determined based on the lack of significant negative impact on the desired biological properties of the chosen compound or pharmaceutical composition of the present invention (e.g., less than a substantial impact (10% or less relative inhibition, 5% or less relative inhibition, etc.)) on antigen binding.

A pharmaceutical composition of the present invention may also include diluents, fillers, salts, buffers, detergents (e. g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

The actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical composition may be administered by any suitable route and mode. Suitable routes of administering a protein, variant or antibody of the present invention in vivo and in vitro are well known in the art and may be selected by those of ordinary skill in the art.

In one embodiment, a pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intra-arterial, intrathecal, intracapsular, intra-orbital, intracardiac, intradermal, intraperitoneal, intratendinous, transtracheal, subcutaneous, subcuticular, intra-articular, subcapsular, subarachnoid, intraspinal, intracranial, intrathoracic, epidural and intrasternal injection and infusion.

In one embodiment that pharmaceutical composition is administered by intravenous or subcutaneous injection or infusion.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

### Therapeutic Applications

In another aspect, the present invention relates to a protein, e.g. antibody, or pharmaceutical composition of the invention as defined in any aspect or embodiment herein described, for use as a medicament.

In another aspect, the present invention relates to a protein, e.g. an antibody, or pharmaceutical composition of the invention as defined in any aspect or embodiment herein described, for use in the treatment of a disease.

In a further aspect, said treatment of disease comprises the treatment of a cancer, an infectious disease, an inflammatory disease, or an autoimmune disease. In another aspect, the present invention relates to a use wherein the disease is cancer. It is understood that it is highly preferred that such use involves the use in humans.

In another aspect, the present invention relates to a method of treatment comprising administering a protein, e.g. antibody, or pharmaceutical composition of the invention as defined in any aspect or embodiment herein described, to a human subject.

In another aspect, the present invention relates to a method of treatment comprising administering a protein, e.g. an antibody, or pharmaceutical composition of the invention as defined in any aspect or embodiment herein described, to a human subject suffering from a disease. In a further aspect, said disease comprises a cancer, an inflammatory, an infectious disease or an autoimmune disease. In another aspect, said disease is cancer.

The protein, variant, antibody, or pharmaceutical composition of the invention can be used as in a treatment wherein immune effector functions of an antibody IgG1 Fc region as found in a wild-type antibody are not desired. For example, the protein, variant, or antibody may be administered to cells in culture, e.g., in vitro or ex vivo, or to human subjects, e.g. in vivo, to treat or prevent disorders such as cancer, infectious disease, inflammatory or autoimmune disorders. As used herein, the term “subject” is typically a human which responds to the protein, variant, antibody, or pharmaceutical composition. Subjects may for instance include human patients having disorders that may be corrected or ameliorated by modulating a target function or by leading to killing of the cell, directly or indirectly.

In another aspect, the present invention provides methods for treating or preventing a disorder, such as cancer, wherein recruitment of T-cells would contribute to the treatment or prevention, which method comprises administration of a therapeutically effective amount of a protein, variant, antibody, or pharmaceutical composition of the present invention to a subject in need thereof. For example, such a protein in accordance with the invention, would be capable of engaging cytotoxic T-cells, e.g. a bispecific antibody targeting CD3 and a cancer antigen. Cells overexpressing tumor-specific targets are particularly good targets for such a protein, variant or antibody of the invention, since recruitment of T-cells by one of the two binding regions of the protein, variant, or antibody can trigger a cytotoxic activity of the T-cells. This mechanism is normally difficult to obtain, as the triggering of a cytotoxic activity may not work properly in elimination of cancer cells.

In another aspect, the proteins, including antibodies and bispecific antibodies, in accordance with the invention such as described herein are conjugated to another molecule. Such protein may be produced by chemically conjugating the other molecule to the N-terminal side or C-terminal end of the protein, or antibody or fragment thereof (see, e.g., Antibody Engineering Handbook, edited by Osamu Kanemitsu, published by Chijin Shokan (1994)). Such conjugated antibody derivatives may also be generated by conjugation at internal residues or sugars, where appropriate. In a preferred aspect, the proteins, including antibodies and bispecific antibodies, in accordance with the invention are conjugated to a therapeutic molecule. Suitable therapeutic molecules may include e.g. nucleic acids, such as an aptamer, ribozyme, antisense molecule, or RNAi inducing agents. Other therapeutic molecules that may be contemplated include a cytotoxin, a chemotherapeutic drug, an immunosuppressant, or a radioisotope. Such conjugates can be referred to as immunoconjugates. Immunoconjugates which include one or more cytotoxins can be referred to as immunotoxins.

Suitable therapeutic molecules for forming immunoconjugates of the present invention can include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, glucocorticoids, procaine, tetracaine, lidocaine, propranolol, and puromycin, antimetabolites (such as methotrexate, 6 mercaptopurine, 6 thioguanine, cytarabine, fludarabin, 5 fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT I, SLT II, SLT IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP S), Momordica charantia inhibitor, curcin, crotin, Sapaonaria officinalis inhibitor, gelonin, mitogellin, restrictocin, phenomycin, or enomycin toxins.

The method typically involves administering to a subject a protein, variant, or antibody in an amount effective to treat or prevent the disorder.

The efficient dosages and dosage regimens for the protein, variant, or antibody depend on the disease or condition to be treated and may be determined by the persons skilled in the art.

For example, an “effective amount” for therapeutic use may be measured by its ability to stabilize the progression of disease. The ability of a compound to inhibit cancer may, for example, be evaluated in an animal model system predictive of efficacy in human tumors. Alternatively, this property of a composition may be evaluated by examining the ability of the protein, variant, or antibody to inhibit cell growth or to induce cytotoxicity by in vitro assays known to the skilled practitioner. A therapeutically effective amount of a therapeutic compound, i.e. a therapeutic protein, variant, antibody, or pharmaceutical composition according to the invention, may decrease tumor size, or otherwise ameliorate symptoms in a subject.

In one embodiment, the protein, antibody or variant may be administered by maintenance therapy, such as, e.g., at regular intervals for a defined period, or until disease progression. A protein, antibody or variant may also be administered prophylactically in order to reduce the risk of developing cancer, delay the onset of the occurrence of an event in cancer progression, and/or reduce the risk of recurrence when a cancer is in remission.

A protein, variant, antibody, or antibody may also be administered prophylactically in order to reduce the risk of developing cancer, delay the onset of the occurrence of an event in cancer progression, and/or reduce the risk of recurrence when a cancer is in remission.

### Diagnostic Applications

The non-activating protein of the invention may also be useful for diagnostic purposes, using a composition comprising a protein as described herein. Accordingly, the invention provides diagnostic methods and compositions using the proteins described herein. Such methods and compositions can be used for purely diagnostic purposes, such as detecting or identifying a disease, as well as for monitoring of the progress of therapeutic treatments, monitoring disease progression, assessing status after treatment, monitoring for recurrence of disease, evaluating risk of developing a disease, and the like. By using such a protein in accordance with the invention this allows e.g. for avoiding any unwanted effects exerted by an Fc region which may interfere in the diagnostic application.

In one aspect, the protein of the present invention is used ex vivo, such as in diagnosing a disease in which cells expressing a specific target of interest and to which the protein binds, are indicative of disease or involved in the pathogenesis, by detecting levels of the target or levels of cells which express the target of interest on their cell surface in a sample taken from a patient. This may be achieved, for example, by contacting the sample to be tested, optionally along with a control sample, with the protein according to the invention under conditions that allow for binding of the protein to the target. Complex formation can then be detected (e.g., using an ELISA). When using a control sample along with the test sample, the level of protein or protein-target complex is analyzed in both samples and a statistically significant higher level of protein or protein-target complex in the test sample indicates a higher level of the target in the test sample compared with the control sample.

Examples of conventional immunoassays in which proteins of the present invention can be used include, without limitation, ELISA, RIA, FACS assays, plasmon resonance assays, chromatographic assays, tissue immunohistochemistry, Western blot, and/or immunoprecipitation.

In one embodiment, the invention relates to a method for detecting the presence of a target, or a cell expressing the target, in a sample comprising:
contacting the sample with a protein of the invention under conditions that allow for binding of the protein to the target in the sample; and
analyzing whether a complex has been formed. Typically, the sample is a biological sample.

In one embodiment, the sample is a tissue sample known or suspected of containing a specific target and/or cells expressing the target. For example, in situ detection of the target expression may be accomplished by removing a histological specimen from a patient and providing the protein of the present invention to such a specimen. The protein may be provided by applying or by overlaying the protein to the specimen, which is then detected using suitable means. It is then possible to determine not only the presence of the target or target-expressing cells, but also the distribution of the target or target-expressing cells in the examined tissue (e.g., in the context of assessing the spread of cancer cells). Using the present invention, those of ordinary skill will readily perceive that any of a wide variety of histological methods (such as staining procedures) may be modified in order to achieve such in situ detection.

In the above assays, the protein can be labeled with a detectable substance to allow bound protein to be detected. Alternatively, bound (primary) specific protein may be detected by an antibody which is labeled with a detectable substance, and which binds to the primary specific protein.

The level of target in a sample can also be estimated by a competition immunoassay utilizing target standards labeled with a detectable substance and an unlabeled target-specific protein. In this type of assay, the biological sample, the labeled target standard(s) and the target-specific protein are combined, and the amount of labeled target standard bound to the unlabeled target-specific protein is determined. The amount of target in the biological sample is inversely proportional to the amount of labeled target standard bound to the target-specific protein.

Suitable labels for the target-specific protein, secondary antibody and/or target standard used in in vitro diagnostic techniques include, without limitation, various enzymes, prosthetic groups, fluorescent materials, luminescent materials, and radioactive materials. Examples of suitable enzymes include horseradish peroxidase, alkaline phosphatase, βgalactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; and examples of suitable radioactive material include ¹²⁵I, ¹³¹I, ³⁵S, and ³H.

In one embodiment, the present invention provides an in vivo imaging method wherein a target-specific protein of the present invention is conjugated to a detection-promoting radio-opaque agent, the conjugated protein is administered to a host, such as by injection into the bloodstream, and the presence and location of the labeled protein in the host is assayed. Through this technique and any other diagnostic method provided herein, the present invention provides a method for screening for the presence of disease-related cells in a human patient or a biological sample taken from a human patient and/or for assessing the distribution of target-specific protein prior to target-specific ADC therapy.

For diagnostic imaging, radioisotopes may be bound to a targets-pecific-protein either directly or indirectly by using an intermediary functional group. Useful intermediary functional groups include chelators, such as ethylenediaminetetraacetic acid and diethylenetriaminepentaacetic acid (see for instance U.S. Pat. No. 5,057,313).

In addition to radioisotopes and radio-opaque agents, diagnostic methods may be performed using target-specific proteins that are conjugated to dyes (such as with the biotin-streptavidin complex), contrast agents, fluorescent compounds or molecules and enhancing agents (e.g. paramagnetic ions) for magnetic resonance imaging (MRI) (see, e.g., U.S. Pat. No. 6,331,175, which describes MRI techniques and the preparation of proteins conjugated to a MRI enhancing agent). Such diagnostic/detection agents may be selected from agents for use in MRI, and fluorescent compounds. Thus, the present invention provides a diagnostic target-specific protein, wherein the target-specific protein is conjugated to a contrast agent (such as for magnetic resonance imaging, computed tomography, or ultrasound contrast-enhancing agent) or a radionuclide that may be, for example, a gamma-, beta-, alpha-, Auger electron-, or positron-emitting isotope.

In a further aspect, the invention relates to a kit for detecting the presence of target antigen or a cell expressing the target, in a sample, comprising:
a target-specific antibody of the invention; and
instructions for use of the kit.

In one embodiment, the present invention provides a kit for diagnosis of cancer comprising a container comprising a target-specific protein, and one or more reagents for detecting binding of the target-specific protein to the target. Reagents may include, for example, fluorescent tags, enzymatic tags, or other detectable tags. The reagents may also include secondary or tertiary antibodies or reagents for enzymatic reactions, wherein the enzymatic reactions produce a product that may be visualized. In one embodiment, the present invention provides a diagnostic kit comprising one or more target-specific proteins of the present invention in labeled or unlabeled form in suitable container(s), reagents for the incubations for an indirect assay, and substrates or derivatizing agents for detection in such an assay, depending on the nature of the label. Control reagent(s) and instructions for use also may be included.

Diagnostic kits may also be supplied for use with a target-specific protein, such as a labeled target-specific protein, for the detection of the presence of the target in a tissue sample or host. In such diagnostic kits, as well as in kits for therapeutic uses described elsewhere herein, a target-specific protein typically may be provided in a lyophilized form in a container, either alone or in conjunction with additional antibodies specific for a target cell or peptide. Typically, a pharmaceutically acceptable carrier (e.g., an inert diluent) and/or components thereof, such as a Tris, phosphate, or carbonate buffer, stabilizers, preservatives, biocides, inert proteins, e.g., serum albumin, or the like, also are included (typically in a separate container for mixing) and additional reagents (also typically in separate container(s)). In certain kits, a secondary antibody capable of binding to the target-specific protein, which typically is present in a separate container, is also included. The second antibody is typically conjugated to a label and formulated in a manner similar to the target-specific protein of the present invention. Using the methods described above and elsewhere herein, target—specific proteins may be used to define subsets of cancer/tumor cells and characterize such cells and related tumor tissues.

TABLE 1
SEQUENCE LISTING

| SEQ ID | | |
|---|---|---|
| NO: | description | sequence |
| 1 | hinge, CH2 and CH3 | EPKSCDKTHTCPPCPAPELLGGPSVFLFPPKPKDTLMISRTPEVTC |
| | region of human IgG1 | VVVDVSHEDPEVKFNWYVDGVEVHNAKTKPREEQYNSTYRVVSV |
| | (allotype G1m(f)) | LTVLHQDWLNGKEYKCKVSNKALPAPIEKTISKAKGQPREPQVYT |
| | | LPPSREEMTKNQVSLTCLVKGFYPSDIAVEWESNGQPENNYKTTP |
| | | PVLDSDGSFFLYSKLTVDKSRWQQGNVFSCSVMHEALHNHYTQK |
| | | SLSLSPGK |
| 2 | Hinge, CH2 and CH3 | EPKSCDKTHTCPPCPAPEFERGPSVFLFPPKPKDTLMISRTPEVTC |
| | region of human IgG1 | VVVDVSHEDPEVKFNWYVDGVEVHNAKTKPREEQYNSTYRVVSV |
| | (allotype G1m(f)) with | LTVLHQDWLNGKEYKCKVSNKALPAPIEKTISKAKGQPREPQVYT |
| | FER substitution (bold) | LPPSREEMTKNQVSLTCLVKGFYPSDIAVEWESNGQPENNYKTTP |
| | | PVLDSDGSFFLYSKLTVDKSRWQQGNVFSCSVMHEALHNHYTQK |
| | | SLSLSPGK |
| 3 | Hinge, CH2 and CH3 | EPKSCDKTHTCPPCPAPEFEGGPSVFLFPPKPKDTLMISRTPEVTC |
| | region of human IgG1 | VVVAVSHEDPEVKFNWYVDGVEVHNAKTKPREEQYNSTYRVVSV |
| | (allotype G1m(f)) with | LTVLHQDWLNGKEYKCKVSNKALPAPIEKTISKAKGQPREPQVYT |
| | FEA substitution (bold) | LPPSREEMTKNQVSLTCLVKGFYPSDIAVEWESNGQPENNYKTTP |
| | | PVLDSDGSFFLYSKLTVDKSRWQQGNVFSCSVMHEALHNHYTQK |
| | | SLSLSPGK |
| 4 | CH1 region of human | ASTKGPSVFPLAPSSKSTSGGTAALGCLVKDYFPEPVTVSWNSGA |
| | IgG1 (allotype G1m(f)) | LTSGVHTFPAVLQSSGLYSLSSVVTVPSSSLGTQTYICNVNHKPS |
| | | NTKVDKRV |
| 5 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 (allotype G1m(f)) | NTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKALPAPIEKTISKAKGQ |
| | | PREPQVYTLPPSREEMTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 6 | constant region of | RTVAAPSVFIFPPSDEQLKSGTASVVCLLNNFYPREAKVQWKVDN |
| | human kappa light | ALQSGNSQESVTEQDSKDSTYSLSSTLTLSKADYEKHKVYACEVT |
| | chain | HQGLSSPVTKSFNRGEC |
| 7 | constant region of | GQPKAAPSVTLFPPSSEELQANKATLVCLISDFYPGAVTVAWKAD |
| | human lambda light | SSPVKAGVETTTPSKQSNNKYAASSYLSLTPEQWKSHRSYSCQV |
| | chain | THEGSTVEKTVAPTECS |
| 8 | Hinge, CH2 and CH3 | ESKYGPPCPSCPAPEFLGGPSVFLFPPKPKDTLMISRTPEVTCVVV |
| | region of constant | DVSQEDPEVQFNWYVDGVEVHNAKTKPREEQFNSTYRVVSVLTV |
| | region of human IgG4 | LHQDWLNGKEYKCKVSNKGLPSSIEKTISKAKGQPREPQVYTLPP |
| | | SQEEMTKNQVSLTCLVKGFYPSDIAVEWESNGQPENNYKTTPPVL |
| | | DSDGSFFLYSRLTVDKSRWQEGNVFSCSVMHEALHNHYTQKSLS |
| | | LSLGK |
| 9 | Hinge, CH2 and CH3 | EPKSCDKTHTCPPCPAPELLGGPSVFLFPPKPKDTLMISRTPEVTC |
| | region of human | VVVDVSHEDPEVKFNWYVDGVEVHNAKTKPREEQYNSTYRVVSV |
| | IgG1(allotype G1m(f)) | LTVLHQDWLNGKEYKCKVSNKALPAPIEKTISKAKGQPREPQVYT |
| | with F405L substitution | LPPSREEMTKNQVSLTCLVKGFYPSDIAVEWESNGQPENNYKTTP |
| | (bold) | PVLDSDGSFLLYSKLTVDKSRWQQGNVFSCSVMHEALHNHYTQ |
| | | KSLSLSPGK |
| 10 | Hinge, CH2 and CH3 | EPKSCDKTHTCPPCPAPELLGGPSVFLFPPKPKDTLMISRTPEVTC |
| | region of human IgG1 | VVVDVSHEDPEVKFNWYVDGVEVHNAKTKPREEQYNSTYRVVSV |
| | (allotype G1m(f)) with | LTVLHQDWLNGKEYKCKVSNKALPAPIEKTISKAKGQPREPQVYT |
| | K409R substitution | LPPSREEMTKNQVSLTCLVKGFYPSDIAVEWESNGQPENNYKTTP |
| | (bold) | PVLDSDGSFFLYSRLTVDKSRWQQGNVFSCSVMHEALHNHYTQ |
| | | KSLSLSPGK |
| 11 | Hinge, CH2 and CH3 | EPKSCDKTHTCPPCPAPEFERGPSVFLFPPKPKDTLMISRTPEVTC |
| | region of human IgG1 | VVVDVSHEDPEVKFNWYVDGVEVHNAKTKPREEQYNSTYRVVSV |
| | (allotype G1m(f)) with | LTVLHQDWLNGKEYKCKVSNKALPAPIEKTISKAKGQPREPQVYT |
| | FER substitution and | LPPSREEMTKNQVSLTCLVKGFYPSDIAVEWESNGQPENNYKTTP |
| | with F405L substitution | PVLDSDGSFLLYSKLTVDKSRWQQGNVFSCSVMHEALHNHYTQ |
| | (bold) | KSLSLSPGK |
| 12 | Hinge, CH2 and CH3 | EPKSCDKTHTCPPCPAPEFERGPSVFLFPPKPKDTLMISRTPEVTC |
| | region of human IgG1 | VVVDVSHEDPEVKFNWYVDGVEVHNAKTKPREEQYNSTYRVVSV |
| | (allotype G1m(f)) with | LTVLHQDWLNGKEYKCKVSNKALPAPIEKTISKAKGQPREPQVYT |
| | FER substitution and | LPPSREEMTKNQVSLTCLVKGFYPSDIAVEWESNGQPENNYKTTP |
| | with K409R substitution | PVLDSDGSFFLYSRLTVDKSRWQQGNVFSCSVMHEALHNHYTQ |
| | (bold) | KSLSLSPGK |
| 13 | Hinge, CH2 and CH3 | EPKSCDKTHTCPPCPAPEFEGGPSVFLFPPKPKDTLMISRTPEVTC |
| | region of human IgG1 | VVVAVSHEDPEVKFNWYVDGVEVHNAKTKPREEQYNSTYRVVSV |
| | (allotype G1m(f)) with | LTVLHQDWLNGKEYKCKVSNKALPAPIEKTISKAKGQPREPQVYT |
| | FEA substitution and | LPPSREEMTKNQVSLTCLVKGFYPSDIAVEWESNGQPENNYKTTP |
| | with F405L substitution | PVLDSDGSFLLYSKLTVDKSRWQQGNVFSCSVMHEALHNHYTQ |
| | (bold) | KSLSLSPGK |
| 14 | Hinge, CH2 and CH3 | EPKSCDKTHTCPPCPAPEFEGGPSVFLFPPKPKDTLMISRTPEVTC |
| | region of human IgG1 | VVVAVSHEDPEVKFNWYVDGVEVHNAKTKPREEQYNSTYRVVSV |
| | (allotype G1m(f)) with | LTVLHQDWLNGKEYKCKVSNKALPAPIEKTISKAKGQPREPQVYT |
| | FEA substitution and | LPPSREEMTKNQVSLTCLVKGFYPSDIAVEWESNGQPENNYKTTP |
| | with K409R substitution | PVLDSDGSFFLYSRLTVDKSRWQQGNVFSCSVMHEALHNHYTQ |
| | (bold) | KSLSLSPGK |
| 15 | FCGR1AECD-HisBAP | MWFLTTLLLWVPVDGQVDTTKAVITLQPPWVSVFQEETVTLHCEV |
| | | LHLPGSSSTQWFLNGTATQTSTPSYRITSASVNDSGEYRCQRGLS |
| | | GRSDPIQLEIHRGWLLLQVSSRVFTEGEPLALRCHAWKDKLVYNV |
| | | LYYRNGKAFKFFHWNSNLTILKTNISHNGTYHCSGMGKHRYTSA |
| | | GISVTVKELFPAPVLNASVTSPLLEGNLVTLSCETKLLLQRPGLQLY |
| | | FSFYMGSKTLRGRNTSSEYQILTARREDSGLYWCEAATEDGNVLK |
| | | RSPELELQVLGLQLPTPVWFHPGSSSHHHHHHPGGGLNDIFEAQ |
| | | KIEWHE |
| 16 | diFCGR2AH-HisBAP | METQMSQNVCPRNLWLLQPLTVLLLLASADSQAAAPPKAVLKLEP |
| | | PWINVLQEDSVTLTCQGARSPESDSIQWFHNGNLIPTHTQPSYRF |
| | | KANNNDSGEYTCQTGQTSLSDPVHLTVLSEWLVLQTPHLEFQEG |
| | | ETIMLRCHSWKDKPLVKVTFFQNGKSQKFSHLDPTFSIPQANHSH |
| | | SGDYHCTGNIGYTLFSSKPVTITVQVPSMGSSSPVAPPKAVLKLEP |
| | | PWINVLQEDSVTLTCQGARSPESDSIQWFHNGNLIPTHTQPSYRF |
| | | KANNNDSGEYTCQTGQTSLSDPVHLTVLSEWLVLQTPHLEFQEG |
| | | ETIMLRCHSWKDKPLVKVTFFQNGKSQKFSHLDPTFSIPQANHSH |
| | | SGDYHCTGNIGYTLFSSKPVTITVQVPSMGPGSSSHHHHHHPGG |
| | | GLNDIFEAQKIEWHE |
| 17 | diFCGR2AR-HisBAP | MVLSLLYLLTALPGILSAAPPKAVLKLEPPWINVLQEDSVTLTCQGA |
| | | RSPESDSIQWFHNGNLIPTHTQPSYRFKANNNDSGEYTCQTGQT |
| | | SLSDPVHLTVLSEWLVLQTPHLEFQEGETIMLRCHSWKDKPLVKV |
| | | TFFQNGKSQKFSRLDPTFSIPQANHSHSGDYHCTGNIGYTLFSSK |
| | | PVTITVQVPSMGSSSPAAPPKAVLKLEPPWINVLQEDSVTLTCQG |
| | | ARSPESDSIQWFHNGNLIPTHTQPSYRFKANNNDSGEYTCQTGQ |
| | | TSLSDPVHLTVLSEWLVLQTPHLEFQEGETIMLRCHSWKDKPLVK |
| | | VTFFQNGKSQKFSRLDPTFSIPQANHSHSGDYHCTGNIGYTLFSS |
| | | KPVTITVQVPSMGSSSPGSSSHHHHHHPGGGLNDIFEAQKIEWH |
| | | E |
| 18 | diFCGR2B-HisBAP | MVLSLLYLLTALPGILSAAPPKAVLKLEPQWINVLQEDSVTLTCRG |
| | | THSPESDSIQWFHNGNLIPTHTQPSYRFKANNNDSGEYTCQTGQ |
| | | TSLSDPVHLTVLSEWLVLQTPHLEFQEGETIVLRCHSWKDKPLVK |
| | | VTFFQNGKSKKFSRSDPNFSIPQANHSHSGDYHCTGNIGYTLYSS |
| | | KPVTITVQAPSSSPMGPAAPPKAVLKLEPQWINVLQEDSVTLTCR |
| | | GTHSPESDSIQWFHNGNLIPTHTQPSYRFKANNNDSGEYTCQTG |
| | | QTSLSDPVHLTVLSEWLVLQTPHLEFQEGETIVLRCHSWKDKPLV |
| | | KVTFFQNGKSKKFSRSDPNFSIPQANHSHSGDYHCTGNIGYTLYS |
| | | SKPVTITVQAPSSSPMGPGSSSHHHHHHPGGGLNDIFEAQKIEW |
| | | HE |
| 19 | diFCGR3AF-HisBAP | MVLSLLYLLTALPGISTEDLPKAVVFLEPQWYRVLEKDSVTLKCQG |
| | | AYSPEDNSTQWFHNESLISSQASSYFIDAATVDDSGEYRCQTNL |
| | | STLSDPVQLEVHIGWLLLQAPRWVFKEEDPIHLRCHSWKNTALH |
| | | KVTYLQNGKGRKYFHHNSDFYIPKATLKDSGSYFCRGLFGSKNVS |
| | | SETVNITITQGPSMGSSSPSEDLPKAVVFLEPQWYRVLEKDSVTL |
| | | KCQGAYSPEDNSTQWFHNESLISSQASSYFIDAATVDDSGEYRC |
| | | QTNLSTLSDPVQLEVHIGWLLLQAPRWVFKEEDPIHLRCHSWKN |
| | | TALHKVTYLQNGKGRKYFHHNSDFYIPKATLKDSGSYFCRGLFGS |
| | | KNVSSETVNITITQGPSMGSSSPGPGSSSHHHHHHPGGGLNDIF |
| | | EAQKIEWHE |
| 20 | diFCGR3AV-HisBAP | MVLSLLYLLTALPGISTEDLPKAVVFLEPQWYRVLEKDSVTLKCQG |
| | | AYSPEDNSTQWFHNESLISSQASSYFIDAATVDDSGEYRCQTNL |
| | | STLSDPVQLEVHIGWLLLQAPRWVFKEEDPIHLRCHSWKNTALH |
| | | KVTYLQNGKGRKYFHHNSDFYIPKATLKDSGSYFCRGLVGSKNV |
| | | SSETVNITITQGPSMGSSSPSEDLPKAVVFLEPQWYRVLEKDSVT |
| | | LKCQGAYSPEDNSTQWFHNESLISSQASSYFIDAATVDDSGEYR |
| | | CQTNLSTLSDPVQLEVHIGWLLLQAPRWVFKEEDPIHLRCHSWK |
| | | NTALHKVTYLQNGKGRKYFHHNSDFYIPKATLKDSGSYFCRGLVG |
| | | SKNVSSETVNITITQGPSMGSSSPGPGSSSHHHHHHPGGGLNDI |
| | | FEAQKIEWHE |
| 21 | FcRnECDHisBAP | MGVPRPQPWALGLLLFLLPGSLGAESHLSLLYHLTAVSSPAPGTPA |
| | | FWVSGWLGPQQYLSYNSLRGEAEPCGAWVWENQVSWYWEKET |
| | | TDLRIKEKLFLEAFKALGGKGPYTLQGLLGCELGPDNTSVPTAKFA |
| | | LNGEEFMNFDLKQGTWGGDWPEALAISQRWQQQDKAANKELTF |
| | | LLFSCPHRLREHLERGRGNLEWKEPPSMRLKARPSSPGFSVLTCS |
| | | AFSFYPPELQLRFLRNGLAAGTGQGDFGPNSDGSFHASSSLTVKS |
| | | GDEHHYCCIVQHAGLAQPLRVELESPAKSSPGSSSHHHHHHPGG |
| | | GLNDIFEAQKIEWHE |
| 22 | B2M | MSRSVALAVLALLSLSGLEAIQRTPKIQVYSRHPAENGKSNFLNCY |
| | | VSGFHPSDIEVDLLKNGERIEKVEHSDLSFSKDWSFYLLYYTEFTP |
| | | TEKDEYACRVNHVTLSQPKIVKWDRDM |
| 23 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(fa) | NTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 24 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(za) | NTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 25 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(zax) | NTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEG |
| | | LHNHYTQKSLSLSPGK |
| 26 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(zav) | NTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYN |
| | | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNIFSCSVMHEA |
| | | LHNHYTQKSLSLSPGK |
| 27 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(fa) | NTKVDKRVEPKSCDKTHTCPPCPAPEFERGPSVFLFPPKPKDTLMI |
| | with FER substitution | SRTPEVTCVVVDVSHEDPEVKFNWYVDGVEVHNAKTKPREEQYN |
| | (bold) | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 28 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(fa) | NTKVDKRVEPKSCDKTHTCPPCPAPEFEGGPSVFLFPPKPKDTLMI |
| | with FEA substitution | SRTPEVTCVVVAVSHEDPEVKFNWYVDGVEVHNAKTKPREEQYN |
| | (bold) | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 29 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(za) | NTKVDKKVEPKSCDKTHTCPPCPAPEFERGPSVFLFPPKPKDTLMI |
| | with FER substitution | SRTPEVTCVVVDVSHEDPEVKFNWYVDGVEVHNAKTKPREEQYN |
| | (bold) | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 30 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(za) | NTKVDKKVEPKSCDKTHTCPPCPAPEFEGGPSVFLFPPKPKDTLMI |
| | with FEA substitution | SRTPEVTCVVVAVSHEDPEVKFNWYVDGVEVHNAKTKPREEQYN |
| | (bold) | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPGK |
| 31 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(zav) | NTKVDKKVEPKSCDKTHTCPPCPAPEFERGPSVFLFPPKPKDTLMI |
| | with FER substitution | SRTPEVTCVVVDVSHEDPEVKFNWYVDGVEVHNAKTKPREEQYN |
| | (bold) | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNIFSCSVMHEA |
| | | LHNHYTQKSLSLSPGK |
| 32 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(zav) | NTKVDKKVEPKSCDKTHTCPPCPAPEFEGGPSVFLFPPKPKDTLMI |
| | with FEA substitution | SRTPEVTCVVVAVSHEDPEVKFNWYVDGVEVHNAKTKPREEQYN |
| | (bold) | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNIFSCSVMHEA |
| | | LHNHYTQKSLSLSPGK |
| 33 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(zax) | NTKVDKKVEPKSCDKTHTCPPCPAPEFERGPSVFLFPPKPKDTLMI |
| | with FER substitution | SRTPEVTCVVVDVSHEDPEVKFNWYVDGVEVHNAKTKPREEQYN |
| | (bold) | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEG |
| | | LHNHYTQKSLSLSPGK |
| 34 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(zax) | NTKVDKKVEPKSCDKTHTCPPCPAPEFEGGPSVFLFPPKPKDTLMI |
| | with FEA substitution | SRTPEVTCVVVAVSHEDPEVKFNWYVDGVEVHNAKTKPREEQYN |
| | (bold) | STYRVVSVLTVLHQDWLNGKEYKCKVSNKALPAPIEKTISKAKGQ |
| | | PREPQVYTLPPSRDELTKNQVSLTCLVKGFYPSDIAVEWESNGQP |
| | | ENNYKTTPPVLDSDGSFFLYSKLTVDKSRWQQGNVFSCSVMHEG |
| | | LHNHYTQKSLSLSPGK |
| 35 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(f) | NTKVDKRVEPKSCDKTHTCPPCPAPELLGGPSVFLFPPKPKDTLMI |
| | with deletion of K447 | SRTPEVTCVVVDVSHEDPEVKFNWYVDGVEVHNAKTKPREEQYN |
| | | STYRVVSVLTVLHQDWLNGKEYKCKVSNKALPAPIEKTISKAKGQ |
| | | PREPQVYTLPPSREEMTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPG |
| 36 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(f) | NTKVDKRVEPKSCDKTHTCPPCPAPEFERGPSVFLFPPKPKDTLMI |
| | with deletion of K447 | SRTPEVTCVVVDVSHEDPEVKFNWYVDGVEVHNAKTKPREEQYN |
| | and with FER | STYRVVSVLTVLHQDWLNGKEYKCKVSNKALPAPIEKTISKAKGQ |
| | substitution (bold) | PREPQVYTLPPSREEMTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPG |
| 37 | CH1, hinge, CH2 and | ASTKGPSVFPLAPSSKSTSGGTAALGCLVKDYFPEPVTVSWNSGA |
| | CH3 region of human | LTSGVHTFPAVLQSSGLYSLSSVVTVPSSSLGTQTYICNVNHKPS |
| | IgG1 allotype G1m(f) | NTKVDKRVEPKSCDKTHTCPPCPAPEFEGGPSVFLFPPKPKDTLMI |
| | with deletion of K447 | SRTPEVTCVVVAVSHEDPEVKFNWYVDGVEVHNAKTKPREEQYN |
| | and with FEA | STYRVVSVLTVLHQDWLNGKEYKCKVSNKALPAPIEKTISKAKGQ |
| | substitution (bold) | PREPQVYTLPPSREEMTKNQVSLTCLVKGFYPSDIAVEWESNGQP |
| | | ENNYKTTPPVLDSDGSFFLYSKLTVDKSRWQQGNVFSCSVMHEA |
| | | LHNHYTQKSLSLSPG |
| 38 | CH1, hinge, CH2 and | AKTTAPSVYPLAPVCGDTTGSSVTLGCLVKGYFPEPVTLTWNSGS |
| | CH3 region of murine | LSSGVHTFPAVLQSDLYTLSSSVTVTSSTWPSQSITCNVAHPASS |
| | IgG2a | TKVDKKIEPRGPTIKPCPPCKCPAPNLLGGPSVFIFPPKIKDVLMIS |
| | | LSPMVTCVVVDVSEDDPDVQISWFVNNVEVLTAQTQTHREDYNS |
| | | TLRVVSALPIQHQDWMSGKEFKCKVNNKALPAPIERTISKPKGSV |
| | | RAPQVYVLPPPEEEMTKKQVTLTCMVTDFMPEDIYVEWTNNGKTE |
| | | LNYKNTEPVLDSDGSYFMYSKLRVEKKNWVERNSYSCSVVHEGL |
| | | HNHHTTKSFSRTPGK |
| 39 | CH1, hinge, CH2 and | AKTTAPSVYPLAPVCGDTTGSSVTLGCLVKGYFPEPVTLTWNSGS |
| | CH3 region of murine | LSSGVHTFPAVLQSDLYTLSSSVTVTSSTWPSQSITCNVAHPASS |
| | IgG2a with FER | TKVDKKIEPRGPTIKPCPPCKCPAPNFERGPSVFIFPPKIKDVLMIS |
| | substitution (bold) | LSPMVTCVVVDVSEDDPDVQISWFVNNVEVLTAQTQTHREDYNS |
| | | TLRVVSALPIQHQDWMSGKEFKCKVNNKALPAPIERTISKPKGSV |
| | | RAPQVYVLPPPEEEMTKKQVTLTCMVTDFMPEDIYVEWTNNGKTE |
| | | LNYKNTEPVLDSDGSYFMYSKLRVEKKNWVERNSYSCSVVHEGL |
| | | HNHHTTKSFSRTPGK |
| 40 | CH1, hinge, CH2 and | ASTKGPSVFPLAPCSRSTSGGTAALGCLVKDYFPEPVTVSWNSGA |
| | CH3 region of IgG3 | LTSGVHTFPAVLQSSGLYSLSSVVTVPSSSLGTQTYTCNVNHKPS |
| | allotype IGHG3*01 | NTKVDKRVELKTPLGDTTHTCPRCPEPKSCDTPPPCPRCPEPKSC |
| | | DTPPPCPRCPEPKSCDTPPPCPRCPAPELLGGPSVFLFPPKPKDTL |
| | | MISRTPEVTCVVVDVSHEDPEVQFKWYVDGVEVHNAKTKPREEQ |
| | | YNSTFRVVSVLTVLHQDWLNGKEYKCKVSNKALPAPIEKTISKTK |
| | | GQPREPQVYTLPPSREEMTKNQVSLTCLVKGFYPSDIAVEWESSG |
| | | QPENNYNTTPPMLDSDGSFFLYSKLTVDKSRWQQGNIFSCSVMH |
| | | EALHNRFTQKSLSLSPGK |
| 41 | CH1, hinge, CH2 and | ASTKGPSVFPLAPCSRSTSGGTAALGCLVKDYFPEPVTVSWNSGA |
| | CH3 region of IgG3 | LTSGVHTFPAVLQSSGLYSLSSVVTVPSSSLGTQTYTCNVNHKPS |
| | allotype IGHG3*04 | NTKVDKRVELKTPLGDTTHTCPRCPEPKSCDTPPPCPRCPAPELLG |
| | | GPSVFLFPPKPKDTLMISRTPEVTCVVVDVSHEDPEVQFKWYVDG |
| | | VEVHNAKTKPREEQYNSTFRVVSVLTVLHQDWLNGKEYKCKVSN |
| | | KALPAPIEKTISKTKGQPREPQVYTLPPSREEMTKNQVSLTCLVKG |
| | | FYPSDIAVEWESSGQPENNYNTTPPMLDSDGSFFLYSKLTVDKSR |
| | | WQQGNIFSCSVMHEALHNRFTQKSLSLSPGK |
| 42 | CH1, hinge, CH2 and | ASTKGPSVFPLAPCSRSTSGGTAALGCLVKDYFPEPVTVSWNSGA |
| | CH3 region of IgG3 | LTSGVHTFPAVLQSSGLYSLSSVVTVPSSSLGTQTYTCNVNHKPS |
| | allotype IGHG3*01 with | NTKVDKRVELKTPLGDTTHTCPRCPEPKSCDTPPPCPRCPEPKSC |
| | FER substitution (bold) | DTPPPCPRCPEPKSCDTPPPCPRCPAPEFERGPSVFLFPPKPKDTL |
| | | MISRTPEVTCVVVDVSHEDPEVQFKWYVDGVEVHNAKTKPREEQ |
| | | YNSTFRVVSVLTVLHQDWLNGKEYKCKVSNKALPAPIEKTISKTK |
| | | GQPREPQVYTLPPSREEMTKNQVSLTCLVKGFYPSDIAVEWESSG |
| | | QPENNYNTTPPMLDSDGSFFLYSKLTVDKSRWQQGNIFSCSVMH |
| | | EALHNRFTQKSLSLSPGK |
| 43 | CH1, hinge, CH2 and | ASTKGPSVFPLAPCSRSTSGGTAALGCLVKDYFPEPVTVSWNSGA |
| | CH3 region of IgG3 | LTSGVHTFPAVLQSSGLYSLSSVVTVPSSSLGTQTYTCNVNHKPS |
| | allotype IGHG3*01 with | NTKVDKRVELKTPLGDTTHTCPRCPEPKSCDTPPPCPRCPEPKSC |
| | FEA substitution (bold) | DTPPPCPRCPEPKSCDTPPPCPRCPAPEFEGGPSVFLFPPKPKDTL |
| | | MISRTPEVTCVVVAVSHEDPEVQFKWYVDGVEVHNAKTKPREEQ |
| | | YNSTFRVVSVLTVLHQDWLNGKEYKCKVSNKALPAPIEKTISKTK |
| | | GQPREPQVYTLPPSREEMTKNQVSLTCLVKGFYPSDIAVEWESSG |
| | | QPENNYNTTPPMLDSDGSFFLYSKLTVDKSRWQQGNIFSCSVMH |
| | | EALHNRFTQKSLSLSPGK |
| 44 | CH1, hinge, CH2 and | ASTKGPSVFPLAPCSRSTSGGTAALGCLVKDYFPEPVTVSWNSGA |
| | CH3 region of IgG3 | LTSGVHTFPAVLQSSGLYSLSSVVTVPSSSLGTQTYTCNVNHKPS |
| | allotype IGHG3*04 with | NTKVDKRVELKTPLGDTTHTCPRCPEPKSCDTPPPCPRCPAPEFE |
| | FER substitution (bold) | RGPSVFLFPPKPKDTLMISRTPEVTCVVVDVSHEDPEVQFKWYVD |
| | | GVEVHNAKTKPREEQYNSTFRVVSVLTVLHQDWLNGKEYKCKVS |
| | | NKALPAPIEKTISKTKGQPREPQVYTLPPSREEMTKNQVSLTCLVK |
| | | GFYPSDIAVEWESSGQPENNYNTTPPMLDSDGSFFLYSKLTVDKS |
| | | RWQQGNIFSCSVMHEALHNRFTQKSLSLSPGK |
| 45 | CH1, hinge, CH2 and | ASTKGPSVFPLAPCSRSTSGGTAALGCLVKDYFPEPVTVSWNSGA |
| | CH3 region of IgG3 | LTSGVHTFPAVLQSSGLYSLSSVVTVPSSSLGTQTYTCNVNHKPS |
| | allotype IGHG3*04 with | NTKVDKRVELKTPLGDTTHTCPRCPEPKSCDTPPPCPRCPAPEFE |
| | FEA substitution (bold) | GGPSVFLFPPKPKDTLMISRTPEVTCVVVAVSHEDPEVQFKWYVD |
| | | GVEVHNAKTKPREEQYNSTFRVVSVLTVLHQDWLNGKEYKCKVS |
| | | NKALPAPIEKTISKTKGQPREPQVYTLPPSREEMTKNQVSLTCLVK |
| | | GFYPSDIAVEWESSGQPENNYNTTPPMLDSDGSFFLYSKLTVDKS |
| | | RWQQGNIFSCSVMHEALHNRFTQKSLSLSPGK |
| 46 | CH1, hinge, CH2 and | ASTKGPSVFPLAPCSRSTSESTAALGCLVKDYFPEPVTVSWNSGA |
| | CH3 region of constant | LTSGVHTFPAVLQSSGLYSLSSVVTVPSSSLGTKTYTCNVDHKPS |
| | region of human IgG4 | NTKVDKRVESKYGPPCPSCPAPEFLGGPSVFLFPPKPKDTLMISRT |
| | | PEVTCVVVDVSQEDPEVQFNWYVDGVEVHNAKTKPREEQFNSTY |
| | | RVVSVLTVLHQDWLNGKEYKCKVSNKGLPSSIEKTISKAKGQPRE |
| | | PQVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLGK |
| 47 | CH1, hinge, CH2 and | ASTKGPSVFPLAPCSRSTSESTAALGCLVKDYFPEPVTVSWNSGA |
| | CH3 region of human | LTSGVHTFPAVLQSSGLYSLSSVVTVPSSSLGTKTYTCNVDHKPS |
| | IgG4 with L235E- | NTKVDKRVESKYGPPCPSCPAPEFERGPSVFLFPPKPKDTLMISRT |
| | G236R substitution | PEVTCVVVDVSQEDPEVQFNWYVDGVEVHNAKTKPREEQFNSTY |
| | (bold) | RVVSVLTVLHQDWLNGKEYKCKVSNKGLPSSIEKTISKAKGQPRE |
| | | PQVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLGK |
| 48 | CH1, hinge, CH2 and | ASTKGPSVFPLAPCSRSTSESTAALGCLVKDYFPEPVTVSWNSGA |
| | CH3 region of constant | LTSGVHTFPAVLQSSGLYSLSSVVTVPSSSLGTKTYTCNVDHKPS |
| | region of human IgG4 | NTKVDKRVESKYGPPCPSCPAPEFEGGPSVFLFPPKPKDTLMISRT |
| | with L235E-D265A | PEVTCVVVAVSQEDPEVQFNWYVDGVEVHNAKTKPREEQFNSTY |
| | substitution (bold) | RVVSVLTVLHQDWLNGKEYKCKVSNKGLPSSIEKTISKAKGQPRE |
| | | PQVYTLPPSQEEMTKNQVSLTCLVKGFYPSDIAVEWESNGQPENN |
| | | YKTTPPVLDSDGSFFLYSRLTVDKSRWQEGNVFSCSVMHEALHN |
| | | HYTQKSLSLSLGK |
| 49 | Constant region of | RADAAPTVSIFPPSSEQLTSGGASVVCFLNNFYPKDINVKWKIDG |
| | murine kappa light | SERQNGVLNSWTDQDSKDSTYSMSSTLTLTKDEYERHNSYTCEA |
| | chain | THKTSTSPIVKSFNRNEC |

### EXAMPLES

### Introduction

When utilizing the FEA inert format (L234F-L235E-D265A) in an antibody of the IgG1 isotype, which, as a wild-type IgG1 is very efficient in inducing complement-mediated cytotoxicity (CDC), CDC activity is strongly suppressed. However, in exploratory experiments, IgG1 antibodies harboring FEA substitutions were shown to occasionally still retain low residual CDC activity, which may be undesirable when adverse effects or dose-limiting toxicity of an envisioned therapeutic antibody can be highly contingent on Fc-mediated effector functions such as CDC. Moreover, we also observed that glycosylation heterogeneity was increased in antibodies harboring the FEA inert format, as well as an increase in galactosylation and the presence of charged glycans, as compared to a wild-type like format. Changes in the glycosylation profile may have an effect on efficacy and pharmacokinetic properties, and this needs to be monitored and controlled in manufacturing. Upon evaluation of the glycan profile of antibodies containing the FEA inert format, we observed that increased glycosylation heterogeneity, increased levels of galactosylation, and increased levels of charged glycans could be assigned to the D265A substitution. In view of possible residual CDC activity of FEA, and in view of the glycosylation profile of FEA, we sought to provide for an improved format.

In seeking for improvements, it was contemplated to combine the L234F, the L235E and the G236R substitutions. However, combining any mutations, let alone any selection of specific mutations, is inherently unpredictable. Moreover, combining the L234F, L235E, and G236R mutation, i.e. having multiple modifications packed close together, is not straightforward as the structural and/or functional impact of such a multitude of changes cannot be predicted. We therefore tested in a pilot experiment what the effect of the combination L234F, L235E and G236R (FER) was on its ability to silence CDC as well as on the glycosylation profile of antibodies. Surprisingly, these initial experiments indicated that the ability to silence CDC by introduction of the FER mutations was highly improved as compared with individual substitutions, and as compared with the FEA substitutions. Moreover, evaluation of the glycosylation profile revealed that antibodies harboring the FER inert format now were provided with a glycan profile similar to corresponding wild-type like antibodies. Hence, these initial experiments prompted us to fully analyze the clinical suitability of the FER inert format, which is described in the examples below. Surprisingly, on all features tested, highly advantageous properties for the FER inert format were observed when compared with the FEA inert format as well as in comparison with other inert formats. This indicates that the new FER format is well suitable for clinical development and clinical use. This non-activating format, which may be useful in contexts other than antibodies as well, such as e.g. fusion proteins, can be regarded to be highly advantageous and a best-in-class non-activating format.

### Example 1: Production and Purification of Antibody Variants and Generation of Bispecific Antibody Variants

Expression Constructs for Antibodies

For the expression of antibodies with human, or humanized binding domains, antibody constructs comprising sequences of variable heavy (VH) chain and variable light (VL) chain domains were prepared by de novo gene synthesis (GeneArt Gene Synthesis; ThermoFisher Scientific, Germany) and cloned into pcDNA3.3 expression vectors (ThermoFisher Scientific, US) containing a human IgG1 heavy chain constant region (i.e. CH1, hinge, CH2 and CH3 region) of the human IgG1m(f) allotype (also referred to as IgG1 throughout the examples section of this application; SEQ ID NO: 5), or human IgG1m(fa) allotype (SEQ ID NO: 23), or human IgG1m(za) allotype (SEQ ID NO: 24), or human IgG1m(zax) allotype (SEQ ID NO: 25), or human IgG1m(zav) allotype (SEQ ID NO: 26), or human IgG1m(f) allotype with recombinant deletion of the C-terminal Lysine (also referred to as IgG1-delK; SEQ ID NO: 35), or variants thereof; a human IgG4 heavy chain constant region (hinge, CH2, CH3, SEQ ID NO: 8; CH1, hinge, CH2, CH3, SEQ ID NO: 46) or variant thereof; a human IgG3 heavy chain constant region of the human IgG3 allotype IGHG3*01 (SEQ ID NO: 40) or human IgG3 allotype IGHG3*04 (also referred to as IgG3rch2; SEQ ID NO: 41), or variants thereof; a murine IgG2a heavy chain constant region (SEQ ID NO: 38) or variant thereof; or the constant region of the human kappa light chain (LC; SEQ ID NO: 6), or human lambda LC (SEQ ID NO: 7), or murine kappa LC (SEQ ID NO: 49), as appropriate for the selected binding domains. Desired substitutions to generate variants were introduced by gene synthesis and are described below. CD20 antibody variants in this application have VH and VL sequences derived from a type I anti-human CD20 antibody previously described (Engelberts et al., 2020). HLA-DR antibody variants in this application have VH and VL sequences derived from previously described HLA-DR antibodies IgG1-HLA-DR-4 (U.S. Pat. No. 6,894,149 B2) and IgG1-HLA-DR-1D09C3 (U.S. Pat. No. 7,521,047 B2). CD3 antibodies in this application, as well as variants thereof, have VH and VL sequences derived from CD3 antibody previously described (i.e., CD3-huCLB-T3/4: Labrijn et al., PNAS, 2013 Mar. 26; 110(13):5145-S0, and Engelberts et al., 2020). HER2 antibody variants in this application comprise VH and VL sequences derived from IgG1-HER2-1014-169 (WO2012143524). A human IgG1 antibody comprising the VH/VL of b12, an HIV1 gp120-specific antibody, was used as a negative control in some experiments (Barbas et al., J Mol Biol. 1993 Apr. 5; 230(3):812-2).

Non-Activating Antibody Variants

Wild-type human IgG1 heavy chain constant regions (i.e., hinge, CH2 and CH3 region) or wild-type-like variants thereof harboring an F405L mutation (SEQ ID NO: 9) or K409R mutation (SEQ ID NO: 10) are used in control antibodies, as indicated. In some examples wild-type antibody variants with constant regions of IgG4 (SEQ ID NO: 8) or IgG4 variants thereof harboring a S228P mutation in SEQ ID NO: 8 are used as controls.

A non-activating Fc domain prevents antibodies from interacting with Fc-receptors present on immune cells, or with C1q, to activate the classical complement pathway. As described herein in the examples below, several non-activating antibody variants were generated and tested for the capacity to silence Fc function. In some examples, a subset of non-activating antibody variants was tested for Pharmacokinetic properties, Immunogenicity, or Developability.

Various non-activating antibody variants were generated with the different VH and VL sequences as indicated and described above. The following substitutions, wherein the amino acids are as defined by Eu numbering, were introduced in an IgG1m(f) HC region of SEQ ID NO: 1, also in combination with either F405L or K409R mutations, to allow generation of bispecific non-activating antibody variants: L234F-L235E-D265A (i.a. U.S. Ser. No. 10/590,206B2) alone (SEQ ID NO: 3), or in combination with either F405L (SEQ ID NO: 13) or K409R (SEQ ID NO: 14), L234F-L235E-G236R alone (SEQ ID NO: 2), or in combination with either F405L (SEQ ID NO: 11) or K409R (SEQ ID NO: 12), L234A-L235A-P329G (Schlothauer et al., 2016, Protein Eng. Design and Selection) alone, or in combination with either F405L or K409R, G236R-L328R (US2006/0235208 A1, Moore et al., 2011, MAbs) in combination with either F405L or K409R, E233P-L234V-L235A-delG236-S267K (Moore at al., 2019, Methods) in combination with either F405L or K409R, N297G (Tao and Morrison, 1989, JI) in combination with either F405L or K409R, and L234A-L235E-G237A-A330S-P331S (US8613926k) in combination with either F405L or K409R. The following mutations were introduced in an IgG1m(f) HC region with recombinant deletion of the HC C-terminal lysine (also referred to as IgG1-delK) of SEQ ID NO: 35: L234F-L235E-G236R (SEQ ID NO: 36), and L234F-L235E-D265A (SEQ ID NO: 37). The following mutations were introduced in an IgG1m(fa) HC region of SEQ ID NO: 23: L234F-L235E-G236R (SEQ ID NO: 27), and L234F-L235E-D265A (SEQ ID NO: 28). The following mutations were introduced in an IgG1m(za) HC region of SEQ ID NO: 24: L234F-L235E-G236R (SEQ ID NO: 29), and L234F-L235E-D265A (SEQ ID NO: 30). The following mutations were introduced in an IgG1m(zax) HC region of SEQ ID NO: 25: L234F-L235E-G236R (SEQ ID NO: 33), and L234F-L235E-D265A (SEQ ID NO: 34). The following mutations were introduced in an IgG1m(zav) HC region of SEQ ID NO: 26: L234F-L235E-G236R (SEQ ID NO: 31), and L234F-L235E-D265A (SEQ ID NO: 32). The following mutations were introduced in an IgG3 (IGHG3*01) HC region of SEQ ID NO: 40: L234F-L235E-G236R (SEQ ID NO: 42), and L234F-L235E-D265A (SEQ ID NO: 43). The following mutations were introduced in an IgG3 (IGHG3*04; also referred to as IgG3rch2) HC region of SEQ ID NO: 41: L234F-L235E-G236R (SEQ ID NO: 44), and L234F-L235E-D265A (SEQ ID NO: 45). The following mutations were introduced in an IgG4 HC region of SEQ ID NO: 8, alone or in combination with F405L-R409K mutations, to allow generation of bispecific non-activating antibody variants: 5228P-E233P-F234V-L235A-delG236 (WO2015/143079) or in combination with F405L-R409K, 5228P-F234A-L235A (Allegre et al., Transplantation, 1994, Jun. 15; 57(11):1537-43 and Angal et al., Mol Immunol, 1993, January; 30(1):105-8) or in combination with F405L-R409K, L235E-G236R (SEQ ID NO: 47), and L235E-D265A (SEQ ID NO: 48). The following mutations were introduced in a murine IgG2a HC region of SEQ ID NO: 38: L234F-L235E-G236R (SEQ ID NO: 39), L234A-L235A (Arduin et al., Mol Immunol, 2015 February; 63(2):456-63), and L234A-L235A-P329G (Lo et al., J Biol Chem, 2017 Mar. 3; 292(9):3900-3908).

Transient Expression

Antibodies were expressed as human IgG1κ, IgG1λ, IgG3κ, IgG4κ, or murine IgG2aκ. Plasmid DNA mixtures that encode both the heavy and light chains of antibodies were transiently transfected in Expi293F™ cells (Thermo Fisher Scientific, Cat #A14527) using ExpiFectamine™ (Thermo Fisher Scientific, Cat #A14525) according to manufacturer's instructions. In short, both DNA (1:1 HC/LC ratio) and ExpiFectamine are separately diluted in Opti-MEM I (Thermo Fisher Scientific, Cat #51985034) to 20 μg/ml and 5.4% (v/v) respectively. Both solutions are incubated for 5 minutes, mixed and incubated for 10-20 minutes before being added to 3×10⁶ Expi293F cells/ml in fresh Opti-MEM supplemented with pen/strep (final DNA concentration 1 μg/ml). Approximately 16-24h post transfection, ExpiFectamine 293 Transfection Enhancer I (0.5 (v/v) %) and II (5 (v/v) %) are added. Supernatant is typically harvested 5 days post transfection. Antibody concentrations in the supernatants were measured by absorbance at 280 nm. Antibodies were purified as described below.

### Antibody Purification and Quality Assessment

Antibodies were purified by Protein A affinity chromatography. Culture supernatants were filtered over a 0.20 μM dead-end filter and loaded on 5 mL MabSelect SuRe columns (GE Healthcare/Cytiva), washed with 0.02 M sodium citrate-NaOH pH 5.0 and eluted with 0.02 M sodium citrate-NaOH, pH 3. The eluates were dialyzed against PBS (8.65 mM Na₂HPO₄ anhydrous, 1.9 mM NaH₂PO₄ monohydrate, 140.3 mM NaCl, pH 7.4 buffer prepared for Genmab by HyClone/Cytiva). When required, proteins were further purified on preparative size-exclusion-chromatography (SEC) to remove aggregates. After buffer exchange or SEC, samples were sterile filtered over 0.2 μm dead-end filters. The quality of the purified proteins was analyzed by mass spectrometry, capillary electrophoresis (non-reduced and reduced CE-SDS) and high-performance size exclusion chromatography (HP-SEC). Concentration was measured by absorbance at 280 nm. Purified antibodies were stored at 2-8° C.

### Generation of Bispecific Antibodies

IgG1 bispecific antibody was generated by controlled Fab-Arm Exchange (cFAE) between a monospecific antibody harboring a F405L mutation in the CH3 domain and a monospecific antibody harboring a K409R mutation in the CH3 domain in addition to non-activating mutations as listed above, essentially as reported previously (Labrijn et al., 2014, Nature Protocols October; 9(10):2450-63). For IgG4 bispecific variants, one monospecific antibody was bearing a natural Arginine (R) on position 409 and the other monospecific antibody harboring F405L-R409K mutations in the CH3 domain of the antibody. Briefly, both monospecific antibodies were mixed at equimolar concentration and incubated with 75 mM 2-mercaptoethylamine-HCl (2-MEA) at 31° C. for 5 hours. Subsequently 2-MEA was removed by buffer-exchanging against PBS using Slide-A-Lyzer Dialysis Cassettes (10K MWCO; ThermoFisher Scientific) over night at 4° C. Dialysis buffer was changed two times. Generated bispecific antibodies were collected from cassettes and concentration was measured by absorbance at 280 nm. Purified bispecific antibodies were stored at 2-8° C. and were typically analyzed by CE-SDS and HP-SEC to assess the monomeric state of the generated bispecific antibodies and by mass spectrometric analysis using an Orbitrap Q-Exactive Plus mass spectrometer (ThermoFischer Scientific) to determine the efficiency of the cFAE and the bispecific antibody content.

### Generation of F(ab′)2 Fragments

F(ab′)2 fragments targeting HLA-DR were generated using FraglT kit (Genovis AB) essentially according to the manufacturer's recommendations. Briefly, spin columns with FraglT, a resin with the FabRICATOR enzyme that digests IgG at a specific site below the hinge region generating a homogenous pool of F(ab′)2 and Fc/2 fragments, coupled to agarose beads, are equilibrated with digestion buffer (Genovis AB). Subsequently, the anti-human HLA-DR antibody variant IgG1-HLA-DR-1D09C3 with E430G mutation was applied to the column and incubated 15 minutes at room temperature (RT). After incubation, the samples are eluted and collected from the columns. Purified F(ab′)2 fragments were analyzed by capillary electrophoresis on sodium dodecyl sulfate-polyacrylamide gels (CE-SDS). Concentration was measured by absorbance at 280 nm. Purified antibodies were stored at 2-8° C.

### Example 2: Target Binding of Anti-Human CD20 Antibodies and Non-Activating Variants Thereof on Raji Cells

Target binding of anti-human CD20 IgG1 antibody with wild-type Fc and variants harboring K409R, L234F-L235E-D265A-K409R, L234F-L235E-G236R-K409R, L234A-L235A-P329G-K409R, G236R-L328R-K409R, E233P-L234V-L235A-G236del-5267K-K409R, N297G-K409R, L234A-L235E-G237A-A330S-P331S-K409R, IgG4 antibody with wild-type Fc and variants harboring S228P, 5228P-F234A-L235A, or S228P-E233P-F234V-L235A-G236del was assessed using Raji cells expressing CD20. Anti-HIV1 gp120 IgG1 wild-type antibody (IgG1-b12) was used as a non-binding control.

For this, Raji cells (3×10 4 cells, Cat #CCL-86, ATCC) in FACS buffer (1×PBS, Cat #BE17-517Q, Lonza; 0.1% bovine serum albumin, BSA, Cat #10735086001, Merck; 0.02% NaN₃, Cat #41920044-3, Bio-world) were incubated in polystyrene round-bottom 96-well plates (Cat #650180, Greiner bio-one) with a concentration series of purified antibodies targeting CD20 (0.0013-20 μg/mL final concentrations; 5-fold dilutions) in a total volume of 50 μL for 30 minutes at 4° C. Subsequently, cells were washed twice by addition of 150 μL FACS buffer followed by pelleting the cells by centrifugation and removing the supernatant. Bound antibodies were stained by addition of 50 μL of goat F(ab′)2 anti-human kappa-PE (final concentration 2.5 μg/mL, Cat #2062-09, Southern Biotech) for 30 minutes at 4° C. Subsequently, cells were washed twice by addition of 150 μL FACS buffer followed by pelleting the cells by centrifugation and removing the supernatant. Binding of the antibody variants to human CD20 was detected by flow cytometry on an Intellicyt iQue screener (Sartorius), by measuring the Median Fluorescence Intensity. The data were analyzed using a non-linear agonist dose-response model in GraphPad PRISM (version 8.4.1, GraphPad Software). Data are mean values ±SEM obtained from four independent experiments.

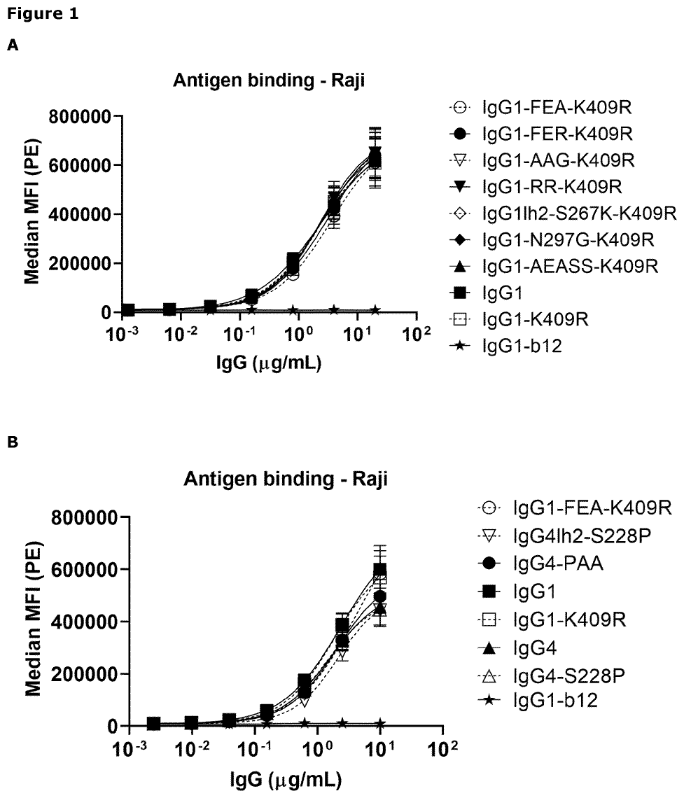

Assessment of target binding of the anti-human CD20 IgG1 and IgG4 antibody variants revealed similar target binding for all anti-human CD20 IgG1 antibody variants harboring mutations in the heavy chain constant region (FIG. 1A-B). All anti-human CD20 IgG4 antibody variants, including those harboring 5228P-F234A-L235A or 5228P-E233P-F234V-L235A-G236del non-activating mutations in the heavy chain constant region, showed somewhat decreased target binding compared to IgG1 antibody variants.

In summary, introduction of non-activating mutations in the heavy chain of anti-human CD20 IgG1- and IgG4 antibodies did not impact their ability to bind their target CD20.

### Example 3: Complement-Dependent Cytotoxicity by Anti-Human CD20 Antibodies and Non-Activating Variants Thereof

For therapeutic monoclonal antibodies where the therapeutic effect is independent of the interaction with the immune system's effector molecules (such as complement protein C1q and Fcγ receptors) and the adverse effects or dose-limiting toxicity is highly dependent on this interaction, a non-activating Fc domain could be considered to increase the therapeutic window. Engagement of an antibody with C1q protein initiates the classical complement pathway leading to Complement-Dependent Cytotoxicity (CDC). Here, the capacity to induce CDC was assessed for type I anti-human CD20 antibodies, chosen for their efficient and potent induction of CDC (Glennie et al., Mol Immunology, 2007, September; 44(16):3823-37), as well as for variants of such antibodies harboring non-activating mutations in the constant heavy chain region.

Anti-human CD20 IgG1 antibodies, either as wild-type, or harboring the K409R mutation, or non-activating variants thereof harboring L234F-L235E-D265A-K409R, L234F-L235E-G236R-K409R, L234A-L235A-P329G-K409R, G236R-L328R-K409R, E233P-L234V-L235A-G236del-5267K-K409R, N297G-K409R, L234A-L235E-G237A-A330S-P331S-K409R mutations, as well as anti-human CD20 IgG4 antibodies, either as wild-type, harboring mutation S228P, and non-activating variants harboring 5228P-F234A-L235A, or 5228P-E233P-F234V-L235A-G236del mutations were tested in a range of concentrations (0.0024-10 μg/mL final concentrations; 4-fold dilutions) in an in vitro CDC assay on Raji cells with 20% normal human serum (NHS, Cat #M0008, Sanquin) as the source of complement. Raji cells (3×10⁴ cells per well) in RPMI-1640 medium (Cat #BE12-115F, Lonza) containing 0.1% (w/v) bovine serum albumin (BSA, Cat #10735086001, Merck) and penicillin-streptomycin (Pen/Strep, final concentration 50 units/mL potassium penicillin and 50 μg/mL streptomycin sulfate, Cat #DE17-603E, Lonza) were incubated in polystyrene round-bottom 96-well plates (Cat #650180, Greiner bio-one) with a concentration series of purified antibodies targeting CD20 in a total volume of 80 μL for 15 min at room temperature (RT). Subsequently, 20 μL NHS (final concentration 20% (v/v)) was added and the cells were incubated at 37° C. for 45 minutes. The reaction was stopped by placing the plates on ice before pelleting the cells by centrifugation and replacing the supernatant by 30 μL of 2 μg/mL propidium iodide (PI, Cat #P4170, Sigma Aldrich) in PBS (Cat #SH3A3830.03, GE Healthcare). The number of PI-positive cells was determined by flow cytometry on an Intellicyt iQue screener (Sartorius). The percentage of PI-positive cells, which corresponds to the percentage of cell lysis, was calculated as (number of PI-positive cells/total number of cells)×100%. The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control as baseline, followed by normalization per experimental replicate to the AUC value measured for the non-binding control antibody IgG1-b12 (0%) and the AUC value measured for the wild-type IgG1 antibody variant (Anti-human CD20 IgG1, 100%). Data are mean values ±SEM obtained from three independent experiments.

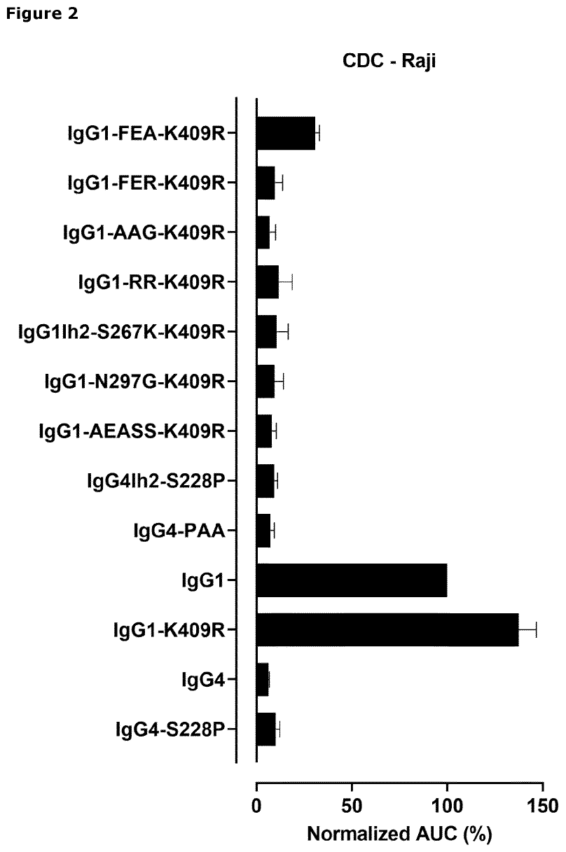

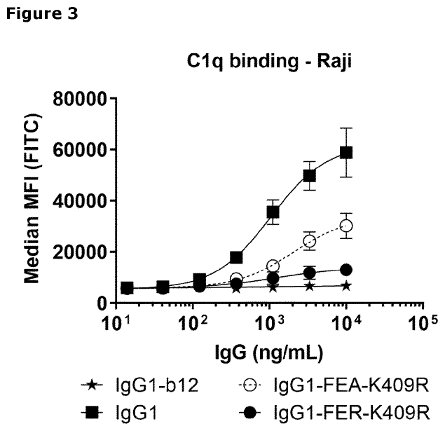

Assessment of CDC capacity of anti-human CD20 IgG1 and IgG4 variants revealed that the capacity to eliminate CDC by introducing non-activating mutations in the heavy chain constant region can be divided into two groups. For anti-human CD20 IgG1 antibody variants harboring L234F-L235E-G236R, L234A-L235A-P329G, G236R-L328R, E233P-L234V-L235A-G236del-S267K, N297G, and L234A-L235E-G237A-A330S-P331S non-activating mutations in addition to a K409R mutation, as well as IgG4 antibody variants harboring 5228P-F234A-L235A, or S228P-E233P-F234V-L235A-G236del non-activating mutations, the capacity to induce CDC was eliminated. However, compared to wild-type IgG1, residual CDC was observed for an anti-human CD20 IgG1 variant harboring mutations L234F-L235E-D265A-K409R (FIG. 2).

In conclusion, an IgG1 antibody with the novel variant L234F-L235E-G236R and K409R mutation showed almost complete absence of activation of the classical complement pathway. Similar effects have been observed with an antibody having only the novel L234F-L235E-G236R mutations and not having a K409R mutation. This represents a significant advancement over i.a. an IgG1 antibody variant harboring L234F-L235E-D265A and K409R mutations, for which residual CDC is observed.

### Example 4: Evaluation of C1q Binding to Anti-Human CD20 Antibodies and Non-Activating Variants Thereof

In Example 3, it was shown that the potency to induce CDC was strongly reduced by anti-human CD20 IgG1 and IgG4 antibody variants harboring mutations that suppress Fc-mediated effector functions. Here, binding of complement protein C1q to anti-human CD20 IgG1 antibody variants harboring non-activating mutations in the constant heavy chain region was assessed using Raji cells expressing CD20.

A C1q binding assay on Raji cells with 20% normal human serum (NHS, M0008, Sanquin) as the source of C1q was used and anti-human CD20 antibodies and non-activating variants thereof were tested in a range of concentrations (0.014-10 μg/mL final concentrations; 3-fold dilutions). Raji cells (1×10⁵ cells per well) in RPMI-1640 medium (Cat #BE12-115F, Lonza) containing 0.1% (w/v) bovine serum albumin (BSA, Cat #10735086001, Merck) and penicillin-streptomycin (Pen/Strep, final concentration 50 units/mL potassium penicillin and 50 μg/mL streptomycin sulfate, Cat #DE17-603E, Lonza) were incubated in polystyrene round-bottom 96-well plates (Cat #650180, Greiner bio-one) with a concentration series of purified antibodies targeting CD20 in a total volume of 80 μL at 37° C. for 15 min. To prevent activation of CDC upon addition of NHS, the cells were cooled by placing the plates on ice. Subsequently, 20 μL NHS (final concentration 20% (v/v)) was added and the cells were incubated at 4° C. for 45 min, followed by pelleting the cells by centrifugation and removing the supernatant. Next, cells were washed twice by addition of 150 μL FACS buffer (lx PBS, Cat #BE17-S17Q, Lonza; 0.1% bovine serum albumin, BSA; 0.02% NaN₃, Cat #41920044-3, Bio-world) followed by pelleting the cells by centrifugation and removing the supernatant. Bound C1q was stained by addition of 50 μL of polyclonal rabbit anti-human C1q Complement-FITC (final concentration 20 μg/mL, Dako, Cat #F0254, Agilent Technologies) for 30 min at 4° C. Subsequently, cells were washed twice by addition of 150 μL FACS buffer followed by pelleting the cells by centrifugation and removing the supernatant. C1q binding to the antibody variants was detected by flow cytometry on an Intellicyt iQue screener (Sartorius) by measuring Median Fluorescence Intensity-FITC. The data were analyzed using a non-linear agonist dose-response model in GraphPad PRISM (version 8.4.1, GraphPad Software). Data are mean values (±SD) obtained from triplicate measurements of a single experiment.

Assessment of binding of C1q to anti-human CD20 IgG1 variants revealed that anti-human CD20 IgG1 with a K409R mutation efficiently engages with the complement protein C1q upon binding to CD20 on target Raji cells, whereas the anti-HIV1 gp120 antibody (IgG1-b12; CD20 non-binding control) did not engage with C1q. Introduction of either the L234F-L235E-G236R or L234F-L235E-D265A non-activating mutations dramatically decreased C1q binding (FIG. 3). Binding of C1q is more strongly decreased to the anti-human CD20 IgG1 variant harboring the L234F-L235E-G236R mutations, compared to the anti-human CD20 IgG1 variant harboring the L234F-L235E-D265A mutations. This is in line with the effect on activation of CDC of these non-activating mutations, as presented in Example 3.

In conclusion, the non-activating anti-human CD20 IgG1 variant harboring the L234F-L235E-G236R non-activating mutations shows strongly decreased C1q binding, in line with the observed decrease in CDC.

### Example 5: Complement-Dependent Cytotoxicity by Anti-Human HLA-DR Antibodies and Non-Activating Variants Thereof

In Example 3, activation of the classical complement pathway by anti-human CD20 antibody variants harboring non-activating mutations in the constant heavy chain was assessed using an in vitro CDC assay. Data in Example 3 revealed that the anti-human CD20 IgG1 variant harboring the novel L234F-L235E-G236R and K409R mutations prevented activation of the classical complement pathway. In this Example, we further assessed the CDC capacity of non-activating antibody variants using two antibodies targeting human HLA-DR, which are potent inducers of CDC when used with an IgG1 backbone.

An in vitro CDC assay was performed, essentially as described in Example 3, on Raji cells with 20% NHS as the source of complement. Briefly, the anti-human HLA-DR antibodies IgG1-HLA-DR-4 and IgG1-HLA-DR-1D09C3 harboring a K409R mutation or variants thereof harboring non-activating mutations L234F-L235E-D265A, or L234F-L235E-G236R in combination with K409R in the constant heavy chain region, as well as an HLA-DR-targeting F(ab′)2 fragment were tested in a range of concentrations (0.014-10 μg/mL final concentrations; 3-fold dilutions). The number of PI-positive cells, as a measure for cell lysis, was determined by flow cytometry on an Intellicyt iQue screener (Sartorius). The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control as baseline, followed by normalization per experimental replicate to the AUC value measured for the wild-type IgG1 antibody variant (100%). Data are based on five (wild-type and L234F-L235E-D265A-K409R variants) or two (L234F-L235E-G236R-K409R variants, or the F(ab′)2 fragment) independent experiments.

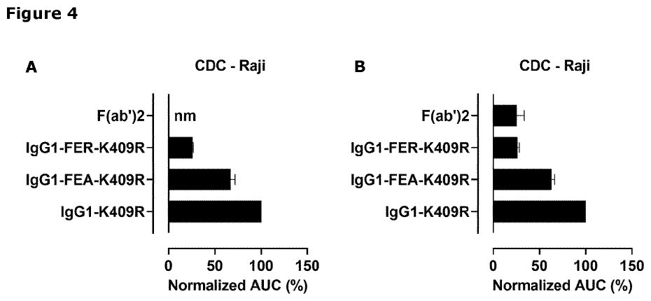

Assessment of the CDC capacity of anti-human HLA-DR antibody variants revealed that antibody variants harboring L234F-L235E-D265A mutations in addition to the K409R mutation retained capacity to induce CDC of Raji cells (FIG. 4A, B). However, introduction of L234F-L235E-G236R non-activating mutations in IgG1-HLA-DR-4-K409R and IgG1-HLA-DR-1D09C3-K409R antibody variants drastically reduced the potency to induce CDC. Moreover, as observed for IgG1-HLA-DR-1D09C3, the potency to induce CDC by the L234F-L235E-G236R-K409R antibody variant is at the same level of CDC induced by a HLA-DR-targeting F(ab′)2 fragment, which lacks the Fc region and therefore does not interact with complement protein C1q (FIG. 4B).

In summary, antibody variants harboring L234F-L235E-G236R mutations efficiently reduced CDC to levels similar to CDC induced by a F(ab′)2 fragment, which cannot engage the human complement system.

### Example 6: Binding to Fcγ Receptors by Anti-Human CD20 Antibodies and Non-Activating Variants Thereof

As said, for therapeutic monoclonal antibodies, where the therapeutic effect is independent on the interaction with the immune system's effector molecules (such as complement protein C1q and Fcγ receptors) and the adverse effects or dose-limiting toxicity is highly dependent on this interaction, a non-activating Fc domain could be considered to increase the therapeutic window. Data in Examples 3-5 showed that introduction of non-activating mutations in the constant heavy chain region of an antibody reduced the capacity to engage with complement protein C1q and reduced induction of CDC. Here we assessed the binding of the IgG1 or IgG4 anti-human CD20 antibodies and antibody variants thereof harboring non-activating mutations in the constant heavy chain region, to the monomeric extracellular domain (ECD) of human FcγRIa (SEQ ID NO: 15), or dimeric ECDs of human FcγRIIa allotype 131H (SEQ ID NO: 16), human FcγRIIa allotype 131R (SEQ ID NO: 17), human FcγRIIb (SEQ ID NO: 18), human FcγRIIIa allotype 158F (SEQ ID NO: 19), and human FcγRIIIa allotype 158V (SEQ ID NO: 20) in ELISA assays.

For this, anti-human CD20 antibodies IgG1 wild-type, IgG1-K409R, non-activating variants thereof harboring L234F-L235E-D265A-K409R, L234F-L235E-G236R-K409R, L234A-L235A-P329G-K409R, G236R-L328R-K409R, E233P-L234V-L235A-G236del-5267K-K409R, N297G-K409R, L234A-L235E-G237A-A330S-P331S-K409R mutations, as well as IgG4 wild-type, IgG4-S228P, and non-activating variants harboring 5228P-F234A-L235A, or S228P-E233P-F234V-L235A-G236del mutations were tested in a range of concentrations (0.0013-20 μg/mL in five-fold dilution steps) for binding to human Fcγ receptors. To detect binding to monomeric and dimeric FcγR variants, 96-well Microlon ELISA plates (Greiner, Germany) were coated overnight at 4° C. with 1 μg/mL goat-F(ab′)2-anti-human-IgG-F(ab′)2 (Jackson Laboratory, Cat #109-006-097) in PBS and subsequently washed and blocked with 200 μL/well PBS supplemented with 0.2% BSA (PBS/0.2% BSA) for 1 h at room temperature (RT). With washings in between incubations (PBS supplemented with 0.05% Tween 20 (Cat #P1379, Sigma); PBST; plus 0.2% BSA; PBST/0.2% BSA), plates were incubated with 100 μl/well of a dilution series of anti-human CD20 IgG1 and IgG4 antibody variants in PBST/0.2% BSA for 1 h at RT while shaking followed by incubation with 100 μL/well of monomeric or dimeric, His-tagged, C-terminally biotinylated FcγR ECD variants (1 μg/mL) in PBST/0.2% BSA for 1 h at RT while shaking. Finally, after washing as described above plates were incubated—with 100 μL/well Streptavidin-polyHRP (CLB, Cat #M2032, 1:10.000) in PBST/0.2% BSA as detecting antibody for 30 min at RT while shaking. Development with 1 mg/mL ABTS (Cat #11112422001 and 11112597001, Roche) was performed for circa 10 min (Ia), 15 min (IIa-131H, IIa-131R, IIIa-158V, IIIa-158F), or 30 min (IIb). Subsequently, reactions were stopped using 100 μL/well of 2% oxalic acid (Riedel de Haen, Cat #33506). Using a microplate reader (BioTek, Winoosky, VT), absorbance was measured at 405 nm.

To measure immobilization of the antibody variants and assess whether introduced non-activating mutations affect capture to the ELISA plate, 96-well Microlon ELISA plates (Cat #655092, Greiner) were coated overnight at 4° C. with 1 μg/mL goat-F(ab′)2-anti-human-IgG-F(ab′)₂ (Cat #109-006-097, Jackson Laboratory) in PBS. Subsequently, ELISA plates were washed and blocked with 200 μL/well PBS supplemented with 0.2% BSA (PBS/0.2% BSA) for 1 h at room temperature (RT). With washings in between the different incubation steps (PBS supplemented with 0.05% Tween 20 (PBST) plus 0.2% BSA; PBST/0.2% BSA), plates were sequentially incubated with 100 μl/well of a dilution series of anti-human CD20 IgG1 and IgG4 antibody variants (0.0013-20 μg/mL in five-fold dilution steps) in PBST/0.2% BSA for 1 h at RT while shaking and 100 μL/well HRP-labelled goat-anti-human-IgG-Fcγ (Cat #109-035-098, Jackson Laboratory; 1:10,000) in PBST/0.2% BSA as detecting antibody for 30 min at RT while shaking. Development with 1 mg/mL ABTS (Cat #11112422001 and 11112597001, Roche) was performed for circa 5 min and subsequently reactions were stopped using 100 μL/well of 2% oxalic acid. Using a microplate reader, absorbance was measured at 405 nm.

The data were analyzed using a non-linear agonist dose-response model and the area under the dose-response curves (AUC) per experimental replicate was calculated using log-transformed concentrations in GraphPad PRISM (version 8.4.1, GraphPad Software) with ELISA background signal (no antibody control) as baseline, followed by normalization per experimental replicate to the AUC value measured for the wild-type IgG1 antibody variant (Anti-human CD20 IgG1, 100%). Data is based on three independent replicates.

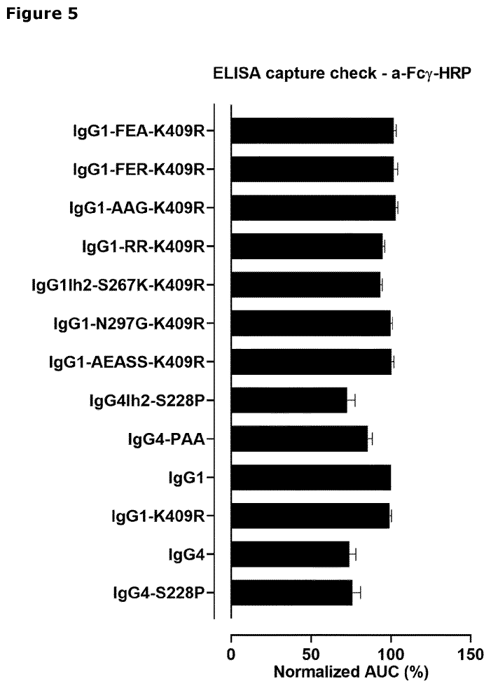

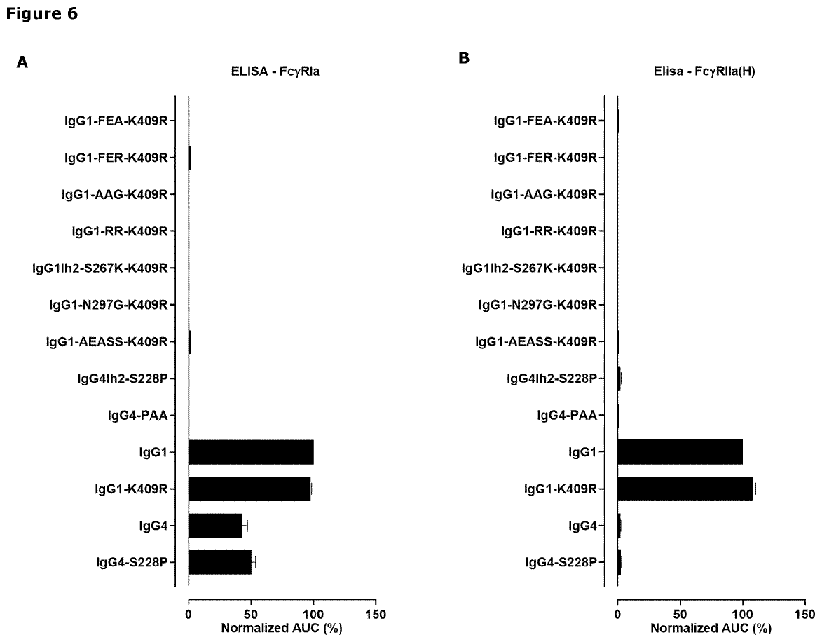

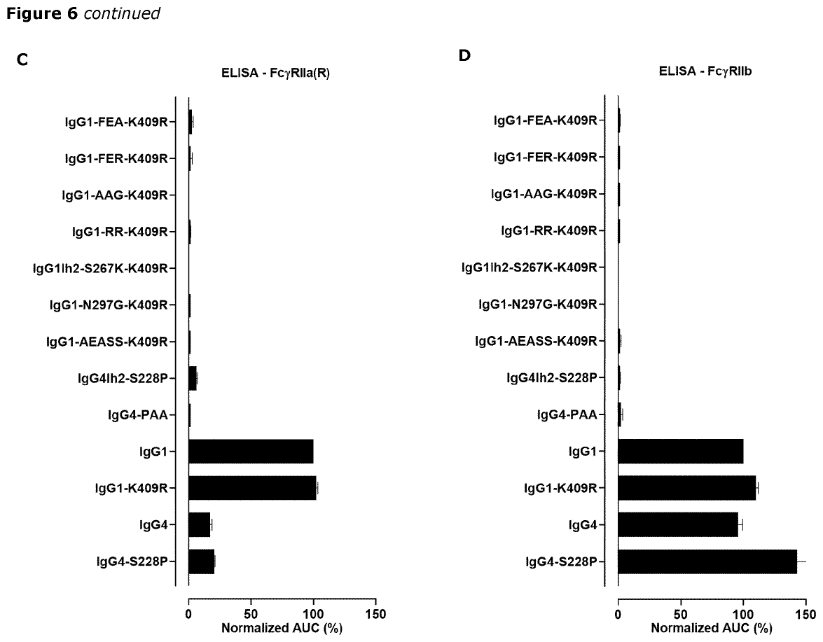

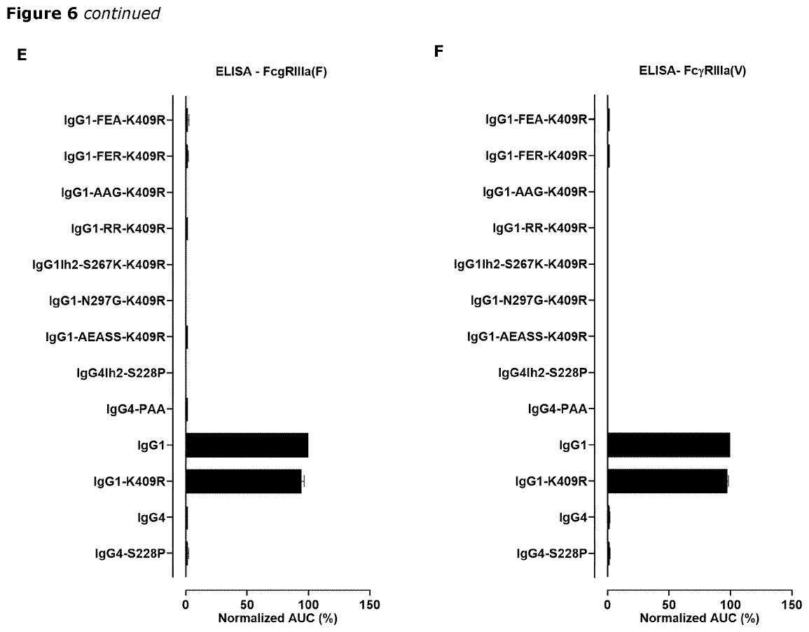

All anti-human CD20 IgG1 and IgG4 antibody variants were immobilized to the ELISA plates in a similar fashion with a minor reduction observed for antibody variants with an IgG4 backbone (FIG. 5). Therefore, it was concluded that introduction of non-activating mutations in the heavy chain regions of anti-human CD20 IgG1- and IgG4 antibody variants does not influence immobilization by anti-human-IgG-F(ab′)2 fragments. Assessment of binding of anti-human CD20 IgG1 variants to FcγRIa, FcγRIIa, FcγRIIb and FcγRIIIa by ELISA revealed that all anti-human CD20 IgG1 antibody variants harboring non-activating mutations in the constant heavy chain region did not interact with Fcγ receptors (FIG. 6A-F). Furthermore, non-activating IgG4 variants also failed to interact with Fcγ receptors, except for the IgG4 antibody variant harboring the S228P-E233P-F234V-L235A-delG236 mutations that showed residual binding to FcγRIIa-131R (FIG. 6C).

In summary, an IgG1 antibody variant harboring the L234F-L235E-G236R non-activating mutations showed no FcγR binding, similar to previously described non-activating Fc variants such as L234F-L235E-D265A and L234A-L235A-P329G.

### Example 7: Activation and Signaling Via Fcγ Receptors by Anti-Human CD20 Antibodies and Non-Activating Variants Thereof

In Example 6, the binding of anti-human CD20 IgG1 or IgG4 antibodies and variants thereof harboring non-activating mutations in the constant heavy chain to FcγRIa, FcγRIIa, FcγRIIb, and FcγRIIIa was studied. All non-activating antibody variants tested displayed no binding to FcγRs tested except for the IgG4 antibody variant harboring the S228P-E233P-F234V-L235A-delG236 mutations, which showed residual binding to FcγRIIa-131R. However, in the ELISA binding assays only effects on direct binding are evaluated. Effects of antigen-binding, target-mediated antibody clustering and subsequent target-mediated clustering of the Fc-receptors on the effector cells are absent. Here, we studied whether introduction of non-activating mutations in the heavy chain constant region of anti-human CD20 IgG1 or IgG4 antibodies, as stated in Example 6, affected FcγR activation and signaling in Promega reporter assays using target-expressing Raji cells and a Jurkat reporter cell line that expresses the indicated FcγR.

Activation of FcγR-mediated signaling, by the anti-human CD20 IgG1 and IgG4 antibody variants mentioned above, was quantified using reporter BioAssays (Promega, FcγRIa: Cat #CS1781C08; FcγRIIa allotype 131H: Cat #G9991; FcγRIIa allotype 131R: Cat #CS1781808; FcγRIIb: Cat #CS1781E04; FcγRIIIa allotype 158F: Cat #G9790; FcγRIIIa allotype 158V: Cat #G7010) with CD20-expressing Raji cells as target cells. As effector cells, the reporter cell kit contains Jurkat human T cells, engineered to stably express the indicated FcγR and a nuclear factor of activated T cells (NFAT)-response element driving the expression of firefly luciferase. The assay is performed according to the manufacturer's recommendations. In short, Raji cells (5,000 cells/well) were seeded in 384-wells white OptiPlates (Perkin Elmer, Cat #6007290) in Assay Buffer (Promega, Cat #G719A) supplemented with 12% low IgG serum (Promega, Cat #G711A) and incubated for 5 hours at 37° C./5% CO₂ in a total volume of 30 μL containing antibody concentration series (FcγRIIa, FcγRIIb, and FcγRIIIa, 0.001-15 μg/mL final concentrations in 5-fold dilutions; FcγRIa, 0.00000006-15 μg/mL final concentrations in 25-fold dilutions) and thawed Promega BioAssay Effector cells (30,000 cells/well). Subsequently, plates were incubated for 15 minutes at room temperature (RT), followed by addition of 30 μL Bio Glo Assay Luciferase Reagent. Plates were then incubated for 5 minutes at RT. Luciferase production was quantified by luminescence read-out on an EnVision Multilabel Reader (Perkin Elmer). Background luminescence signal determined from medium-only (no Raji cells, no antibody, no effector cells) samples were used to normalize the luminescence signals. The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control (only Raji cells and effector cells) as baseline. Per experiment, AUC values were normalized relative to reporter activity observed for cells incubated with a non-binding control IgG1-b12 (0%) and the activity of wild-type anti-human CD20 IgG1 (100%). Data is based on three independent replicates.

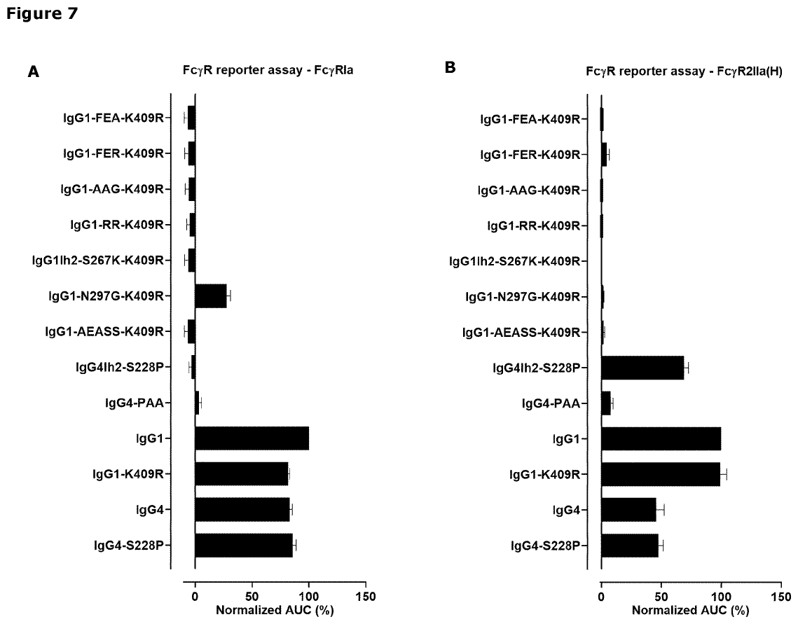

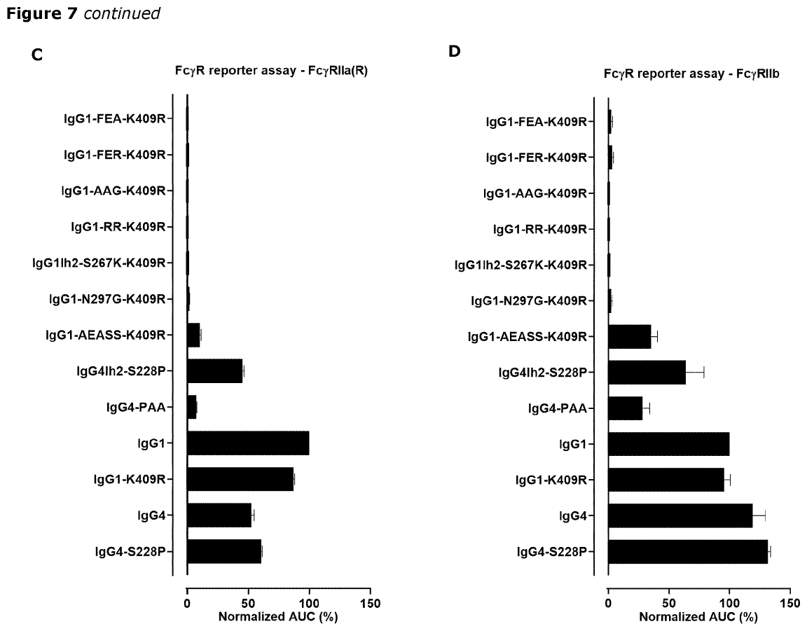

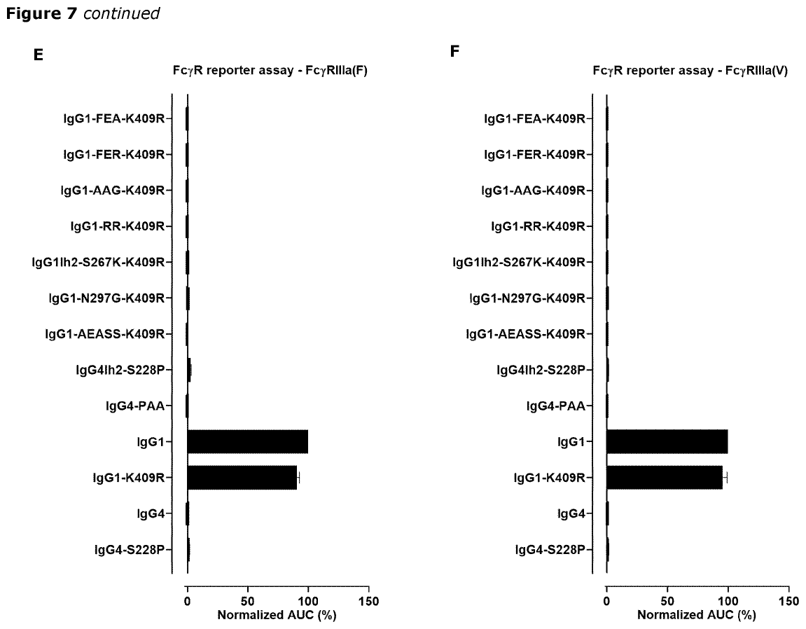

Assessment of FcγRIa-, FcγRIIa-, FcγRIIb-, and FcγRIIIa-mediated activation using Promega reporter assays revealed that introduction of L234F-L235E-G236R in combination with K409R mutations in the heavy chain constant region of IgG1 prevented FcγR-mediated activation, similar to the anti-human CD20 IgG1 non-activating variants L234F-L235E-D265A-K409R, L234A-L235A-P329G-K409R, G236R-L328R-K409R, and E233P-L234V-L235A-G236del-5267K-K409R (FIG. 7A-F). Absence of FcγR-mediated activation was also observed for other IgG1- or IgG4 antibody variants harboring non-activating mutations in the heavy chain constant regions, except for partial activation mediated via FcγRIa by the anti-human CD20 IgG1 antibody variant harboring the N297G and K409R mutations (FIG. 7A), and partial FcγRIIa- and FcγRIIb-mediated activation by IgG4 antibody variants harboring 5228P-F234A-L235A or S228P-E233P-F234V-L235A-G236del mutations, and by anti-human CD20 IgG1 antibody variant harboring L234A-L235E-G237A-A330S-P331S-K409R mutations (FIG. 7B-D). As controls, antibody variants anti-human CD20 IgG1 and anti-human CD20 IgG1-K409R induced activation of all FcγRs tested, while antibody variants IgG4 and IgG4-S228P induced FcγRIIa-, FcγRIIb-, and FcγRIa-mediated activation but lacked FcγRIIIa-mediated activation (FIG. 7A-F).

Whereas for several variants, for one or more of the Fcγ receptors, partial FcγR-mediated activation was observed, i.e. IgG1 variants harboring N297G-K409R or L234A-L235E-G237A-A330S-P331S-K409R mutations, and IgG4 antibody variants harboring 5228P-F234A-L235A or S228P-E233P-F234V-L235A-G236del mutations, advantageously, the capacity to induce FcγR-mediated activation by IgG1 antibody variants harboring the novel L234F-L235E-G236R non-activating mutations in the heavy chain constant region in addition to the K409R mutation was efficiently abrogated.

### Example 8: Binding to Neonatal Fc Receptor by Anti-Human CD20 Antibodies and Non-Activating Variants Thereof

The neonatal Fc receptor (FcRn) is responsible for the long plasma half-life of IgG by protecting IgG from degradation. Upon internalization of the antibody, FcRn engages with the antibody Fc regions in endosomes, where the interaction is stable in the mildly acidic environment (pH 6.0). Upon recycling to the plasma membrane, where the environment is neutral (pH 7.4), the interaction is disrupted and the antibody is released back into the circulation. This influences the plasma half-life of IgG. Here, an ELISA was performed to evaluate binding to human FcRn of anti-human CD20 IgG1 and IgG4 antibodies and variants thereof, as stated in Example 6 and 7, containing non-activating mutations in the constant heavy chain region.

Streptawell 96 well plates (Roche, Cat #1734776001) were coated with 5 μg/mL (100 μL/well) recombinantly produced biotinylated extracellular domain of human FcRn (FcRnECDHis-B2M-BIO), i.e. the extracellular domain of human FcRn with a C-terminal His tag (FcRnECDHis; SEQ ID NO: 21) as dimer with beta2microglobulin (B2M; SEQ ID NO: 22), diluted in PBS supplemented with 0.05% Tween 20 (PBST) plus 0.2% BSA for 2 hours while shaking at room temperature (RT). Plates were subsequently washed three times with PBST. Serially diluted antibody samples (0.0013-20 μg/mL final concentrations in 5-fold dilutions in PBST/0.2% BSA, pH 6.0 or pH 7.4) were added and incubated for 1 hour while shaking at RT. Plates were washed with PBST/0.2% BSA, pH 6.0 or pH 7.4. Horseradish Peroxidase (HRP)-conjugated polyclonal Goat-anti-Human kappa light chain (1:5,000; Sigma, Cat #A-7164) diluted in PBST/0.2% BSA, pH 6.0 or pH 7.4 was added, and plates were incubated for 1 hour at RT while shaking. After washing with PBST/0.2% BSA, pH 6.0 or pH 7.4., 100 μL 2,2′-Azino-bis(3-ethylbenzthiazoline-6-sulfonic acid (ABTS; 1 mg/mL; Roche, Cat #11112422001 and 11112597001) was added as substrate and plates were incubated for 15 minutes at RT protected from light. The reaction was stopped using 100 μL 2% oxalic acid (Riedel de Haen, Cat #33506), incubated for 10 minutes at RT. Using a microplate reader (BioTek, Winoosky, VT), absorbance was read at 405 nm. The data were analyzed using a non-linear agonist dose-response model and the area under the dose-response curves (AUC) per experimental replicate was calculated using log-transformed concentrations in GraphPad PRISM (version 8.4.1, GraphPad Software) with ELISA background signal (no antibody control) as baseline. Data is based on three independent replicates.

The FcRn binding ELISA assay showed that introduction of non-activating mutations in the constant heavy chain region of the anti-human CD20 IgG1 or IgG4 antibodies did not inhibit FcRn binding at pH 6.0. Conversely, at pH 7.4, all tested IgG1 or IgG4 antibody variants, including anti-human CD20 antibodies IgG1 and IgG4 wild-type, showed no binding to human FcRn.

Taken together, these results show that, similar to other variants tested, the antibody IgG1 variant harboring the novel L234F-L235E-G236R non-activating mutations retained FcRn binding properties similar to a wild-type IgG1 antibody.

### Example 9: Induction of Antibody-Dependent Cellular Cytotoxicity by Anti-Human CD20 Antibodies and Non-Activating Variants Thereof

FcγR binding to and activation and signaling by non-activating anti-human CD20 antibodies was assessed in Examples 6 and 7 respectively. Natural killer (NK) cell-mediated antibody dependent cellular cytotoxicity (ADCC) is dependent on engagement with FcγRIIIa. Here, the capacity to induce ADCC by anti-human CD20 IgG1 wild-type, IgG1-K409R, non-activating variants thereof harboring L234F-L235E-D265A-K409R, L234F-L235E-G236R-K409R, L234A-L235A-P329G-K409R, G236R-L328R-K409R, E233P-L234V-L235A-G236del-5267K-K409R, N297G-K409R, L234A-L235E-G237A-A330S-P331S-K409R mutations, as well as IgG4 wild-type, IgG4-S228P, and non-activating variants harboring 5228P-F234A-L235A, or 5228P-E233P-F234V-L235A-G236del mutations was assessed using the Perkin Elmer DELFIA® EuTDA TRF (time-resolved fluorescence) cytotoxicity assay with CD20-expressing Raji cells and peripheral blood mononuclear cells (PBMC) as a source for NK cells.

Buffy coats (Sanquin Blood Bank) were obtained from whole blood drawn from healthy volunteers, anticoagulated with citrate phosphate dextrose (20% final concentration (v/v)) to prevent coagulation activation, and 3.6-fold diluted in phosphate-buffered saline solution (PBS, Cat #SH3A3830.03, GE Healthcare). Peripheral blood mononuclear cells (PBMCs) were isolated from the PBS-diluted buffy coats by density gradient centrifugation using Leucosep™ tubes (Cat #227290, Greiner Bio-One) containing lymphocyte separation medium (Cat #25-072-Cl, Corning), as described in the manufacturer's instructions with some modifications. In short, density gradient centrifugation was performed by centrifugation 20 minutes at 800×g at room temperature (RT) with the brakes of the centrifuge set to 3. The enriched cell fraction was subsequently washed 3 times with 50 mL of PBS followed by centrifugation for 10 minutes at 300×g. Isolated PBMCs were resuspended in culture medium (RPMI-1640 with 2 mM L-glutamine and 25 mM Hepes, Cat #BE12-115F, Lonza; supplemented with 10% donor bovine serum with iron, DBSI, Cat #20371, Life Technologies). To determine the ADCC capacity of anti-CD20 wild-type antibodies and non-activating variants thereof, the DELFIA® EuTDA TRF (time-resolved fluorescence) cytotoxicity kit (Cat #AD0116, Perkin Elmer) was used, according to manufacturer's instructions. Two separate sets of experiments were performed.

For the first set of experiments, Raji cells were resuspended at a concentration of 1×10⁶ cells/mL in culture medium and labeled with 0.16% (v/v) bis(acetoxymethyl)2,2′:6′,2″-terpyridine-6,6″-dicarboxylate reagent solution (DELFIA BATDA reagent, Cat #C136-100, Perkin Elmer) for 20 min at 37° C. in a water bath. The hydrophobic BATDA label can freely pass the cell membrane and is intracellularly converted into the hydrophilic TDA label (2,2′:6′,2″-terpyridine-6,6″-dicarboxylic acid) which no longer passes the cell membrane. Labeled cells were subsequently washed three times with culture medium to remove excess BATDA reagent. Subsequently, cells were mixed with a concentration range of wild-type anti-CD20 antibodies and non-activating variants thereof (0.01-10000 ng/mL final concentrations; 10-fold dilutions), incubated for 15 minutes at RT, and freshly isolated PBMCs were added to the mixture at an E:T ratio of 100:1 in culture medium in a total volume of 200 μL in a V-bottom 96-well plate (Cat #651101, Greiner bio-one). Plates were incubated for 2 h at 37° C. in a humidified (85%) air atmosphere with 5% CO₂. Spontaneous BATDA release from labeled cells, representing 0% ADCC, was determined in the absence of PBMCs and antibodies and maximum BATDA release, representing 100% ADCC, was determined by incubating labelled cells with DELFIA® lysis buffer (5% final concentration (v/v), Cat #4005-0010, Perkin Elmer). After two hours plates were spun down for 5 minutes at 500×g and 20 μL of cell-free supernatant was transferred into a flat bottom 96-well white opaque OptiPlate (Cat #6005290, Perkin Elmer). Subsequently, 200 μL of DELFIA Europium Solution (Cat #C135-100, Perkin Elmer) was added to the transferred supernatant and samples were incubated for 15 min at RT in the dark. The fluorescence of the EuTDA chelates formed from the released TDA label was measured in with an EnVision multilabel plate reader (Perkin Elmer). Percentage specific release, as a measure for ADCC, was calculated using the following formula:

% Specific release=((fluorescence sample−fluorescence spontaneous release)/(fluorescence maximal−fluorescence spontaneous release))×100%

The data were analyzed using a non-linear agonist dose-response model and the area under the dose-response curves (AUC) per experimental replicate was calculated using log-transformed concentrations in GraphPad PRISM (version 8.4.1, GraphPad Software) with background stain (no antibody control) as baseline, followed by normalization per experimental replicate to the AUC value measured for the non-binding negative control IgG1-b12 (0%) and the AUC value measured for the wild-type IgG1 antibody variant (Anti-human CD20 IgG1, 100%). Data are mean values (±SEM) obtained from four (wild-type and K409R variants) or two (L234F-L235E-D265A-K409R and L234F-L235E-G236R-K409R variants) independent donors.

The second set of experiments was performed and analyzed essentially as described above. However, instead of a concentration range, the capacity to induce ADCC by anti-human CD20 IgG1 or IgG4 antibodies and variants thereof harboring non-activating mutations in the constant heavy chain region was assessed at 10 ug/mL final antibody concentrations. The data were normalized per experimental replicate to the non-binding negative control IgG1-b12 (0%) and the wild-type IgG1 antibody variant (Anti-human CD20 IgG1, 100%) and visualized in GraphPad PRISM (version 8.4.1, GraphPad Software). Data are mean values (±SEM) obtained from six donors from 2 independent experiments.

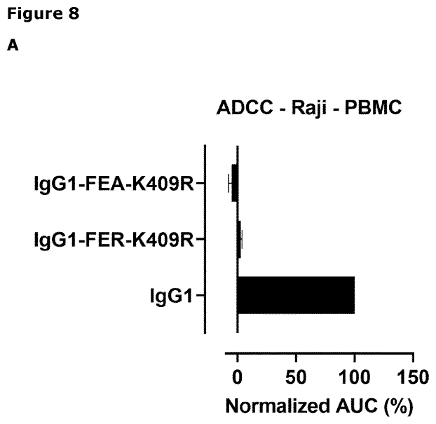

Assessment of NK-cell-mediated ADCC using the Perkin Elmer DELFIA® EuTDA TRF cytotoxicity kit revealed that wild-type anti-human CD20 IgG1 or a variant harboring a K409R mutation efficiently induced ADCC of CD20-expressing Raji cells (FIG. 8). In contrast, in line with the absence of binding to and activation and signaling via FcγRIIIa (Example 6 and 7), anti-human CD20 wild-type IgG4 antibody, or a variant harboring a S228P hinge stabilizing mutation, does not induce NK-cell-mediated ADCC (FIG. 8B). Assessment of the capacity to induce ADCC by non-activating antibody variants revealed that introduction of L234F-L235E-G236R and K409R mutations in the heavy chain constant region of anti-human CD20 IgG1 reduced ADCC of CD20-expressing Raji cells by −69-98%, similar to anti-human CD20 IgG1 non-activating variant L234F-L235E-D265A-K409R (FIG. 8A, B), and anti-human CD20 IgG1 non-activating variants L234A-L235A-P329G-K409R, G236R-L328R-K409R, E233P-L234V-L235A-G236del-5267K-K409R, N297G-K409R, L234A-L235E-G237A-A330S-P331S-K409R mutations (FIG. 8B). Moreover, similar to wild-type IgG4, IgG4 non-activating antibody variants 5228P-F234A-L235A or S228P-E233P-F234V-L235A-G236del also failed to induce ADCC (FIG. 8B).

In conclusion, the capacity to induce ADCC was efficiently abrogated by antibody variants harboring the novel L234F-L235E-G236R non-activating mutations in the heavy chain constant region.

### Example 10: T-Cell Activation in PBMC Culture by Anti-Human CD3 Antibodies and Non-Activating Variants Thereof

The upregulation of CD69 on T cells was evaluated as a measurement for early activation of T cells by anti-human CD3 huCLB-T3/4 IgG1 and IgG4 antibodies and variants thereof harboring non-activating mutations in the constant heavy chain region by FACS analysis.

PBMCs were isolated from buffy coats by density gradient separation as described in Example 9, washed with PBS, and resuspended in culture medium (RPMI-1640 with 2 mM L-glutamine and 25 mM Hepes, Cat #BE12-115F, Lonza; supplemented with 10% donor bovine serum with iron, DBSI, Cat #20371, Life Technologies). A dose response series of anti-human CD3 IgG1-F405L and non-activation variants thereof harboring L234F-L235E-D265A, L234F-L235E-G236R, L234A-L235A-P329G, G236R-L328R, E233P-L234V-L235A-G236del-S267K, or L234A-L235E-G237A-A330S-P331S mutations in addition to F405L as well as anti-human CD3 IgG4 with a hinge stabilizing mutation S228P or non-activating variants harboring 5228P-E233P-F234V-L235A-G236del or 5228P-F234A-L235A mutations in addition to F405L and R409K was prepared in culture medium (0.001-1000 ng/ml final concentrations, 10-fold dilutions) and added to the wells of a 96-well round bottom plate containing the PBMCs (1.5×10⁵ cells/well) in culture medium. After 16-24 hours incubation, cells were pelleted by centrifugation. Subsequently, cells were then washed with FACS buffer (1×PBS, Cat #BE17-517Q, Lonza; 0.1% bovine serum albumin, BSA; 0.02% Na N3, Cat #41920044-3, Bio-world) and stained for 30 minutes at 4° C. with a mouse-anti-human CD28-PE (Cat #130-092-921; Miltenyi Biotec; T-cell marker) and mouse-anti-human CD69-APC antibody (Cat #340560; BD Biosciences). Unbound antibodies were removed by washing twice with FACS buffer and subsequently cells were resuspended in FACS buffer (150 μL/well) and the percentage of CD69-positive cells of the CD28-positive cells in the PBMC mixture was measured on a Fortessa flow cytometer (BD). The data was visualized as dose response vs percentage CD69+ of CD28+ cells and the area under the dose-response curves (AUC) per PBMC donor of each experimental replicate was calculated using log-transformed concentrations in GraphPad PRISM (version 8.4.1, GraphPad Software) with background stain (no antibody control) as baseline, followed by normalization for each donor per experimental replicate to the AUC value measured for the wild-type IgG1 antibody variant (IgG1-F405L, 100%). Data are mean values ±SEM obtained from 6 donors from three independent replicates.

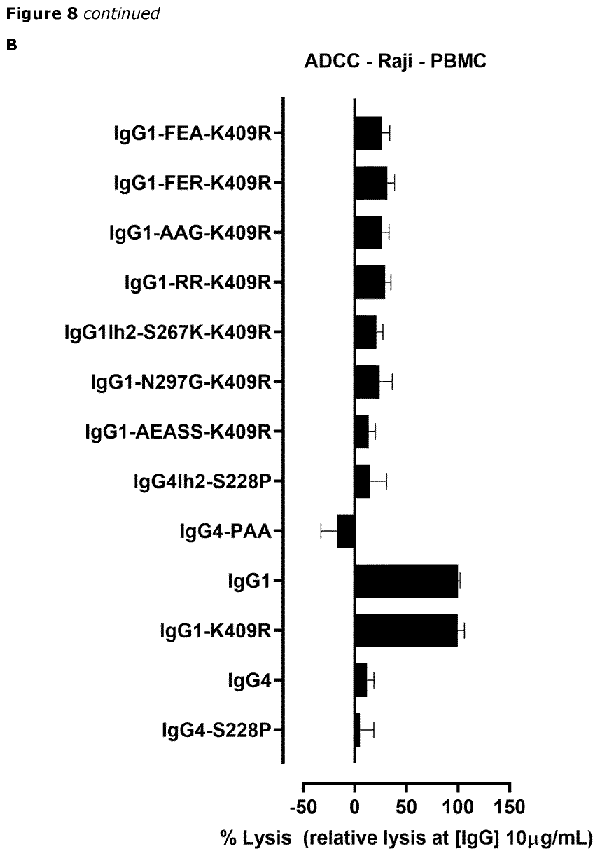

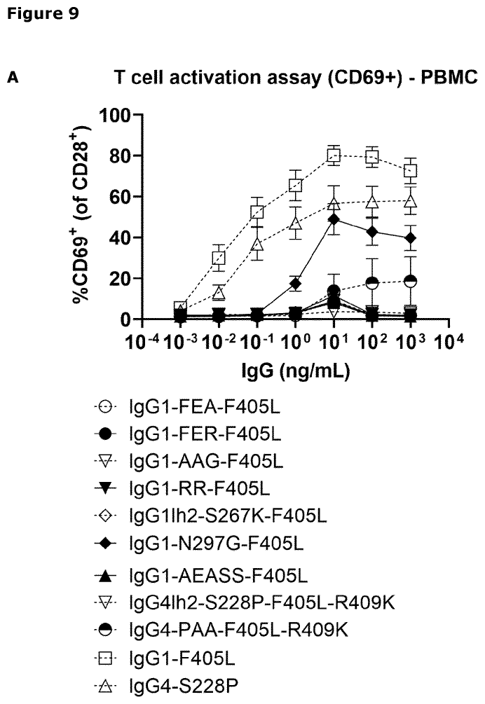

Assessment of CD69 upregulation on T cells as a measure for early T-cell activation shows that the anti-human CD3 antibody IgG1 harboring the L234F-L235E-G236R non-activating mutations in addition to a F405L mutation prevented upregulation of CD69 on T cells in PBMC co-cultures, similar to other variants tested, i.e. anti-human CD3 IgG1-F405L variants that include the non-activating mutations L234F-L235E-D265A, L234A-L235A-P329G, G236R-L328R, E233P-L234V-L235A-G236del-5267K, or L234A-L235E-G237A-A330S-P331S, as well as the anti-human CD3 IgG4-F405L-R409K variant that includes the non-activating mutations 5228P-E233P-F234V-L235A-G236del (FIG. 9A, B). In contrast, an anti-human CD3 IgG1 antibody variant harboring the N297G with F405L mutations and an anti-human CD3 IgG4 antibody variant harboring the 5228P-F234A-L235A with F405L-R409K mutations failed to prevent CD69 upregulation on T cell in a PBMC co-culture.

In summary, activation of T cells, as measured by CD69 upregulation, in a PBMC co-culture can be prevented by introduction of the novel L234F-L235E-G236R non-activating mutations in an anti-human CD3 IgG1 type antibody.

### Example 11: In Vitro T-Cell-Mediated Cytotoxicity Induced by Non-Activating Antibody Variants

In Example 10, it was shown that the introduction of non-activating mutations L234F-L235E-G236R in the constant heavy chain region of IgG1 variants of a CD3-targeting antibody could efficiently prevent T-cell activation. Here, T-cell-mediated cytotoxicity was assessed for CD3×HER2, CD3×b12, and b12×HER2 bispecific wild-type IgG1 and IgG4 antibodies and variants thereof harboring non-activating mutations in the constant heavy chain region.

Bispecific molecules were generated by controlled Fab-arm exchange (cFAE), as described in Example 1. T-cell-mediated cytotoxicity by the wild-type bispecific antibodies CD3×HER2 (anti-human CD3 (huCLB-T3/4) IgG1 or IgG4 and anti-human HER2 IgG1 or IgG4), CD3×b12 (anti-human CD3 (huCLB-T3/4) IgG1 or IgG4 and non-binding control antibody anti-HIV1 gp120 (b12) IgG1 or IgG4), or b12×HER2 (non-binding control antibody anti-HIV1 gp120 (b12) IgG1 or IgG4 and anti-human HER2 IgG1 or IgG4) and variants thereof harboring non-activating mutations in the constant heavy chain region was evaluated. PBMCs were isolated from buffy coats derived from healthy donors by density gradient separation as described in Example 9, washed with PBS, and resuspended in culture medium (RPMI-1640 with 2 mM L-glutamine and 25 mM Hepes; supplemented with 10% donor bovine serum with iron (DBSI)). HER2-expressing SK-OV-3 cells (Cat #HTB-77, ATCC) were cultured in McCoy's 5A medium (Lonza, Cat #BE12-168F) supplemented with 10% (vol/vol) heat inactivated DBSI, and penicillin-streptomycin (Pen/Strep, final concentration 50 units/mL potassium penicillin and 50 μg/mL streptomycin sulfate (Lonza, Cat #DE17-603E) and maintained at 37° C. in a 5% (vol/vol) CO₂ humidified incubator. SK-OV-3 cells were cultured to near confluency. Cells were trypsinized and resuspended in culture medium and subsequently passed through a cell strainer to obtain a single cell suspension. 2.5×10⁴ SK-OV-3 cells were seeded to each well of a 96-well culture plate, and cells were incubated 4 hours at 37° C., 5% CO₂ to allow adherence to the plate. Subsequently, 1×10⁵ PBMCs were added to each well of the 96-well plate containing the SK-OV-3 target cells resulting in a effector to target (E:T) ratio of 4:1. Subsequently, a dose response series of bispecific CD3×HER2, CD3×b12, and b12×HER2 wild-type and non-activating variants thereof, as mentioned above, was prepared in culture medium (0.001-1000 ng/mL final concentration, 10-fold dilutions) and added to the wells of a 96-well culture plates containing the SK-OV-3 cells and PBMCs. Incubation of SK-OV-3 target cells with 2 μM staurosporin (Cat #S6942-200, Sigma) was used as reference for 100% tumor cell kill. Medium control (SK-OV-3 cell, no antibody, no PBMC) was used as a reference for 0% tumor cell kill. Plates were incubated for 3 days at 37° C., 5% CO₂. After three days, plates were washed twice with PBS, and 150 μL culture medium containing 10% Alamar blue (Invitrogen, Cat #DAL1100) was added to each well. Plates were incubated for 4 hours at 37° C., 5% CO₂. Absorbance at 590 nm was measured (Envision, Perkin Elmer, Waltham, MA). The data was visualized in GraphPad PRISM (version 8.4.1, GraphPad Software) as dose response vs percentage viable SK-OV-3 cells, calculated for each donor per experimental replicate. Data are mean values ±SEM obtained from 6 donors from three independent replicates.

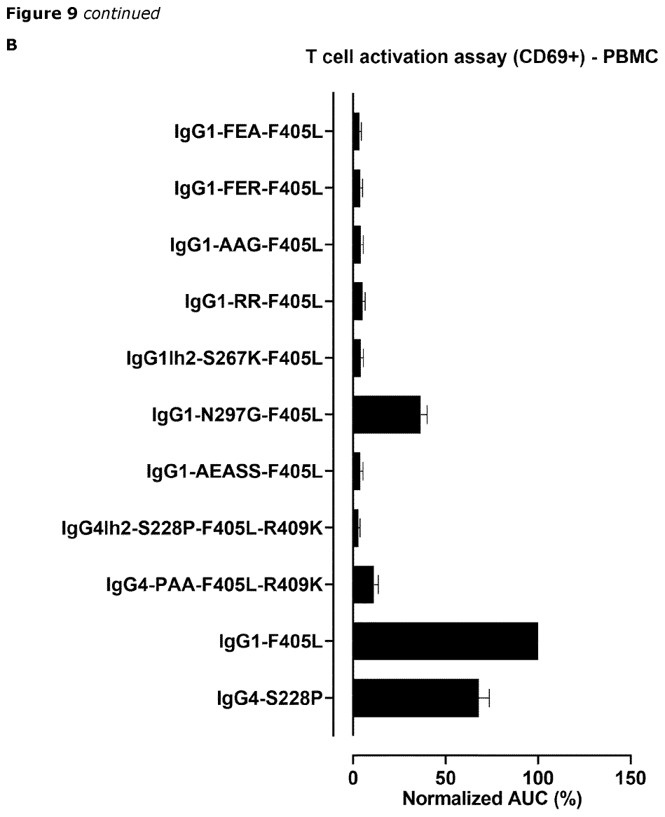

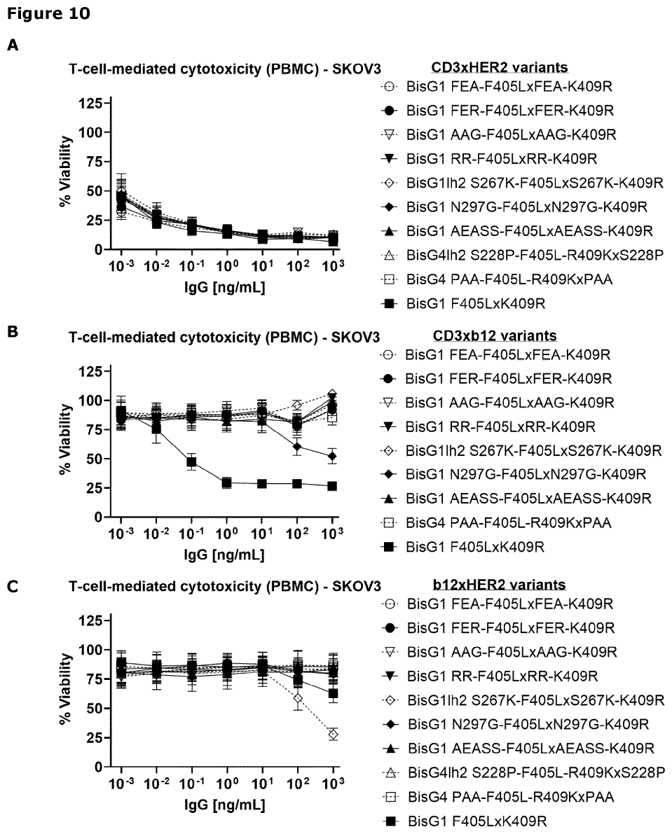

All bispecific CD3×HER2 antibody variants harboring non-activating mutations in the constant heavy chain region induced dose-dependent cytotoxicity of SK-OV-3 cells with comparable efficiency to a bispecific CD3×HER2 antibody variant harboring an Fc region with wild-type-like function, thus without non-activating mutations (FIG. 10A). The wild-type-like bispecific CD3×b12 antibody induced non-specific killing of SK-OV-3 cells, albeit to a lesser extent than the wild-type like bispecific CD3×HER2 antibody variant. In contrast, the bispecific CD3×b12 antibody variant harboring the L234F-L235E-G236R non-activating mutations showed no cytotoxicity of SK-OV-3 cells, similar to other non-activating bispecific CD3×b12 variants tested, except for the CD3×b12 bispecific antibody harboring an N297G mutation which still showed partial non-specific cytotoxicity of SK-OV-3 cells at the highest concentrations tested (FIG. 10B). This is in line with the observed activation (upregulation of CD69) of T cells in a PBMC culture as observed in Example 10 for this variant. A minor level of non-specific cytotoxicity of SK-OV-3 cells was observed for a wild-type-like bispecific b12×HER2 antibody variant. Non-specific cytotoxicity was not observed for other bispecific b12×HER2 antibody variants tested except for residual non-specific cytotoxicity by the bispecific b12×HER2 variant harboring the E233P-L234V-L235A-G236del-S267K mutations (FIG. 10C).

Overall, introduction of the novel L234F-L235E-G236R mutations in the constant heavy chain region of bispecific antibody variants, targeting a cancer antigen and a T-cell, allows to efficiently avoid non-specific cytotoxicity and retaining the capacity to induce specific T-cell-mediated cytotoxicity.

### Example 12: Pharmacokinetic (PK) Analysis of Non-Activating Antibody Variants

The pharmacokinetic properties of anti-human CD3 (huCLB-T3/4) and anti-human CD20 IgG1 antibody variants harboring non-activating mutations in the constant heavy chain region were analyzed in a mouse study.

The mice in this study were housed in the Central Laboratory Animal Facility (Utrecht, the Netherlands). All mice were kept individually in ventilated cages with food and water provided ad libitum. All experiments were in compliance with the Dutch animal protection law (WoD) translated from the directives (2010/63/EU) and were approved by the Dutch Central Commission for animal experiments and by the local Ethical committee). SCID mice (C.B-17/IcrHan@Hsd-Prkdc<scid, Envigo) were injected intravenously with 500 μg antibody (wild-type anti-human CD3 IgG1, variants thereof harboring the F405L mutation alone or in combination with non-activating mutations L234F-L235E-D265A or L234F-L235E-G236R, wild-type anti-human CD20 IgG1, and variants harboring the K409R mutation alone or in combination with non-activating mutations L234F-L235E-D265A or L234F-L235E-G236R) using 3 mice per group. Blood samples (50 μL) were collected from the facial vein at 10 min, 4 hours, 1 day, 2 days, 8 days, 14 days and 21 days after antibody administration. Blood was collected into vials containing heparin and subsequently centrifuged for 5 min at 10,000 g. Plasma was stored at −20° C. until determination of antibody concentrations.

By a total hIgG ELISA, specific human IgG concentrations were determined. Anti-human IgG (Cat #M9105, Lot #8000260395 Sanquin, The Netherlands), coated on a 96-well Microlon ELISA plates (Greiner, Germany) at a concentration of 2 μg/mL, was used as capturing antibody. Plates were subsequently blocked with PBS supplemented with 0.2% BSA, followed by addition of samples, serially diluted in ELISA buffer (PBS supplemented with 0.05% Tween 20 and 0.2% bovine serum albumin), and incubated on a plate shaker for 1 h at room temperature (RT). Subsequently, plates were incubated with goat anti-human IgG immunoglobulin (Cat #109-035-098, Jackson) and developed with 2,2′-azino-bis (3-ethylbenzthiazoline-6-sulfonic acid; ABTS; Roche). The reaction was stopped by addition of 2% Oxalic Acid (Riedel de Haen, Cat #33506). The respective materials used for injection were used as the reference curve. Absorbance was measured in a microplate reader (Biotek, Winooski, VT) at 405 nm.

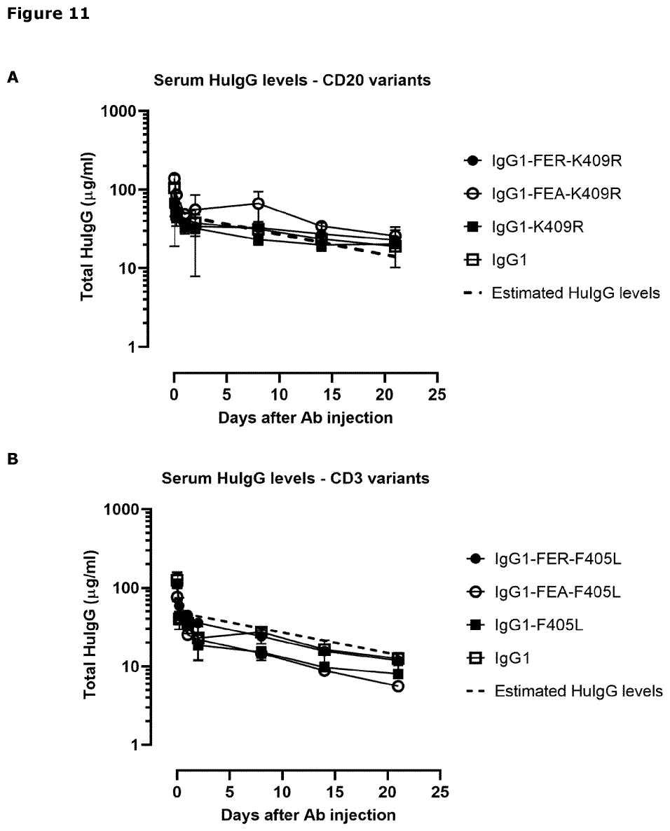

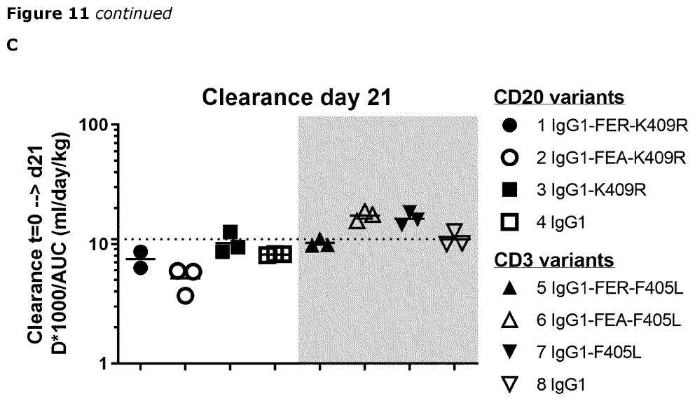

Serum IgG concentrations of anti-human CD20 IgG1 antibody and variants thereof harboring, K409R, L234F-L235E-D265A-K409R, or L234F-L235E-G236R-K409R mutations (FIG. 11A) as well as serum IgG concentrations of anti-human CD3 IgG1 antibody and variants thereof harboring F405L, L234F-L235E-D265A-F405L, or L234F-L235E-G236R-F405L mutations (FIG. 11B) were comparable. The measured IgG concentration in plasma for all anti-human CD20 IgG1 and anti-human CD3 IgG1 antibody variants injected in mice was in line with the concentrations predicted by the 2-compartment model pharmacokinetics for wild-type human IgG1 in SCID mice. Calculated clearance values for all anti-human CD20 IgG1 and anti-human CD3 IgG1 antibody variants were similar to their wild-type counterparts (FIG. 11C; day 21 post injection). Variation between the groups mainly reflects differences in the distribution phase, which could be caused by differences in glycosylation between the batches and may not be related to the format. The t½beta appeared to be comparable for all the mutations.

In summary, the introduction of non-activating mutations, including the novel L234F-L235E-G236R mutations does not affect the pharmacokinetics of IgG1 antibody variants in mice.

### Example 13: Immunogenicity Assessment of Antibody Variants Harboring L234F-L235A-G236R Non-Activating Mutations in the Fc Reqion

Risk of clinical immunogenicity of the constant domain of an IgG1 antibody variant harboring L234F-L235E-G236R mutations (IgG1-FER) was assessed using Abzena's iTope™ and TCED™ in silico technologies.

The iTope™ software predicts favourable interactions between the amino acid side chains of all possible 9-mer peptides in the test protein sequence and the open-ended binding grooves of 34 human MHC class II alleles (Perry et al., 2008, Drugs RD 9(6), pp. 385-96). The selected alleles represent the most common HLA-DR alleles found worldwide, with no weighing attributed to those found most prevalent in any particular ethnic population. By comparing these predictions to the MHC class II binding sequences present in the wild-type human IgG1(m)f reference, it's possible to identify novel binding sequences. Peptides predicted to bind with moderate and high affinity to 50°/c) MHC class II alleles are thought to correlate with the presence of T-cell epitopes (Hill et al., 2003, Arthritis Res Ther 5(1), pp. R40-8). TCED™ is a database of known T cell epitopes identified by ex vivo immunogenicity studies using a variety of proteins, predominantly antibodies (Bryson et al., 2010, BioDrugs 24(1), pp. 1-8). The database is interrogated by a BLAST search to confirm whether peptides with predicted promiscuous moderate or high affinity are also present in the database.

Each 9-mer was scored based on the potential ‘fit’ and interactions with the MHC class II molecules. The peptide scores calculated by the software lie between 0 and 1. Peptides that produced a high mean binding score (>0.55 in the iTope™ scoring function) were highlighted and, if 50°/o of the MHC class II binding peptides (i.e. 17 out of 34 alleles) had a high binding affinity (score>0.6), such peptides were defined as ‘promiscuous high affinity’ MHC class II binding peptides which are considered a high risk for containing CD4⁺ T cell epitopes. Promiscuous moderate affinity MHC class II binding peptides bind 50% alleles with a binding score>0.55 (but without a majority>0.6). Further analysis of the sequences was performed using the TCED™. These criteria were altered in the case of a large aromatic amino acid (i.e. F, W, Y) occurring in the p1 anchor position where the open p1 pocket of 20 of the 34 alleles allows the binding of a large aromatic residue. Where this occurs, a promiscuous peptide is defined as binding to 10 or more of the subset of 20 alleles. The sequences were used to interrogate the TCED™ by BLAST search in order to identify any high sequence homology between peptides (T-cell epitopes) from unrelated proteins/antibodies that stimulated T cell responses in previous ex vivo studies.

iTope™ analysis did not identify any promiscuous moderate or high affinity MHC class II binding sequences which were present in IgG1-FER and absent in the wild-type IgG1(m)f. Therefore, based on this analysis, there is no apparent increased risk of clinical immunogenicity for antibodies based on IgG1-FER.

### Example 14: Glyco-analysis of Non-Activating Antibody Variants

IgG molecules have a single conserved Asn (N)-linked glycosylation site in the CH2 region (at position N297 for IgG1). The core structure of this Asn-linked glycan is comprised of N-Acetylglucosamine (GlcNAc) and mannose residues. Further extension can take place with galactose, sialic acid, core fucosylation, and bi-secting GlcNAc (Vidarsson et al., 2014, Front. Immunology October 20; 5:520), resulting in heterogeneously glycosylated IgG1 molecules at N297. Glycans play an important role in protein conformation, stability and biological function (Costa et al., Crit Rev Biotechnol 34(4): 281-99 (2014)). Therefore, glycosylation changes in IgG1 molecules are unwanted as the efficacy and pharmacokinetic properties may be impacted. Furthermore, glycan heterogeneity needs to be analyzed and controlled during manufacturing and charged glycans may further increase the complexity of charge-based analytical assays such as imaged capillary isoelectric focusing (iCIEF) that are used for release testing or characterization.

In this Example, N-linked glycan profiling was performed on wild-type anti-human CD20 IgG1 antibody and non-activating variants thereof that harbor L234F-L235E-D265A or L234F-L235E-G236R mutations in addition to a K409R mutation. In addition, glycan profiling was performed on anti-human CD3 (huCLB-T3/4) IgG1 antibody harboring L234F-L235E-F405L mutations or variants thereof further harboring the D265A or G236R mutation.

Assessment of IgG1 N-linked glycosylation was performed by two different methods as indicated (Table 2). Anti-human CD20 IgG1 wild-type, anti-human CD20 IgG1 harboring L234F-L235E-D265A-K409R mutations, and anti-human CD3 IgG1 variants harboring either L234F-L235E-F405L or L234F-L235E-D265A-F405L mutations were analyzed by 2-aminobenzamide (2-AB) labeling. N-linked glycan profiling was performed by Normal phase HPLC analysis, using a Waters Alliance 2695 Separations Module (Waters), of 2-AB-labeled glycans. N-linked glycans were released from the antibodies by incubation with peptide-N-glycosidase F (Cat #GKE-5006D, PROzyme). Subsequently, the antibodies were ethanol-precipitated (ice-cold) and removed. The supernatants containing the released glycans were vacuum-dried. The obtained glycans were solubilized and subsequently labelled with the fluorophore 2-AB (from LudgerTag 2-AB Glycan Labeling Kit, Cat #LT-KAB-A2, Ludger) label on the reducing end by reductive amination for HPLC analysis. HPLC profiles were then obtained with gradient elution in conjunction with fluorescence detection. The integration of the HPLC peak intensities was a measure for percentages of molar abundances of the individual N-linked glycans (e.g. G0F, G1F, G2F, etc.) relative to the total population of oligosaccharides. Peaks were assigned based on an external glycan standard mix (prepared by mixing separate glycans from Ludger: NGA2 (=G0, #CN-NGA2-20U), NGA2F (=G0F, #CN-NGA2F-20U), NA2 (=G2, #CN-NA2-20U), NA2F (=G2F, #CN-NA2F-20U), MANS (#CN-MANS-20U), MANE (#CN-MAN6-20U), MAN8 (#CN-MAN8-20U), MANS (#CN-MAN9-20U), A1 (#CN-A1-20U), A1F (#CN-A1F-20U), A2 (#CN-A2-20U), A2F (#CN-A2F-20U).

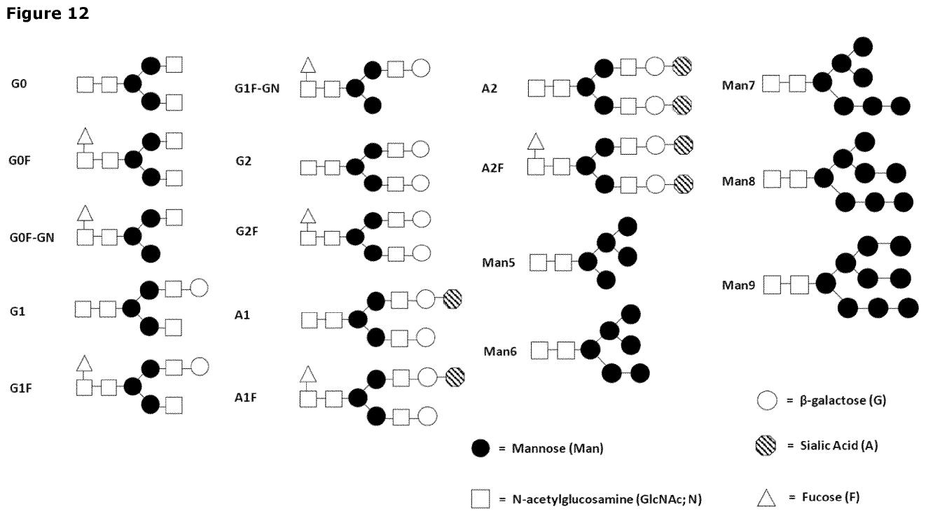

The anti-human CD20 IgG1 variant harboring the L234F-L235E-G236R mutations in addition to K409R and the anti-human CD3 IgG1 variant harboring the L234F-L235E-G236R mutations in addition to F405L were analysed by liquid chromatography-mass spectrometry (LC-MS) analysis using an Orbitrap Q-Exactive Plus mass spectrometer (Thermo Fischer). Identification and relative quantitation of the N-linked glycosylation was performed on the reduced glycosylated antibody heavy chain. Given the known primary amino acid sequence of the heavy chain the mass identity of the attached N-linked glycans could be identified. Briefly, antibody samples were diluted to 200 μg/mL in PBS pH 7.4 (Cat #3623140, B. Braun) to a total volume of SOUL. Next, 1 μL 1M Dithiothreitol (DTT; Cat #D9163, Sigma) was added to SOUL sample and incubated for 1 hour at 37° C. Subsequently, the sample was transferred to a glass Qsert vial (Cat #186001128c, Waters) and placed into the LC-MS., 1 μL was injected onto the LC column and the antibody was eluted using a mobile phase A (Milli-Q water with 0.1% Formic Acid, Cat #56302-50ml-F, Fluke)—mobile phase B (0.1% Formic Acid in Acetonitrile, Cat #0001934101135, Bio Solve) gradient from 23% (B) to 95% (B) in 2 minutes at 0.2 mL/min flow rate. The obtained raw m/z spectra were deconvoluted with Protein Deconvolution 4.0 (Thermo Fisher) software. As a result, the deconvoluted spectra provided reduced glycosylated heavy chain masses. The obtained peak intensities (after spectral deconvolution) was a measure for the percentages calculated of molar abundances of the individual N-linked glycans (e.g. G0F, G1F, G2F, etc.) relative to the total population of oligosaccharides. FIG. 12 shows schematic representations of the detected glycan species.

Knowledge of the relative abundance of the different oligosaccharides coupled with knowledge of the structure thereof (see FIG. 12) allows calculation of the total amount of galactosylation and charged glycans relative to the amounts in an A2F structure. The amounts calculated here as percentages represent molar amounts, i.e. representative of number of molecules and not of mass. From table 2, it is calculated that a wildtype IgG1 sequence has a percentage of abundance of charged glycans and percentage of galactosylation between 0-1% and 15-25% respectively. The percentage of charged glycans and galactosylation of non-activating L234F-L235E-D265A mutations was about 10% and about 60%.

Evaluation of glycosylation (Table 2) of anti-human CD20 IgG1 antibody variants revealed that introduction of non-activating L234F-L235E-D265A mutations in combination with a K409R mutation increased galactosylation, indicated as G1 or G2 and variants thereof (G: Galactose), and increased presence of charged glycans, indicated as A1 or A2 and variants thereof (A: Sialic Acid), as compared to an anti-human CD20 IgG1 wild-type antibody. A similar pattern was observed for the anti-human CD3 IgG1 antibody variant harboring non-activating L234F-L235E-D265A mutations in addition to a F405L mutation. Increased galactosylation and presence of charged glycans can be assigned to the presence of the D265A mutation because the anti-human CD3 IgG1 antibody variant harboring the L234F-L235E-F405L mutations showed patterns of glycosylation similar to the anti-human CD20 IgG1 wild-type antibody. In line with this, introduction of non-activating mutations L234F-L235E-G236R in either anti-human CD20 IgG1-K409R or anti-human CD3 IgG1-F405L also did not result in increased galactosylation and increased presence of charged glycans.

In summary, whereas introduction of non-activating L234F-L235E-D265A mutations in the constant heavy chain region of IgG1 antibodies increases antibody glycosylation heterogeneity, with an increase in galactosylation and increase in the presence of charged glycans, introduction of non-activating L234F-L235E-G236R mutations in IgG1 antibodies allows for retaining a glycan profile which is comparable to a wild-type constant region of IgG1.

TABLE 2

| | | Glycan species¹ (%) | | | | | | | | | | | | | | | | | | |
|---|---|---|---|---|---|---|---|---|---|---|---|---|---|---|---|---|---|---|---|---|
| | | | | G0F- | | | | G1F- | G2/ | | | | | | | | | A2F/ | | |
| | Variant | G0 | G0F | GN | Man5 | G1 | G1F | GN | Man6 | G2 | Man6 | G2F | A1 | A1F | Man7 | Man8 | A2 | Man9 | A2F | Man9 |
| IgG1- | FEA- | 0.6 | 12.2 | na | 1.2 | 0.0 | 36.3 | na | 0.7 | na | Na | 24.7 | 4.5 | 7.5 | na | 0.5 | 3.4 | 0.8 | na | na |
| CD20 | K409R² | | | | | | | | | | | | | | | | | | | |
| | WT² | 4.5 | 61.4 | na | 2.9 | 1.2 | 24.2 | na | 0.8 | na | Na | 2.4 | 0.6 | 0.2 | na | 0.2 | na | na | na | na |
| | FER- | 0.0 | 58.1 | 6.1 | 3.6 | 0.0 | 25.3 | 4.2 | na | 0.0 | 0.0 | 2.8 | 0.0 | 0.0 | 0.0 | 0.0 | 0.0 | na | 0.0 | 0.0 |
| | K409R³ | | | | | | | | | | | | | | | | | | | |
| IgG1- | FEA- | 0.3 | 13.2 | na | 0.9 | 0.0 | 36.8 | na | 0.6 | na | Na | 25.1 | 5.8 | 8.3 | na | 0.5 | 3.1 | 1.0 | na | na |
| CD3 | F405L² | | | | | | | | | | | | | | | | | | | |
| | FE- | 1.5 | 53.7 | na | 1.6 | 0.4 | 34.5 | na | 0.5 | na | Na | 5.6 | 0.5 | 0.3 | na | 0.0 | <0.1 | 0.0 | na | na |
| | F405L² | | | | | | | | | | | | | | | | | | | |
| | FER- | 0.0 | 57.3 | 6.2 | 2.9 | 0.0 | 26.1 | 3.2 | na | 0.0 | 0.0 | 2.0 | 0.0 | 0.0 | 2.2 | 0.0 | 0.0 | na | 0.0 | 0.0 |
| | F405L³ | | | | | | | | | | | | | | | | | | | |

¹Schematic drawings of the glycan species are shown in FIG. 12
²Analyzed by 2-AB labeling. G2, Man6, A2F, Man9 cannot be detected separately (not applicable, na), sum of both pairs is shown. G0F-GN, G1F-GN and Man7 were not assessed with this method (not applicable; na)
³Analyzed by Orbitrap mass spectrometry. G2, Man6, A2F, and Man9 detected separately. Sums: G2/Man6 and A2F/Man9 are not applicable (na)

Table 2 shows analysis of glycosylation for anti-human CD20 IgG1 and anti-human CD3 IgG1 antibody variants harboring non-activating mutations in the constant heavy chain region. Anti-human CD20 IgG1 wild-type (wt) or a variant with L234F-L235E-D265A-K409R mutations, as well as anti-human CD3 IgG1 harboring L234F-L235E-F405L or L234F-L235E-D265A-F405L mutations were analyzed by a 2-AB labeling method. Glycan profiles of anti-human CD20 IgG1 and anti-human CD3 IgG1 antibody variants harboring L234F-L235E-G236R-K409R or L234F-L235E-G236R-F405L mutations respectively were assessed by liquid chromatography-mass spectrometry (LC-MS). In contrast to the Orbitrap LC-MS method, 2-AB labelling method cannot separately assign G2 and Mannose 6 as well as A2F and Mannose 9 (not applicable; na) and therefore the sum of both is shown. GOF-GN, G1F-GN, and Man7 were not assessed with 2-AB method and are stated as not applicable (na). Variants tested are IgG1, IgG1-FER-K409R, IgG1-FEA-K409R, IgG1-FE-F405L, IgG1-FER-F405L, IgG1-FEA-F405L wherein FER: L234F-L235A-G236R, FE: L234F-L235E, and FEA: L234F-L235E-D265A. Schematic representations of detected glycan species are shown in FIG. 12.

### Example 15: Efficiency of Controlled Fab-Arm-Exchange by Non-Activating Antibody Variants

Certain applications, such as T-cell-mediated cytotoxicity of target cells as shown in Example 11, require the generation and use of bispecific antibody (bsAb) variants where one F(ab) arm can engage with target A and the other F(ab) arm engages with target B.

Here, we evaluated the efficiency by which bsAb variants were generated, by controlled Fab-arm-exchange (cFAE), as described in Example 1, upon introduction of non-activating mutations in the constant heavy chain region of parental monospecific antibodies in addition to either the F405L or K409R mutation, each present in one of the monospecific antibodies, required for efficient heterodimer formation. Briefly, antibodies were mixed at equimolar concentration and incubated with 75 mM 2-mercaptoethylamine-HCl (2-MEA; Cat #30078, Sigma Aldrich) at 31° C. for 5 hours. Subsequently buffer-exchanging against PBS was achieved as described in Example 1 and the concentration was measured by absorbance at 280 nm using a NanoDrop ND-2000-EU Spectrophotometer (Thermo Fisher). Mass spectrometric analysis was performed to determine the bispecific and residual homodimer content using an Orbitrap Q-Exactive Plus mass spectrometer (Thermo Fischer).

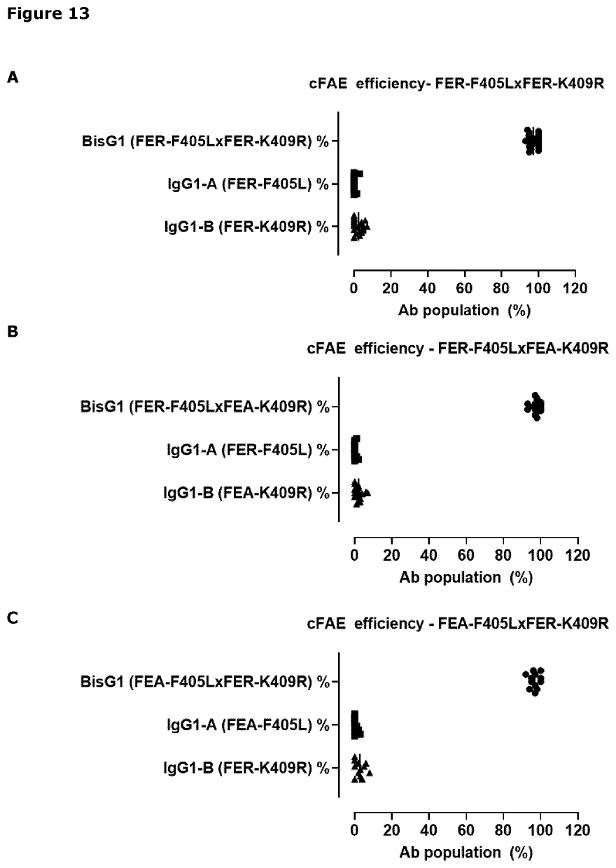

Evaluation of the efficiency of cFAE revealed that introduction of non-activating mutations L234F-L235E-G236R in addition to a F405L or K409R mutation, each present in one of the monospecific antibodies, resulted in efficient cFAE with at least 95% of the population being an IgG1 bsAb (BisG1) with the remaining populations being one or both monospecific antibodies (indicated in the figure as IgG1-A and IgG1-B); FIG. 13A). Moreover, efficiency of cFAE was assessed when bsAb variants were created with an asymmetric distribution of the non-activating mutations, i.e. L234F-L235E-G236R present in one monospecific antibody variant in addition to F405L or K409R mutations, and L234F-L235E-D265A present in the other monospecific antibody variant in addition to F405L or K409R mutations. Analysis revealed similar efficiency in generating bsAb variants (FIG. 13B, C), as shown for variants harboring L234F-L235E-G236R non-activating mutations in both arms of the bsAb, in addition to a F405L or K409R mutation, each present in one of the monospecific antibodies (FIG. 13A).

In summary, antibody variants harboring the novel L234F-L235E-G236R non-activating mutations in the IgG1 constant heavy chain region retain the capacity to efficiently form bsAb variants by controlled Fab-arm-exchange using the F405L and K409R mutation, each present in one of the monospecific antibodies, allowing heterodimerization and preventing formation of monospecific homodimers.

### Example 16: Production Levels of Non-Activating Antibody Variants

In this Example, production levels of antibody variants harboring either L234F-L235E-G236R or L234F-L235E-D265A non-activating mutations in the constant heavy chain region in addition to either F405L or K409R mutations were assessed.

All antibody variants were produced in Expi293F cells as described in Example 1. To avoid differences in production titer due to usage of particular clones for one non-activating antibody variant but not the other, and to allow direct comparison of production titer of antibody variants harboring either L234F-L235E-D265A or L234F-L235E-G236R non-activating mutations in the constant heavy chain region, the same antibody clones are represented within each group, i.e. F405L antibody variants or K409R antibody variants.

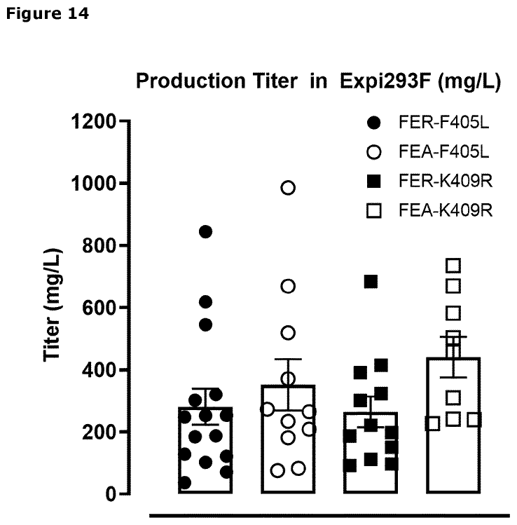

Analysis of production levels revealed that non-activating antibody variants harboring L234F-L235E-G236R mutations in addition to a F405L mutation (closed circles) were produced at similar levels to antibody variants harboring the non-activating mutations L234F-L235E-D265A in addition to a F405L mutations (open circles; FIG. 14). Whereas production levels of antibody variants harboring L234F-L235E-G236R non-activating mutations in addition to a K409R mutation (closed squares) were similar to antibody variants that included a F405L mutation in addition to non-activating mutations, a small increase in average production levels was observed for antibody variants harboring L234F-L235E-D265A non-activating mutations in addition to a K409R mutation (open squares; FIG. 14).

In summary, no major differences in production levels of non-activating antibody variants harboring L234F-L235E-G236R mutations in addition to either F405L or K409R mutations compared to variants harboring L234F-L235E-D265A non-activating mutations in addition to F405L or K409R mutations were observed.

### Example 17: Assessment of Stability and Solubility of IgG1 Non-Activating Antibody Variants by PEG Midpoint, DLS and DSF Analysis

Protein stability and solubility characteristics of anti-CD20 and anti-CD3 IgG1 antibody variants harboring non-activating mutations in the constant heavy chain region were assessed using a PEG-induced precipitation assay, differential scanning fluorimetry (DSF) and dynamic light scattering (DLS) assays.

Samples of anti-human CD20 and anti-human CD3 IgG1 (huCLB-T3/4) antibody variants were formulated in PBS pH 7.4 at a concentration of approximately 20 mg/mL (concentration range 18.7-21.6 mg/mL; filtration applied for anti-human CD3 IgG1 antibody variants). PBS was used as non-optimal formulation to allow better comparisons of protein stability.

To assess conformational protein stability, DSF was performed in an iQ5 Multicolor Real-Time PCR detection system (Bio-Rad) capable of detecting changes in fluorescence intensity caused by binding of the extrinsic dye Sypro-Orange (5000×concentrate in DMSO, Cat #S5692, Sigma-Aldrich) to hydrophobic regions exposed by denatured IgG. Sypro-Orange was diluted 320-fold in PBS (Hyclone GE Healthcare, Cat #SH3A383.03) pH 7.4 or in 30 mM sodium acetate pH 4 (Cat #25022-1KG-R, Sigma-Aldrich) to a concentration of 75 mM. A thermal melt curve can be derived from measuring the increasing fluorescence during controlled, stepwise thermal denaturation of the analyzed IgG. Therefore, 5 μL samples (diluted in PBS; concentration range 1 mg/mL+/−10%) of anti-human CD20 IgG1 antibody variants harboring either the K409R mutation, the L234F-L235E-D265A-K409R or L234F-L235E-G236R-K409R mutations, or anti-human CD3 IgG1 antibody variants harboring the F405L mutation, the L234F-L235E-D265A-F405L or L234F-L235E-G236R-F405L mutations, mixed with 20 μL of 75 mM Sypro-Orange (in either PBS pH 7.4 or 30 mM sodium acetate pH 4), were prepared in duplicate in iCycler iQ 96-well PCR plates. Fluorescence (Excitation 485 nm, Emission 575 nm) was recorded at increasing temperatures ranging from 25° C. to 95° C., in stepwise increments of 0.5° C. per increment and 15 second duration plus the time necessary to record the fluorescence of all wells. The data was analyzed using Bio-Rad CFX Manager Software 3.0 and melting points were determined from the fluorescence versus temperature graphs by the software.

DLS analysis was performed to assess the propensity of the antibody variants mentioned above to aggregate in solution, as a measure of colloidal stability. 20 μL of the abovementioned antibody variants (concentration range 1 mg/mL+/−10%) in PBS pH 7.4 was analyzed using a DynaPro Plate Reader II (Wyatt Technology) with Dynamics 7 software. Samples were applied in triplicate in round 384-wells IQ-LV plates (Aurora Biotechnologies, Cat #1011-00110), centrifuged for 3 min at 2,111 xg and covered with paraffin oil. Prior to the measurement the plates were centrifuged again for 3 min at 2,111 xg. Thermal scan measurements were performed with a continuously increasing temperature (1° C. increase/data point for each specific sample) throughout the experiment. The cumulant fit procedure was used to analyze the data, resulting in determination of the apparent radius. A cut-off value of 2.1 nm was used, excluding the peaks with lower radius which are often caused by software artefacts and not consistently observed in each acquisition. A refractive index of 1.333 and a viscosity of 1.019 cP were used for PBS buffer at 25° C. (standard values supplied in the Dynamics software). Data was processed in Microsoft Excel to determine the onset of aggregation (T_{agg}). T_{agg} was determined by calculating the mean and standard deviation (n=10, first 10 measurements) of the radius for every well. By setting a 99.99%-confidence interval for each measurement individually, the first sequential value (starting from 25° C. to 80° C.) that would fall outside the confidence interval was tagged as T_{agg}. Five consecutive measurements were performed in triplicate per experiment.

The PEG-induced precipitation assay was performed to assess relative protein solubility. Two buffers were prepared; Buffer A: 50 mM Phosphate buffer pH 7.0 (Sodium Phosphate monobasic; Fluka, Cat #17844+Sodium Phosphate dibasic dihydrate, Fluka Cat #71633); Buffer B: 50 mM Phosphate buffer pH 7.0+40% (w/v) PEG 8000 (Sigma-Aldrich, Cat #P5413). Different amounts of Buffer B were mixed with Buffer A to generate a series of 11 different PEG concentrations, ranging from 0% to 40% PEG. Aliquots (80 μL) of each PEG concentration buffer were added to different wells of a 96-wells plate (UV Star® 96 wells plate, half area, Greiner Bio-one, Cat #675801). Of each antibody sample (diluted to 1 mg/mL in PBS), 20 μL is added to wells containing the series of PEG-containing buffer. The plates were covered with a seal and shaken for 5 min at 750 rpm. The plate was left overnight at RT and subsequently shaken for 5 min. The plate was then centrifuged for 20 min at 4000 rpm to remove precipitated antibody. From each well, 80 μL was transferred carefully to a new UV Star 96-wells plate without disturbing the centrifuged precipitate. Absorbance at 280 nm (A280, correlating with the amount of solubilized protein) was measured on a Synergy™ 2 Multi-mode Microplate Reader (Biotek Instruments, BioSPX) and recalculated to a volume of 100 μL (path length correction) and blank values (PEG without antibody) were subtracted. The corrected A280 values were plotted versus the PEG concentration in Graphpad Prism 8. The data was analyzed using non-linear regression, in which the PEG midpoint (%) reflects the concentration of the test sample at which 50% of the antibody was precipitated (i.e. 50% loss in A280 compared to 0% PEG). The PEG midpoint is used as a measure of solubility, with a higher PEG midpoint corresponding to better solubility.

The results of the assays described above are summarized in Tables 3, 4. DSF analysis revealed a melting temperature (Tₘ) of 68.0° C. at pH 7.4, which was decreased to a Tₘ1 of 56.0° C. and Tₘ2 of 62.0° C. at pH 4.0. The Tₘ recorded for anti-human CD20 IgG1-L234F-L235E-G236R-K409R at pH 7.4 was comparable to the K409R-containing variant (67.8° C. vs 68.0° C., respectively). At pH 4.0, the variant harboring the L234F-L235E-G236R-K409R mutations demonstrated one Tₘ at 58.0° C. In contrast, the anti-human CD20 IgG1 variant harboring the L234F-L235E-D265A-K409R mutations had a decreased Tₘ1 of 63.3° C. and Tₘ2 of 68.5° C. at pH 7.4, while at pH 4.0 three Tₘ were recorded, namely 48.5° C., 57.0° C., and 61.0° C. For the anti-human CD3 IgG1 antibody, the variants harboring either the F405L mutation alone, or the L234F-L235E-G236R-F405L mutations had the same Tₘ at pH 7.4 (68.0° C.) and highly similar Tₘ1 and Tₘ2 at pH 4.0 (F405L: 53.5 and 70.5° C.; L234F-L235E-G236R-F405L: 54.5 and 70.8° C.). At pH 7.4, the Tₘ of the IgG1-huCLB-T3/4-L234F-L235E-D265A-F405L antibody variant was decreased (63.0° C.) as compared to the F405L- and L234F-L235E-G236R-F405L-containing variants. Also, at pH 4.0, the first Tₘ recorded for variant IgG1-huCLB-T3/4-L234F-L235E-D265A-F405L was decreased (48.5° C.) as compared to the other IgG1-huCLB-T3/4 variants, while a second Tₘ of 71.0° C. was observed for IgG1-huCLB-T3/4-L234F-L235E-D265A-F405L which is in line with the other IgG1-huCLB-T3/4 variants.

The anti-human CD20 IgG1-K409R and anti-human CD20 IgG1-L234F-L235E-D265A-K409R antibody variants showed the lowest aggregation temperature (T_{agg}; 57.9° C. and 58.5° C., respectively), followed by the L234F-L235E-G236R-K409R-containing variant (59.9° C.). For the IgG1-huCLB-T3/4 variants, the lowest T_{agg} was observed for the variant harboring the L234F-L235E-D265A-F405L mutations (58.7° C.), followed by the F405L-containing and L234F-L235E-G236R-F405L-containing variants (61.7° C. and 62.0° C., respectively).

The anti-human CD20 IgG1 antibody variants harboring either the K409R mutation, the L234F-L235E-D265A-K409R or L234F-L235E-G236R-K409R mutations demonstrated a comparable relative solubility profile as determined through the PEG-induced precipitation assay. Overall, anti-human CD3 IgG1 variants demonstrated a lower relative solubility as compared to the anti-human CD20 IgG1 variants. The anti-human CD3 IgG1 variants harboring the L234F-L235E-D265A-F405L or L2345F-L23E-G236R-F405L mutations were comparable in terms of solubility. Both these variants were relatively slightly less soluble than the anti-human CD3 IgG1-F405L variant.

Taken together, anti-human CD20 IgG1 antibody variants harboring non-activating mutations demonstrated a comparable profile in terms of solubility and propensity to aggregate as compared to the K409R-containing control variant. However, while the variants harboring the L234F-L235E-G236R-K409R mutations showed a comparable protein stability profile to the K409R-containing control variant, the anti-human CD20 IgG1-L234F-L235E-D265A-K409R variant demonstrated a lower protein stability profile. For anti-human CD3 IgG1 variants, the variants harboring the F405L or L234F-L235E-G236R-F405L mutations showed a comparable propensity to aggregate and protein stability profile. Solubility of both non-activating variants was decreased as compared to the F405L-containing control variant. The variant harboring the L234F-L235E-D265A-F405L mutations demonstrated decreased protein stability and a slightly higher propensity to aggregate as compared to the variants harboring the F405L or L234F-L235E-G236R-F405L mutations. Solubility of the L234F-L235E-D265A-F405L-containing variant was comparable to the L234F-L235E-G236R-F405L-containing variant.

To conclude, when formulated at high concentrations in PBS, anti-human CD20 and CD3 IgG1 variants harboring the L234F-L235E-G236R mutations demonstrated a more robust protein stability profile than L234F-L235E-D265A-containing variants. In addition, anti-human CD3 IgG1 variants harboring the L234F-L235E-G236R mutations showed a slightly lower propensity to aggregate than L234F-L235E-D265A-containing variants.

Table 3 shows the protein conformational stability, as determined through DSF analysis, for anti-human CD20 IgG1 and anti-CD3 IgG1 (huCLB-T3/4) antibody variants harboring non-activating mutations in the constant heavy chain region. DSF: Differential Scanning Fluorimetry; Tₘ: melting temperature.

Table 4 shows the protein solubility, as determined through PEG midpoint determination assay, and propensity to aggregate, as determined through DLS analysis, for anti-human CD20 IgG1 and anti-human CD3 IgG1 antibody variants harboring non-activating mutations in the constant heavy chain region. T_{agg}: Aggregation temperature; PEG: polyethylene glycol; DLS: dynamic light scattering.

TABLE 3

| | | DSF¹ | | | | | |
|---|---|---|---|---|---|---|---|
| | | PBS pH 7.4 | | | Sodium Acetate pH 4.0 | | |
| | | Tₘ1 | Tₘ2 | Tₘ3 | Tₘ1 | Tₘ2 | Tₘ3 |
| Antibody Variant | | (° C.) | (° C.) | (° C.) | (° C.) | (° C.) | (° C.) |
| IgG1- | L234F-L235E- | 63.3 | 68.5 | — | 48.5 | 57.0 | 61.0 |
| CD20² | D265A-K409R | | | | | | |
| | K409R | 68.0 | — | — | 56.0 | 62.0 | — |
| | L234F-L235E- | 67.8 | — | — | 58.0 | — | — |
| | G236R-K409R | | | | | | |
| IgG1- | L234F-L235E- | 63.0 | 72.0 | — | 48.5 | 71.0 | — |
| CD3² | D265A-F405L | | | | | | |
| | F405L | 68.0 | — | — | 53.5 | 70.5 | — |
| | L234F-L235E- | 68.0 | — | — | 54.5 | 70.8 | — |
| | G236R-F405L | | | | | | |

¹Differential Scanning Fluorimetry
²IgG concentration in assay ~0.2 mg/mL

TABLE 4

| | | DLS^{1,2} | PEG^{3,4} |
|---|---|---|---|
| Antibody Variant | | T_{agg} (° C.) | Midpoint (%) |
| IgG1-CD20³ | L234F-L235E-D265A- | 58.5 | 19.4 |
| | K409R | | |
| | K409R | 57.9 | 19.7 |
| | L234F-L235E-G236R- | 59.9 | 19.2 |
| | K409R | | |
| IqG1-CD3³ | L234F-L235E-D265A-F405L | 58.7 | 11.0 |
| | F405L | 61.7 | 14.3 |
| | L234F-L235E-G236R-F405L | 62.0 | 12.0 |

¹Dynamic Light Scattering
²IgG concentration in assay~1 mg/mL
³Polyethylene glycol
⁴IgG concentration in assay~0.2 mg/mL

### Example 18: Impact on Protein Stability of IgG1 Non-Activating Antibody Variants after Storage at Different Temperatures for 1 or 4 Months

In Example 17, the protein stability and solubility profile of anti-CD20 and anti-CD3 IgG1 antibody variants harboring non-activating mutations in the constant heavy chain region were assessed. Here, protein stability of such antibody variants was assessed after storage at 2-8° C. or 40° C. for 1 or 4 months using different assays.

Samples of anti-human CD20 IgG1 and anti-human CD3 IgG1 (huCLB-T3/4) antibody variants were formulated in PBS pH 7.4 at a concentration of approximately 20 mg/mL (concentration range 18.7-21.6 mg/mL; filtration applied for anti-human CD3 IgG1 antibody variants). PBS was used as non-optimal formulation to allow better comparisons of protein stability. The anti-human CD20 IgG1 variants harbored either the K409R mutation, the L234F-L235E-D265A-K409R or L234F-L235E-G236R-K409R mutations, while the anti-human CD3 IgG1 antibody variants harbored either the F405L mutation, the L234F-L235E-D265A-F405L or L234F-L235E-G236R-F405L mutations. Samples were incubated for 1 month at 2-8° C., 1 month at 40° C., 4 months at 2-8° C., 4 months at 40° C. All samples were subsequently analyzed by High-Performance Size-Exclusion Chromatography (HP-SEC), capillary Isoelectric Focusing (cIEF), Capillary Electrophoresis-Sodium Dodecyl Sulfate (CE-SDS) and Dynamic Light Scattering (DLS).

HP-SEC analysis was performed using a Waters Alliance 2795 separation module (Waters) equipped with a Waters 2487 dual A absorbance detector (Waters), using a TSK column (G3000SWxl; Tosoh Biosciences, Cat #6095006) and a TSK-gel SWxl guard column (Tosoh Biosciences, Cat #6095007). The samples (diluted to 5 mg/mL in PBS pH 7.4) were run at 1 mL/min using mobile phase 0.1 M sodium sulfate (Na₂SO₄, Sigma-Aldrich, Cat #31481)/0.1 M sodium phosphate pH 6.8 (NaH₂PO₄, Sigma-Aldrich Cat #17844/Na₂HPO₄·2H₂O, Sigma-Aldrich Cat #71633). Results were processed using Empower 3 software and expressed per peak as percentage of total peak height.

The cIEF analysis was performed using an ICE3 Analyzer (ProteinSimple). Each anti-human CD20 IgG1 variant was mixed with an assay mix ultimately containing 0.3 mg/mL antibody, 0.35% Methyl Cellulose (ProteinSimple, Cat #101876); 2% Pharmalytes 3-10(GE Healthcare, Cat #17-0456-01); 6% Pharmalytes 8-10.5 (GE Healthcare, Cat #17-0455-01); 0.5% pI marker 7.65 and 0.5% pI marker 10.10 (ProteinSimple, Cat #102407 and Cat #102232, respectively). Focusing was performed for 1 minute at 1500 V (prefocusing) and 7 minutes at 3000 V. Anti-human CD3 IgG1 variants were mixed with an assay mix ultimately containing 0.3 mg/mL antibody, 3.2M Urea (Sigma-Aldrich, Cat #33247-1 kg), 0.35% Methyl Cellulose; 2% Pharmalytes 3-10; 6% Pharmalytes 8-10.5; 0.5% pI marker 7.65 and 0.5% pI marker 10.10. Focusing was performed for 2 minutes at 1500 V (prefocusing) and 9 minutes at 3000 V. The whole-capillary absorption image was captured by a charge-coupled device camera. After calibration of the peak profiles, the data was analyzed for pI and area (%) by Empower 3 software (Waters).

CE-SDS was performed using a LabChip GXII Touch (Perkin Elmer, Cat #CLS138160) on a HT Protein Express LabChip (Perkin Elmer, Cat #760499) using the HT Protein Express Reagent kit (Perkin Elmer, Cat #CLS960008) with few modifications. Samples were diluted to 1 mg/mL in PBS pH 7.4 and samples were prepared by 2 μL diluted sample+7 μL denaturing solution+35 μL MilliQ water. Samples were prepared in 96-well Bio-Rad HSP9601 plates (Cat #4TI-0960). Analysis was performed under both non-reducing and reducing conditions (addition of DTT). Samples were denatured by incubation at 70° C. for 3 minutes. The chip was prepared according to manufacturer's instructions and the samples were run with the HT antibody analysis 200 high sensitivity settings. Protein size (kDa) and purity (%) was analyzed using the Labchip GXII software V5.3.2115.0.

Dynamic light scattering (DLS) was performed essentially as described in Example 17, however here, DLS was performed at a constant temperature of 25° C. to determine the average particle size.

All results are summarized in Tables 5-7. As detected by HP-SEC, storage of Anti-human CD20 IgG1 and IgG1-huCLB-T3/4 antibody variants at 2-8° C. for 4 months did not considerably affect the percentage of multimers detected in the samples as compared to 1 month exposure. The percentage of multimers present was highest in the samples containing anti-human CD20 and CD3 IgG1 variants subjected to 40° C. for 4 months and to a lesser extent to 40° C. for 1 month. Of the anti-human CD20 IgG1 samples at 40° C., the samples of the variant harboring the L234F-L235E-D265A-K409R mutations contained higher percentages of multimers than the samples of variants harboring either the K409R or L234F-L235E-G236R-K409R mutations. Besides, no differences in the percentages of degradation were observed between samples containing the anti-human CD20 IgG1 variants. For anti-human CD3 IgG1 antibody variants, an enhanced percentage of multimers was detected in samples of the L234F-L235E-G236R-F405L-containing variant after subjecting the samples to 40° C. for 4 months and to a lesser extent to 40° C. for 1 month, while the percentage of multimers was comparable for samples of the variants harboring the L234F-L235E-D265A-F405L or F405L mutations. Upon exposing the anti-human CD3 IgG1-L234F-L235E-G236R-F405L variant to 40° C. for 1 month, a relatively high percentage of protein was degraded, and increasingly so after 4 months of exposure.

cIEF analysis was used to study alterations in the percentages of acidic, neutral, and basic peaks of the antibody variants in response to the different stress conditions. The change in the percentage of acidic protein present in a sample is used as a surrogate measure for deamidation. Of note, the neutral peak was split in two peaks for the samples stored for 1 month at either of the temperatures, which were summed up as the percentage of neutral protein. An increase in the percentage of acidic protein was observed in all samples between 1 and 4 months of storage at 40° C., while for samples stored at 2-8° C., such an increase in the percentage of acidic protein between 1 and 4 months of storage was not observed. The percentages of acidic protein were comparable for all tested anti-human CD20 IgG1 antibody variants. The anti-human CD3 IgG1 variants harboring either the F405L mutation alone, the L234F-L235E-D265A-F405L or L234F-L235E-G236R-F405L mutations also showed comparable percentages of acidic protein in all tested conditions, except for the F405L-containing variant which reached a maximum of 79% acidic protein after 4 months of storage at 40° C. while the variants harboring the non-activating mutations reached 98% and 96% in this condition, respectively.

The percentage of intact protein detected by CE-SDS served as a measure for protein integrity and degradation in response to the tested stress conditions. The anti-human CD20 IgG1 variants largely retained their intact structure after storing the samples at 2-8° C. for 1 or 4 months, or storing at 40° C. for 1 month. However, the percentages of detected intact IgG were decreased after exposure of the samples to 40° C. for 4 months for all anti-human CD20 IgG1 variants, with the strongest degradation observed for the K409R-containing variant. A similar pattern was observed when studying the percentage of HC and LC, although to a lesser extent and with the exception that the variants harboring the K409R mutation or L234F-L235E-D265A-K409R mutations showed a similar percentage of HC and LC. The anti-human CD3 IgG1 antibody variants demonstrated a similar degradation pattern, with the L234F-L235E-G236R-F405L-containing variant showing the lowest percentage of intact IgG protein after exposure to 40° C. While the percentages of HC and LC of the anti-human CD3 IgG1 variants showed a similar pattern overall, the lowest percentage of HC and LC was detected for the variant harboring the L234F-L235E-D265A-F405L mutations.

DLS analysis was performed to determine the average particle size (radius) after subjecting the antibody variant samples to the indicated stress conditions, which is a surrogate measure for the level of aggregation. For all anti-human CD20 IgG1 variants, exposure of the samples to 40° C. for 4 months substantially increased the average particle radius as compared to the other conditions tested. The highest average particle radii were detected for the variant harboring the L234F-L235E-G236R-K409R mutations as compared to the K409R and L234F-L235E-D265A-K409R variants in all tested conditions. Particles were already present in the starting material of the sample containing the L234F-L235E-G236R-K409R-containing variant. For the anti-human CD3 IgG1 variants, a relatively large average particle size was detected for the anti-human CD3 IgG1-F405L variant in all tested conditions. The cause of these larger radii is probably unrelated to the applied stress conditions as particles were already present in the starting material. No increase in average particle size is observed after stress. Substantially lower average particles sizes were detected for the variants harboring the non-activating mutations L234F-L235E-D265A-F405L or L234F-L235E-G236R-F405L.

In summary, incubation at 40° C. for 1 to 4 months of anti-human CD20 and CD3 IgG1 antibody variants harboring non-activating mutations in the constant heavy chain region resulted in increased multimerization as compared to variants stored at 2-8° C. While more multimerization was observed for the anti-human CD20 IgG1 variants harboring the L234F-L235E-D265A-K409R than variants harboring the L234F-L235E-G236R-K409R mutations, variants of anti-human CD3 IgG1 harboring the L234F-L235E-G236R-F405L mutations showed more multimerization than variants harboring the L234F-L235E-D265A-F405L mutations. In general, no increase in the percentage of acidic protein, used as a surrogate measure of deamidated protein, was observed for any of the tested antibody variants when stored at 2-8° C. for 1 or 4 months. While samples stored at 40° C. for 1 month contained relatively more acidic protein as compared to storage at 2-8° C., a further increase in the percentage of acidic protein was observed for all tested variants after storage at 40° C. for 4 months. After storage at 40° C., less intact protein was detected of all antibody variants, which was most pronounced after 4 months of storage. The anti-human CD3 IgG1-L234F-L235E-G236R-F405L variant showed a slightly stronger decrease in the percentage of intact protein detected after storage at 40° C. for 4 months. Finally, more aggregation was observed for all anti-human CD20 IgG1 variants when stored at 40° C. for 4 months. Of the anti-human CD3 IgG1 variants, both variants harboring the non-activating mutations contained substantially less aggregates than the F405L-containing variant.

An acceptable stability profile was observed for both anti-human CD20 and CD3 IgG1 variants harboring either the L234F-L235E-D265A or L234F-L235E-G236R non-activating mutations. Storage at 40° C. for 4 months resulted in a lower stability profile of all tested antibody variants.

Table 5 shows the percentages of multimers, monomers and degraded protein as detected through HP-SEC analysis, in samples containing anti-human CD20 and CD3 IgG1 (huCLB-T3/4) antibody variants harboring non-activating mutations or a single heterodimerization promoting mutation in the constant heavy chain region that have been stored at 2-8° C. or 40° C. for 1 or 4 months. HP-SEC: High Performance Size Exclusion Chromatography.

Table 6 shows the percentages of acidic, neutral and basic isoforms present in samples containing anti-human CD20 and CD3 IgG1 variants harboring non-activating mutations or a single heterodimerization promoting mutation in the constant heavy chain region that have been stored at 2-8° C. or 40° C. for 1 or 4 months, as determined by cIEF analysis. The change in percentage of acidic isoform present in a sample is a surrogate for the level of sample deamidation. CIEF: capillary Isoelectric Focusing.

Table 7 shows the percentages of intact protein and sum HC and LC, as determined by non-reducing and reducing CE-SDS analysis, and the average radius (in nm) of particles, as determined by DLS analysis, detected in samples containing anti-human CD20 and CD3 IgG1 variants harboring non-activating mutations or a single heterodimerization promoting mutation in the constant heavy chain region that have been stored at 2-8° C. or 40° C. for 1 or 4 months. CE-SDS: Capillary Electrophoresis Sodium Dodecyl Sulfate; DLS: Dynamic Light Scattering.

TABLE 5

| | | | HP-SEC | | |
|---|---|---|---|---|---|
| Antibody Variant | | Treatment | Multimer(%) | Monomer(%) | Degradation(%) |
| IgG1-CD20¹ | L234F-L235E- | 1 m 2-8° C. | 1.1 | 98.6 | 0.3 |
| | D265A-K409R | 1 m 40° C. | 3.1 | 95.7 | 1.3 |
| | | 4 m 2-8° C. | 1.3 | 98.3 | 0.3 |
| | | 4 m 40° C. | 6.6 | 90.6 | 2.8 |
| | K409R | 1 m 2-8° C. | 1.2 | 98.4 | 0.4 |
| | | 1 m 40° C. | 1.8 | 97.1 | 1.1 |
| | | 4 m 2-8° C. | 1.1 | 98.5 | 0.4 |
| | | 4 m 40° C. | 3.4 | 93.9 | 2.7 |
| | L234F-L235E- | 1 m 2-8° C. | 0.7 | 99.3 | 0 |
| | G236R-K409R | 1 m 40° C. | 1.5 | 97.6 | 0.9 |
| | | 4 m 2-8° C. | 10.7 | 99.3 | 0 |
| | | 4 m 40° C. | 3.5 | 94 | 2.6 |
| IgG1-CD3¹ | L234F-L235E- | 1 m 2-8° C. | 10.3 | 99.5 | 0.2 |
| | D265A-F405L | 1 m 40° C. | 1.4 | 97.6 | 1.1 |
| | | 4 m 2-8° C. | 0.2 | 99.7 | 0.1 |
| | | 4 m 40° C. | 2 | 95.6 | 2.4 |
| | F405L | 1 m 2-8° C. | 10.3 | 99.5 | 0.2 |
| | | 1 m 40° C. | 1.1 | 97.9 | 0.9 |
| | | 4 m 2-8° C. | 0.1 | 99.9 | 0.1 |
| | | 4 m 40° C. | 1.7 | 196 | 2.3 |
| | L234F-L235E- | 1 m 2-8° C. | 0.4 | 99.6 | 0 |
| | G236R-F405L | 1 m 40° C. | 2.7 | 87.6 | 9.7 |
| | | 4 m 2-8° C. | 10.3 | 99.7 | 0 |
| | | 4 m 40° C. | 3.6 | 81.4 | 15.3 |

¹Stress conditions tested at IgG concentration~20 mg/mL

TABLE 6

| | | | CIEF | | |
|---|---|---|---|---|---|
| Antibody Variant | | Treatment | Acidic (%) | Neutral (%) | Basic (%) |
| IgG1-CD201 | L234F-L235E- | 1 m 2-8° C. | 49 | 48 | 3 |
| | D265A-K409R | 1 m 40° C. | 76 | 19 | 4 |
| | | 4 m 2-8° C. | 47 | 49 | 4 |
| | | 4 m 40° C. | 95 | 3 | 3 |
| | K409R | 1 m 2-8° C. | 39 | 54 | 7 |
| | | 1 m 40° C. | 72 | 22 | 7 |
| | | 4 m 2-8° C. | 41 | 51 | 9 |
| | | 4 m 40° C. | 92 | 4 | 3 |
| | L234F-L235E- | 1 m 2-8° C. | 20 | 76 | 4 |
| | G236R-K409R | 1 m 40° C. | 65 | 27 | 8 |
| | | 4 m 2-8° C. | 17 | 80 | 4 |
| | | 4 m 40° C. | 94 | 5 | 2 |
| IgG1-CD31 | L234F-L235E- | 1 m 2-8° C.² | 30 | 67 | 3 |
| | D265A-F405L | 1 m 40° C.² | 66 | 34 | 0 |
| | | 4 m 2-8° C. | 30 | 67 | 3 |
| | | 4 m 40° C. | 98 | 2 | 0 |
| | F405L | 1 m 2-8° C.² | 20 | 80 | 1 |
| | | 1 m 40° C.² | 56 | 43 | 1 |
| | | 4 m 2-8° C. | 23 | 73 | 5 |
| | | 4 m 40° C. | 79 | 15 | 7 |
| | L234F-L235E- | 1 m 2-8° C.² | 23 | 78 | 0 |
| | G236R-F405L | 1 m 40° C.² | 63 | 36 | 0 |
| | | 4 m 2-8° C. | 22 | 74 | 5 |
| | | 4 m 40° C. | 96 | 4 | 1 |

¹Stress conditions tested at IgG concentration~20 mg/mL
²Neutral peak was split due to technical artifact. Peak % of both peaks was summed up.

TABLE 7

| | | | CE-SDS | | DLS |
|---|---|---|---|---|---|
| Antibody Variant | | Treatment | Intact IgG1 (%) | HC + LC (%) | Radius (nm) |
| IgG1-CD20¹ | L234F-L235E- | 1 m 2-8° C. | 96 | 99 | 6.5 |
| | D265A-K409R | 1 m 40° C. | c.i.² | 96 | 7.6 |
| | | 4 m 2-8° C. | 98 | 100 | 6.3 |
| | | 4 m 40° C. | 66 | 89 | 21.6³ |
| | K409R | 1 m 2-8° C. | 92 | 98 | 6.1 |
| | | 1 m 40° C. | 82 | 96 | 6.5 |
| | | 4 m 2-8° C. | 98 | 99 | 6.1 |
| | | 4 m 40° C. | 57 | 90 | 41.5³ |
| | L234F-L235E- | 1 m 2-8° C. | 98 | 100 | 12.4³ |
| | G236R-K409R | 1 m 40° C. | 89 | 98 | 27.0³ |
| | | 4 m 2-8° C. | 99 | 100 | 10.7³ |
| | | 4 m 40° C. | 67 | 92 | 74.5³ |
| IqG1-CD3¹ | L234F-L235E- | 1 m 2-8° C. | 97 | 100 | 8.7 |
| | D265A-F405L | 1 m 40° C. | 92 | 97 | 9.1 |
| | | 4 m 2-8° C. | 98 | 99 | 8.8 |
| | | 4 m 40° C. | 78 | 91 | 9.3 |
| | F405L | 1 m 2-8° C. | 97 | 100 | 19.3³ |
| | | 1 m 40° C. | 93 | 99 | 22.1³ |
| | | 4 m 2-8° C. | 99 | 100 | 16.4³ |
| | | 4 m 40° C. | 80 | 95 | 18.0³ |
| | L234F-L235E- | 1 m 2-8° C. | 98 | 100 | 7.5 |
| | G236R-F405L | 1 m 40° C. | 86 | 97 | 7.4 |
| | | 4 m 2-8° C. | 99 | 99 | 7.5 |
| | | 4 m 40° C. | 69 | 93 | 7.6 |

¹Stress conditions tested at IgG concentration~20 mg/mL
²Intact IgG with shoulder
³Multimodal: several populations with varying radii detected
m: month(s)
c.i .: complex interpretation

### Example 19: Impact of Freeze/Thawing on Stability of IgG1 Non-Activating Antibody Variants

The impact of freeze/thaw cycles on the stability of IgG1 antibody variants harboring non-activating mutations in the constant heavy chain region was assessed using the assays described in Example 18.

Samples of anti-human CD20 and CD3 IgG1 (huCLB-T3/4) antibody variants were formulated in PBS pH 7.4 at a concentration of approximately 20 mg/mL (concentration range 18.7-21.6 mg/mL; filtration applied for anti-human CD3 IgG1 antibody variants). PBS was used as non-optimal formulation to allow better comparisons of protein stability. The anti-human CD20 IgG1 variants harbored either the K409R mutation, the L234F-L235E-D265A-K409R or L234F-L235E-G236R-K409R mutations, while the anti-human CD3 IgG1 antibody variants harbored either the F405L mutation, the L234F-L235E-D265A-F405L or L234F-L235E-G236R-F405L mutations. Two independent identical samples were frozen at <−65° C. and thawed to room temperature (RT) in three cycles. Subsequently, the protein stability was studied using HP-SEC, cIEF, CE-SDS and DLS, as described in Example 18.

After three cycles of freezing and thawing, an increase in the percentage of multimers is observed in the samples containing the anti-human CD20 IgG1 variants, as determined by HP-SEC, although the percentages of multimers present in the samples are low (ranging from 0.7% to 1,2% for the reference samples, and 1.8% to 2.8% for the freeze-thawed samples). For the anti-human CD3 IgG1 antibody variants, the percentages of detected multimers, ranging from 0.2% to 0.7%, were considered too low to allow reliable conclusions.

No substantial differences in the percentages of acidic protein were detected upon freezing and thawing the anti-human CD20 and CD3 IgG1 antibody variants, as determined by cIEF analysis. Overall, the variants harboring the L234F-L235E-D265A mutations in combination with either K409R or F405L showed the highest percentages of acidic protein, also in the reference samples, probably due to sialylation of D265A.

Freezing and thawing of the antibody variant samples did also not affect the percentages of intact protein for any of the IgG1 variants, with percentages of intact protein ranging from 99% to 100%. Similarly, the percentages of HC and LC were also not affected by freezing and thawing the samples.

The average particle size (radius), as assessed by DLS analysis, was not affected by freezing and thawing of the samples. Relatively more aggregates of anti-human CD20 IgG1 variants were detected for the variants harboring the L234F-L235E-G236R-K409R mutations, while for the anti-human CD3 IgG1 variants relatively most aggregates were detected for the F405L-containing variant.

In summary, freezing and thawing of samples containing anti-human CD20 and CD3 IgG1 antibody variants harboring the non-activating mutations L234F-L235E-D265A or L234F-L235E-G236R in combination with either K409R or F405L, did not result in relevant enhancements of multimerization, deamidation, or protein degradation as compared to the reference samples. Therefore, it was concluded that antibody variants harboring either the L234F-L235E-G236R or L234F-L235E-D265A mutations were similarly capable of retaining protein stability upon repeated freeze/thaw cycles.

Table 8 shows the percentages of multimers and monomers as detected by HP-SEC analysis, in samples containing anti-human CD20 IgG1 and anti-human CD3 IgG1 antibody variants harboring non-activating mutations in the constant heavy chain region that have been subjected to three freeze/thaw cycles. Indicated values are averages of 2 individual samples subjected to 3 freeze/thaw cycles. HP-SEC: High Performance Size Exclusion Chromatography.

Table 9 shows the percentages of acidic, neutral and basic isoform present in samples containing anti-human CD20 and CD3 IgG1 antibody variants harboring non-activating mutations in the constant heavy chain region that have been subjected to one or two freeze/thaw cycles, as determined by cIEF analysis. The change in the percentage of acidic protein present in a sample is used as a surrogate measure for deamidation. Indicated values are averages of 2 individual samples subjected to 3 freeze/thaw cycles. CIEF: capillary Isoelectric Focusing.

Table 10 shows the percentages of intact protein and sum HC+LC, as determined by CE-SDS analysis, and the average radius (in nm), as determined by DLS analysis, detected in samples containing anti-human CD20 and CD3 IgG1 antibody variants harboring non-activating mutations in the constant heavy chain region that have been subjected to three freeze/thaw cycles. Indicated values are averages of 2 individual samples subjected to 3 freeze/thaw cycles. CE-SDS: Capillary Electrophoresis Sodium Dodecyl Sulfate; DLS: Dynamic Light Scattering.

TABLE 8

| | | | HP-SEC | | |
|---|---|---|---|---|---|
| Antibody Variant | | Treatment | Multimer(%) | Monomer(%) | Degradation(%) |
| IgG1-CD20¹ | L234F-L235E- | Reference | 1.1 | 98.6 | 0.3 |
| | D265A-K409R | Freeze/thaw | 1.8 | 98.0 | 0.4 |
| | K409R | Reference | 1.2 | 98.4 | 0.4 |
| | | Freeze/thaw | 2.8 | 96.8 | 0.4 |
| | L234F-L235E- | Reference | 0.7 | 99.3 | 0.0 |
| | G236R-K409R | Freeze/thaw | 2.0 | 98.0 | 0.0 |
| IqG1-CD3¹ | L234F-L235E- | Reference | 0.2 | 99.6 | 0.2 |
| | D265A-F405L | Freeze/thaw | 0.3 | 99.5 | 0.2 |
| | F405L | Reference | 0.2 | 99.6 | 0.2 |
| | | Freeze/thaw | 0.7 | 99.2 | 0.2 |
| | L234F-L235E- | Reference | 0.4 | 99.6 | 0.0 |
| | G236R-F405L | Freeze/thaw | 0.6 | 99.4 | 0.0 |

¹Stress conditions tested at IgG concentration~20 mg/mL

TABLE 9

| | | | CIEF | | |
|---|---|---|---|---|---|
| Antibody Variant | | Treatment | Acidic (%) | Neutral (%) | Basic (%) |
| IqG1-CD20¹ | L234F-L235E- | Reference | 45 | 51 | 4 |
| | D265A-K409R | Freeze/thaw | 45 | 53 | 2 |
| | K409R | Reference | 39 | 54 | 7 |
| | | Freeze/thaw | 39 | 54 | 8 |
| | L234F-L235E- | Reference | 20 | 76 | 4 |
| | G236R-K409R | Freeze/thaw | 20 | 75 | 5 |
| IgG1-CD3¹ | L234F-L235E- | Reference | 48 | 52 | 0 |
| | D265A-F405L | Freeze/thaw | 46 | 54 | 0 |
| | F405L | Reference | 20 | 77 | 3 |
| | | Freeze/thaw | 19 | 79 | 2 |
| | L234F-L235E- | Reference | 16 | 82 | 2 |
| | G236R-F405L | Freeze/thaw | 17 | 82 | 1 |

¹Stress conditions tested at IgG concentration~20 mg/mL

TABLE 10

| | | | CE-SDS | | DLS |
|---|---|---|---|---|---|
| Antibody Variant | | Treatment | Intact IgG1 (%) | HC + LC (%) | Radius (nm) |
| IqG1-CD20¹ | L234F-L235E- | Reference | 99 | 100 | 6.4 |
| | D265A-K409R | Freeze/thaw | 199 | 199.5 | 6.7 |
| | K409R | Reference | 100 | 99 | 6.1 |
| | | Freeze/thaw | 100 | 98 | 6.2 |
| | L234F-L235E- | Reference | 100 | 100 | 11.7 |
| | G236R-K409R | Freeze/thaw | 100 | 100 | 12.1 |
| IqG1-CD3¹ | L234F-L235E- | Reference | 99 | 100 | 18.8 |
| | D265A-F405L | Freeze/thaw | 199 | 99 | 8.9 |
| | F405L | Reference | 99 | 100 | 21.0 |
| | | Freeze/thaw | 99 | 99.5 | 7.5 |
| | L234F-L235E- | Reference | 99 | 100 | 7.5 |
| | G236R-F405L | Freeze/thaw | 99 | 100 | 7.5 |

¹Stress conditions tested at IgG concentration~20 mg/mL

### Example 20: Impact of Low pH-Induced Stress on Stability of IgG1 Non-Activating Antibody Variants

In Example 18, the impact of low or high temperature storage of IgG1 antibody variants harboring non-activating mutations in the constant heavy chain region was assessed using a range of protein stability assays. The same assays were applied in Example 19 to assess the impact of freeze/thaw cycles on the stability of such IgG1 antibody variants. Here, the impact of low pH-induced stress is assessed using the assays described in Example 18, as viral inactivation during antibody therapeutic development is often performed under low pH conditions.

Samples of anti-human CD20 IgG1 and anti-human CD3 (huCLB-T3/4) IgG1 antibody variants were formulated in PBS pH 7.4 at a concentration of approximately 20 mg/mL (concentration range 18.7-21.6 mg/mL; filtration applied for anti-human CD3 antibody variants). The anti-human CD20 IgG1 variants harbored either the K409R mutation, the L234F-L235E-D265A-K409R or L234F-L235E-G236R-K409R mutations, while the anti-human IgG1 antibody variants harbored either the F405L mutation, the L234F-L235E-D265A-F405L or L234F-L235E-G236R-F405L mutations. The indicated antibody variants were formulated in PBS (reference) and buffer exchanged to 0.02 M sodium citrate buffer (pH 3.0; Sigma-Aldrich, Cat #C1909-500G) for 1h (at room temperature) or 24h (at 2-8° C.) followed by another buffer exchange back to PBS. Subsequently, protein stability was studied using HP-SEC, cIEF, CE-SDS and DLS, as described in Example 18.

While the percentage of multimers, as determined by HP-SEC analysis, present in anti-human CD20 IgG1 samples was substantially increased at pH 3.0 for variants harboring the L234F-L235E-D265A-K409R mutations, the K409R- and L234F-L235E-G236R-K409R-containing variants both showed a lower increase in the percentage of multimers. The percentages of multimers observed in samples kept under pH 3.0 conditions for 1h or 24h was similar. For the anti-human CD3 IgG1 variants, samples containing variants harboring the L234F-L235E-D265A-F405L or L234F-L235E-G236R-F405L mutations both showed an increase in the presence of multimers at pH 3.0 (1h and 24h). These percentages were higher than detected in the sample containing the F405L-containing variant.

Data generated by cIEF analysis showed tailing at the basic side of the neutral peak for all anti-human CD20 IgG1 variants incubated at pH 3.0 for either 1 or 24 h. The anti-human CD3 IgG1 variants harboring non-activating mutations L234F-L235E-D265A-F405L or L234F-L235E-G236R-F405L showed an increase in the percentage of acidic protein at pH 3.0, which was not observed for the F405L-containing variant. While an increase in acidic protein was observed at pH 3.0 for both the L234F-L235E-D265A-F405L- and L234F-L235E-G236R-F405L-containing variants as compared to the reference sample (pH 7.4), the L234F-L235E-D265A-F405L-containing variant already contained a higher percentage of acidic protein at pH 7.4 than the L234F-L235E-G236R-F405L-containing variant, probably due to sialylation of D265A. Of note, the increased percentages of acidic protein in the samples containing the anti-human CD3 IgG1 variants harboring the non-activating mutations may reflect an increase in spikes observed in these samples at pH 3.0, which may be due to an increase in aggregates formed in the sample.

CE-SDS analysis did not reveal any differences between samples formulated either in PBS or in a buffer at pH 3.0 in the percentages of intact IgG1 or sum HC+LC. In addition, an increase in the average particle size, as detected using DLS analysis, was observed upon keeping Anti-human CD20 IgG1-L234F-L235E-D265A-K409R at pH 3.0 for 1h or 24 h. The larger average particle size in the PBS reference sample containing the anti-human CD20 IgG1-L234F-L235E-G236R-K409R variant and a sample containing the anti-human CD3 IgG1-F405L variant may be explained by removal of aggregates during buffer exchange. Aside from these observations, no substantial differences in particle size was observed between samples formulated in PBS alone or in PBS after incubation at pH 3.0.

In summary, an increase in the presence of multimers was detected in samples containing anti-human CD20 or CD3 IgG1 antibody variants harboring either the F405L or K409R mutation and/or non-activating mutations in the constant heavy chain region, when samples were incubated at pH 3.0 as compared to samples formulated in PBS at physiological pH. The anti-human CD20 IgG1 variant harboring the L234F-L235E-D265A-K409R mutations showed a larger increase in multimerization than the variant harboring the L234F-L235E-G236R-K409R mutations. Tailing of the neutral peak at the basic side was observed for all tested anti-human CD20 IgG1 variants at pH 3.0, while the results for anti-human CD3 IgG1 antibody variants harboring non-activating mutations were inconclusive due to spikes detected in samples kept to pH 3.0. While acidic conditions (pH 3.0) did not impact protein integrity, a relative increase in the average particle size was detected for anti-human CD20 IgG1-L234F-L235E-D265A-K409R after incubation at pH 3.0.

Hence, when incubated at low pH, anti-human CD20 IgG1 antibody variants harboring the L234F-L235E-G236R mutations showed less aggregation than variants harboring the L234F-L235E-D265A mutations. This indicates that L234F-L235E-G236R-containing antibody variants may be preferred over L234F-L235E-D265A-containing variants during viral inactivation procedures at low pH.

Table 11 shows the percentages of multimers and monomers as detected by HP-SEC analysis, in samples containing anti-human CD20 and CD3 IgG1 (huCLB-T3/4) antibody variants harboring non-activating mutations in the constant heavy chain region that were formulated in PBS, or upon incubation in a 0.02 M sodium citrate buffer (pH 3.0) for 1 or 24h.

Table 12 shows the percentages of acidic, neutral and basic isoforms present in samples containing Anti-human CD20 IgG1 and anti-CD3 IgG1(-huCLB-T3/4) antibody variants harboring non-activating mutations in the constant heavy chain region that were formulated in PBS, or a 0.02M sodium citrate buffer (pH 3.0) for 1 or 24h, as determined by cIEF analysis.

Table 13 shows the percentages of intact IgG and sum HC+LC, as determined by CE-SDS analysis, and the average particle radius (in nm), as determined by DLS analysis, detected in samples containing Anti-human CD20 IgG1 and anti-CD3 IgG1-huCLB-T3/4 antibody variants harboring non-activating mutations in the constant heavy chain region that were formulated in PBS, or a 0.02M sodium citrate buffer (pH 3.0) for 1 or 24h.

TABLE 11

| | | | HP-SEC | | |
|---|---|---|---|---|---|
| Antibody Variant | | Treatment | Multimer(%) | Monomer(%) | Degradation(%) |
| IgG1-CD20¹ | L234F-L235E- | reference | 1.1 | 98.6 | 0.3 |
| | D265A-K409R | pH 3.0 1 hr | 57.9 | 41.7 | 0.3 |
| | | pH 3.0 24 hr | 59.9 | 39.7 | 0.4 |
| | K409R | reference | 1.2 | 98.4 | 0.4 |
| | | pH 3.0 1 hr | 16.1 | 83.4 | 0.4 |
| | | pH 3.0 24 hr | 14.5 | 85.1 | 0.4 |
| | L234F-L235E- | reference | 0.7 | 99.3 | 0 |
| | G236R-K409R | pH 3.0 1 hr | 14.3 | 85.7 | 0 |
| | | pH 3.0 24 hr | 17.1 | 82.9 | 0 |
| IqG1-CD3¹ | L234F-L235E- | reference | 0.2 | 99.6 | 10.2 |
| | D265A-F405L | pH 3.0 1 hr | 26.2 | 73.6 | 0.3 |
| | | pH 3.0 24 hr | 19 | 80.2 | 0.9 |
| | F405L | reference | 0.2 | 99.6 | 0.2 |
| | | pH 3.0 1 hr | 17.2 | 82.5 | 0.2 |
| | | pH 3.0 24 hr | 16.2 | 83.6 | 0.2 |
| | L234F-L235E- | reference | 0.4 | 99.6 | 0 |
| | G236R-F405L | pH 3.0 1 hr | 22.4 | 77.6 | 0 |
| | | pH 3.0 24 hr | 20.3 | 79.7 | 0 |

¹Stress conditions tested at IgG concentration~20 mg/mL

TABLE 12

| | | | CIEF | | |
|---|---|---|---|---|---|
| Antibody Variant | | Treatment | Acidic (%) | Neutral (%) | Basic (%) |
| IgG1-CD20¹ | L234F-L235E- | reference | 45 | 51 | 4 |
| | D265A-K409R | pH 3.0 1 hr | 36 | 48 | 16 |
| | | pH 3.0 24 hr | 33 | 50 | 17 |
| | K409R | reference | 39 | 54 | 7 |
| | | pH 3.0 1 hr | 34 | 53 | 12 |
| | | pH 3.0 24 hr | 35 | 54 | 11 |
| | L234F-L235E- | reference | 20 | 76 | 4 |
| | G236R-K409R | pH 3.0 1 hr | 18 | 69 | 13 |
| | | pH 3.0 24 hr | 18 | 69 | 13 |
| IgG1-CD3¹ | L234F-L235E- | reference | 48 | 52 | 0 |
| | D265A-F405L | pH 3.0 1 hr | 652 | 35 | 0 |
| | | pH 3.0 24 hr | 612 | 39 | 0 |
| | F405L | reference | 20 | 77 | 3 |
| | | pH 3.0 1 hr | 19 | 81 | 0 |
| | | pH 3.0 24 hr | 18 | 81 | 1 |
| | L234F-L235E- | reference | 16 | 82 | 2 |
| | G236R-F405L | pH 3.0 1 hr | 283 | 71 | 1 |
| | | pH 3.0 24 hr | 293 | 69 | 2 |

¹Stress conditions tested at IgG concentration~20 mg/mL
²Presence of spikes
³Presence of additional species on acidic side

TABLE 13

| | | | CE-SDS | | DLS |
|---|---|---|---|---|---|
| Antibody Variant | | Treatment | Intact IgG1 (%) | HC + LC (%) | Radius (nm) |
| IqG1-CD20¹ | L234F-L235E- | reference | 99 | 100 | 6.4 |
| | D265A-K409R | pH 3.0 1 hr | 100 | 99 | 10.2 |
| | | pH 3.0 24 hr | 99 | 100 | 12.3 |
| | K409R | reference | 100 | 99 | 6.1 |
| | | pH 3.0 1 hr | 100 | 99 | 7.1 |
| | | pH 3.0 24 hr | 100 | 100 | 8.1 |
| | | reference | 100 | 100 | 11.7² |
| | L234F-L235E- | pH 3.0 1 hr | 100 | 100 | 7 |
| | G236R-K409R | pH 3.0 24 hr | 100 | 100 | 8.1 |
| IqG1-CD3¹ | L234F-L235E- | reference | 99 | 100 | 8.8 |
| | D265A-F405L | pH 3.0 1 hr | 100 | 99 | 9.4 |
| | | pH 3.0 24 hr | 100 | 99 | 9.8 |
| | F405L | reference | 99 | 100 | 21.0² |
| | | pH 3.0 1 hr | 100 | 99 | 8 |
| | | pH 3.0 24 hr | 99 | 100 | 8.5 |
| | L234F-L235E- | reference | 99 | 100 | 7.5 |
| | G236R-F405L | pH 3.0 1 hr | 99 | 100 | 9.1 |
| | | pH 3.0 24 hr | 99 | 99 | 9.4 |

¹Stress conditions tested at IgG concentration~20 mg/mL
²Multimodal

### Example 21: Assessment of Stability of IgG1 Non-Activating Antibody Variants at Low pH

In Example 20, the impact of low pH on the stability of IgG1 antibody variants harboring non-activating mutations in the constant heavy chain region was assessed using a range of protein stability assays. Here, the impact of low pH-induced stress (pH 3.5) is assessed after 0.5, 1 and 4h at RT using the assays described in Example 18.

Samples of anti-human CD20 and human CD3 (huCLB-T3/4) antibody variants were formulated in PBS pH 7.4 at a concentration of approximately 5 mg/mL (concentration range 4.98-5.3 mg/mL). The IgG1-CD20 variants harbored either the L234F-L235E-D265A-K409R or L234F-L235E-G236R-K409R mutations, while the IgG1-CD3 antibody variants harbored either the L234F-L235E-D265A-F405L or L234F-L235E-G236R-F405L mutations. To assess the impact of exposure to low pH (3.5) conditions, samples containing the antibody variants were buffer-exchanged with 0.02M sodium citrate buffer (pH 3.5) for 0.5h, 1h and 4h at room temperature, followed by another buffer exchange back to PBS. Protein stability was studied using HP-SEC, CE-SDS and DLS, as described in Example 19.

No increase in the percentage of multimers in samples containing anti-human CD20 IgG1 antibody variants harboring non-activating mutations was observed at the 0.5h, 1h and 4h timepoints when samples were incubated at pH 3.5, as determined by HP-SEC analysis. The percentage of multimers for the anti-human CD3 IgG1 variants harboring the L234F-L235E-G236R-F405L mutations increased slightly with time. The percentage of detected multimers strongly increased with time in samples containing the IgG1-huCLB-T3/4-L234F-L235E-D265A-F405L variant at pH 3.5.

CE-SDS analysis did not reveal any changes in the percentages of intact IgG or sum HC+LC in any of the tested samples when samples were incubated either at pH 7.4 (PBS) or pH 3.5. Also, no substantial differences in average particle size were detected between any of the samples either kept at pH 7.4 or pH 3.5, as analyzed by DLS. The enhanced particle size measured for the reference sample of IgG1-CD20-L234F-L235E-G236R-K409R could be explained by the presence of aggregated particles in the applied reference batch which were removed during the buffer exchange process, and which were therefore not detected in the pH-stressed samples.

In summary, no increase in multimerization was observed for anti-human CD20 IgG1 antibody variants harboring non-activating mutations in the constant heavy chain region after exposure to pH 3.5 for 0.5h, 1h or 4h. While the anti-human CD3 IgG1 variant harboring the L234F-L235E-G236R-F405L mutations demonstrated a slight increase in multimerization at pH 3.5, this increase was more pronounced for the variant harboring the L234F-L235E-D265A-F405L mutations. Therefore, it was confirmed that, in line with data presented in Example 20 and despite clone-dependent differences in assay outcome, L234F-L235E-G236R-containing antibody variants can have an advantage over L234F-L235E-D265A-containing variants with regard to viral inactivation procedures at low pH.

Table 14 shows the percentages of multimers and monomers (degradation in all samples<0.2%) as detected by HP-SEC analysis, and the percentages of intact IgG and sum HC+LC, as determined by CE-SDS analysis, in samples containing anti-human CD20 and anti-human CD3 antibody variants harboring non-activating mutations in the constant heavy chain region that were dissolved in PBS or a 0.02M sodium citrate buffer (pH 3.5) for 0.5h, 1h or 4h.

Table 14 also shows the average particle radius (in nm), as determined by DLS analysis, detected in samples containing IgG1-CD20 and IgG1-CD3 antibody variants harboring non-activating mutations in the constant heavy chain region that were dissolved in PBS or a 0.02M sodium citrate buffer (pH 3.5) for 0.5h, 1h or 4h.

TABLE 14

| | | | | | CE-SDS | | |
|---|---|---|---|---|---|---|---|
| | | | HP-SEC | | Intact | | DLS |
| Antibody Variant | | Treatment | Multimer(%) | Monomer(%) | IgG1(%) | HC + LC(%) | Radius (nm) |
| IgG1- | L234F-L235E- | reference | 1.1 | 98.9 | 99 | 99 | 4.8 |
| CD20¹ | D265A-K409R | 0.5 hr pH 3.5 | 1.1 | 98.8 | 99 | 100 | 4.7 |
| | | 1 hr pH 3.5 | 1.1 | 98.8 | 99 | 100 | 4.7 |
| | | 4 hr pH 3.5 | 1.1 | 98.8 | 99 | 100 | 4.8 |
| | L234F-L235E- | reference | 0.8 | 99.1 | 99 | 199 | 10.8² |
| | G236R-K409R | 0.5 hr pH 3.5 | 0.8 | 99.1 | 99 | 100 | 5.4 |
| | | 1 hr pH 3.5 | 0.8 | 99.1 | 99 | 100 | 5.1 |
| | | 4 hr pH 3.5 | 0.8 | 99.1 | 99 | 100 | 5 |
| IgG1- | L234F-L235E- | reference | 1.3 | 98.6 | 97 | 98 | 5 |
| CD31 | D265A-F405L | 0.5 hr pH 3.5 | 4.8 | 95.1 | 97 | 199 | 5 |
| | | 1 hr pH 3.5 | 6.6 | 93.3 | 97 | 99 | 5 |
| | | 4 hr pH 3.5 | 11.0 | 89.0 | 97 | 99 | 5.4 |
| | L234F-L235E- | reference | 1.5 | 98.4 | 97 | 100 | 5 |
| | G236R-F405L | 0.5 hr pH 3.5 | 1.6 | 98.4 | 97 | 100 | 4.9 |
| | | 1 hr pH 3.5 | 1.7 | 98.3 | 97 | 100 | 4.8 |
| | | 4 hr pH 3.5 | 2.2 | 97.8 | 97 | 99 | 4.9 |

¹Stress conditions tested at IgG concentration ~5 mg/mL
²Difference in radius and Monomer % Mass for the IgG1-CD20-FERR reference is due to particles present in the batch which were removed during the buffer exchange procedure and therefore not detected for the pH stressed samples

### Example 22: Assessment of Stability at Low pH of Bispecific IgG1 Antibody Variants Harboring Non-Activating Mutations

In Example 21, the impact of low pH-induced stress (pH 3.5) on the stability of IgG1 antibody variants harboring non-activating mutations in the constant heavy chain region was assessed after 0.5, 1h and 4h. Here, the impact of low pH-induced stress (pH 3.5) on the stability of anti-CD3/CD20 bispecific antibodies harboring non-activating mutations is assessed after 0.5, 1 and 4h using the assays described in Example 18.

Bispecific antibodies (bsAb) were generated from the anti-human CD3 and anti-human CD20 IgG1 antibodies harboring non-activating mutations L234F-L235E-D265A or L234F-L235E-G236R, in addition to either the K409R or F405L mutations which promote half-molecule hetero-dimerization with a complementary half-molecule only under controlled reducing conditions, using the controlled Fab-arm exchange procedure which is described in Example 1. This yielded bsIgG1-CD3×CD20 antibodies harboring either the L234F-L235E-D265A in both arms, or L234F-L235E-G236R in both arms (hereafter indicated as symmetric backbone), or bispecific antibodies harboring a combination of the L234F-L235E-D265A mutations in one arm and the L234F-L235E-G236R mutations in the other arm (hereafter indicated as asymmetric backbone). Samples of the bsIgG1-CD3×CD20 antibody variants were formulated in PBS (pH 7.4) at a concentration of approximately 5 mg/mL (concentration range 3.209-5.304 mg/mL). To assess the impact of exposure to low pH (3.5) conditions, samples containing the bsIgG1-CD3×CD20 antibodies were buffer-exchanged with 0.02 M sodium citrate buffer (pH 3.5) for 0.5h, 1h and 4h at room temperature, followed by another buffer exchange back to PBS. Subsequently, protein stability was studied using HP-SEC, CE-SDS and DLS, as described in Example 18.

HP-SEC analysis did not reveal a notable effect on multimerization upon exposing any of the bsIgG1-CD3×CD20 antibody variants harboring non-activating mutations to pH 3.5 for any of the timepoints tested, as compared to their respective reference samples. Using CE-SDS, also no impact of pH 3.5 exposure was observed for any of the tested bsAb variants, indicating that all bsAbs remained intact upon exposure to pH 3.5. Analysis of the average size by DLS showed that exposure to pH 3.5 of the symmetric bsAb harboring the L234F-L235E-D265A mutations as well as the asymmetric bsAb harboring the L234F-L235E-G236R mutations in the F405L-containing arm did not induce altered levels of aggregation. The small decrease in the average size observed at pH 3.5 at any of the analyzed timepoints for the symmetric bsAb harboring the L234F-L235E-G236R mutations and the asymmetric bsAb harboring the L234F-L235E-G236R mutations in the K409R-containing arm may be due to removal of aggregates during the buffer exchange process.

In summary, IgG1 bsAb variants harboring non-activating mutations in the constant heavy chain region retain their intact structure and are not increasingly sensitive to multimerization after being exposed to low pH conditions for up to 4h at a concentration of approximately 5 mg/mL. These results show that bispecific antibody variants harboring either the L234F-L235E-G236R or L234F-L235E-D265A non-activating mutations were equally capable of retaining monomericity upon exposure to low pH conditions, which is a favorable property during therapeutic development in viral inactivation procedures performed at low pH.

Table 15 shows the percentages of multimers and monomers as detected by HP-SEC analysis, and the percentages of intact IgG and sum HC+LC, as determined by CE-SDS analysis, detected in samples containing bispecific antibodies (bsAb) generated from anti-human CD20 IgG1 and anti-human CD3 antibodies harboring non-activating mutations in the constant heavy chain region. BsAbs were generated harboring either the L234F-L235E-D265A non-activating mutations in both arms, or L234F-L235E-G236R non-activating mutations in both arms, or a combination of the L234F-L235E-D265A mutations in one arm and the L234F-L235E-G236R mutations in the other arm. Antibody variants were dissolved in PBS or a 0.02M sodium citrate buffer (pH 3.5) for 0.5h, 1h or 4h before analysis.

Table 16 shows the average radius (in nm) and the percentage of monomer mass, as determined by DLS analysis, detected in samples containing bsIgG1-CD3×CD20 antibody variants harboring non-activating mutations in the constant heavy chain region. BsAbs were generated harboring either the L234F-L235E-D265A non-activating mutations in both arms, or L234F-L235E-G236R non-activating mutations in both arms, or a combination of the L234F-L235E-D265A mutations in one arm and the L234F-L235E-G236R mutations in the other arm. Antibody variants were dissolved in PBS or a 0.02M sodium citrate buffer (pH 3.5) for 0.5h, 1 h or 4h before analysis.

TABLE 15

| Antibody Variants of | | HP-SEC | | CE-SDS | |
|---|---|---|---|---|---|
| bsIgG1-CD3 × CD20¹ | Treatment | Multimer(%) | Monomer(%) | Intact IgG1(%) | HC + LC(%) |
| L234F-L235E-G236R-F405L × | reference | 0.9 | 99 | 98 | 100 |
| L234F-L235E-G236R-K409R | 0.5 hr pH 3.5 | 1 | 199 | 99 | 100 |
| | 1 hr pH 3.5 | 1 | 99 | 100 | 100 |
| | 4 hr pH 3.5 | 1 | 99 | 99 | 100 |
| L234F-L235E-D265A-F405L × | reference | 1.4 | 98.5 | 98 | 100 |
| L234F-L235E-D265A-K409R | 0.5 hr pH 3.5 | 1.4 | 98.6 | 99 | 99 |
| | 1 hr pH 3.5 | 1.4 | 98.5 | 100 | 99 |
| | 4 hr pH 3.5 | 1.4 | 98.6 | 99 | 100 |
| L234F-L235E-G236R-F405L × | reference | 1.2 | 98.7 | 98 | 100 |
| L234F-L235E-D265A-K409R | 0.5 hr pH 3.5 | 1.1 | 98.8 | 98 | 100 |
| | 1 hr pH 3.5 | 1.1 | 98.8 | 99 | 100 |
| | 4 hr pH 3.5 | 1.1 | 98.8 | 99 | 100 |
| L234F-L235E-D265A-F405L × | reference | 1.3 | 98.7 | 98 | 99 |
| | 0.5 hr pH 3.5 | 1.2 | 98.8 | 98 | 98 |
| L234F-L235E-G236R-K409R | 1 hr pH 3.5 | 1.2 | 98.8 | 99 | 100 |
| | 4 hr pH 3.5 | 1.1 | 98.9 | 99 | 99 |

¹Stress conditions tested at IgG concentration~5 mg/mL

TABLE 16

| Antibody Variants of bsIgG1- | | DLS |
|---|---|---|
| CD3 × CD201 | Treatment | Radius (nm) |
| L234F-L235E-G236R-F405L × | reference | 5.9 |
| L234F-L235E-G236R-K409R | 0.5 hr pH 3.5 | 5.1 |
| | 1 hr pH 3.5 | 4.9 |
| | 4 hr pH 3.5 | 4.9 |
| L234F-L235E-D265A-F405L × | reference | 4.9 |
| L234F-L235E-D265A-K409R | 0.5 hr pH 3.5 | 4.8 |
| | 1 hr pH 3.5 | 4.8 |
| | 4 hr pH 3.5 | 4.9 |
| L234F-L235E-G236R-F405L × | reference | 4.9 |
| L234F-L235E-D265A-K409R | 0.5 hr pH 3.5 | 4.8 |
| | 1 hr pH 3.5 | 4.8 |
| | 4 hr pH 3.5 | 5.1 |
| L234F-L235E-D265A-F405L × | reference | 6.7 |
| L234F-L235E-G236R-K409R | 0.5 hr pH 3.5 | 5.3 |
| | 1 hr pH 3.5 | 5 |
| | 4 hr pH 3.5 | 4.9 |

1Stress conditions tested at IgG concentration~5 mg/mL

### Example 23: Assessment of Stability at Low pH of Anti-CD20 and Anti-Gp120 IgG1 Antibody Variants Harboring Non-Activating Mutations

In Example 21, the impact of low pH-induced stress (pH 3.5) on the stability of IgG1 antibody variants harboring non-activating mutations in the constant heavy chain region was assessed after 0.5h, 1h and 4h. Here, the impact of low pH-induced stress (pH 3.5) on the stability of anti-human CD20 IgG1 and anti-gp120 (HIV1) IgG1-b12 antibodies harboring non-activating mutations in the constant heavy chain region, in combination with the K409R mutation, is assessed after 0.5, 1h, 2h, 4h and 24h using the HP-SEC assay described in Example 18. The extent of multimerization under low pH conditions was analyzed for these antibody variants as a measure of protein instability, which is of importance in the context of viral inactivation procedures during antibody therapeutic development.

Samples of anti-human CD20 IgG1 and IgG1-b12 antibody variants were formulated in PBS at a concentration of approximately 0.5 mg/mL (concentration range 0.435-0.5 mg/mL). The anti-human CD20 IgG1 and IgG1-b12 variants harbored either the L234F-L235E-D265A-K409R or L234F-L235E-G236R-K409R mutations. To assess the impact of exposure to low pH (3.5) conditions, samples containing the antibody variants in PBS were acidified by dropwise addition of 2M acetic acid (Fluka, Cat #33209) to pH 3.5, and subsequent incubation at RT for 0.5h, 1h, 2h, 4h or 24h using a plate shaker. After incubation, a sample from each sample tube was transferred to a tube containing 2M Tris-HCl (pH 9.0; Sigma-Aldrich, Cat #T6066) to recover the pH to 7.4. Subsequently, protein stability was studied using HP-SEC, as described in Example 18.

HP-SEC analysis revealed a rapid increase in multimerization occurring in samples containing Anti-human CD20 IgG1 variant harboring the L234F-L235E-D265A-K409R mutations in response to incubation at pH 3.5, which reached a top after 2h of incubation with 35.2% of multimers detected in the sample. In contrast, exposure of the Anti-human CD20 IgG1 variant harboring the L234F-L235E-G236R-K409R mutations to pH 3.5 resulted in a steady and relatively slow increase in multimerization with time, with a maximum of 10.8% of multimers detected in the sample analyzed after 24h of incubation.

A highly similar pattern was observed for the IgG1-b12 variants harboring non-activating mutations. While the variant harboring the L234F-L235E-D265A-K409R mutations showed a fast increase in multimerization with a maximum of 27.6% of multimers detected after 2h of incubation at pH 3.5, a slower and lower increase in multimerization was observed for the variant harboring the L234F-L235E-G236R-K409R mutations at pH 3.5, with a maximum of 18% of multimers detected after 24h of incubation at pH 3.5.

In summary, HP-SEC analysis revealed that the process of multimerization occurs more rapidly and more extensively in antibody variants harboring the L234F-L235E-D265A-K409R mutations than variants harboring the L234F-L235E-G236R-K409R mutations. Therefore, it was concluded that also other antibody clone variants harboring the L234F-L235E-G236R non-activating mutations had a higher capacity to retain monomericity at low pH conditions than L234F-L235E-D265A-containing variants. Retention of monomericity at low pH is a favorable characteristic during viral inactivation procedures performed at low pH.

Table 17 shows the percentages of multimers, monomers and degradation as detected by HP-SEC analysis, in samples containing anti-human CD20 IgG1 and anti-gp120 (HIV1) IgG1-b12 antibody variants harboring non-activating mutations in the constant heavy chain region that were dissolved in PBS or a sodium acetate buffer (pH 3.5) for 0.5h, 1h, 2h, 4h or 24h.

TABLE 17

| | | | HP-SEC | | |
|---|---|---|---|---|---|
| Antibody Variant | | Treatment | Multimer(%) | Monomer(%) | Degradation (%) |
| IqG1-CD20¹ | L234F-L235E- | reference | 0.9 | 98.9 | 0.3 |
| | D265A-K409R | 0.5 hr pH 3.5 | 24.8 | 74.9 | 0.3 |
| | | 1 hr pH 3.5 | 34.1 | 65.6 | 0.3 |
| | | 2 hr pH 3.5 | 35.2 | 64.5 | 0.3 |
| | | 4 hr pH 3.5 | 33.8 | 65.9 | 0.3 |
| | | 24 hr pH 3.5 | 29.6 | 70 | 0.3 |
| | L234F-L235E- | reference | 0.5 | 99.5 | 0 |
| | G236R-K409R | 0.5 hr pH 3.5 | 1.4 | 98.6 | 0 |
| | | 1 hr pH 3.5 | 3.6 | 96.4 | 0 |
| | | 2 hr pH 3.5 | 5.3 | 94.7 | 0 |
| | | 4 hr pH 3.5 | 6.5 | 93.5 | 0 |
| | | 24 hr pH 3.5 | 10.8 | 89.2 | 0 |
| IgG1-gp120¹ | L234F-L235E- | reference | 1.8 | 98.2 | 0 |
| | D265A-K409R | 0.5 hr pH 3.5 | 18.2 | 81.8 | 0 |
| | | 1 hr pH 3.5 | 26.7 | 73.3 | 0 |
| | | 2 hr pH 3.5 | 27.6 | 72.4 | 0 |
| | | 4 hr pH 3.5 | 26.7 | 73.3 | 0 |
| | | 24 hr pH 3.5 | 17.3 | 82.7 | 0 |
| | L234F-L235E- | reference | 1.1 | 98.9 | 0 |
| | G236R-K409R | 0.5 hr pH 3.5 | 3.4 | 96.7 | 0 |
| | | 1 hr pH 3.5 | 5.5 | 94.5 | 0 |
| | | 2 hr pH 3.5 | 7.2 | 92.8 | 0 |
| | | 4 hr pH 3.5 | 8.1 | 91.9 | 0 |
| | | 24 hr pH 3.5 | 18 | 81.6 | 0.4 |

¹Stress conditions tested at IgG concentration~0.5 mg/mL 0.5 mg/ml for reference, 0.435 mg/ml for pH 3.5)

### Antibody Therapeutic Developability Aspects of FER

In Examples 15-23, a range of aspects related to antibody therapeutic developability were assessed. Firstly, no apparent differences were observed in the capacity to form bispecific antibodies using antibody variants harboring either the L234F-L235E-D265A (FEA) or L234F-L235E-G236R (FER) mutations. Also, production levels of antibody variants harboring either the FEA or FER mutations were similar. Highly concentrated samples of antibody variants harboring the FER mutations demonstrated improved protein stability and less propensity to aggregate as compared to antibody variants harboring the FEA mutations. Exposing antibody variants to 40° C. for 4 months resulted in increased multimerization of all tested variants, the extent of which was antibody clone-dependent. Furthermore, freeze-thaw cycles did not affect protein stability of antibody variants harboring either the FEA or FER mutations. Importantly, antibody variants harboring the FER mutations demonstrated higher resistance to low pH-induced stress conditions as compared to the variants harboring the FEA mutations.

Introduction of the FEA mutations in the Fc region of IgG1 antibodies results in antibody variants that can readily be developed and such antibody variants are currently well-accepted for clinical product development and for clinical use. In view of the above, the protein stability profile and developability of antibody variants when desiring an inert format could be further improved by introducing the FER mutations instead. Therefore, FER-containing variants can be preferred for the development of monospecific or multispecific antibodies for clinical use. In particular, antibody variants harboring the FER mutations were shown to be less sensitive to exposure to low pH conditions, which is a beneficial property in procedures for viral inactivation that are often applied and required during development of therapeutic antibodies for human use.

### Example 24: C1q Binding and Complement-Dependent Cytotoxicity by Anti-Human CD20 Antibodies and Variants Thereof Containing Different Sets of Non-Activating Mutations in Each Heavy Chain

In Examples 3 and 5, it was shown that introduction of the L234F-L235E-G236R (FER) mutations in the heavy chain (HC) constant region of wild-type-like IgG1 anti-human CD20 and HLA-DR antibodies resulted in a near-complete abrogation of the capacity to induce CDC. This was in line with a reduction in C1q binding by antibody variants harboring the FER mutations, as shown in Example 4. Here, the capacity to bind complement factor C1q was assessed for anti-human CD20 antibodies containing two HCs that harbored either the L234F-L235E-D265A (FEA) or the FER mutations in both HCs, and for variants that contained the FEA mutations in one HC and the FER mutations in the other HC. Also, the capacity of the antibody variants above to induce CDC was assessed.

Wild-type anti-human CD20 antibody IgG1-CD20 and variants thereof harboring the FEA or FER non-activating mutations in both HCs, or variants containing the FEA mutations in one HC and the FER mutations in the other HC (hereafter designated as “asymmetric variants”), in addition to K409R or F405L mutations, were tested in a C1q binding assay on Raji cells with 20% normal human serum (NHS, M0008, Sanquin) as the source of C1q, following the procedure described in Example 4. Asymmetric variants of anti-human CD20 antibodies were generated through controlled Fab-arm exchange, essentially as described in Example 1. C1q binding to the antibody variants was detected by flow cytometry on an Intellicyt iQue screener (Sartorius) by measuring Median Fluorescence Intensity-FITC. The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control as baseline, followed by normalization per experimental replicate to the AUC value measured for the non-binding control antibody IgG1-b12 (0%) and the AUC value measured for the wild-type IgG1 antibody variant (IgG1-CD20, 100%). Data are mean values ±SEM obtained from 3 independent experiments.

The same antibody variants tested in the C1q binding assay were tested in an in vitro CDC assay. Antibody variants were tested in a range of concentrations (0.014-10 μg/mL final concentrations; 3-fold dilutions) using 5×10⁴ Raji cells per well, essentially as further described in Example 3.

The number of PI-positive cells was determined by flow cytometry on an Intellicyt iQue screener (Sartorius). The percentage of PI-positive cells, which corresponds to the percentage of cell lysis, was calculated as (number of PI-positive cells/total number of cells)×100%. The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control as baseline, followed by normalization per experimental replicate to the AUC value measured for the non-binding control antibody IgG1-b12 (0%) and the AUC value measured for the wild-type IgG1 antibody variant (IgG1-CD20, 100%). Data are mean values ±SEM obtained from 3 independent experiments.

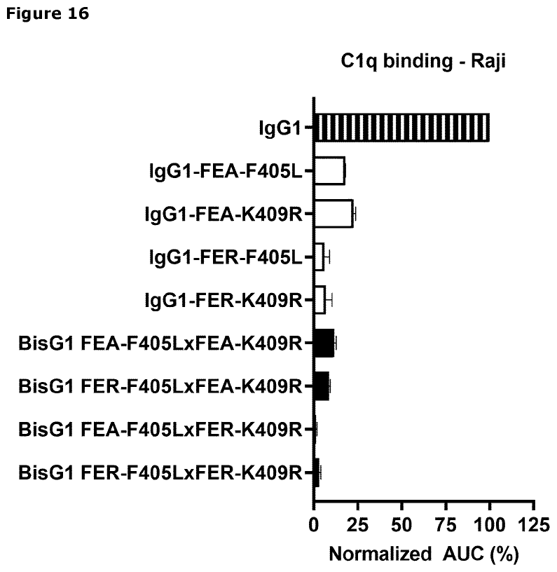

Assessment of binding of C1q to IgG1-CD20 variants revealed that wild-type IgG1-CD20 antibody efficiently engages with the complement protein C1q upon binding to CD20 on target Raji cells. Introduction of either the FEA or FER non-activating mutations, combined with either the F405L or K409R mutation, dramatically decreased C1q binding (FIG. 16). Binding of C1q was more strongly decreased to the IgG1-CD20 variants harboring the FER mutations, than to the IgG1-CD20 variants harboring the FEA mutations, irrespective of whether the HCs both contained either the K409R or the F405L mutation, or whether one HC contained the K409R mutation and the other HC contained the F405L mutation. Full abrogation of C1q binding was observed for asymmetric variant BisG1-CD20 FEA-F405L×FER-K409R, while a strong reduction in C1q binding was observed for the other asymmetric variant, BisG1-CD20 FER-F405L×FEA-K409R (FIG. 16).

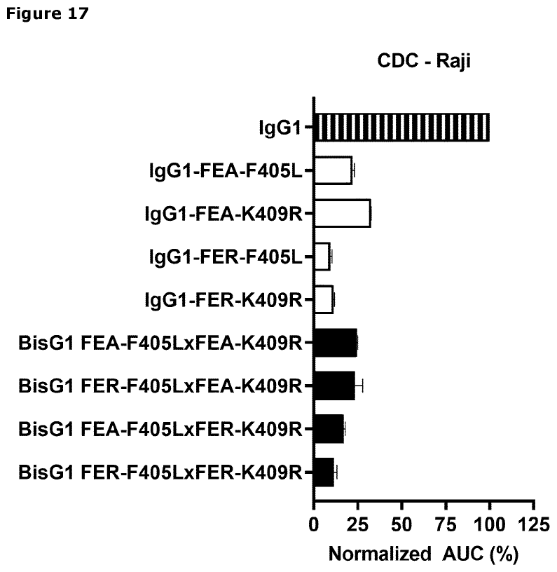

Assessment of the CDC-inducing capacity of the same IgG1-CD20 variants revealed that the capacity to eliminate CDC was most strongly suppressed by introducing the FER mutations, irrespective of whether both HCs contained either the K409R or F405L mutation, or whether one HC contained the K409R mutation and the other HC contained the F405L mutation (FIG. 17). Residual CDC was observed for an IgG1-CD20 variant containing mutations FEA-K409R and to a lesser extent for IgG1-CD20-FEA-F405L, as compared to wild-type IgG1-CD20. Low residual CDC was observed for asymmetric variant BisG1-CD20 FEA-F405L×FER-K409R. The reduction in CDC-inducing capacity of BisG1-CD20 FER-F405L×FEA-K409R was comparable to that observed for BisG1-CD20 FEA-F405L×FEA-K409R.

In conclusion, anti-human CD20 IgG1 antibody binding to C1q was most strongly suppressed by introducing the FER mutations in both HCs, or upon introduction of these mutations in one HC that further contained the K409R mutation, and the FEA-F405L mutations in the other HC. The capacity to induce CDC was more strongly suppressed by introducing the FER mutations than the FEA mutations. Asymmetric variants, containing the FER mutations in one HC and the FEA mutations in the other HC, also demonstrated a strong reduction of CDC-inducing capacity down to a level intermediate to variants harboring either the FER or FEA mutations in both HCs.

### Example 25: In Vitro T-Cell-Mediated Cytotoxicity Induced by Antibody Variants Containing Different Sets of Non-Activating Mutations in Each Heavy Chain

Example 11 showed that introduction of the L234F-L235E-G236R (FER) mutations in the heavy chain (HC) constant region of bispecific antibody variants, targeting a cancer antigen and a T-cell, efficiently avoided non-specific cytotoxicity but retained the capacity to induce specific T-cell-mediated cytotoxicity. In these experiments, the non-activating mutations were present in a symmetric fashion, i.e. both HCs harbored the FER non-activating mutations in addition to either a F405L or K409R mutation. Here, T-cell-mediated cytotoxicity was assessed for CD3×HER2 and CD3×b12 bispecific IgG1 antibodies harboring asymmetric non-activating mutations in the Fc region, i.e. one HC harbored the FER mutations while the other HC harbored the alternative non-activating mutations (e.g. L234F-L235E-D265A [FEA], L234A-L235A-P329G [AAG], or N297G).

Bispecific antibody variants, including those harboring asymmetric non-activating mutations, were generated by controlled Fab-arm exchange (cFAE), as described in Example 1. T-cell-mediated cytotoxicity by the wild-type bispecific antibodies CD3×HER2 (anti-human CD3 [huCLB-T3/4] IgG1-F405L and anti-human HER2 IgG1-K409R) or CD3×b12 (anti-human CD3 [huCLB-T3/4] IgG1-F405L and non-binding control antibody anti-HIV1 gp120 [b12] IgG1-K409R) and variants thereof harboring asymmetric non-activating mutations in the Fc region were evaluated. The bispecific variants tested harbored the FER non-activating mutations, in addition to a F405L mutation in one HC, combined with either FEA, AAG, or N279G non-activating mutations, in addition to a K409R mutation in the second HC. Furthermore, bispecific antibody variants were tested that harbored FER non-activating mutations, in addition to a F405L mutation in one HC, combined with a second HC that does not harbor non-activating mutations, but only the K409R mutation (required for efficient generation of bispecific antibody variants). As controls, bispecific antibody variants without non-activating mutations in the HC, as well as the non-binding control antibody IgG1-b12 were tested. PBMCs were isolated from buffy coats derived from healthy donors by density gradient separation as described in Example 9, washed with PBS, and resuspended in culture medium (RPMI-1640 with 2 mM L-glutamine and 25 mM HEPES; supplemented with 10% donor bovine serum with iron (DBSI)). Subsequently, PBMCs were frozen at −150° C., at a concentration of 30-50×10⁶ cells/ml, by resuspending the PBMCs in cryoprotective medium, which consisted of one part culture medium (RPMI-1640 with 2 mM L-glutamine and 25 mM HEPES; supplemented with 10% donor bovine serum with iron (DBSI)) and one part of a mixture of 80% DBSI and 20% Dimethyl sulfoxide (DMSO; Sigma-Aldrich, Cat #41644); final concentration of DMSO in the cryoprotective medium was 10%). HER2-expressing SK-OV-3 cells (ATCC, Cat #HTB-77) were cultured in McCoy's 5A medium (Lonza, Cat #BE12-168F) supplemented with 10% (vol/vol) heat inactivated DBSI, and penicillin-streptomycin (Pen/Strep, final concentration 50 units/mL potassium penicillin and 50 μg/mL streptomycin sulfate [Lonza, Cat #DE17-603E]) and maintained at 37° C. in a 5% (vol/vol) CO₂ humidified incubator. SK-OV-3 cells were cultured to near-confluency. Cells were trypsinized and resuspended in culture medium and subsequently passed through a cell strainer to obtain a single cell suspension. 2.5×10⁴ SK-OV-3 cells were seeded to each well of a 96-well culture plate, and cells were incubated 4 hours at 37° C., 5% CO₂, to allow adherence to the plate. PBMCs that were frozen after isolation as described above, were thawed. PBMCs were then washed twice with PBS followed by resuspension in culture medium. Subsequently, 1×10⁵ PBMCs were added to each well of the 96-well plate containing the SK-OV-3 target cells resulting in an effector to target (E:T) ratio of 4:1. Subsequently, a dose response series of bispecific CD3×HER2 and CD3×b12 wild-type and non-activating variants thereof, as mentioned above, was prepared in culture medium (0.001-1000 ng/mL final concentration, 10-fold dilutions) and added to the wells of the 96-well culture plates containing the SK-OV-3 cells and PBMCs. Incubation of SK-OV-3 target cells with 2 μM staurosporin (Sigma-Aldrich, Cat #S6942-200, Sigma) was used as reference for 100% tumor cell kill. Medium control (SK-OV-3 cells, no antibody, no PBMC) was used as a reference for 0% tumor cell kill. Plates were incubated for 3 days at 37° C., 5% CO₂. After three days, plates were washed twice with PBS, and 150 μL culture medium containing 10% Alamar blue (Invitrogen, Cat #DAL1100) was added to each well. Plates were incubated for 4 hours at 37° C., 5% CO₂. Absorbance at 590 nm was measured (Envision, Perkin Elmer, Waltham, MA). The data was visualized in GraphPad PRISM (version 8.4.1, GraphPad Software) as dose response vs. percentage viable SK-OV-3 cells, calculated for each donor per experimental replicate. Data are mean values ±SEM obtained from four donors from two independent experiments.

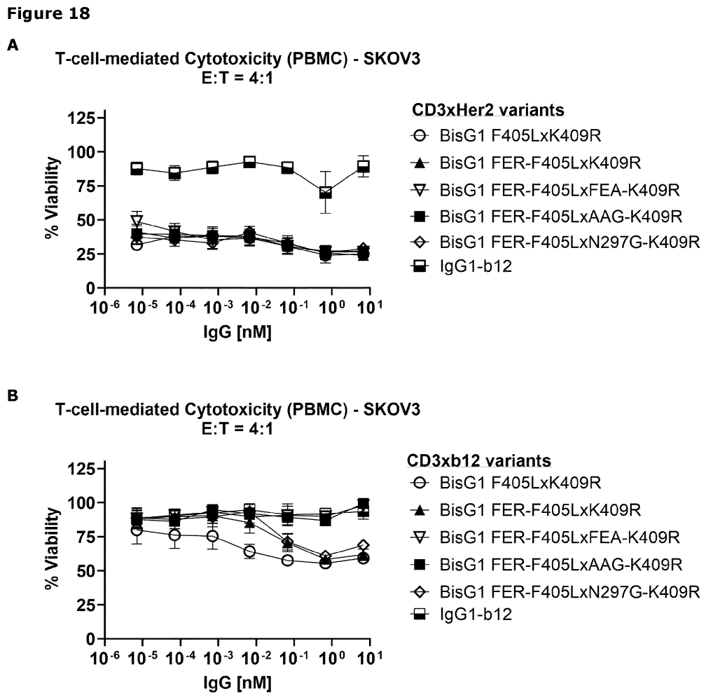

All bispecific CD3×HER2 antibody variants, including those harboring asymmetric non-activating mutations in the Fc region, induced dose-dependent cytotoxicity of SK-OV-3 cells with comparable efficiency to a bispecific CD3×HER2 antibody variant harboring an Fc region with wild-type-like function, thus without non-activating mutations (FIG. 18A). Similar to the data shown in Example 11, the wild-type-like bispecific CD3×b12 antibody (BisG1 F405L×K409R) induced non-specific killing of SK-OV-3 cells, albeit to a lesser extent than the wild-type-like bispecific CD3×HER2 antibody variant (FIG. 18B). The bispecific CD3×b12 antibody variants harboring FER non-activating mutations in one HC combined with FEA (BisG1 FER-F405L×FEA-K409R) or AAG (BisG1 FER-F405L×AAG-K409R) mutations in the other HC showed no cytotoxicity of SK-OV-3 cells (FIG. 18B), similar to the bispecific CD3×b12 variants harboring symmetrically distributed FER, FEA, or AAG mutations (FIG. 10B). In contrast, the bispecific CD3×b12 antibody variants harboring FER non-activating mutations in one HC combined with the other HC harboring a N297G non-activating mutation (BisG1 FER-F405L×N297G-K409R) induced non-specific killing of SK-OV-3 cells, albeit to a lesser extent than a wild-type-like bispecific CD3×b12 antibody variant (FIG. 18B). This result is in line with the observation that a bispecific CD3×b12 antibody variant with symmetric N297G non-activating mutations also induced partial non-specific killing of SK-OV-3 cells (FIG. 10B). Furthermore, a bispecific CD3×b12 antibody variant harboring FER non-activating mutations in one HC combined with the other HC region with wild-type-like function (BisG1 FER-F405L×K409R) also induced non-specific killing at similar levels to the CD3×b12 variant BisG1 FER-F405L×N297G-K409R (FIG. 18B).

Overall, bispecific antibody variants, targeting a cancer antigen and a T-cell, with asymmetric non-activating mutations in the Fc region, i.e. the FER non-activating mutations in one HC and the other HC harboring either FEA or AAG non-activating mutations, retained the capacity to induce specific T-cell-mediated cytotoxicity but efficiently avoided non-specific cytotoxicity.

### Example 26: T-Cell Activation in PBMC Culture Induced by Antibody Variants Containing Different Sets of Non-Activating Mutations in Each Heavy Chain

Example 10 showed that introduction of the L234F-L235E-G236R (FER) mutations in the heavy chain (HC) constant region of an anti-human CD3 IgG1 antibody prevented activation of T cells, as measured by upregulation of CD69, in a human PBMC co-culture. In these experiments, the non-activating mutations were present in a symmetric fashion, i.e., both HCs harbored the FER non-activating mutations in addition to either a F405L or K409R mutation. Here, T-cell activation was assessed for CD3×HER2 bispecific IgG1 antibodies harboring asymmetric non-activating mutations in the Fc region, i.e., one HC harboring the FER mutations and the other HC harboring the different non-activating mutations.

Activation of T cells in a PBMC co-culture by the wild-type bispecific IgG1 antibodies CD3×HER2 and variants thereof, as indicated in Example 25, harboring asymmetric non-activating mutations in the Fc region was evaluated. As controls, a bispecific antibody variant harboring symmetric non-activating FER mutations in the HC, a bispecific antibody variant without non-activating mutations in the HC, as well as the non-binding control antibody IgG1-b12 were tested. Upregulation of CD69 on T cells, as a measure for T-cell activation, by these antibody variants was assessed essentially following the procedure described in Example 10. In short, a dose response series of bispecific CD3×HER2 and CD3×b12 wild-type and non-activating variants thereof, as mentioned above, was prepared in culture medium (0.001-1000 ng/mL final concentration, 10-fold dilutions) and added to the wells of a 96-well round bottom plate containing the PBMCs (1.5×10⁵ cells/well) in culture medium. After 16-24 hours incubation, the percentage of CD69-positive cells of the CD28-positive cells in the PBMC mixture was measured on a Fortessa flow cytometer (BD). The data was analyzed as dose-response vs. percentage CD69-positive of CD28-positive cells. The area under the dose-response curves (AUC) per PBMC donor of each experimental replicate was calculated using log-transformed concentrations in GraphPad PRISM (version 8.4.1, GraphPad Software) with background stain (no antibody control) as baseline, followed by normalization for each donor per experimental replicate to the AUC value measured for the non-binding negative control IgG1-b12 (0%) and the wild-type like IgG1 bispecific antibody variant (BisG1 F405L×K409R, 100%). Data are mean values ±SEM obtained from 4 donors in two independent replicate experiments.

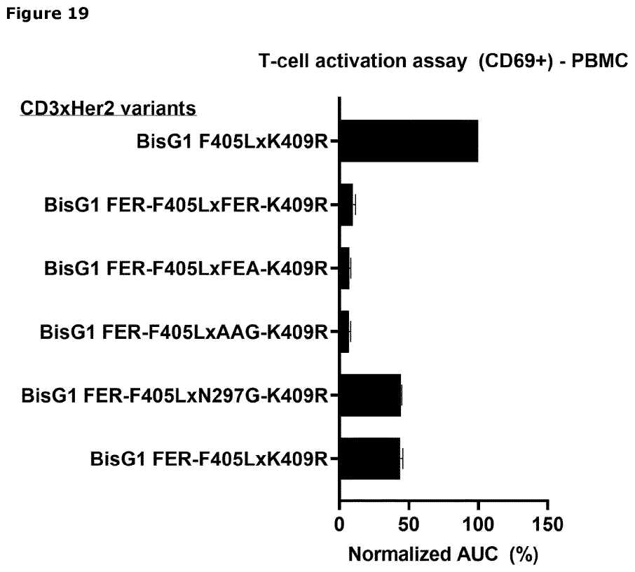

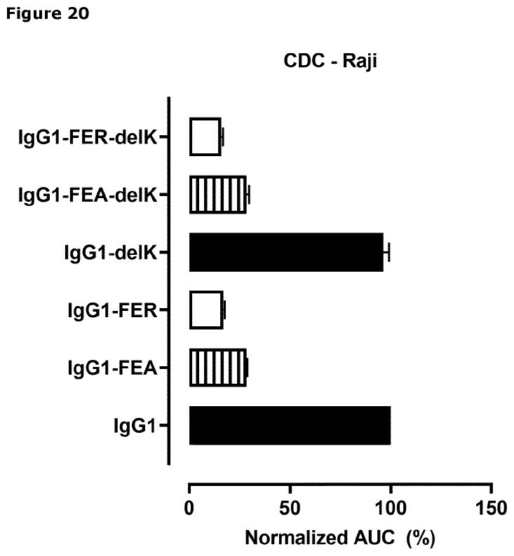

Assessment of CD69 upregulation on T cells as a measure for early T-cell activation shows that a bispecific antibody variant harboring symmetric non-activating FER mutations in the HC prevented upregulation of CD69 on T cells in PBMC co-cultures, as compared to the wild-type-like CD3×HER2 bispecific antibody variant (BisG1 F405L×K409R) (FIG. 19). This is in line with data described in Example 10 for an anti-human CD3 IgG1 antibody harboring the FER non-activating mutations in addition to a F405L mutation. The bispecific CD3×HER2 antibody variants harboring FER non-activating mutations in one HC combined with FEA (BisG1 FER-F405L×FEA-K409R) or AAG (BisG1 FER-F405L×AAG-K409R) mutations in the other HC also showed near-complete abrogation of CD69 upregulation on T cells in PBMC co-cultures (FIG. 19). In contrast, the bispecific CD3×HER2 antibody variants harboring FER non-activating mutations in one HC combined with the other HC harboring a N297G non-activating mutation (BisG1 FER-F405L×N297G-K409R) or combined with another HC region with wild-type-like function (BisG1 FER-F405L×K409R) induced residual activation of T cells albeit to a lesser extent than a wild-type-like bispecific CD3×HER2 antibody variant (FIG. 19).

In summary, bispecific antibody variants, targeting a cancer antigen and a T cell, with asymmetric non-activating mutations in the Fc region, i.e., the FER non-activating mutations in one HC and the other HC harboring either FEA or AAG non-activating mutations, efficiently prevented activation of T cells, as measured by CD69 upregulation, in a human PBMC co-culture.

### Example 27: Binding Affinity of Anti-Human CD20 IgG1 Antibodies and Non-Activating Variants Thereof to Human Fcγ Receptors Measured by Biolayer Interferometry

Data in Example 6 showed that introducing the non-activating L234F-L235E-G236R (FER) mutations in the heavy chain (HC) constant region prevented binding to the monomeric extracellular domain (ECD) of FcγRIa, or dimeric ECDs of human FcγRIIa allotype 131H, human FcγRIIa allotype 131R, human FcγRIIb, human FcγRIIIa allotype 158F, and human FcγRIIIa allotype 158V, as measured by an ELISA assay.

Here, we quantified the binding affinity of wild-type anti-human CD20 IgG1 antibodies and non-activating variants thereof to the monomeric extracellular domains (ECD) of human FcγRIa, FcγRIIa allotype 131H, FcγRIIb, and FcγRIIIa allotype 158V using biolayer interferometry (BLI) on an Octet HTX Instrument (FortéBio). Non-activating variants that were tested harbored mutations L234F-L235E-D265A, L234F-L235E-G236R, or L234A-L235A-P329G. All steps were performed at 30° C. For human FcγRIa, after acclimatization (600 s at 30° C.) of the Ni-NTA biosensors (FortéBio, cat. no. 18-S101), as well as the proteins diluted in Sample Diluent (FortéBio, cat. no. 18-1104), an initial baseline measurement was performed by incubating the NiNTA biosensors in Sample Diluent for 100 s. Subsequently, His-tagged human FcγRIa (Sino Biological, 10256-H085-B, 1 μg/ml) was immobilized on Ni-NTA biosensors for 600 s. After a baseline measurement (100 s) in Sample Diluent, the association (300 s) and dissociation (1000 s) of the wild-type anti-human CD20 IgG1 antibody and non-activating variants thereof was determined. Binding of the antibodies to human FcγRIa was tested using a concentration range of 1.56-100 nM (for IgG1 wild-type; 2-fold dilutions) or 15.6-1000 nM (for IgG1-L234F-L235E-D265A, IgG1-L234F-L235E-G236R, and IgG1-L234A-L235A-P329G; 2-fold dilutions). Data was analyzed with Data Analysis Software v11.1 (FortéBio). In short, data traces were corrected by subtraction of a reference curve (FcγR on sensor, measurement with Sample Diluent only), the Y-axis was aligned to the last 10 s of the baseline measured, and interstep correction as well as Savitzky-Golay filtering was applied. To determine the K_{D} (M), as well as the kₒₙ (1/Ms) and k_{off} (Vs), a 1:1 Model was chosen using a Global (Full) fit. Response values<0.05 were excluded from the analysis. 400 s dissociation was used as window of interest for the analysis for all antibody variants. Optimal fit was determined by a full R² value of >0.99, indicating the fit and experimental data correlated significantly. Furthermore, the Global (Full) fit is based on >3 curve fits (reliable analysis). Data shown are mean values ±SD of 2 independent replicates.

For human FcγRIIa allotype 131H, FcγRIIb, and FcγRIIIa allotype 158V, after acclimatization (600 s at 30° C.) of the Streptavidin (SA) biosensors (FortéBio, cat. no. 18-5019), as well as the proteins diluted in Sample Diluent, an initial baseline measurement was performed by incubating the SA biosensors in Sample Diluent for 100 s. Subsequently, His-tagged biotinylated human FcγRIIa allotype 131H (Sino Biological, 10374-H27H1-B, 1 μg/ml), FcγRIIb (Sino Biological, 10259-H27H-B, 1 μg/ml), or FcγRIIIa allotype 158V (Sino Biological, 10389-H27H1-B, 3 μg/ml) were immobilized on SA biosensors for 65 s (FcγRIIa or FcγRIIb) or 600 s (FcγRIIIa). After a baseline measurement (100 s) in Sample Diluent, the association (50 s, FcγRIIa or FcγRIIb; 300 s, FcγRIIIa) and dissociation (1000 s) of the wild-type anti-human CD20 IgG1 antibody and non-activating variants thereof was determined. Binding of the antibodies to FcγRIIa allotype 131H, FcγRIIb, and FcγRIIIa allotype 158V was tested using a range of concentrations (FcγRIIa, 156.25-10000 nM, 2-fold dilutions; FcγRIIb, 250-16000 nM, 2-fold dilutions; FcγRIIIa, 125-8000 nM, 2-fold dilutions). Data was analyzed with Data Analysis Software v11.1 (ForteNo). In short, data traces were corrected by subtraction of a reference curve (FcγR on sensor, measurement with Sample Diluent only), the Y-axis was aligned to the last 10 s of the baseline measured, and interstep correction was applied. Due to the low affinity between human IgG1 and the low affinity Fcγ receptors FcγRIIa allotype 131H, FcγRIIb, and FcγRIIIa allotype 158V, Steady State Analysis (SSA) was chosen to determine the K_{D} (M). In short, Response values<0.05 were excluded from the analysis. The steady-state responses (where the sensogram reached a plateau in the association phase) for the different antibody concentrations were calculated using the R-equilibrium (R_{eq}) function of the Data Analysis Software, subsequently the steady-state responses were plotted against the antibody concentration, and finally a Langmuir model was used to calculate the K_{D} (M). Optimal fit was determined by a full R² value of >0.99, indicating the fit and experimental data correlate significantly. Furthermore, SSA is based on >3 association curve fits and the plotted steady-state responses for each antibody concentration reached a plateau allowing proper calculation of the maximum binding response (reliable analysis). Data shown are mean values ±SD of 2 independent replicates.

Assessment of the binding of anti-human CD20 human IgG1 antibody variants to human FcγRIa, FcγRIIa allotype 131H, FcγRIIb, and FcγRIIIa allotype 158V using biolayer interferometry revealed that wild-type IgG1 showed binding to all tested Fcγ receptors with a binding affinity of 1.8 nM for FcγRIa, 1.9 μM for FcγRIIa allotype 131H, 7.3 μM for FcγRIIb, and 0.6 μM for FcγRIIIa allotype 158V (Table 18). In contrast, the human IgG1 antibody variants harboring L234F-L235E-G236R, L234F-L235E-D265A, or L234A-L235A-P329G non-activating mutations in the heavy chain constant region did not show binding to human FcγRIa, FcγRIIa allotype 131H, FcγRIIb, and FcγRIIIa allotype 158V (Table 18).

TABLE 18
human FcγR binding affinity by anti-human CD20 IgG1 antibody
variants harboring non-activating mutations in the heavy chain constant
region, measured by biolayer interferometry. For the high affinity receptor
FcγRIa, the K_{D} (M), the kₒₙ (1/Ms), and k_{off} (1/s) are shown based on a 1:1
Model using a Global (Full) fit. For the low affinity receptors FcγRIIa
allotype 131H, FcγRIIb, and FcγRIIIa allotype 158V, the K_{D} (M) is shown
based on the Steady State Analysis. Data shown are mean values ± SD of
2 independent replicates. Variants tested are IgG1, IgG1-FER, IgG1-FEA,
and IgG1-LALAPG wherein FER: L234F-L235E-G236R, FEA: L234F-
L235E-D265A, and LALAPG: L234A-L235A-P329G. SSA: Steady State
Analysis, BLI: biolayer interferometry, nb: no binding, n/a: not applicable.
Binding affinity human FcγR (BLI)

| | | | | IgG1- | IgG1- | IgG1- |
|---|---|---|---|---|---|---|
| FcγR ↓ | Antibody variant → | | IgG1 | FER | FEA | LALAPG |
| FcγRIa | K_{D} | Mean | 1.84E−09 | nb | nb | nb |
| (Global | (M) | SD | 7.14E−10 | n/a | n/a | n/a |
| fit) | kₒₙ | Mean | 3.54E+05 | nb | nb | nb |
| | (1/Ms) | SD | 9.76E+04 | n/a | n/a | n/a |
| | k_{off} | Mean | 6.16E−04 | nb | nb | nb |
| | (1/s) | SD | 7.50E−05 | n/a | n/a | n/a |
| FcγRIIa(H) | K_{D} | Mean | 1.90E−06 | nb | nb | nb |
| (SSA) | (M) | SD | 4.24E−07 | n/a | n/a | n/a |
| FcγRIIb | K_{D} | Mean | 7.30E−06 | nb | nb | nb |
| (SSA) | (M) | SD | 9.90E−07 | n/a | n/a | n/a |
| FcγRIIIa(V) | K_{D} | Mean | 5.65E−07 | nb | nb | nb |
| (SSA) | (M) | SD | 1.20E−07 | n/a | n/a | n/a |

### Example 28: Binding Affinity of Anti-Human CD20 IgG1 Antibodies and Non-Activating Variants Thereof to Murine Fcγ Receptors Measured by Biolayer Interferometry

Data in Example 27 showed the binding affinity, or lack thereof, of an anti-human CD20 IgG1 antibody and non-activating variants thereof to human FcγRIa, FcγRIIa allotype 131H, FcγRIIb, and FcγRIIIa allotype 158V as measured by biolayer interferometry. However, when assessing the in vivo therapeutic effects of human antibodies in murine models, depending on the murine model chosen, human Fcγ receptors may not be present. Instead, assessment of the therapeutic effect of the human antibody may rely on the presence of endogenously expressed murine Fcγ receptors. In such cases, if the therapeutic antibody benefits from a non-activating Fc domain it is key to ensure absence of binding to murine Fcγ receptors by the non-activating antibody variant.

Here we assessed the binding affinity of the wild-type anti-human CD20 IgG1 antibody and non-activating variants thereof harboring L234F-L235E-D265A, L234F-L235E-G236R, L234A-L235A-P329G mutations to the monomeric extracellular domains (ECD) of murine FcγRI, FcγRIIb, FcγRIII, and FcγRIV using biolayer interferometry (BLI) on an Octet HTX instrument (FortéBio). All steps were performed at 30° C. For murine FcγRI, after acclimatization (600 s at 30° C.) of the Ni-NTA biosensors (FortéBio, cat. no. 18-5101), as well as the proteins diluted in Sample Diluent (FortéBio, cat. no. 18-1104), an initial baseline measurement was performed by incubating the Ni-NTA biosensors in Sample Diluent for 100 s. Subsequently, His-tagged murine FcγRI (Sino Biological, cat. no. 50086-M27H-B, 2 μg/ml) was immobilized on Ni-NTA biosensors for a duration of 600 s. After a baseline measurement (100 s) in Sample Diluent, the association (300 s) and dissociation (1000 s) of the wild-type anti-human CD20 IgG1 antibody and non-activating variants thereof was determined. Binding of the antibodies to murine FcγRI was tested using a concentration range (15.6-1000 nM; 2-fold dilutions). Data was analyzed with Data Analysis Software v11.1 (FortéBio). In short, data traces were corrected by subtraction of a reference curve (FcγR on sensor, measurement with Sample Diluent only), followed by alignment of the Y-axis to the last 10 s of the baseline. Finally, an interstep correction as well as Savitzky-Golay filtering was applied. To determine the K_{D} (M), as well as the kₒₙ (1/Ms) and k_{off} (1/s), a 1:1 Model was chosen using a Global (Full) fit. Response values<0.05 were excluded from the analysis. 100 s dissociation was used as window of interest for the analysis for all antibody variants. Optimal fit was determined by a full R² value of >0.98, indicating the fit and experimental data correlate significantly. Furthermore, the Global (Full) fit is based on >3 curve fits (reliable analysis). Data shown are mean values ±SD of 2 independent replicates.

For murine FcγRIIb, FcγRIII, and FcγRIV, after acclimatization (600 s at 30° C.) of the Streptavidin (SA) biosensors (FortéBio, cat. no. 18-S019), as well as the proteins diluted in Sample Diluent, an initial baseline measurement was performed by incubating the SA biosensors in Sample Diluent for 100 s. Subsequently, His-tagged biotinylated murine FcγRIIb (Sino Biological, cat. no. 50030-M27H-B, 1 μg/ml), FcγRIII (Sino Biological, cat. no. 50326-M27H-B, 1 μg/ml), or FcγRIV (Sino Biological, cat. no. 50036-M27H-B, 1 μg/ml) were immobilized on SA biosensors for 90 s (FcγRIIb) or 250 s (FcγRIII and FcγRIV). After a baseline measurement (100 s) in Sample Diluent, the association (50 s, FcγRIIb or FcγRIII; 300 s, FcγRIV) and dissociation (1000 s) of the wild-type anti-human CD20 IgG1 antibody and non-activating variants thereof was determined. Binding of the antibodies to murine FcγRIIb, FcγRIII, and FcγRIV was tested using a range of concentrations (FcγRIIb, 187.5-12000 nM, 2-fold dilutions; FcγRIII, 156.25-10000 nM, 2-fold dilutions; FcγRIV, 78.13-S000 nM, 2-fold dilutions). Data was analyzed with Data Analysis Software v11.1 (FortéBio). In short, data traces were corrected by subtraction of a reference curve (FcγR on sensor, measurement with Sample Diluent only), the Y-axis was aligned to the last 10 s of the baseline measurement, and interstep correction was applied. Due to the low affinity between human IgG1 and the low affinity murine Fcγ receptors FcγRIIb, FcγRIII, and FcγRIV, Steady State Analysis (SSA) was chosen to determine the K_{D} (M). In short, Response values<0.05 were excluded from the analysis. The steady-state responses (where the sensogram reached a plateau in the association phase) for the different antibody concentrations were calculated using the R-equilibrium (R_{eq}) function of the Data Analysis Software, subsequently the steady-state responses were plotted against the antibody concentration, and finally a Langmuir model was used to calculate the K_{D} (M). Optimal fit was determined by a full R² value of >0.98, indicating the fit and experimental data correlate significantly. Furthermore, SSA is based on >3 association curve fits and the plotted steady-state responses for each antibody concentration reached a plateau allowing proper calculation of the maximum binding response (reliable analysis). Data shown are mean values ±SD of 2 independent replicates.

Assessment of the binding of anti-human CD20 human IgG1 antibody variants to murine FcγRI, FcγRIIb, FcγRIII, and FcγRIV using biolayer interferometry revealed that wild-type human IgG1 showed binding to all tested murine Fcγ receptors with a binding affinity of 0.12 μM for FcγRIa, 2.8 μM for FcγRIIb, 7.5 μM for FcγRIII, and 1 μM for FcγRIV (Table 19). In contrast, the human IgG1 antibody variants harboring L234F-L235E-G236R, L234F-L235E-D265A, or L234A-L235A-P329G non-activating mutations in the heavy chain constant region did not show binding to murine FcγRI, FcγRIIb, FcγRIII, and FcγRIV (Table 19).

TABLE 19
Murine FcγR binding affinity by anti-human CD20 human IgG1 antibody
variants, harboring non-activating mutations in the heavy chain constant
region, as measured by biolayer interferometry. For the high affinity
receptor FcγRI, the K_{D} (M), the kₒₙ (1/Ms), and k_{off} (1/s) are shown based
on a 1:1 Model using a Global (Full) fit. For the low affinity
receptors FcγRIIb, FcγRIII, and FcγRIV, the K_{D} (M) is shown
based on the Steady State Analysis. Data shown are mean values ± SD of
2 independent replicates. Variants tested are IgG1, IgG1-FER, IgG1-FEA,
and IgG1-LALAPG wherein FER: L234F-L235E-G236R, FEA: L234F-
L235E-D265A, and LALAPG: L234A-L235A-P329G. SSA: Steady State
Analysis, BLI: biolayer interferometry, nb: no binding, n/a: not applicable.
Binding affinity murine FcγR (BLI)

| | | | | IgG1- | IgG1- | IgG1- |
|---|---|---|---|---|---|---|
| FcγR ↓ | Antibody variant → | | IgG1 | FER | FEA | LALAPG |
| FcγRI | K_{D} | Mean | 1.24E−07 | nb | nb | nb |
| (Global | (M) | SD | 1.34E−08 | n/a | n/a | n/a |
| fit) | kₒₙ | Mean | 1.16E+05 | nb | nb | nb |
| | (1/Ms) | SD | 5.66E+03 | n/a | n/a | n/a |
| | k_{off} | Mean | 1.44E−02 | nb | nb | nb |
| | (1/s) | SD | 2.26E−03 | n/a | n/a | n/a |
| FcγRIIb | K_{D} | Mean | 2.85E−06 | nb | nb | nb |
| (SSA) | (M) | SD | 1.20E−06 | n/a | n/a | n/a |
| FcγRIII | K_{D} | Mean | 7.45E−06 | nb | nb | nb |
| (SSA) | (M) | SD | 1.06E−06 | n/a | n/a | n/a |
| FcγRIV | K_{D} | Mean | 1.04E−06 | nb | nb | nb |
| (SSA) | (M) | SD | 8.49E−08 | n/a | n/a | n/a |

### Example 29: Binding Affinity of Anti-Human CD20 IgG1 Antibodies and Non-Activating Variants Thereof to Cynomolgus Fcγ Receptors Measured by Biolayer Interferometry

During preclinical development, studies involving non-human primates (Cynomolgus monkey, Macaca fascicularis) or experiments involving biological samples derived from non-human primates may be initiated to investigate the therapeutic effect, as well as safety and pharmacokinetics, of an antibody therapeutic candidate. Data in Example 27 showed the binding affinity, or lack thereof, of an anti-human CD20 IgG1 antibody harboring the non-activating L234F-L235E-G236R mutations to human FcγRIa, FcγRIIa allotype 131H, FcγRIIb, and FcγRIIIa allotype 158V as measured by biolayer interferometry. Despite the strong sequence similarity between human and cynomolgus Fcγ receptors, a particular non-activating variant that did not show binding to human Fcγ receptors may still show binding to cynomolgus Fcγ receptors. Therefore, to minimize the risk of unwanted adverse events and dose-limiting toxicity, we assessed the binding affinity of wild-type anti-human CD20 IgG1 antibody and non-activating variants thereof harboring L234F-L235E-D265A, L234F-L235E-G236R, L234A-L235A-P329G mutations to the monomeric extracellular domains (ECD) of cynomolgus FcγRI, FcγRIIa, FcγRIIb, and FcγRIII using biolayer interferometry (BLI) on an Octet HTX instrument (FortéBio).

All steps were performed at 30° C. For cynomolgus FcγRI, after acclimatization (600 s at 30° C.) of the Ni-NTA biosensors (FortéBio, cat. no. 18-S101), as well as the proteins diluted in Sample Diluent (FortéBio, cat. no. 18-1104), an initial baseline measurement was performed by incubating the Ni-NTA biosensors in Sample Diluent for 100 s. Subsequently, His-tagged cynomolgus FcγRI (R&D Systems, cat. no. CF9239-Fc, 1 μg/ml) was immobilized on Ni-NTA biosensors for a duration of 600 s. After a baseline measurement (100 s) in Sample Diluent, the association (300 s) and dissociation (1000 s) of the wild-type anti-human CD20 IgG1 antibody and non-activating variants thereof was determined. Binding of the antibodies to cynomolgus FcγRI was tested using a concentration range of 1.56-100 nM (for IgG1 wild-type; 2-fold dilutions) or 15.6-1000 nM (for IgG1-L234F-L235E-D265A, IgG1-L234F-L235E-G236R, and IgG1-L234A-L235A-P329G; 2-fold dilutions. Data was analyzed with Data Analysis Software v11.1 (FortéBio). In short, data traces were corrected by subtraction of a reference curve (FcγR on sensor, measurement with Sample Diluent only), followed by alignment of the y-axis to the last 10 s of the baseline. Finally, an interstep correction as well as Savitzky-Golay filtering was applied. To determine the K_{D} (M), as well as the kₒₙ (1/Ms) and k_{off} (Vs), a 1:1 Model was chosen using a Global (Full) fit. Response values<0.05 were excluded from the analysis. 400 s dissociation was used as window of interest for the analysis for all antibody variants. Optimal fit was determined by a full R² value of >0.98, indicating the fit and experimental data correlate significantly. Furthermore, the Global (Full) fit is based on >3 concentration curve fits (reliable analysis) unless indicated differently. Data shown are mean values ±SD of 2 independent replicates.

For cynomolgus FcγRIIa, FcγRIIb, and FcγRIII, after acclimatization (600 s at 30° C.) of the Streptavidin (SA) biosensors (FortéBio, cat. no. 18-S019), as well as the proteins diluted in Sample Diluent, an initial baseline measurement was performed by incubating the SA biosensors in Sample Diluent for 100 s. Subsequently, His-tagged biotinylated cynomolgus FcγRIIa (Sino Biological, cat. no. 90015-C27H-B, 1 μg/ml), FcγRIIb (Sino Biological, cat. no. 90014-C27H-B, 1 μg/ml), or FcγRIII (ACROBiosystems, cat. no. FC6-C82E0, 1 μg/ml) were immobilized on SA biosensors for 80 s (FcγRIIa and FcγRIIb) or 100 s (FcγRIII). After a baseline measurement (100 s) in Sample Diluent, the association (50 s, FcγRIIa or FcγRIIb; 300 s, FcγRIII) and dissociation (1000 s) of the wild-type anti-human CD20 IgG1 antibody and non-activating variants thereof was determined. Binding of the antibodies to cynomolgus FcγRIIa, FcγRIIb, and FcγRIII was tested using a range of concentrations (FcγRIIa, 156.25-10000 nM, 2-fold dilutions; FcγRIIb, 187.5-12000 nM, 2-fold dilutions; FcγRIII, 31.25-2000 nM, 2-fold dilutions). Data was analyzed with Data Analysis Software v11.1 (FortéBio). In short, data traces were corrected by subtraction of a reference curve (FcγR on sensor, measurement with Sample Diluent only), the y-axis was aligned to the last 10 s of the baseline measurement, and interstep correction was applied. Due to the low affinity between human IgG1 and the low affinity cynomolgus Fcγ receptors FcγRIIa, FcγRIIb, and FcγRIII, Steady State Analysis (SSA) was chosen to determine the K_{D} (M). In short, Response values<0.05 were excluded from the analysis. The steady-state responses (where the sensogram reached a plateau in the association phase) for the different antibody concentrations were calculated using the R-equilibrium (R_{eq}) function of the Data Analysis Software, subsequently the steady-state responses were plotted against the antibody concentration, and finally a Langmuir model was used to calculate the K_{D} (M). Optimal fit was determined by a full R² value of >0.98, indicating the fit and experimental data correlate significantly. Furthermore, SSA is based on >3 association curve fits and the plotted steady-state responses for each antibody concentration reached a plateau allowing proper calculation of the maximum binding response (reliable analysis). Data shown are mean values ±SD of 2 independent replicates.

Assessment of the binding of anti-human CD20 human IgG1 antibody variants to cynomolgus FcγRI, FcγRIIa, FcγRIIb, and FcγRIII using biolayer interferometry revealed that wild-type human IgG1 showed binding to all tested cynomolgus Fcγ receptors with a binding affinity of 0.6 nM for FcγRIa, 4.3 μM for FcγRIIa, 3.9 μM for FcγRIIb, and 0.4 μM for FcγRIII (Table 20). In contrast, the human IgG1 antibody variants harboring L234F-L235E-G236R or L234F-L235E-D265A non-activating mutations in the heavy chain constant region did not show binding to cynomolgus FcγRI, FcγRIIa, FcγRIIb, and FcγRIII (Table 20). The non-activating variant IgG1-L234A-L235A-P329G did not show binding to the low affinity receptors FcγRIIa, FcγRIIb, and FcγRIII. In contrast, although the Global (Full) fit analysis was not optimal (only 3 concentration curve fits), the analysis does indicate residual but greatly reduced (— 3500-fold) binding of IgG1-L234A-L235A-P329G to the high affinity receptor FcγRI with a binding affinity of approximately 2.2 μM (Table 20).

In summary, the human IgG1 antibody variant harboring the L234F-L235-G236R non-activating mutations showed no cynomolgus FcγR binding, similar to the previously described non-activating Fc variant L234F-L235E-D265A. In contrast, L234A-L235A-P329G, which did not show any human or murine FcγR binding, did show residual binding to cynomolgus FcγRI, but not FcγRIIa, FcγRIIb, or FcγRIII.

TABLE 20
Cynomolgus FcγR binding affinity by anti-human CD20 human IgG1
antibody variants, harboring non-activating mutations in the heavy chain
constant region, as measured by biolayer interferometry. For the high
affinity receptor FcγRI, the K_{D} (M), the kₒₙ (1/Ms), and k_{off} (1/s) are
shown based on a 1:1 Model using a Global (Full) fit. For the low
affinity receptors FcγRIIa, FcγRIIb, and FcγRIII, the K_{D} (M) is shown
based on the Steady State Analysis. Data shown are mean values ± SD of
2 independent replicates. Variants tested are IgG1, IgG1-FER, IgG1-
FEA, and IgG1-LALAPG wherein FER: L234F-L235E-G236R, FEA:
L234F-L235E-D265A, and LALAPG: L234A-L235A-P329G. SSA:
Steady State Analysis, BLI: biolayer interferometry,
nb: no binding, n/a: not applicable.
Binding affinity cynomolgus FcγR (BLI)

| | | | | IgG1- | IgG1- | IgG1- |
|---|---|---|---|---|---|---|
| FcγR ↓ | Antibody variant → | | IgG1 | FER | FEA | LALAPG |
| FcγRI | K_{D} | Mean | 6.26E−10 | nb | nb | 2.19E−06¹ |
| (Global | (M) | SD | 7.35E−11 | n/a | n/a | 3.18E−07¹ |
| fit) | kₒₙ | Mean | 1.23E+05 | nb | nb | 1.56E+04¹ |
| | (1/Ms) | SD | 8.49E+03 | n/a | n/a | 4.31E+03¹ |
| | k_{off} | Mean | 7.72E−05 | nb | nb | 3.33E−02¹ |
| | (1/s) | SD | 1.46E−05 | n/a | n/a | 4.45E−03¹ |
| FcγRIIa | K_{D} | Mean | 4.25E−06 | nb | nb | nb |
| (SSA) | (M) | SD | 2.19E−06 | n/a | n/a | n/a |
| FcγRIIb | K_{D} | Mean | 3.90E−06 | nb | nb | nb |
| (SSA) | (M) | SD | 2.83E−07 | n/a | n/a | n/a |
| FcγRIII | K_{D} | Mean | 4.15E−07 | nb | nb | nb |
| (SSA) | (M) | SD | 3.54E−08 | n/a | n/a | n/a |

¹IgG1-LALAPG binding to FcγRI - analysis not optimal, <4 fits for Global (Full) fit analysis

### Example 30: Impact of Genetic Sequence Including or Excluding Coding Sequence for C-Terminal Lysine on Capacity to Induce Complement-Dependent Cytotoxicity by Anti-Human CD20 Antibodies and Variants Thereof Containing Non-Activating Mutations in the Heavy Chain Region

The genetic sequence of IgG antibodies encodes a lysine at the C-terminus of the heavy chain, which is (partially) cleaved off from the produced IgG antibody in culture medium or circulation by carboxypeptidases (Van den Bremer et al. MAbs; 2015; 7(4):672-80), leading to potential heterogeneity in end product therapeutics. In addition, presence of the HC C-terminal lysine has been shown to negatively affect the capacity to induce CDC. Here, the capacity to induce CDC by anti-human CD20 antibodies containing the non-activating L234F-L235E-D265A (FEA) or L234F-L235E-G236R (FER) mutations in the heavy chain constant region was assessed, comparing antibody variants that were based on a genetic sequence encoding the HC C-terminal lysine and variants based on a genetic sequence in which the HC C-terminal lysine was recombinantly deleted. In theory, the C-terminal lysine may be cleaved off from the former variants during production or post-production.

An in vitro CDC assay was performed, essentially as described in Example 3, on Raji cells with 20% NHS as the source of complement. Briefly, the anti-human CD20 antibody IgG1-CD20 or variants thereof harboring non-activating mutations FEA or FER in the heavy chain constant region were tested in a range of concentrations (0.014-10 μg/mL final concentrations; 3-fold dilutions) using 50,000 Raji cells/well. The number of PI-positive cells, as a measure for cell lysis, was determined by flow cytometry on an Intellicyt iQue screener (Sartorius). The data were analyzed using a non-linear agonist dose-response model and the area under the dose-response curves (AUC) per experimental replicate was calculated using log-transformed concentrations in GraphPad PRISM with no antibody control as baseline, followed by normalization per experimental replicate to the AUC value measured for the wild-type IgG1-CD20 antibody variant (100%). Data are mean values ±SEM obtained from 3 independent experiments.

As shown in FIG. 20, no difference in the capacity to induce CDC on Raji cells was observed between antibody variants that were produced based on a genetic sequence either containing or lacking the nucleotide sequence encoding the HC C-terminal lysine. This was shown for both wild-type IgG1-CD20 antibodies and the variants containing the FEA or FER non-activating mutations.

### Example 31: Activation and Signaling Via Fcγ Receptors by Anti-Human CD20 Antibodies and Non-Activating Variants Thereof with Recombinant Deletion of the HC C-Terminal Lysine

Example 30 assessed the impact of recombinant deletion of the heavy chain (HC) C-terminal lysine (delK) of a wild-type anti-human CD20 IgG1 antibody and non-activating variants thereof on the capacity to induce or inhibit induction of CDC. Here, we assessed human FcγR activation and signaling in Promega reporter assays, using target-expressing Raji cells and a Jurkat reporter cell line that expresses the indicated FcγR, by these anti-human CD20 IgG1 antibodies and variants thereof as described in Example 30.

Activation of human FcγR-mediated signaling, by the anti-human CD20 IgG1 antibody variants indicated in Example 30, was quantified using reporter BioAssays (Promega, FcγRIa: Cat #CS1781C01; FcγRIIa allotype 131H: Cat #G988A; FcγRIIb: Cat #CS1781E01; FcγRIIIa allotype 158V: Cat #G701A) with CD20-expressing Raji cells as target cells following the procedure described in Example 7. The background luminescence signal, as determined by medium-only control samples (no Raji cells, no antibody, no effector cells), was subtracted from all samples prior to further analysis. The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control (only Raji cells and effector cells) as baseline. Per experiment, AUC values were normalized relative to reporter activity observed for cells incubated with a non-binding control IgG1-b12 (0%) and the activity of wild-type IgG1 (100%). Data are mean values ±SEM from two independent replicates.

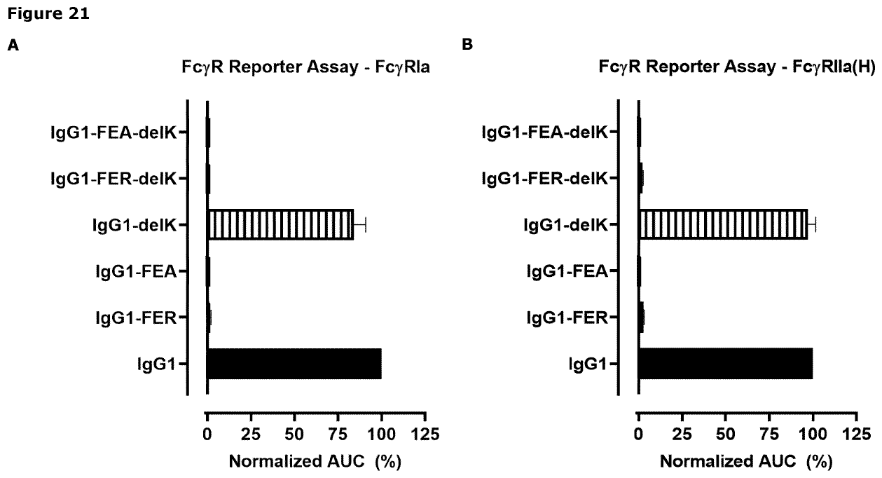

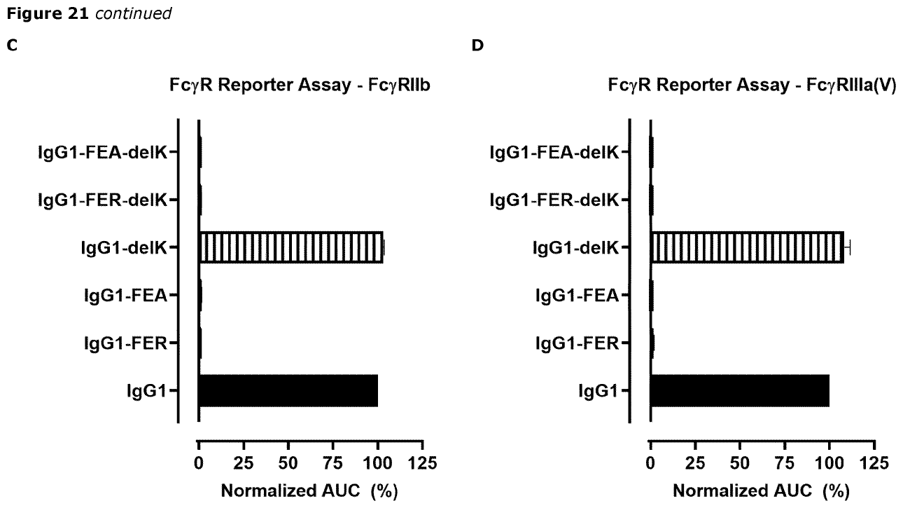

Assessment of human FcγR activation using Promega reporter assays revealed no difference in the efficiency to induce FcγRIa-, FcγRIIa-, FcγRIIb-, and FcγRIIIa-mediated activation between a variant where the HC C-terminal lysine was recombinantly deleted (IgG1-delK) or a variant where this C-terminal lysine was cleaved off during production or post-production (IgG1; FIG. 21). Furthermore, the capacity of the non-activating variants L234F-L235E-G236R (FER) and L234F-L235E-D265A (FEA) to abrogate FcγRIa-, FcγRIIa-, FcγRIIb-, and FcγRIIIa-mediated activation was also not affected (FIG. 21).

In summary, recombinant deletion of the HC C-terminal lysine of an anti-human IgG1-CD20 antibody and non-activating variants thereof does not impact the capacity, or lack thereof, of these antibodies to induce CDC when compared to variants where the C-terminal lysine was cleaved off during production or post-production.

### Example 32: Complement-Dependent Cytotoxicity by Allotypic Variants of Anti-Human CD20 IgG1 Antibodies and Non-Activating Variants Thereof

In previous Examples, the capacity to induce CDC was assessed for anti-human CD20 IgG1 antibody variants belonging to allotype IgG1(f). Here, we assessed the capacity of different allotypes of anti-human CD20 IgG1 antibodies and variants thereof harboring L234F-L235E-G236R or L234F-L235E-D265A non-activating mutations in the heavy chain (HC) constant region.

An in vitro CDC assay was performed, essentially as described in Example 3, on Raji cells with 20% NHS as the source of complement. Different allotypes of anti-human CD20 IgG1 antibody variants were tested, including IgG1(fa), IgG1(zax), IgG1(zav), IgG1(za), and IgG1(f). Briefly, allotypic variants of an anti-human CD20 IgG1 antibody or variants thereof harboring non-activating mutations were tested in a range of concentrations (0.014-10 μg/mL final concentrations; 3-fold dilutions) using 50,000 Raji cells/well. The number of PI-positive cells, as a measure for cell lysis, was determined by flow cytometry on an Intellicyt iQue screener (Sartorius). The data were analyzed using a non-linear agonist dose-response model and the area under the dose-response curves (AUC) per experimental replicate was calculated using log-transformed concentrations in GraphPad PRISM with no antibody control as baseline, followed by normalization per experimental replicate to the AUC value measured for the wild-type IgG1(f)-CD20 antibody variant (100%). Data are mean values ±SEM obtained from 3 independent experiments.

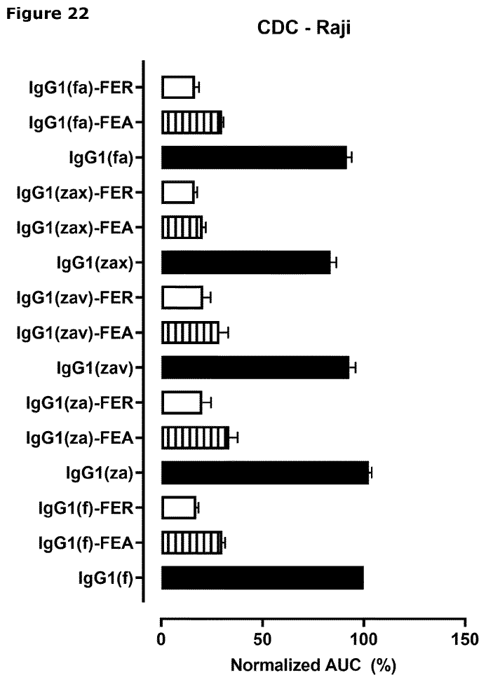

As shown in FIG. 22, no difference in the capacity to induce CDC on Raji cells was observed between wild-type allotypic IgG1 variants. The capacity to induce CDC was greatly reduced for all tested allotypes upon introduction of the FER or FEA non-activating mutations, with the FER mutations resulting in a stronger suppression of CDC than the FEA mutations in all tested allotypes.

### Example 33: Activation and Signaling Via Fcγ Receptors by Anti-Human CD20 Antibodies and Non-Activating Variants Thereof with Different IgG1 Allotype Constant Regions

Example 32 assessed the impact of introducing L234F-L235E-G236R non-activating mutations in the heavy chain (HC) of anti-human CD20 antibodies, with different IgG1 allotype constant regions on the capacity to induce CDC. Here, we assessed human FcγR activation and signaling in Promega reporter assays, using target-expressing Raji cells and a Jurkat reporter cell line that expresses the indicated FcγR, by these different IgG1 allotypic anti-human CD20 antibodies and variants thereof harboring L234F-L235E-G236R or L234F-L235E-D265A non-activating mutations in the heavy chain constant region.

Activation of human FcγR-mediated signaling, by the anti-human CD20 antibody variants indicated in Example 32, was quantified using reporter BioAssays (Promega, FcγRIa: Cat #CS1781C01; FcγRIIa allotype 131H: Cat #G988A; FcγRIIb: Cat #CS1781E01; FcγRIIIa allotype 158V: Cat #G701A) with CD20-expressing Raji cells as target cells following the procedure described in Example 7. The background luminescence signal, as determined by medium-only control samples (no Raji cells, no antibody, no effector cells), was subtracted from all samples prior to further analysis. The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control (only Raji cells and effector cells) as baseline. Per experiment, AUC values were normalized relative to reporter activity observed for cells incubated with a non-binding control IgG1-b12 (0%) and the activity of wild-type IgG1(f) (100%). Data are mean values ±SEM from two independent replicates.

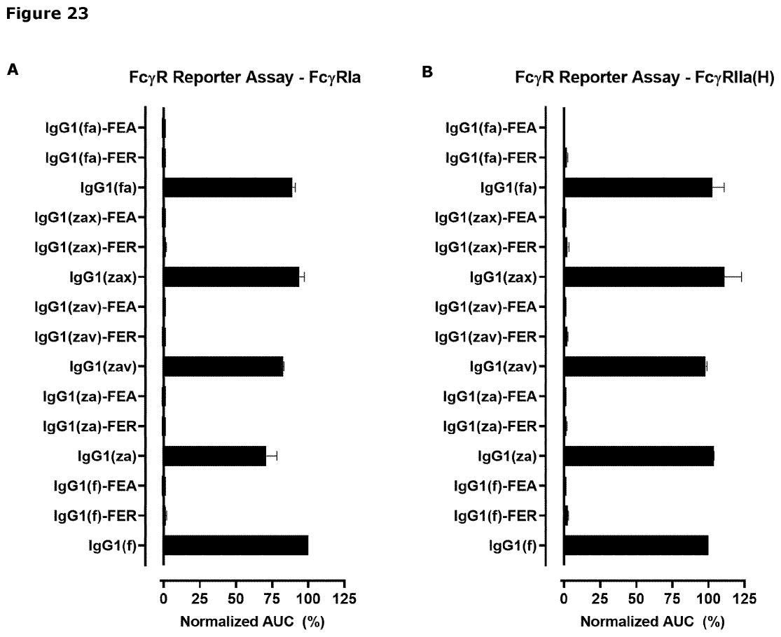

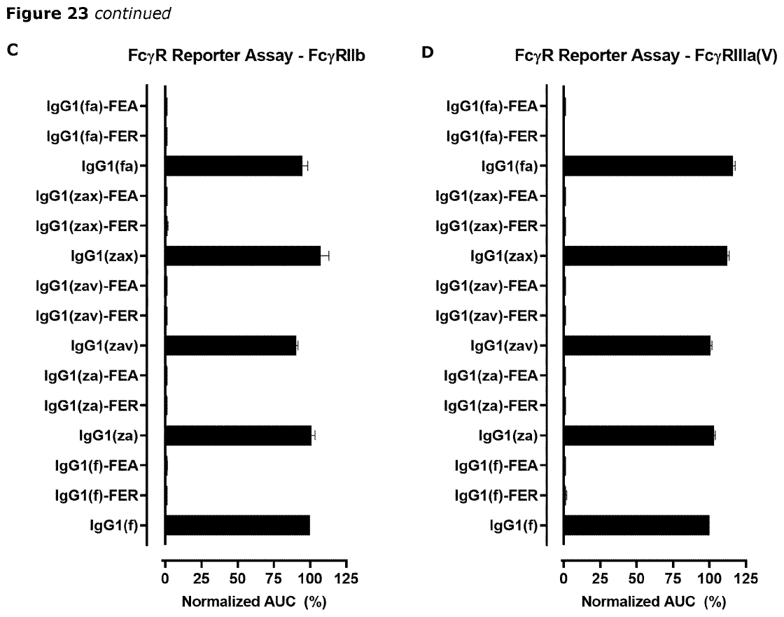

Assessment of human FcγRIa-, FcγRIIa-, FcγRIIb-, and FcγRIIIa-mediated activation using Promega reporter assays revealed that, although some variation was observed, all wild-type anti-human CD20 IgG1 allotypic antibody variants tested showed efficient human FcγR activation (FIG. 23). Moreover, introduction of either L234F-L235E-G236R or L234F-L235E-D265A non-activating mutations in the heavy chain constant region of the different IgG1 allotypic variants efficiently abrogated FcγRIa-, FcγRIIa-, FcγRIIb-, and FcγRIIIa-mediated activation (FIG. 23).

In summary, the non-activating mutations L234F-L235E-G236R efficiently abrogated FcγR-mediated activation by anti-human CD20 antibody variants with different IgG1 allotype constant regions.

### Example 34: Complement-Dependent Cytotoxicity by IgG1, IgG3, and IgG4 Anti-Human CD20 Antibodies and Non-Activating Variants Thereof

Here, we assessed the capacity to induce CDC upon introduction of L234F-L235E-G236R or L234F-L235E-D265A non-activating mutations in the HC of anti-human CD20 IgG1 or IgG3 antibodies, and upon introduction of L235E-G236R or L235E-D265A non-activating mutations in the HC of an anti-human CD20 IgG4 antibody, which naturally has a phenylalanine (F) at position 234.

An in vitro CDC assay on Raji cells was performed, essentially as described in Example 3, with 20% NHS as the source of complement. Wild-type anti-human CD20 IgG1, IgG3 (allotypes IGHG3*01 and IGHG3*04 [IgG3rch2]), and IgG4 antibodies were compared to variants thereof harboring the non-activating mutations mentioned above. Briefly, antibody variants were tested in a range of concentrations (0.014-10 μg/mL final concentrations; 3-fold dilutions) using 50,000 Raji cells/well. The number of PI-positive cells, as a measure for cell lysis, was determined by flow cytometry on an Intellicyt iQue screener (Sartorius). The data were analyzed using a non-linear agonist dose-response model and the area under the dose-response curves (AUC) per experimental replicate was calculated using log-transformed concentrations in GraphPad PRISM with no antibody control as baseline, followed by normalization per experimental replicate to the AUC value measured for the wild-type IgG1-CD20 antibody variant (100%). Data are mean values ±SEM obtained from 3 independent experiments.

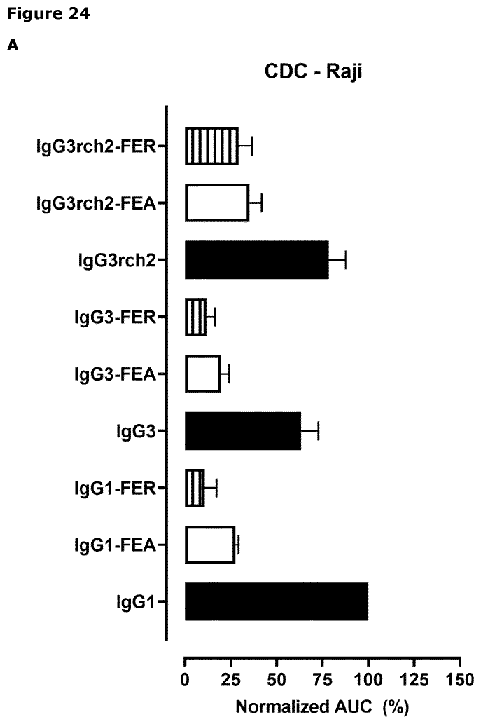

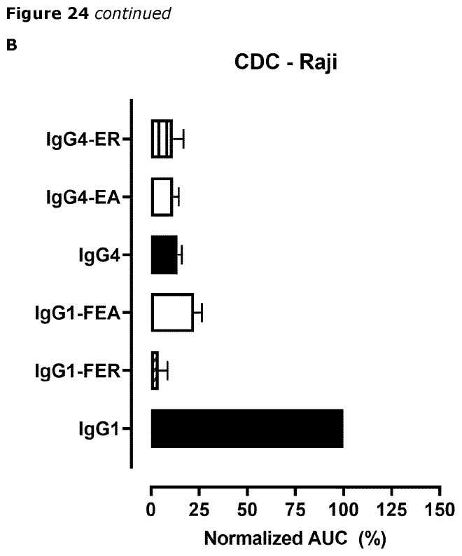

As shown in FIG. 24A, the wild-type anti-human CD20 IgG1 antibody induced stronger CDC than both wild-type allotypes of IgG3. Introduction of non-activating mutations L234F-L235E-D265A and L234F-L235E-G236R strongly suppressed the capacity to induce CDC in both IgG1 and IgG3 variants, with the strongest suppression of CDC activity observed for the variants harboring the L234F-L235E-G236R mutations. Wild-type IgG4 showed a very low intrinsic capacity to induce CDC, which was not further suppressed by the introduction of the L235E-G236R or L235E-D265A non-activating mutations (FIG. 24B).

In summary, the introduction of the L234F-L235E-G236R non-activating mutations in anti-human CD20 antibodies of the IgG1 and IgG3 subclasses reduced the capacity to induce CDC of Raji cells more strongly than the L234F-L235E-D265A non-activating mutations.

### Example 35: Activation and Signaling Via Fcγ Receptors by IgG1, IgG3, and IgG4 Anti-Human CD20 Antibodies and Non-Activating Variants Thereof

In Example 34, the capacity to induce CDC by anti-human CD20 antibodies was assessed upon introduction of L234F-L235E-G236R or L234F-L235E-D265A non-activating mutations in the heavy chain (HC) constant region of IgG3 or upon introduction of L235E-G236R or L235E-D265A non-activating mutations in the HC constant region of IgG4, which naturally has a phenylalanine (F) at position 234. Here, we assessed human FcγR activation and signaling in Promega reporter assays using target-expressing Raji cells and a Jurkat reporter cell line that expresses the indicated FcγR, by anti-human CD20 IgG1, IgG3, and IgG4 antibodies and variants thereof harboring non-activating mutations in the HC constant region.

Human FcγR activation and signaling by the anti-human CD20 antibody variants, as indicated in Example 34, was quantified using reporter BioAssays (Promega, FcγRIa: Cat #CS1781C01; FcγRIIa allotype 131H: Cat #G988A; FcγRIIb: Cat #CS1781E01; FcγRIIIa allotype 158V: Cat #G701A) with CD20-expressing Raji cells as target cells following the procedure described in Example 7. The background luminescence signal, as determined by medium-only control samples (no Raji cells, no antibody, no effector cells), was subtracted from all samples prior to further analysis. The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control (only Raji cells and effector cells) as baseline. Per experiment, AUC values were normalized relative to reporter activity observed for cells incubated with a non-binding control IgG1-b12 (0%) and the activity of the wild-type IgG1 (100%). Data are mean values ±SEM from two independent replicates.

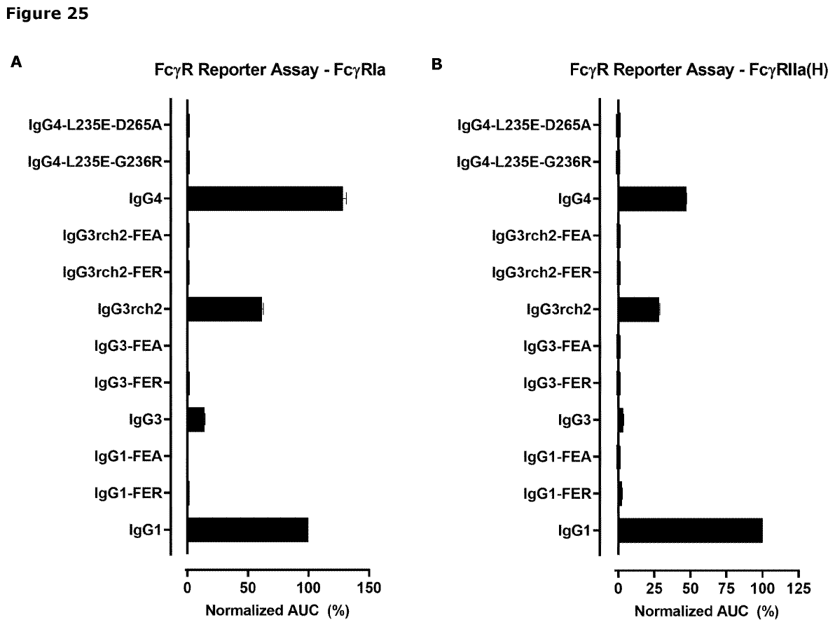

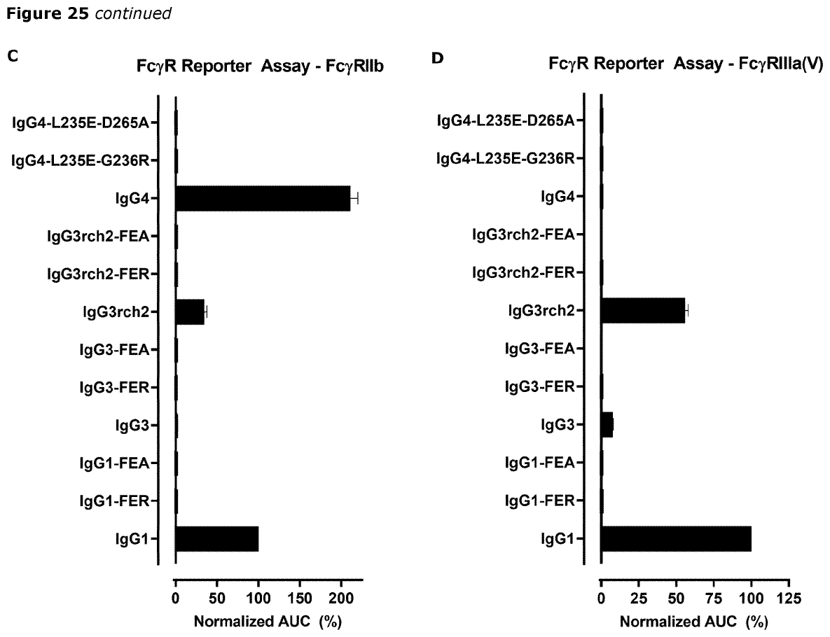

Assessment of human FcγR-mediated activation, using Promega reporter assays, revealed that wild-type human IgG1 efficiently induced FcγRIa-, FcγRIIa-, FcγRIIb-, and FcγRIIIa-mediated activation (FIG. 25). When compared to IgG1, assessment of FcγR-mediated activation by wild-type human IgG4 revealed increased FcγRIa- and FcγRIIb-mediated activation (FIG. 25A, C), decreased FcγRIIa-mediated activation (FIG. 25B), and lack of FcγRIIIa-mediated activation (FIG. 25D). Assessment of FcγR-mediated activation by the human IgG3 allotype IGHG3*01 (IgG3) wild-type antibody variant revealed lack of FcγRIIa- and FcγRIIb-mediated activation (FIG. 25B, C) and only a minor induction of FcγRIa- and FcγRIIIa-mediated activation (FIG. 25A, D). In contrast, the human IgG3 allotype IGHG3*04 (IgG3rch2) wild-type antibody variant showed increased FcγRIa-, FcγRIIa-, FcγRIIb-, and FcγRIIIa-mediated activation when compared to FcγRIa-, FcγRIIa-, FcγRIIb-, and FcγRIIIa-mediated activation by human IgG3 allotype IGHG3*01, although the level of FcγR-mediated activation for FcγRIa, FcγRIIa, FcγRIIb, and FcγRIIIa was still reduced compared to a wild-type human IgG1 (FIG. 25). Assessment of FcγRIa-, FcγRIIa-, FcγRIIb-, and FcγRIIIa-mediated activation by anti-human CD20 IgG1, IgG3, or IgG4 antibody variants revealed that introduction of L234F-L235E-G236R or L234F-L235E-D265A (IgG1, IgG3, and IgG3rch2) or introduction of L235E-G236R or L235E-D265A (IgG4) non-activating mutations in the heavy chain constant region efficiently abrogated activation of all tested FcγRs (FIG. 25).

In summary, the non-activating mutations L234F-L235E-G236R (or L235E-G236R for IgG4) efficiently abrogated FcγR-mediated activation by anti-human CD20 antibody variants irrespective of whether these non-activating mutations were introduced in an IgG1, IgG3, or IgG4 heavy chain constant region.

### Example 36: Binding Affinity of Anti-Human CD20 Murine IgG2a Antibodies and Non-Activating Variants Thereof to Human Fcγ Receptors Measured by Biolayer Interferometry

Preclinical xenograft models using immunodeficient mice are often used to establish therapeutic concepts using tumor-specific antibodies. However, more complex therapeutic questions require the use of immunocompetent mice that accurately capture the biology and efficacy of a therapeutic antibody. In such cases, a surrogate mouse antibody is required to allow natural interactions of the antibody with the murine effector molecules. Whereas in Examples 27-29 the binding affinity of a human IgG1 antibody and non-activating variants thereof to human, murine, and cynomolgus Fcγ receptors was assessed, here we investigated whether introducing the non-activating mutations L234F-L235E-G236R in the heavy chain (HC) constant region of a murine IgG2a antibody prevented binding to human FcγRs. Binding affinity of these variants to murine FcγRs will be evaluated in Example 37.

Here, we assessed the binding affinity of the wild-type murine anti-human CD20 IgG2a antibody and non-activating variants thereof harboring L234A-L235A, L234F-L235E-G236R, or L234A-L235A-P329G mutations to the monomeric extracellular domains (ECD) of human FcγRIa, FcγRIIa allotype 131H, FcγRIIb, and FcγRIIIa allotype 158V using biolayer interferometry (BLI) on an Octet HTX instrument (FortéBio), essentially as described in Example 27. In short, for human FcγRIa, after loading of the Ni-NTA sensors with human FcγRIa a baseline measurement (100 s) in Sample Diluent was performed. Subsequently, the association (300 s) and dissociation (1000 s) of the wild-type murine anti-human CD20 IgG2a antibody non-activating variants thereof was determined. Binding of the antibodies to human FcγRIa was tested using a concentration range of 1.56-100 nM (for wild-type IgG2a; 2-fold dilutions) or 15.6-1000 nM (for IgG2a-L234A-L235A, IgG2a-L234F-L235E-G236R, and IgG2a-L234A-L235A-P329G; 2-fold dilutions). Data was analyzed as described in Example 27 with response values<0.05 being excluded from the analysis. 400 s dissociation was used as window of interest for the analysis for all antibody variants except IgG2a-L234A-L235A for which a 50 s dissociation was used as a window of interest. Optimal fit was determined by a full R2 value of >0.98, indicating the fit and experimental data correlated significantly. Furthermore, the Global (Full) fit is based on >3 curve fits (reliable analysis) unless indicated differently. Data shown are mean values ±SD of 2 independent replicates.

Assessment of binding to human FcγRIIa allotype 131H, FcγRIIb, and FcγRIIIa allotype 158V was performed essentially as described in Example 27. In short, after loading of the Streptavidin (SA) biosensors with human FcγRIIa allotype 131H, FcγRIIb, or FcγRIIIa allotype 158V, a baseline measurement (100 s) in Sample Diluent was performed. Subsequently, the association (50 s, FcγRIIa or FcγRIIb; 300 s, FcγRIIIa) and dissociation (1000 s) of the murine anti-human CD20 antibodies IgG2a wild-type and non-activating variants thereof was determined. Binding of the antibodies to FcγRIIa allotype 131H, FcγRIIb, and FcγRIIIa allotype 158V was tested using a range of concentrations (FcγRIIa, 156.25-10000 nM, 2-fold dilutions; FcγRIIb, 250-16000 nM, 2-fold dilutions; FcγRIIIa, 125-8000 nM, 2-fold dilutions). Data was analyzed as described in Example 27. Due to the low affinity between murine IgG2a and the low affinity Fcγ receptors FcγRIIa allotype 131H, FcγRIIb, and FcγRIIIa allotype 158V, Steady State Analysis (SSA) was chosen to determine the K_{D} (M). In short, Response values<0.05 were excluded from the analysis. The steady-state responses (where the sensogram reached a plateau in the association phase) for the different antibody concentrations were calculated using the R-equilibrium (R_{eq}) function of the Data Analysis Software, subsequently the steady-state responses were plotted against the antibody concentration, and finally a Langmuir model was used to calculate the K_{D} (M). Optimal fit was determined by a full R2 value of >0.98, indicating the fit and experimental data correlate significantly. Furthermore, SSA is based on >3 association curve fits and the plotted steady-state responses for each antibody concentration reached a plateau allowing proper calculation of the maximum binding response (reliable analysis) unless indicated differently. Data shown are mean values ±SD of 2 independent replicates.

Assessment of the binding of murine anti-human CD20 IgG2a antibody variants to human FcγRIa, FcγRIIa allotype 131H, FcγRIIb, and FcγRIIIa allotype 158V using biolayer interferometry revealed that wild-type murine IgG2a showed binding to all tested human Fcγ receptors with a binding affinity of 1.27 nM for FcγRIa, 1.85 μM for FcγRIIa allotype 131H, 17 μM for FcγRIIb, and 1.95 μM for FcγRIIIa allotype 158V (Table 21). In contrast, the murine IgG2a antibody variants harboring L234F-L235E-G236R or L234A-L235A-P329G non-activating mutations in the heavy chain constant region did not show binding to human FcγRIa, FcγRIIa allotype 131H, FcγRIIb, and FcγRIIIa allotype 158V (Table 21). Murine IgG2a harboring L234A-L235A non-activating mutations in the heavy chain constant region did not show binding to human FcγRIIa allotype 131H and FcγRIIb. The analysis indicated low residual binding of IgG2a-L234A-L235A to human FcγRIa (K D=3.6 μM; Global (Full) fit analysis based on only 3 concentration curve fits) and human FcγRIIIa allotype 158V (K_{D}=13.8 μM; steady-state responses did not reach a plateau) (Table 21).

In summary, introducing L234F-L235-G236R non-activating mutations in the heavy chain constant region of a murine IgG2a antibody prevented binding to human FcγRIa, FcγRIIa allotype 131H, FcγRIIb, and FcγRIIIa allotype 158V, similar to a murine IgG2a antibody harboring L234A-L235A-P329G non-activating mutations.

TABLE 21
Human FcγR binding affinity by murine anti-human CD20 IgG2a antibody
variants, harboring non-activating mutations in the heavy chain constant region, as
measured by biolayer interferometry. For the high affinity receptor FcγRIa, the
K_{D} (M), the kₒₙ (1/Ms), and k_{off} (1/s) are shown based on a 1:1 Model
using a Global (Full) fit. For the low-affinity receptors FcγRIIa allotype 131H,
FcγRIIb, and FcγRIIIa allotype 158V, the K_{D} (M) is shown based on the Steady State
Analysis. Data shown are mean values ± SD of 2 independent replicates. Variants tested
are IgG2a, IgG2a-FER, IgG2a-LALA, and IgG2a-LALAPG wherein FER: L234F-
L235E-G236R, LALA: L234A-L235A, and LALAPG: L234A-L235A-P329G. SSA:
Steady State Analysis, BLI: biolayer interferometry, nb: no binding, n/a: not applicable.
Binding affinity human FcγR (BLI)

| | | | | IgG2a- | IgG2a- | IgG2a- |
|---|---|---|---|---|---|---|
| FcγR ↓ | Antibody variant → | | IgG2a | FER | LALA | LALAPG |
| FcγRIa | K_{D} | Mean | 1.27E−09 | nb | 3.61E−06¹ | nb |
| (Global | (M) | SD | 3.39E−10 | n/a | 2.47E−07¹ | n/a |
| fit) | kₒₙ | Mean | 3.11E+05 | nb | 4.53E+03¹ | nb |
| | (1/Ms) | SD | 2.62E+04 | n/a | 3.85E+02¹ | n/a |
| | k_{off} | Mean | 3.90E−04 | nb | 1.63E−02¹ | nb |
| | (1/s) | SD | 7.21E−05 | n/a | 2.55E−04¹ | n/a |
| FcγRIIa(H) | K_{D} | Mean | 1.85E−06 | nb | nb | nb |
| (SSA) | (M) | SD | 3.54E−07 | n/a | n/a | n/a |
| FcγRIIb | K_{D} | Mean | 1.70E−05 | nb | nb | nb |
| (SSA) | (M) | SD | 1.41E−06 | n/a | n/a | n/a |
| FcγRIIIa(V) | K_{D} | Mean | 1.95E−06 | nb | 1.17E−05² | nb |
| (SSA) | (M) | SD | 2.12E−07 | n/a | 8.98E−06² | n/a |

¹IgG2a-LALA binding to FcγRIa - analysis not optimal, <4 fits for Global (Full) fit analysis
²IgG2a-LALA binding to FcγRIIIa(V) - analysis not optimal, steady-state responses did not reach a plateau

### Example 37: Binding Affinity of Anti-Human CD20 Murine IgG2a Antibodies and Non-Activating Variants Thereof to Murine Fcγ Receptors Measured by Biolayer Interferometry

In Example 36, the binding of affinity of anti-human CD20 murine IgG2a non-activating antibodies to human FcγRs was evaluated. Here, we investigated whether introducing the non-activating mutations L234F-L235E-G236R in the heavy chain (HC) constant region of a murine IgG2a antibody prevented binding to murine FcγRs.

The binding affinity of the murine anti-human CD20 antibodies described in Example 36 to the monomeric extracellular domains (ECD) of murine FcγRI, FcγRIIb, FcγRIII, and FcγRIV was assessed using biolayer interferometry (BLI) on an Octet HTX instrument (FortéBio), essentially as described in Example 28. In short, for murine FcγRI, after loading of the Ni-NTA sensors with murine FcγRI, a baseline measurement (100 s) in Sample Diluent was performed. Subsequently, the association (300 s) and dissociation (1000 s) of the wild-type murine anti-human CD20 IgG2a antibody and non-activating variants thereof harboring L234A-L235A, L234F-L235E-G236R, or L234A-L235A-P329G mutations was determined. Binding of the antibodies to murine FcγRI was tested using a concentration range of 1.56-100 nM (for wild-type IgG2a; 2-fold dilutions) or 15.6-1000 nM (for IgG2a-L234A-L235A, IgG2a-L234F-L235E-G236R, and IgG2a-L234A-L235A-P329G; 2-fold dilutions). Data was analyzed as described in Example 28 with response values<0.05 excluded from the analysis. 400 s dissociation was used as window of interest for the analysis for all antibody variants. Optimal fit was determined by a full R² value of >0.98, indicating the fit and experimental data correlate significantly. Furthermore, the Global (Full) fit is based on >3 curve fits (reliable analysis) unless indicated differently. Data shown are mean values ±SD of 2 independent replicates.

Assessment of binding affinity to murine FcγRIIb, FcγRIII, and FcγRIV was performed essentially as described in Example 28. In short, after loading of the Streptavidin (SA) biosensors with murine FcγRIIb, FcγRIII, or FcγRIV, a baseline measurement (100 s) in Sample Diluent was performed. Subsequently, the association (50 s, FcγRIIb or FcγRIII; 300 s, FcγRIV) and dissociation (1000 s) of the wild-type murine anti-human CD20 IgG2a antibody and non-activating variants thereof harboring L234A-L235A, L234F-L235E-G236R, or L234A-L235A-P329G mutations, was determined. Binding of the antibodies to murine FcγRIIb, FcγRIII, and FcγRIV was tested using a range of concentrations (FcγRIIb, 187.5-12,000 nM, 2-fold dilutions; FcγRIII, 156.25-10,000 nM, 2-fold dilutions; FcγRIV, 15.63-1,000 nM for IgG2a, IgG2a-L234F-L235E-G236R, and IgG2a-L234A-L235A-P329G or 156.3-10,000 nM for IgG2a-L234A-L235A, 2-fold dilutions). Data was analyzed as described in Example 28. Due to the low affinity between murine IgG2a and the low affinity Fcγ receptors FcγRIIb, FcγRIII, and FcγRIV, Steady State Analysis (SSA) was chosen to determine the K_{D} (M). In short, Response values<0.05 were excluded from the analysis. The steady-state responses (where the sensorgram reached a plateau in the association phase) for the different antibody concentrations were calculated using the R-equilibrium (R_{eq}) function of the Data Analysis Software, subsequently the steady-state responses were plotted against the antibody concentration, and finally a Langmuir model was used to calculate the K_{D} (M). Optimal fit was determined by a full R² value of >0.98, indicating the fit and experimental data correlate significantly. Furthermore, SSA is based on >3 association curve fits and the plotted steady-state responses for each antibody concentration reached a plateau allowing proper calculation of the maximum binding response (reliable analysis) unless indicated differently. Data shown are mean values ±SD of 2 independent replicates.

Assessment of the binding of murine anti-human CD20 IgG2a antibody variants to murine FcγRI, FcγRIIb, FcγRIII, and FcγRIV using biolayer interferometry revealed that wild-type murine IgG2a showed binding to all tested murine Fcγ receptors with a binding affinity of approximately 3.8 nM for FcγRI, 6.4 μM for FcγRIIb, 6.9 μM for FcγRIII, and 0.15 μM for FcγRIV (Table 22). In contrast, the murine IgG2a antibody variants harboring L234F-L235E-G236R or L234A-L235A-P329G non-activating mutations in the heavy chain constant region did not show binding to murine FcγRIIb, FcγRIII, and FcγRIV (Table 22). Furthermore, the binding affinity of IgG2a-L234F-L235E-G236R or IgG2a-L234A-L235A-P329G to murine FcγRI was greatly reduced (100-fold) compared to wild-type IgG2a, although the analysis was not optimal (<4 fits for Global (Full) fit analysis) (Table 22). Murine IgG2a-L234A-L235A did not show binding to murine FcγRIIb and FcγRIII but did show low residual binding to murine FcγRI (100-150-fold reduction compared to wt IgG2a) and FcγRIV (100-fold reduction compared to wt IgG2a) (Table 22). In summary, the murine IgG2a antibody variant harboring the L234F-L235-G236R non-activating mutations showed no (FcγRIIb, FcγRIII, and FcγRIV) or greatly reduced (FcγRI) murine FcγR binding, similar to a murine IgG2a antibody harboring L234A-L235A-P329G non-activating mutations.

TABLE 22
Murine FcγR binding affinity by murine anti-human CD20 IgG2a antibody variants,
harboring non-activating mutations in the heavy chain constant region, as measured by
biolayer interferometry. For the high-affinity receptor FcγRI, the K_{D} (M), the kₒₙ
(1/Ms), and k_{off} (1/S) are shown based on a 1:1 Model using a Global (Full) fit.
For the low-affinity receptors FcγRIIb, FcγRIII, and FcγRIV, the K_{D} (M) is shown
based on the Steady State Analysis. Data shown are mean values ± SD of 2
independent replicates. Variants tested are IgG2a, IgG2a-FER, IgG2a-LALA, and
IgG2a-LALAPG wherein FER: L234F-L235E-G236R, LALA: L234A-L235A, and
LALAPG: L234A-L235A-P329G. SSA: Steady State Analysis,
BLI: biolayer interferometry, nb: no binding, n/a: not applicable.
Binding affinity murine FcγR (BLI)

| | | | | IgG2a- | IgG2a- | IgG2a- |
|---|---|---|---|---|---|---|
| FcγR ↓ | Antibody variant → | | IgG2a | FER | LALA | LALAPG |
| FcγRI | K_{D} | Mean | 3.82E−09 | 4.30E−07¹ | 2.38E−07 | 3.64E−07¹ |
| (Global | (M) | SD | 8.49E−11 | 2.26E−08¹ | 1.06E−08 | 1.48E−08¹ |
| fit) | kₒₙ | Mean | 1.62E+05 | 5.30E+03¹ | 9.73E+03 | 5.50E+03¹ |
| | (1/Ms) | SD | 1.41E+03 | 4.17E+02¹ | 1.84E+02 | 2.19E+02¹ |
| | k_{off} | Mean | 6.19E−04 | 2.27E−03¹ | 2.31E−03 | 2.00E−03¹ |
| | (1/s) | SD | 8.49E−06 | 5.66E−05¹ | 5.66E−05 | 1.63E−04¹ |
| FcγRIIb | K_{D} | Mean | 6.40E−06 | nb | nb | nb |
| (SSA) | (M) | SD | 8.49E−07 | n/a | n/a | n/a |
| FcγRIII | K_{D} | Mean | 6.90E−06 | nb | nb | nb |
| (SSA) | (M) | SD | 9.90E−07 | n/a | n/a | n/a |
| FcγRIV | K_{D} | Mean | 1.45E−07 | nb | 1.38E−05² | nb |
| (SSA) | (M) | SD | 2.12E−08 | n/a | 1.03E−05² | n/a |

¹IgG2a-FER & -LALAPG binding to FcγRI - analysis not optimal, <4 fits for Global (Full) fit analysis
²IgG2a-LALA binding to FcγRIV - analysis not optimal, steady-state responses did not reach a plateau

### Example 38: Activation and Signaling Via Human Fcγ Receptors by Anti-Human CD20 Murine IgG2a Antibodies and Non-Activating Variants Thereof

Example 36 showed that introducing L234F-L235E-G236R non-activating mutations in the heavy chain (HC) constant region of a murine IgG2a antibody efficiently prevented binding to human FcγRIa, FcγRIIa allotype 131H, FcγRIIb, and FcγRIIIa allotype 158V, as measured by biolayer interferometry. However, in such assays, effects of antigen-binding, target-mediated antibody clustering and subsequent target-mediated clustering of the Fc-receptors on the effector cells were absent. Here, we assessed human FcγR activation and signaling in Promega reporter assays using target-expressing Raji cells and a Jurkat reporter cell line that expresses the indicated FcγR, by a wild-type anti-human CD20 murine IgG2a antibody and variants thereof harboring L234F-L235E-G236R, L234A-L235A, or L234A-L235A-P329G non-activating mutations in the heavy chain constant region.

Activation of human FcγR-mediated signaling by the murine IgG2a anti-human CD20 antibody variants mentioned above, was quantified using reporter BioAssays Promega, FcγRIa: Cat #CS1781C01; FcγRIIa allotype 131H: Cat #G988A; FcγRIIb: Cat #CS1781E01; FcγRIIIa allotype 158V: Cat #G701A) with CD20-expressing Raji cells as target cells following the procedure described in Example 7. The background luminescence signal, as determined by medium-only control samples (no Raji cells, no antibody, no effector cells), was subtracted from all samples prior to further analysis. The data were analyzed using a non-linear agonist dose-response model and the area under the dose-response curves (AUC) per experimental replicate was calculated using log-transformed concentrations in GraphPad PRISM (version 8.4.1, GraphPad Software) with no antibody control (only Raji cells and effector cells) as baseline. Per experiment, AUC values were normalized relative to reporter activity observed for cells incubated with a non-binding control IgG2a-b12 (0%) and the activity of wild-type IgG2a-CD20 (100%). Data are mean values ±SEM from two independent replicates.

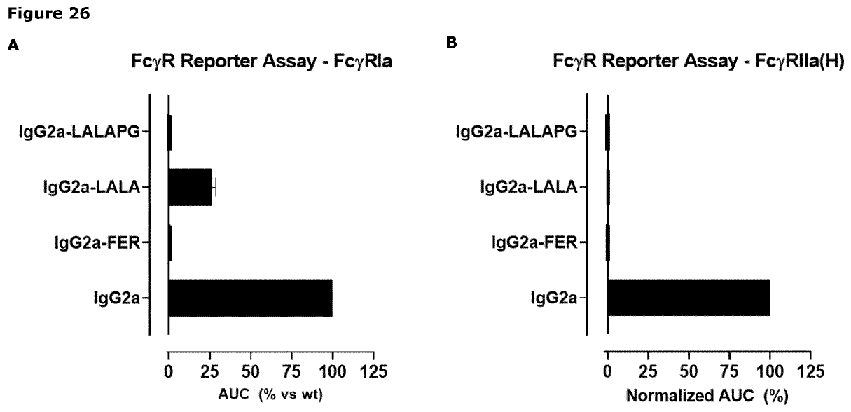

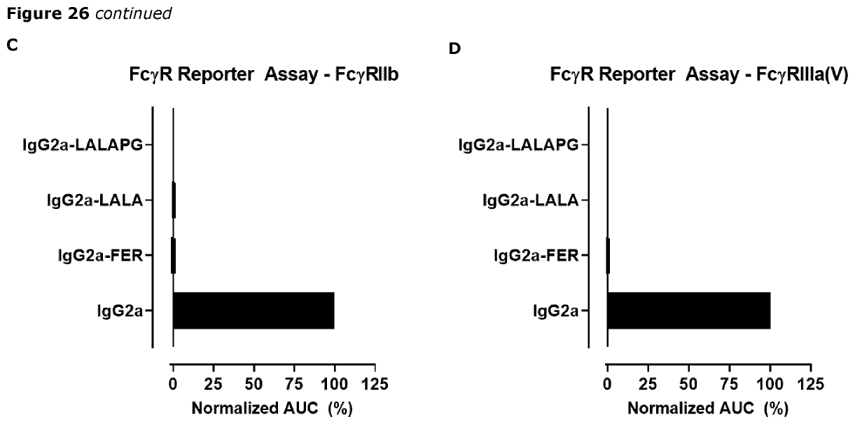

Assessment of human FcγRIa-, FcγRIIa-, FcγRIIb-, and FcγRIIIa-mediated activation using Promega reporter assays revealed that introduction of L234F-L235E-G236R non-activating mutations in the heavy chain constant region of a murine IgG2a antibody efficiently inhibited FcγR-mediated activation, comparable to the IgG2a non-activating variant L234A-L235A-P329G (FIG. 26). Lack of FcγR-mediated activation was also observed for IgG2a-L234A-L235A (FIG. 26B-D), except for partial activation mediated via FcγRIa (FIG. 26A), which is in line with the low residual binding observed for IgG2a-L234A-L235A to human FcγRIa as measured by biolayer interferometry (Example 36).

In summary, introduction of L234F-L235E-G236R non-activating mutations in the heavy chain constant region of an anti-human CD20 murine IgG2a antibody efficiently abrogated human FcγR-mediated activation comparable to the non-activating antibody variant IgG2a-L234A-L235A-P329G.

### Example 39: C1q Binding to and Complement-Dependent Cytotoxicity by Anti-Human CD20 Murine IgG2a Antibodies and Non-Activating Variants Thereof

In previous Examples, it was shown that introduction of L234F-L235E-G236R (FER) non-activating mutations in the heavy chain (HC) constant region of an anti-human CD20 human IgG1 antibody resulted in a near-complete abrogation of the capacity to engage with the complement factor C1q, as well the capacity to induce CDC. Here, the capacity of a wild-type anti-human CD20 murine IgG2a antibody and non-activating variants thereof harboring FER, L234A-L235A, or L234A-L235A-P329G mutations in the HC to bind human complement factor C1q was assessed, as well as the capacity to induce CDC.

A wild-type anti-human CD20 murine IgG2a antibody and variants thereof harboring non-activating mutations in the HC, as described above, were tested in a C1q binding assay on Raji cells (3×10⁴ cells/well), using a range of concentrations (0.0024-10 μg/mL final concentrations; 4-fold dilutions) with 20% normal human serum (NHS, cat. no. M0008, Sanquin) as the source of C1q, essentially following the procedure described in Example 4. C1q binding to the antibody variants was detected by flow cytometry on an Intellicyt iQue screener (Sartorius) by measuring Median Fluorescence Intensity-FITC. The data were analyzed using a non-linear agonist dose-response model and the Area Under the dose-response Curves (AUC) per experimental replicate was calculated using log-transformed concentrations in GraphPad PRISM (version 8.4.1, GraphPad Software) with no antibody control as baseline, followed by normalization per experimental replicate to the AUC value measured for the non-binding control antibody IgG2a-b12 (0%) and the AUC value measured for the wild-type anti-human CD20 IgG2a antibody variant (100%). Data are mean values ±SEM obtained from three independent experiments.

The same antibody variants tested in the C1q binding assay were tested in an in vitro CDC assay. Antibody variants were tested in a range of concentrations (0.0024-10 μg/mL final concentrations; 4-fold dilutions) using 3×10⁴ Raji cells per well, essentially as further described in Example 3. The number of PI-positive cells was determined by flow cytometry on an Intellicyt iQue screener (Sartorius). The percentage of PI-positive cells, which corresponds to the percentage of cell lysis, was calculated as (number of PI-positive cells/total number of cells)×100%. The data were analyzed using a non-linear agonist dose-response model and the AUC per experimental replicate was calculated using log-transformed concentrations in GraphPad PRISM (version 8.4.1, GraphPad Software) with no antibody control as baseline, followed by normalization per experimental replicate to the AUC value measured for the non-binding control antibody IgG2a-b12 (0%) and the AUC value measured for the wild-type anti-human CD20 IgG2a antibody variant (100%). Data are mean values ±SEM obtained from three independent experiments.

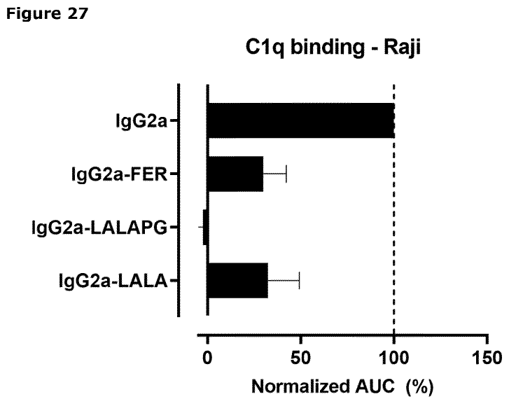

Assessment of binding of C1q to murine IgG2a-CD20 variants revealed that wild-type IgG2a-CD20 efficiently engages with the complement protein C1q upon binding to CD20 on target Raji cells (FIG. 27). The non-activating variant harboring L234A-L235A-P329G mutations in the HC efficiently abrogated binding to the complement protein C1q (FIG. 27). However, upon introduction of L234F-L235E-G236R or L234A-L235A non-activating mutations in the HC of a murine IgG2a antibody, reduced but residual binding to C1q was observed (FIG. 27).

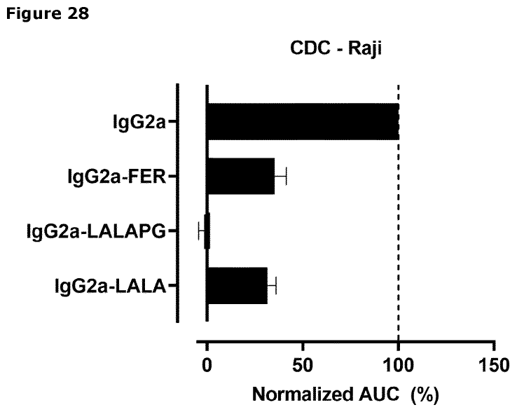

Assessment of the CDC-inducing capacity of the same IgG2a-CD20 variants revealed results in line with results for C1q binding. Introduction of the non-activating L234A-L235A-P329G mutations in the HC of a murine IgG2a antibody resulted in near-complete abrogation of the capacity to induce CDC (FIG. 28). Furthermore, introduction of L234F-L235E-G236R or L234A-L235A non-activating mutations in the HC of a murine IgG2a antibody resulted in a reduction of CDC, however, residual CDC was still observed (FIG. 28).

In summary, introduction of the non-activating mutations L234F-L235E-G236R in a murine IgG2a antibody resulted in reduced but no full inhibition of the ability to engage with the complement factor C1q as well the capacity to induce CDC. It was therefore concluded that introduction of the FER non-activating mutations reduced the capacity to engage with human C1q and induce CDC of both human and murine IgG antibodies.

1. A protein comprising a first polypeptide and a second polypeptide, wherein said first and second polypeptide each comprise at least a hinge region, a CH2 region and a CH3 region, respectively, of a human IgG1 immunoglobulin heavy chain, wherein at least one of said first and second polypeptides is modified and comprises a substitution of amino acids corresponding with amino acids at the positions L234, L235 and G236, wherein amino acid positions are as defined by Eu numbering.

2. The protein according to claim 1, wherein the amino acids at positions L234, L235 and G236 in at least one of said first and second polypeptide are substituted with F, E and R, respectively.

3. The protein according to claim 1 or claim 2, wherein one of the first and second polypeptides comprises said substitution of amino acids corresponding with amino acids at positions L234, L235 and G236, and the other is modified and comprises a substitution of amino acids corresponding with amino acids at positions L234, L235, and D265, wherein preferably, said substitutions are F, E and A, respectively.

4. The protein according to claim 1 or claim 2, wherein both first and second polypeptides comprise said substitution of amino acids corresponding with amino acids L234, L235 and G236.

5. The protein according to any one of claims 1-4, wherein each of said first and second polypeptides comprises an immunoglobulin CH1 region.

6. The protein according to any one of claims 1-5, wherein said protein comprises a first and a second binding region.

7. The protein according to claim 6, wherein said first and second binding regions comprise respectively a first immunoglobulin heavy chain variable region and a first immunoglobulin light chain variable region, and wherein said second binding region comprises a second immunoglobulin heavy chain variable region and a second immunoglobulin light chain variable region.

8. The protein according to claim 7, wherein said immunoglobulin heavy and light chain variable regions are human or humanized immunoglobulin heavy and light chain variable regions.

9. The protein according to claim 7 or 8, wherein said first and second polypeptides are immunoglobulin heavy chains, and wherein said first and second polypeptides comprise said respective first and second immunoglobulin heavy chain variable regions.

10. The protein according to any of claims 6-9, wherein said protein comprises a first immunoglobulin light chain constant region and a second immunoglobulin light chain constant region.

11. The protein according to claim 10, wherein said protein comprises first and second immunoglobulin light chains, said immunoglobulin light chains comprising said respective first and second immunoglobulin light chain variable regions and said respective first and second immunoglobulin constant light chain regions.

12. The protein according to claim 11, wherein said first immunoglobulin light chain is connected with said first immunoglobulin heavy chain via disulfide bridges and said second immunoglobulin light chain is connected with said second immunoglobulin heavy chain via disulfide bridges, thereby forming said first binding region and said second binding region, respectively, and wherein said first and second immunoglobulin heavy chains are connected via disulfide bridges.

13. The protein according to any one of claims 1-12, which is an antibody.

14. The protein in accordance with any one of claims 4-12, which is a monospecific antibody.

15. The protein in accordance with claim 4-14, wherein said first and second polypeptide chains have an identical amino acid sequence.

16. The protein in accordance with claim 15, wherein said first and second polypeptide comprise a further amino acid substitution, preferably a substitution of an amino acid selected from the group consisting of T366, L368, K370, D399, F405, Y407, and K409, such as F405L or K409R.

17. The protein according to any of claims 14-16, wherein said monospecific antibody binds an antigen selected from the group consisting of cellular targets, cytokines, toxins, pathogens, cancer antigens, plasma proteins.

18. The protein according to any one of claims 1-13, wherein said protein is a bispecific antibody.

19. The bispecific antibody according to claim 18, wherein said first and second polypeptide comprise further substitutions in said respective CH2 and CH3 regions such that the sequences of the respective CH2 and CH3 regions from said first and second polypeptides are different, said substitutions allowing to obtain said polypeptide comprising said first and second polypeptide.

20. The bispecific antibody according to claim 19, wherein in said first polypeptide at least one of the amino acids in the positions corresponding to a position selected from the group consisting of T366, L368, K370, D399, F405, Y407, and K409 in a human IgG1 heavy chain has been substituted, and in said second polypeptide at least one of the amino acids in the positions corresponding to a position selected from the group consisting of; T366, L368, K370, D399, F405, Y407, and K409 in a human IgG1 heavy chain has been substituted, and wherein said substitutions of said first and said second polypeptides are not in the same positions.

21. The bispecific antibody according to claim 19, wherein the amino acid in the position corresponding to F405 is L in said first polypeptide, and the amino acid in the position corresponding to K409 is R in said second polypeptide, or vice versa.

22. The bispecific antibody according to any one of claims 18-21, wherein said bispecific antibody has modifications in both of said first and second polypeptide consisting of substitutions of the amino acids at positions L234, L235 and G236 with F, E and R, and substitutions of the amino acid at position F405 with is L in said first polypeptide, and at K409 with R in said second polypeptide, or vice versa.

23. The bispecific antibody according to any one of claims 18-22, wherein said first and second polypeptides have substitutions consisting of substitutions as defined in any one of claims 1-4 and 19-22.

24. The protein according to any one of claims 1-17 or bispecific antibody according to any one of claims 18-23, wherein said first and second polypeptides comprise an amino acid sequence in accordance with SEQ ID NO: 1, wherein said amino acid sequence which is comprised in said first and second polypeptides have amino acid substitutions as defined in claims 1-4 and 18-22.

25. The protein or bispecific antibody according to claim 24, wherein said amino acid sequence in accordance with SEQ ID NO:1 does not comprise a terminal lysine.

26. The bispecific antibody according to any one of claims 18-25, wherein one of said binding region binds a cancer antigen.

27. The bispecific antibody according to any one of claims 18-25, wherein one of said binding regions binds an effector cell, such as a T-cell, NK cell, dendritic cell, monocyte, macrophage or neutrophil.

28. The bispecific antibody according to any one of claims 18-25, wherein one of said binding regions binds an effector cell, such as a T-cell, NK cell, dendritic cell, monocyte, macrophage or neutrophil, and the other binding region binds a cancer antigen.

29. A nucleic acid encoding said first or second polypeptide as defined in any one of claims 4-17 and 24-25, wherein said first or second polypeptide comprises said substitution of amino acids corresponding with amino acids L234, L235 and G236, preferably wherein said substitutions of positions L234, L235 and G236 are with F, E and R, respectively.

30. A nucleic acid in accordance with claim 29, wherein said first or second polypeptide is an immunoglobulin heavy chain.

31. A host cell comprising a nucleic acid in accordance with claim 29 or 30.

32. A pharmaceutical composition comprising the protein according to any of claims 1 to 17, or bispecific antibody according to any of claims 18-28 and a pharmaceutical acceptable carrier.

33. The protein according to any of claims 1 to 17, or bispecific antibody according to any of claims 18-28, or the pharmaceutical composition according to claim 32, for use in the treatment of a disease.

34. The protein, bispecific antibody, or pharmaceutical composition for use in accordance with claim 33, wherein said use comprises the treatment of a cancer, an infectious disease, or an autoimmune disease.

35. A method of treatment comprising administering the protein according to any of claims 1 to 17, or bispecific antibody according to any of claims 18-28, or the pharmaceutical composition according to claim 32, to a subject.

36. The method of treatment according to claim 35, wherein the subject is suffering from a disease, such as a cancer, an infectious disease, or an autoimmune disease.

37. A method of preparing a bispecific antibody comprising
f) providing a first antibody, comprising
a. an immunoglobulin heavy chain comprising at least a hinge region, a CH2 region and a CH3 region, respectively of a human IgG1 immunoglobulin heavy chain, comprising substitutions of amino acids at positions L234, L235 and G236, with F, E and R, respectively,
b. an immunoglobulin light chain;
g) providing a second antibody, comprising
a. an immunoglobulin heavy chain comprising at least a hinge region, a CH2 region and a CH3 region, respectively, of a human IgG1 immunoglobulin heavy chain, comprising substitutions of amino acids at
positions L234, L235, and D265, wherein preferably, said substitutions are F, E and A, respectively,
or,
comprising substitutions of amino acids at positions L234, L235 and G236, with F, E and R, respectively,
b. an immunoglobulin light chain;
h) wherein the sequences of said first and second CH3 regions of said respective first and second antibodies are different and are such that the heterodimeric interaction between said first and second CH3 regions is stronger than each of the homodimeric interactions of said first and second CH3 regions;
i) incubating said first antibody together with said second antibody under reducing conditions sufficient to allow the cysteines in the hinge regions to undergo disulfide-bond isomerization; and
j) obtaining said bispecific antibody comprising said first immunoglobulin heavy chain and said first immunoglobulin light chain of said first antibody and said second immunoglobulin heavy chain and said second immunoglobulin light chain of said second antibody.

38. The method of claim 37, wherein in step c) said differences in sequence are in accordance with any one of claims 19-25.

39. The method of claim 37 or claim 38, for preparing a bispecific antibody as defined in any of claims 18-28.


SEQUENCE LISTING (49 sequences)

SEQ ID NO: 1 - protein, 232 aa, Homo Sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 232 aa, Artificial sequence
  FER
EPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 232 aa, Artificial sequence
  FEA
EPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 98 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 5 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 107 aa, Homo Sapiens [sequence omitted]

SEQ ID NO: 7 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 229 aa, Homo Sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 232 aa, Artificial sequence
  F405L
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 10 - protein, 232 aa, Artificial sequence
  K409R
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 11 - protein, 232 aa, Artificial sequence
  FERL
EPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 12 - protein, 232 aa, Artificial sequence
  FERR
EPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 13 - protein, 232 aa, Artificial sequence
  FEAL
EPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 14 - protein, 232 aa, Artificial sequence
  FEAR
EPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 15 - protein, 321 aa, Artificial sequence
  Extracellular domain FcGR with C-terminal His tag
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHPGSSSHHHHHHPGGGLNDIFEAQKIEWHE

SEQ ID NO: 16 - protein, 418 aa, Artificial sequence
  Extracellular domain FcGR with C-terminal His tag
METQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPVAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGPGSSSHHHHHHPGGGLNDIFEAQKIEWHE

SEQ ID NO: 17 - protein, 405 aa, Artificial sequence
  Extracellular domain FcGR with C-terminal His tag
MVLSLLYLLTALPGILSAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPGSSSHHHHHHPGGGLNDIFEAQKIEWHE

SEQ ID NO: 18 - protein, 405 aa, Artificial sequence
  Extracellular domain FcGR with C-terminal His tag
MVLSLLYLLTALPGILSAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGPGSSSHHHHHHPGGGLNDIFEAQKIEWHE

SEQ ID NO: 19 - protein, 410 aa, Artificial sequence
  Extracellular domain FcGR with C-terminal His tag
MVLSLLYLLTALPGISTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGPSMGSSSPSEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGPSMGSSSPGPGSSSHHHHHHPGGGLNDIFEAQKIEWHE

SEQ ID NO: 20 - protein, 410 aa, Artificial sequence
  Extracellular domain FcGR with C-terminal His tag
MVLSLLYLLTALPGISTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPSMGSSSPSEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPSMGSSSPGPGSSSHHHHHHPGGGLNDIFEAQKIEWHE

SEQ ID NO: 21 - protein, 326 aa, Artificial sequence
  Extracellular domain FcGR with C-terminal His tag
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSPGSSSHHHHHHPGGGLNDIFEAQKIEWHE

SEQ ID NO: 22 - protein, 119 aa, Artificial sequence
  Beta2 microglobulin
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 23 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 330 aa, Homo Sapiens [sequence omitted]

SEQ ID NO: 25 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 26 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 27 - protein, 330 aa, Artificial sequence
  CH1, hinge, CH2 and CH3 region of human IgG1 allotype G1m(fa) with FER substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 330 aa, Artificial sequence
  CH1, hinge, CH2 and CH3 region of human IgG1 allotype G1m(fa) with FEA substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 330 aa, Artificial sequence
  CH1, hinge, CH2 and CH3 region of human IgG1 allotype G1m(za) with FER substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 330 aa, Artificial sequence
  CH1, hinge, CH2 and CH3 region of human IgG1 allotype G1m(za) with FEA substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 330 aa, Artificial sequence
  CH1, hinge, CH2 and CH3 region of human IgG1 allotype G1m(zav) with FER substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 32 - protein, 330 aa, Artificial sequence
  CH1, hinge, CH2 and CH3 region of human IgG1 allotype G1m(zav) with FEA substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 33 - protein, 330 aa, Artificial sequence
  CH1, hinge, CH2 and CH3 region of human IgG1 allotype G1m(zax) with FER substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 34 - protein, 330 aa, Artificial sequence
  CH1, hinge, CH2 and CH3 region of human IgG1 allotype G1m(zax) with FEA substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 35 - protein, 329 aa, Artificial sequence
  CH1, hinge, CH2 and CH3 region of human IgG1 allotype G1m(f) with deletion of K447
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 329 aa, Artificial sequence
  CH1, hinge, CH2 and CH3 region of human IgG1 allotype G1m(f) with deletion of K447 and with FER substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 329 aa, Artificial sequence
  CH1, hinge, CH2 and CH3 region of human IgG1 allotype G1m(f) with deletion of K447 and with FEA substitution
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 39 - protein, 330 aa, Artificial sequence
  CH1, hinge, CH2 and CH3 region of murine IgG2a with FER substitution
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNFERGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 40 - protein, 377 aa, Homo Sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 41 - protein, 347 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 42 - protein, 377 aa, Artificial sequence
  CH1, hinge, CH2 and CH3 region of IgG3 allotype IGHG3*01 with FER substitution
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPEFERGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 43 - protein, 377 aa, Artificial sequence
  CH1, hinge, CH2 and CH3 region of IgG3 allotype IGHG3*01 with FEA substitution
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPEFEGGPSVFLFPPKPKDTLMISRTPEVTCVVVAVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 44 - protein, 347 aa, Artificial sequence
  CH1, hinge, CH2 and CH3 region of IgG3 allotype IGHG3*04 with FER substitution
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPAPEFERGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 45 - protein, 347 aa, Artificial sequence
  CH1, hinge, CH2 and CH3 region of IgG3 allotype IGHG3*04 with FEA substitution
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPAPEFEGGPSVFLFPPKPKDTLMISRTPEVTCVVVAVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 46 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 327 aa, Artificial sequence
  CH1, hinge, CH2 and CH3 region of human IgG4 with L235E-G236R substitution
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFERGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 327 aa, Artificial sequence
  CH1, hinge, CH2 and CH3 region of constant region of human IgG4 with L235E-D265A substitution
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFEGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC